(19) United States
(12) Patent Application Publication
(10) Pub. No.: US 2020/0128830 A1
(43) Pub. Date: Apr. 30, 2020

(54) FUSED BICYCLIC HETEROCYCLE DERIVATIVES AS PESTICIDES

(71) Applicant: Bayer Aktiengesellschaft, Leverkusen (DE)

(72) Inventors: Nina KAUSCH-BUSIES, Bergisch Gladbach (DE); Ruediger FISCHER, Pulheim (DE); Dominik HAGER, Monheim (DE); Laura HOFFMEISTER, Duesseldorf (DE); Marc MOSRIN, Koeln (DE); David WILCKE, Duesseldorf (DE); Matthieu WILLOT, Duesseldorf (DE); Kerstin ILG, Koeln (DE); Matthew WEBBER, Duesseldorf (DE); Anton LISHCHYNSKYI, Langen (DE); Ulrich GOERGENS, Ratingen (DE); Andreas TURBERG, Haan (DE)

(21) Appl. No.: 16/605,815

(22) PCT Filed: Apr. 17, 2018
(86) PCT No.: PCT/EP2018/059727

§ 371 Date: Oct. 17, 2019

(30) Foreign Application Priority Data
Apr. 24, 2017 (EP) 17167707.3

(51) Int. Cl. A01N 43/90; C07D 519/00; C07D 471/04

(52) U.S. Cl. CPC A01N 43/90; C07D 471/04; C07D 519/00

(57) ABSTRACT

The present invention relates to novel fused bicyclic heterocycle derivatives of the formula (I)

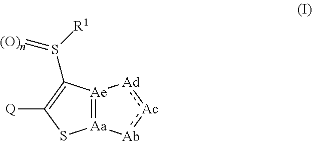

in which Aa, Ab, Ac, Ad, Ae, R¹, Q and n have the meanings mentioned above,
to agrochemical formulations comprising the compounds of formula (I) and to their use as acaricides and/or insecticides for controlling animal pests, especially arthropods and in particular insects and arachnids.

The present invention relates to novel fused bicyclic heterocycle derivatives of the formula (I), to agrochemical formulations comprising the compounds of formula (I) and their use as acaricides and/or insecticides for controlling animal pests, especially arthropods and in particular insects and arachnids.

Fused bicyclic heterocycle derivatives having insecticidal properties have already been described in the literature, for example in WO 2010/125985, WO 2013/180193, WO 2016/039444, WO 2016/129684, WO 2016/142327, WO 2016/169882, WO 2016/169886, WO 2017/001311, WO 2016/162318, WO 2017/093180, WO 2017/125340, WO 2017/144341, EP16184163.0, EP 16189445.6 and EP 16200177.0.

Modern insecticides and acaricides have to meet many demands, for example in relation to extent, persistence and spectrum of their action and possible use. Questions of toxicity, sparing of beneficial species and pollinators, environmental properties, application rates, combinability with other active ingredients or formulation auxiliaries play a role, as does the question of the complexity involved in the synthesis of an active ingredient, and resistances can also occur, to mention just a few parameters. For all these reasons alone, the search for novel crop protection compositions cannot be considered complete, and there is a constant need for novel compounds having improved properties compared to the known compounds, at least in relation to individual aspects.

It was an object of the present invention to provide compounds for use for controlling animal pests, which compounds widen the spectrum of the pesticides in various aspects.

Novel fused bicyclic heterocycle derivatives have now been found, these having advantages over the compounds already known, examples of which include better biological or environmental properties, a wider range of application methods, better insecticidal or acaricidal action, and good compatibility with crop plants. The fused bicyclic heterocycle derivatives can be used in combination with further agents for improving efficacy, especially against insects that are difficult to control.

The subject matter of the present invention is therefore novel compounds of the formula (I) (Configuration 1-1)

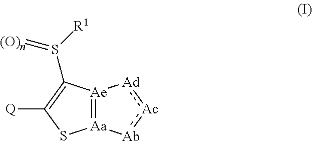

where [symbol] represents two single bonds or one double bond and one single bond,
in which,
if [symbol] represents a double bond and a single bond, and Aa to Ae thus form an aromatic ring,
Aa represents nitrogen or carbon,
Ab represents nitrogen, oxygen, sulfur, N(R⁸) or C(R¹¹),
Ac represents nitrogen, oxygen, sulfur or C(R¹²),
Ad represents nitrogen, oxygen, sulfur, N(R⁸) or C(R¹³)
Ae represents nitrogen or carbon,
where R⁸ represents (C₁-C₄)-alkyl, cyclopropyl or hydrogen and
where at most three of the groups Aa, Ab, Ac, Ad and Ae may represent nitrogen and at most one of the groups Ab, Ac and Ad may represent oxygen or at most one of the groups Ab, Ac and Ad may represent sulfur or at most one of the groups Ab and Ad may represent N(R⁸),
or, in the case that [symbol] represent exclusively single bonds,
Aa represents carbon,
Ab represents sulfur, oxygen or C(R¹¹)(R¹⁵),
Ac represents sulfur, oxygen or C(R¹²)(R¹⁶) and
Ad represents sulfur, oxygen or C(R¹³)(R¹⁷),
Ae represents carbon,
where at most one of the groups Aa, Ab, Ac, Ad and Ae may represent oxygen or sulfur,
R¹ represents (C₁-C₆)-alkyl, (C₁-C₆)-haloalkyl, (C₁-C₆)-cyanoalkyl, (C₁-C₆)-hydroxyalkyl, (C₁-C₆)-alkoxy-(C₁-C₆)-alkyl, (C₁-C₆)-haloalkoxy-(C₁-C₆)-alkyl, (C₂-C₆)-alkenyl, (C₂-C₆)-alkenyloxy-(C₁-C₆)-alkyl, (C₂-C₆)-haloalkenyloxy-(C₁-C₆)-alkyl, (C₂-C₆)-haloalkenyl, (C₂-C₆)-cyanoalkenyl, (C₂-C₆)-alkynyl, (C₂-C₆)-alkynyloxy-(C₁-C₆)-alkyl, (C₂-C₆)-haloalkynyloxy-(C₁-C₆)-alkyl, (C₂-C₆)-haloalkynyl, (C₂-C₆)-cyanoalkynyl, (C₃-C₈)-cycloalkyl, (C₃-C₈)-cycloalkyl-(C₃-C₈)-cycloalkyl, (C₁-C₆)-alkyl-(C₃-C₈)-cycloalkyl, halo-(C₃-C₈)-cycloalkyl, amino, (C₁-C₆)-alkylamino, di-(C₁-C₆)-alkylamino, (C₃-C₈)-cycloalkylamino, (C₁-C₆)-alkylcarbonylamino, (C₁-C₆)-alkylthio-(C₁-C₆)-alkyl, (C₁-C₆)-haloalkylthio-(C₁-C₆)-alkyl, (C₁-C₆)-alkylsulfinyl-(C₁-C₆)-alkyl, (C₁-C₆)-haloalkylsulfinyl-(C₁-C₆)-alkyl, (C₁-C₆)-alkylsulfonyl-(C₁-C₆)-alkyl, (C₁-C₆)-haloalkylsulfonyl-(C₁-C₆)-alkyl, (C₁-C₆)-alkoxy-(C₁-C₆)-alkylthio-(C₁-C₆)-alkyl, (C₁-C₆)-alkoxy-(C₁-C₆)-alkylsulfinyl-(C₁-C₆)-alkyl, (C₁-C₆)-alkoxy-(C₁-C₆)-alkylsulfonyl-(C₁-C₆)-alkyl, (C₁-C₆)-alkylcarbonyl-(C₁-C₆)-alkyl, (C₁-C₆)-haloalkylcarbonyl-(C₁-C₆)-alkyl, (C₁-C₆)-alkoxycarbonyl-(C₁-C₆)-alkyl, (C₁-C₆)-haloalkoxycarbonyl-(C₁-C₆)-alkyl, (C₁-C₆)-alkylsulfonylamino, aminosulfonyl-(C₁-C₆)-alkyl, (C₁-C₆)-alkylaminosulfonyl-(C₁-C₆)-alkyl, di-(C₁-C₆)-alkylaminosulfonyl-(C₁-C₆)-alkyl,
   or represents (C₁-C₆)-alkyl, (C₁-C₆)-alkoxy, (C₂-C₆)-alkenyl, (C₂-C₆)-alkynyl, (C₃-C₈)-cycloalkyl, each of which is optionally mono- or polysubstituted by identical or different substituents from the group consisting of aryl, hetaryl and heterocyclyl, where aryl, hetaryl or heterocyclyl may each optionally be mono- or polysubstituted by identical or different substituents from the group consisting of halogen, cyano, nitro, hydroxy, amino, carboxy, carbamoyl, aminosulfonyl, (C₁-C₆)-alkyl, (C₃-C₆)-cycloalkyl, (C₁-C₆)-alkoxy, (C₁-C₆)-haloalkyl, (C₁-C₆)-haloalkoxy, (C₁-C₆)-alkylthio, (C₁-C₆)-alkylsulfinyl, (C₁-C₆)-alkylsulfonyl, (C₁-C₆)-alkylsulfimino, (C₁-C₆)-alkylsulfimino-(C₁-C₆)-alkyl, (C₁-C₆)-alkylsulfimino-(C₂-C₆)-alkylcarbonyl, (C₁-C₆)-alkylsulfoximino, (C₁-C₆)-alkylsulfoximino-(C₁-C₆)-alkyl, (C₁-C₆)-alkylsulfoximino-(C₂-C₆)-alkylcarbonyl, (C₁-C₆)-alkoxycarbonyl, (C₁-C₆)-alkylcarbonyl, (C₃-C₆)-trialkylsilyl and benzyl,
R¹¹, R¹², R¹³, R¹⁵, R¹⁶, R¹⁷ independently of one another represent hydrogen, cyano, halogen, nitro, hydroxy, amino, tri-(C₁-C₆)-alkylsilyl, (C₃-C₈)-cycloalkyl, (C₃-C₈)-cycloalkyl-(C₃-C₈)-cycloalkyl, (C₁-C₆)-alkyl-(C₃-C₈)-cycloalkyl, halo-(C₃-C₈)-cycloalkyl, (C₁-C₆)-alkyl, (C₁-C₆)-haloalkyl, (C₁-C₆)-cyanoalkyl, (C₁-C₆)-hydroxyalkyl, hydroxycarbonyl-(C₁-C₆)-alkoxy, (C₁-C₆)-alkoxycarbonyl-(C₁-C₆)-alkyl, (C₁-C₆)-alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)-haloalkyl-(C₁-C₆)-alkoxyimino, (C₁-C₆)-alkylthio, (C₁-C₆)-haloalkylthio, (C₁-C₆)-alkoxy-(C₁-C₆)-alkylthio, (C₁-C₆)-alkylthio-(C₁-C₆)-alkyl, (C₁-C₆)-alkylsulfinyl, (C₁-C₆)-haloalkylsulfinyl, (C₁-C₆)-alkoxy-(C₁-C₆)-alkylsulfinyl, (C₁-C₆)-alkylsulfinyl-(C₁-C₆)-alkyl, (C₁-C₆)-alkylsulfonyl, (C₁-C₆)-haloalkylsulfonyl, (C₁-C₆)-alkoxy-(C₁-C₆)-alkylsulfonyl, (C₁-C₆)-alkylsulfonyl-(C₁-C₆)-alkyl, (C₁-C₆)-alkylsulfonyloxy, (C₁-C₆)-alkylcarbonyl, (C₁-C₆)-alkylthiocarbonyl, (C₁-C₆)-haloalkylcarbonyl, (C₁-C₆)-alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)-alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulfonylamino, (C₁-C₆)-alkylamino, di-(C₁-C₆)-alkylamino, (C₃-C₈)-cycloalkylamino, aminosulfonyl, (C₁-C₆)-alkylaminosulfonyl, di-(C₁-C₆)-alkylaminosulfonyl, (C₁-C₆)-alkylsulfoximino, aminothiocarbonyl, (C₁-C₆)-alkylaminothiocarbonyl, di-(C₁-C₆)-alkylaminothiocarbonyl, (C₁-C₆)-alkylcarbonylamino (—NHCO—(C₁-C₆)-alkyl), (C₃-C₈)-cycloalkylcarbonylamino, (C₁-C₆)-alkylcarbonyl-(C₁-C₆)-alkylamino (—N—(C₁-C₆)-alkyl-CO—(C₁-C₆)-alkyl), ((C₁-C₆)-alkoxycarbonyl)amino (—NHCOO—(C₁-C₆)-alkyl), ((C₁-C₆)-alkoxycarbonyl)-(C₁-C₆)-alkylamino (—N—(C₁-C₆)-alkyl-COO—(C₁-C₆)-alkyl), (C₁-C₆)-alkylcarbamoyloxy (—OCONH—(C₁-C₆)-alkyl), di-(C₁-C₆)-alkylcarbamoyloxy (—OCON—(C₁-C₆)-dialkyl), (N—(C₁-C₆)-alkylcarbamoyl)amino (—NHCONH—(C₁-C₆)-alkyl), (N,N-di-(C₁-C₆)-alkylcarbamoyl)amino (—NHCON—(C₁-C₆)-dialkyl), (N—(C₁-C₆)-alkylcarbamoyl)-(C₁-C₆)-alkylamino (—N—(C₁-C₆)-alkyl-CONH—(C₁-C₆)-alkyl), (N,N-di-(C₁-C₆)-alkylcarbamoyl)-(C₁-C₆)-alkylamino (—N—(C₁-C₆)-alkyl-CON—(C₁-C₆)-dialkyl), carbamoylamino (—NHCONH₂), (carbamoyl)-N—(C₁-C₆)-alkylamino (—N—(C₁-C₆)-alkyl-CONH₂) or
   represent aryl or hetaryl, each of which is optionally mono- or polysubstituted by identical or different substituents, where (in the case of hetaryl) at least one carbonyl group may optionally be present and where possible substituents are in each case as follows: cyano, carboxyl, halogen, nitro, hydroxy, amino, tri-(C₁-C₆)-alkylsilyl, (C₃-C₈)-cycloalkyl, (C₃-C₈)-cycloalkyl-(C₃-C₈)-cycloalkyl, (C₁-C₆)-alkyl-(C₃-C₈)-cycloalkyl, halo-(C₃-C₈)-cycloalkyl, (C₁-C₆)-alkyl, (C₁-C₆)-haloalkyl, (C₁-C₆)-cyanoalkyl, (C₁-C₆)-hydroxyalkyl, hydroxycarbonyl-(C₁-C₆)-alkoxy, (C₁-C₆)-alkoxycarbonyl-(C₁-C₆)-alkyl, (C₁-C₆)-alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)-haloalkyl-(C₁-C₆)-alkoxyimino, (C₁-C₆)-alkylthio, (C₁-C₆)-haloalkylthio, (C₁-C₆)-alkoxy-(C₁-C₆)-alkylthio, (C₁-C₆)-alkylthio-(C₁-C₆)-alkyl, (C₁-C₆)-alkylsulfinyl, (C₁-C₆)-haloalkylsulfinyl, (C₁-C₆)-alkoxy-(C₁-C₆)-alkylsulfinyl, (C₁-C₆)-alkylsulfinyl-(C₁-C₆)-alkyl, (C₁-C₆)-alkylsulfonyl, (C₁-C₆)-haloalkylsulfonyl, (C₁-C₆)-alkoxy-(C₁-C₆)-alkylsulfonyl, (C₁-C₆)-alkylsulfonyl-(C₁-C₆)-alkyl, (C₁-C₆)-alkylsulfonyloxy, (C₁-C₆)-alkylcarbonyl, (C₁-C₆)-haloalkylcarbonyl, (C₁-C₆)-alkylcarbonyloxy, (C₁-C₆)-alkoxycarbonyl, (C₁-C₆)-haloalkoxycarbonyl, aminocarbonyl, (C₁-C₆)-alkylaminocarbonyl, di-(C₁-C₆)-alkylaminocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulfonylamino, (C₁-C₆)-alkylamino, di-(C₁-C₆)-alkylamino, aminosulfonyl, (C₁-C₆)-alkylaminosulfonyl, di-(C₁-C₆)-alkylaminosulfonyl, (C₁-C₆)-alkylsulfoximino, aminothiocarbonyl, (C₁-C₆)-alkylaminothiocarbonyl, di-(C₁-C₆)-alkylaminothiocarbonyl, (C₃-C₈)-cycloalkylamino or (C₁-C₆)-alkylcarbonylamino,
   where at most two of the radicals R¹¹, R¹², R¹³, R¹⁵, R¹⁶, R¹⁷ represent a substituent other than hydrogen,
   and, in the case that neither R¹¹ nor R¹⁵, neither R¹² nor R¹⁶ or neither R¹³ nor R¹⁷ represents hydrogen, R¹⁵, R¹⁶ and R¹⁷ each independently of one another only represent cyano, halogen, (C₃-C₆)-cycloalkyl, (C₃-C₆)-cycloalkyl-(C₃-C₆)-cycloalkyl, (C₁-C₄)-alkyl-(C₃-C₆)-cycloalkyl, halo-(C₃-C₆)-cycloalkyl, (C₁-C₄)-alkyl, (C₁-C₄)-haloalkyl or (C₁-C₄)-cyanoalkyl,
   or, in the case that [symbol] exclusively represent single bonds, additionally R¹¹ and R¹⁵ and/or R¹³ and R¹⁷ in each case together represent oxygen (═O),
Q represents a partly saturated or saturated heterocyclic or heteroaromatic 8-, 9-, 10-, 11- or 12-membered fused bicyclic or tricyclic ring system which may optionally contain at least one carbonyl group and/or where the ring system is optionally mono- or polysubstituted by identical or different substituents, where the substituents independently of one another may be selected from cyano, halogen, nitro, acetyl, hydroxy, amino, SCN, tri-(C₁-C₆)-alkylsilyl, halo-(C₃-C₈)-cycloalkyl, (C₁-C₆)-alkyl, (C₁-C₆)-haloalkyl, (C₁-C₆)-cyanoalkyl, (C₁-C₆)-hydroxyalkyl, hydroxycarbonyl-(C₁-C₆)-alkoxy, (C₁-C₆)-alkoxycarbonyl-(C₁-C₆)-alkyl, (C₁-C₆)-alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-alkynyloxy-(C₁-C₄)-alkyl, (C₂-C₆)-haloalkynyl, (C₂-C₆)-cyanoalkynyl, (C₁-C₆)-alkoxy, (C₁-C₆)-haloalkoxy, (C₁-C₆)-haloalkoxy-(C₁-C₆)-alkyl, (C₂-C₆)-alkenyloxy-(C₁-C₆)-alkyl, (C₂-C₆)-haloalkenyloxy-(C₁-C₆)-alkyl, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)-haloalkyl-(C₁-C₆)-alkoxyimino, (C₁-C₆)-alkylthio, (C₁-C₆)-haloalkylthio, (C₁-C₆)-alkoxy-(C₁-C₆)-alkylthio, (C₁-C₆)-alkylthio-(C₁-C₆)-alkyl, (C₁-C₆)-alkylsulfinyl, (C₁-C₆)-haloalkylsulfinyl, (C₁-C₆)-alkoxy-(C₁-C₆)-alkylsulfinyl, (C₁-C₆)-alkylsulfinyl-(C₁-C₆)-alkyl, (C₁-C₆)-alkylsulfonyl, (C₁-C₆)-haloalkylsulfonyl, (C₁-C₆)-alkoxy-(C₁-C₆)-alkylsulfonyl, (C₁-C₆)-alkylsulfonyl-(C₁-C₆)-alkyl, (C₁-C₆)-alkylsulfonyloxy, (C₁-C₆)-alkylcarbonyl, (C₁-C₆)-alkylcarbonyl-(C₁-C₆)-alkyl, (C₁-C₆)-alkylthiocarbonyl, (C₁-C₆)-haloalkylcarbonyl, (C₁-C₆)-alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)-alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulfonylamino, (C₁-C₆)-alkylamino, di-(C₁-C₆)-alkylamino, aminosulfonyl, (C₁-C₆)-alkylaminosulfonyl, di-(C₁-C₆)-alkyl-aminosulfonyl, (C₁-C₆)-alkylsulfoximino, aminothiocarbonyl, (C₁-C₆)-alkylaminothiocarbonyl, di-(C₁-C₆)-alkylaminothiocarbonyl, (C₃-C₈)-cycloalkylamino, NHCO—(C₁-C₆)-alkyl ((C₁-C₆)-alkylcarbonylamino), or optionally monocyano-substituted (C₃-C₈)-cycloalkyl, (C₃-C₈)-cycloalkyl-(C₃-C₈)-cycloalkyl or (C₁-C₄)-alkyl-(C₃—C)-cycloalkyl,
   or where the substituents independently of one another may be selected from phenyl and a 5- or 6-membered heteroaromatic ring, where phenyl and the ring may optionally be mono- or polysubstituted by identical or different substituents selected from the group consisting of C₁-C₆-alkyl, C₂-C₆-alkenyl, C₂-C₆-alkynyl, C₃-C₆-cycloalkyl, C₁-C₆-haloalkyl, C₂-C₆-haloalkenyl, C₂-C₆-haloalkynyl, C₃-C₆-halocycloalkyl, halogen, CN, NO₂, C₁-C₄-alkoxy, C₁-C₄-haloalkoxy, and
n represents 0, 1 or 2.

It has additionally been found that the compounds of the formula (I) have very good efficacy as pesticides, preferably as insecticides and/or acaricides, and additionally generally have very good plant compatibility, in particular with respect to crop plants.

The compounds according to the invention are defined in general terms by the formula (I). Preferred substituents or ranges of the radicals given in the formulae mentioned above and below are illustrated hereinafter:

### Configuration 2-1

Compounds of the formula (I) in which preferably

in the case that [symbol] represents a single bond and a double bond, and Aa to Ae thus form an aromatic ring,
Aa represents nitrogen or carbon,
Ab represents nitrogen, oxygen, sulfur, N(R⁸) or C(R¹¹),
Ac represents nitrogen, oxygen, sulfur or C(R¹²),
Ad represents nitrogen, oxygen, sulfur, N(R⁸) or C(R¹³),
Ae represents nitrogen or carbon,
where R⁸ represents methyl, ethyl, cyclopropyl or hydrogen and
where at most three of the groups Aa, Ab, Ac, Ad and Ae may represent nitrogen and at most one of the groups Ab, Ac and Ad may represent oxygen or at most one of the groups Ab, Ac and Ad may represent sulfur or at most one of the groups Ab and Ad may represent N(R⁸),
or, if [symbol] represent exclusively single bonds,
Aa represents carbon,
Ab represents sulfur, oxygen or C(R¹¹)(R¹⁵),
Ac represents sulfur, oxygen or C(R¹²)(R¹⁶) and
Ad represents sulfur, oxygen or C(R¹³)(R¹⁷),
Ae represents carbon,
where at most one of the groups Aa, Ab, Ac, Ad and Ae may represent oxygen or sulfur,
R¹ preferably represents (C₁-C₄)-alkyl, (C₁-C₄)-hydroxyalkyl, (C₁-C₄)-haloalkyl, (C₂-C₄)-alkenyl, (C₂-C₄)-haloalkenyl, (C₂-C₄)-alkynyl, (C₂-C₄)-haloalkynyl, (C₃-C₆)-cycloalkyl, (C₁-C₄)-alkylthio-(C₁-C₄)-alkyl, (C₁-C₄)-haloalkylthio-(C₁-C₄)-alkyl, (C₁-C₄)-alkylsulfinyl-(C₁-C₄)-alkyl or (C₁-C₄)-alkylsulfonyl-(C₁-C₄)-alkyl,
R¹¹, R¹², R¹³, R¹⁵, R¹⁶, R¹⁷ independently of one another preferably represent hydrogen, cyano, halogen, nitro, hydroxy, amino, tri-(C₁-C₄)-alkylsilyl, (C₃-C₆)-cycloalkyl, (C₃-C₆)-cycloalkyl-(C₃-C₆)-cycloalkyl, (C₁-C₄)-alkyl-(C₃-C₆)-cycloalkyl, halo-(C₃-C₆)-cycloalkyl, (C₁-C₄)-alkyl, (C₁-C₄)-haloalkyl, (C₁-C₄)-cyanoalkyl, (C₁-C₄)-hydroxyalkyl, (C₁-C₄)-alkoxy-(C₁-C₄)-alkyl, (C₂-C₄)-alkenyl, (C₂-C₄)-haloalkenyl, (C₂-C₄)-cyanoalkenyl, (C₂-C₄)-alkynyl, (C₂-C₄)-haloalkynyl, (C₂-C₄)-cyanoalkynyl, (C₁-C₄)-alkoxy, (C₁-C₄)-haloalkoxy, (C₁-C₄)-cyanoalkoxy, (C₁-C₄)-alkoxy-(C₁-C₄)-alkoxy, (C₁-C₄)-alkylhydroxyimino, (C₁-C₄)-alkoxyimino, (C₁-C₄)-alkyl-(C₁-C₄)-alkoxyimino, (C₁-C₄)-haloalkyl-(C₁-C₄)-alkoxyimino, (C₁-C₄)-alkylthio, (C₁-C₄)-haloalkylthio, (C₁-C₄)-alkylthio-(C₁-C₄)-alkyl, (C₁-C₄)-alkylsulfinyl, (C₁-C₄)-haloalkylsulfinyl, (C₁-C₄)-alkylsulfinyl-(C₁-C₄)-alkyl, (C₁-C₄)-alkylsulfonyl, (C₁-C₄)-haloalkylsulfonyl, (C₁-C₄)-alkylsulfonyl-(C₁-C₄)-alkyl, (C₁-C₄)-alkylsulfonyloxy, (C₁-C₄)-alkylcarbonyl, (C₁-C₄)-haloalkylcarbonyl, aminocarbonyl, aminothiocarbonyl, (C₁-C₄)-alkylaminocarbonyl, di-(C₁-C₄)-alkylaminocarbonyl, (C₃-C₆)-cycloalkylaminocarbonyl, (C₁-C₄)-alkylsulfonylamino, (C₁-C₄)-alkylamino, di-(C₁-C₄)-alkylamino, aminosulfonyl, (C₁-C₄)-alkylaminosulfonyl, di-(C₁-C₄)-alkylaminosulfonyl, aminothiocarbonyl, (C₁-C₄)-alkylcarbonylamino, (C₁-C₄)-alkylcarbonylamino (—NHCO—(C₁-C₄)-alkyl), (C₃-C₆)-cycloalkylcarbonylamino, (C₁-C₄)-alkylcarbonyl-(C₁-C₄)-alkylamino (—N—(C₁-C₄)-alkyl-CO—(C₁-C₆)-alkyl), ((C₁-C₄)-alkoxycarbonyl)amino (—NHCOO—(C₁-C₄)-alkyl), ((C₁-C₄)-alkoxycarbonyl)-(C₁-C₄)-alkylamino (—N—(C₁-C₄)-alkyl-COO—(C₁-C₆)-alkyl), (C₁-C₄)-alkylcarbamoyloxy (—OCONH—(C₁-C₄)-alkyl), di-(C₁-C₄)-alkylcarbamoyloxy (—OCON—(C₁-C₄)-dialkyl), (N—(C₁-C₄)-alkylcarbamoyl)amino (—NHCONH—(C₁-C₄)-alkyl), (N,N-di-(C₁-C₄)-alkylcarbamoyl)amino (—NHCON—(C₁-C₄)-dialkyl), (N—(C₁-C₄)-alkylcarbamoyl)-(C₁-C₄)-alkylamino (—N—(C₁-C₄)-alkyl-CONH—(C₁-C₄)-alkyl), (N,N-di-(C₁-C₄)-alkylcarbamoyl)-(C₁-C₄)-alkylamino (—N—(C₁-C₄)-alkyl-CON—(C₁-C₄)-dialkyl), carbamoylamino (—NHCONH₂), (carbamoyl)-N—(C₁-C₄)-alkylamino (—N(C₁-C₄)-alkyl-CONH₂) or preferably represent phenyl or 5- or 6-membered hetaryl, each of which is optionally mono- or disubstituted by identical or different substituents, where (in the case of hetaryl) optionally at least one carbonyl group may be present and the heteroatoms are selected from N, S and O, and where possible substituents are in each case as follows: cyano, halogen, acetyl, (C₃-C₆)-cycloalkyl, (C₃-C₆)-cycloalkyl-(C₃-C₆)-cycloalkyl, (C₁-C₄)-alkyl-(C₃-C₆)-cycloalkyl, halo-(C₃-C₆)-cycloalkyl, (C₁-C₄)-alkyl, (C₁-C₄)-haloalkyl, (C₁-C₄)-cyanoalkyl, (C₁-C₄)-alkoxy-(C₁-C₄)-alkyl, (C₂-C₄)-alkenyl, (C₂-C₄)-alkynyl, (C₂— (C₁-C₄)-alkoxy, (C₁-C₄)-alkylthio, (C₁-C₄)-haloalkylthio, (C₁-C₄)-alkylthio-(C₁-C₄)-alkyl, (C₁-C₄)-alkylsulfinyl, (C₁-C₄)-haloalkylsulfinyl, (C₁-C₄)-alkylsulfinyl-(C₁-C₄)-alkyl, (C₁-C₄)-alkylsulfonyl, (C₁-C₄)-haloalkylsulfonyl, (C₁-C₄)-alkylsulfonyl-(C₁-C₄)-alkyl, (C₁-C₄)-alkylsulfonyloxy, (C₁-C₄)-alkylcarbonyl, aminocarbonyl, (C₁-C₄)-alkylaminocarbonyl, di-(C₁-C₄)-alkylaminocarbonyl, (C₁-C₄)-alkylsulfonylamino, aminosulfonyl, (C₁-C₄)-alkylaminosulfonyl, di-(C₁-C₄)-alkylaminosulfonyl or (C₁-C₄)-alkylcarbonylamino,
   where at most two of the radicals R¹¹, R¹², R¹³, R¹⁵, R¹⁶, R¹⁷ represent a substituent other than hydrogen,
   and, in the case that neither R¹¹ nor R¹⁵, neither R¹² nor R¹⁶ or neither R¹³ nor R¹⁷ represents hydrogen, R¹⁵, R¹⁶ and R¹⁷ each independently of one another only represent cyano, halogen, (C₃-C₆)-cycloalkyl, (C₃-C₆)-cycloalkyl-(C₃-C₆)-cycloalkyl, (C₁-C₄)-alkyl-(C₃-C₆)-cycloalkyl, halo-(C₃-C₆)-cycloalkyl, (C₁-C₄)-alkyl, (C₁-C₄)-haloalkyl or (C₁-C₄)-cyanoalkyl,
   or, in the case that [symbol] exclusively represent single bonds, additionally R¹¹ and R¹⁵ and/or R¹³ and R¹⁷ in each case together preferably represent oxygen (═O),
Q preferably represents a heteroaromatic 9-membered or 12-membered fused bicyclic or tricyclic ring system from the group consisting of Q1 to Q13 and Q15 to Q21

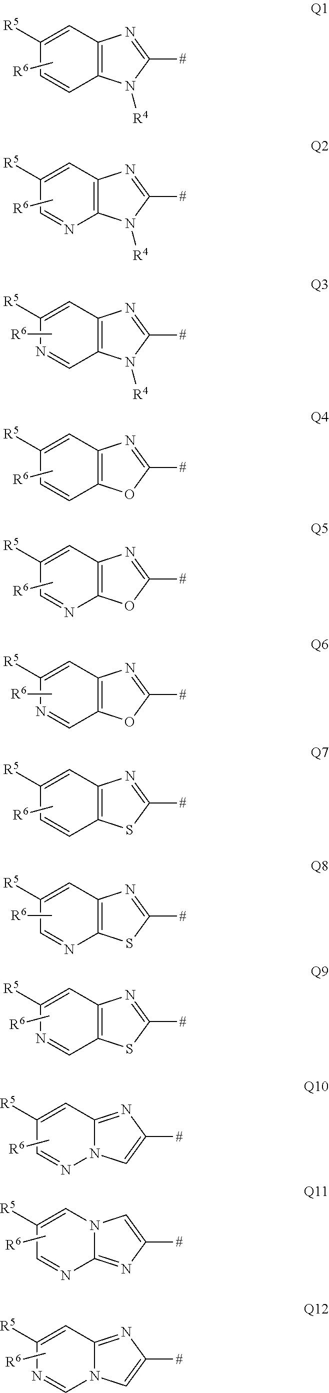

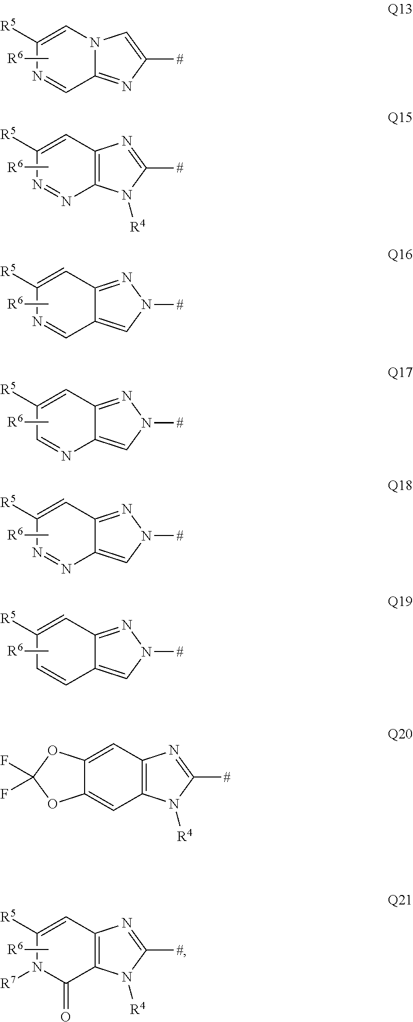

where
R⁴ preferably represents (C₁-C₄)-alkyl, (C₁-C₄)-haloalkyl, (C₁-C₄)-cyanoalkyl, (C₁-C₄)-hydroxyalkyl, (C₁-C₄)-alkoxy-(C₁-C₄)-alkyl, (C₁-C₄)-haloalkoxy-(C₁-C₄)-alkyl, (C₂-C₄)-alkenyl, (C₂-C₄)-alkenyloxy-(C₁-C₄)-alkyl, (C₂-C₄)-haloalkenyloxy-(C₁-C₄)-alkyl, (C₂-C₄)-haloalkenyl, (C₂-C₄)-cyanoalkenyl, (C₂-C₄)-alkynyl, (C₂-C₄)-alkynyloxy-(C₁-C₄)-alkyl, (C₂-C₄)-haloalkynyl, (C₃-C₆)-cycloalkyl, (C₃-C₆)-cycloalkyl-(C₃-C₆)-cycloalkyl, (C₁-C₄)-alkyl-(C₃-C₆)-Cycloalkyl, halo-(C₃-C₆)-cycloalkyl, (C₁-C₄)-alkylthio-(C₁-C₄)-alkyl, (C₁-C₄)-alkylsulfinyl-(C₁-C₄)-alkyl, (C₁-C₄)-alkylsulfonyl-(C₁-C₄)-alkyl or (C₁-C₄)-alkylcarbonyl-(C₁-C₄)-alkyl,
R⁵, R⁶ independently of one another preferably represent hydrogen, cyano, halogen, (C₁-C₄)-alkyl, (C₁-C₄)-haloalkyl, (C₂-C₄)-alkenyl, (C₂-C₄)-haloalkenyl, (C₂-C₄)-alkynyl, (C₂-C₄)-haloalkynyl, (C₁-C₄)-alkoxy, (C₁-C₄)-haloalkoxy, (C₁-C₄)-alkoxyimino, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, (C₁-C₄)-alkylsulfonyloxy, (C₁-C₄)-alkylcarbonyl, (C₁-C₄)-haloalkylcarbonyl, aminocarbonyl, (C₁-C₄)-alkylaminocarbonyl, di-(C₁-C₄)-alkylaminocarbonyl, (C₁-C₄)-alkylsulfonylamino, (C₁-C₄)-alkylamino, di-(C₁-C₄)-alkylamino, aminosulfonyl, (C₁-C₄)-alkylaminosulfonyl, di-(C₁-C₄)-alkylaminosulfonyl or optionally monocyano-substituted (C₃-C₆)-cycloalkyl, (C₃-C₆)-cycloalkyl-(C₃-C₆)-cycloalkyl or (C₁-C₄)-alkyl-(C₃-C₆)-cycloalkyl,
R⁷ preferably represents hydrogen, (C₁-C₄)-alkyl, (C₁-C₄)-haloalkyl, (C₁-C₄)-cyanoalkyl, (C₁-C₄)-hydroxyalkyl, (C₁-C₄)-alkoxy-(C₁-C₄)-alkyl, (C₁-C₄)-haloalkoxy-(C₁-C₄)-alkyl, (C₂-C₄)-alkenyl, (C₂-C₄)-alkenyloxy-(C₁-C₄)-alkyl, (C₂-C₄)-haloalkenyloxy-(C₁-C₄)-alkyl, (C₂-C₄)-haloalkenyl, (C₂-C₄)-cyanoalkenyl, (C₂-C₄)-alkynyl, (C₂-C₄)-haloalkynyl, (C₃-C₆)-cycloalkyl, (C₃-C₆)-cycloalkyl-(C₃-C₆)-cycloalkyl, (C₁-C₄)-alkyl-(C₃-C₆)-cycloakyl, halo-(C₃-C₆)-cycloalkyl, (C₁-C₄)-alkylthio-(C₁-C₄)-alkyl, (C₁-C₄)-alkylsulfinyl-(C₁-C₄)-alkyl or (C₁-C₄)-alkylsulfonyl-(C₁-C₄)-alkyl,
   and
n is preferably 0, 1 or 2.

### Configuration 3-1

Compounds of the formulae (Ia), (Ib), (Id), (Ie), (Ig) to (Ik) and (Im) to (It),

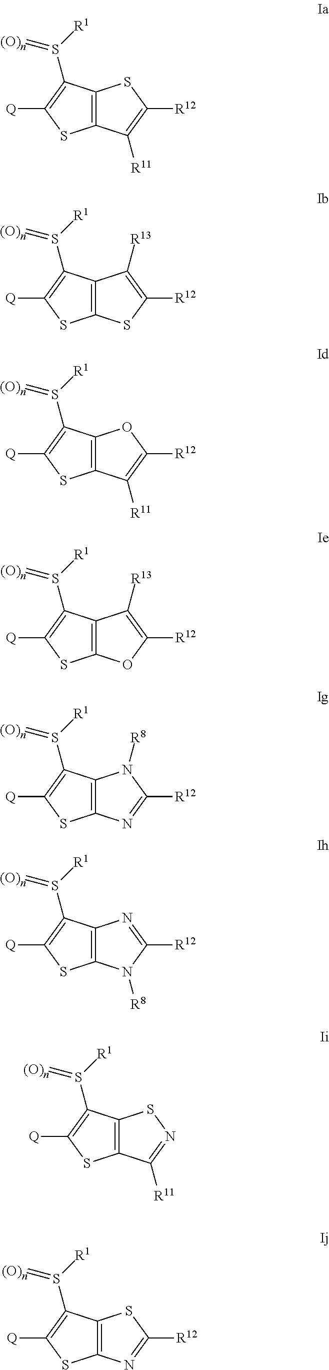

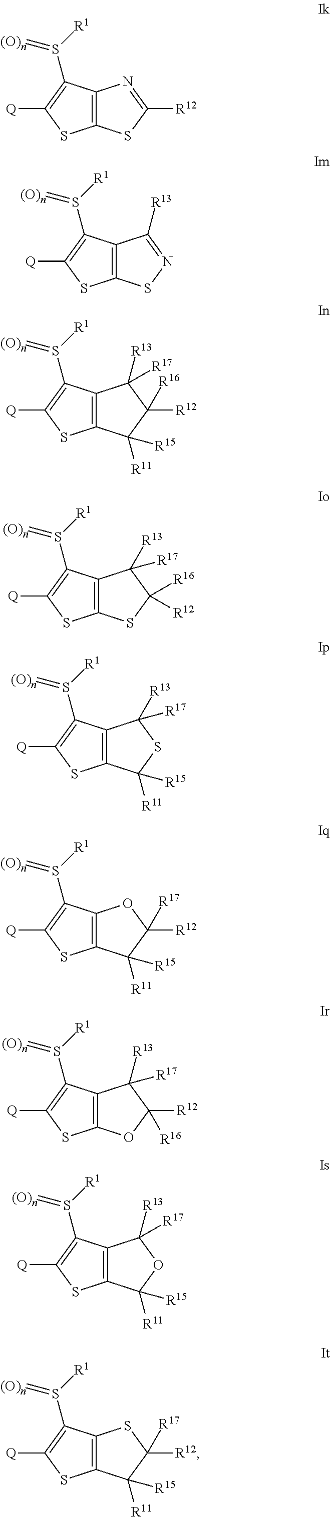

where
R¹ particularly preferably represents (C₁-C₄)-alkyl, (C₁-C₄)-haloalkyl or (C₃-C₆)-cycloalkyl,
R⁸ particularly preferably represents methyl, ethyl, cyclopropyl or hydrogen,
R¹¹, R¹², R¹³, R¹⁵, R¹⁶, R¹⁷ independently of one another particularly preferably represent hydrogen, cyano, halogen, nitro, hydroxy, (C₃-C₆)-cycloalkyl, (C₁-C₄)-alkyl-(C₃-C₆)-cycloalkyl, halo-(C₃-C₆)-cycloalkyl, (C₁-C₄)-alkyl, (C₁-C₄)-haloalkyl, (C₁-C₄)-alkoxy-(C₁-C₄)-alkyl, (C₁-C₄)-alkoxy, (C₁-C₄)-haloalkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, aminosulfonyl, (C₁-C₄)-alkylaminosulfonyl, di-(C₁-C₄)-alkylaminosulfonyl, aminocarbonyl, (C₁-C₄)-alkylaminocarbonyl, di-(C₁-C₄)-alkylaminocarbonyl, (C₃-C₆)-cycloalkylaminocarbonyl, (C₁-C₄)-alkylcarbonylamino (—NHCO—(C₁-C₄)-alkyl), (C₃-C₆)-cycloalkylcarbonylamino, (C₁-C₄)-alkylcarbonyl-(C₁-C₄)-alkylamino (—N—(C₁-C₄)-alkyl-CO—(C₁-C₆)-alkyl), ((C₁-C₄)-alkoxycarbonyl)amino (—NHCOO—(C₁-C₄)-alkyl), (N—(C₁-C₄)-alkylcarbamoyl)amino (—NHCONH—(C₁-C₄)-alkyl), (N,N-di-(C₁-C₄)-alkylcarbamoyl)amino (—NHCON—(C₁-C₄)-dialkyl), (N—(C₁-C₄)-alkylcarbamoyl)-(C₁-C₄)-alkylamino (—N(C₁-C₄)-alkyl-CONH—(C₁-C₄)-alkyl), (N,N-di-(C₁-C₄)-alkylcarbamoyl)-(C₁-C₄)-alkylamino (—N(C₁-C₄)-alkyl-CON—(C₁-C₄)-dialkyl), carbamoylamino (—NHCONH₂), (carbamoyl)-N—(C₁-C₄)-alkylamino (—N—(C₁-C₄)-alkyl-CONH₂)
   or particularly preferably represent phenyl, triazole or pyrazole, each of which is optionally mono- or disubstituted by identical or different substituents, possible substituents being in each case: cyano, halogen, (C₃-C₆)-cycloalkyl, (C₃-C₆)-cycloalkyl-(C₃-C₆)-cycloalkyl, (C₁-C₄)-alkyl-(C₃-C₆)-cycloalkyl, halo-(C₃-C₆)-cycloalkyl, (C₁-C₄)-alkyl, (C₁-C₄)-haloalkyl, (C₁-C₄)-cyanoalkyl, (C₂-C₄)-alkenyl, (C₂-C₄)-alkynyl, (C₁-C₄)-alkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, (C₁-C₄)-alkylsulfonyloxy, (C₁-C₄)-alkylcarbonyl, aminocarbonyl, (C₁-C₄)-alkylaminocarbonyl, di-(C₁-C₄)-alkylaminocarbonyl, (C₁-C₄)-alkylsulfonylamino, aminosulfonyl, (C₁-C₄)-alkylaminosulfonyl, di-(C₁-C₄)-alkylaminosulfonyl or (C₁-C₄)-alkylcarbonylamino,
   where at most two of the radicals R¹¹, R¹², R¹³, R¹⁵, R¹⁶, R¹⁷ represent a substituent other than hydrogen,
   and, in the case that neither R¹¹ nor R¹⁵, neither R¹² nor R¹⁶ or neither R¹³ nor R¹⁷ represents hydrogen, R¹⁴, R¹⁵, R¹⁶ and R¹⁷ each independently of one another only represent cyano, halogen, (C₃-C₆)-cycloalkyl, (C₃-C₆)-cycloalkyl-(C₃-C₆)-cycloalkyl, (C₁-C₄)-alkyl-(C₃-C₆)-cycloalkyl, halo-(C₃-C₆)-cycloalkyl, (C₁-C₄)-alkyl or (C₁-C₄)-haloalkyl,
   or, in the case of formulae of the structures (In) to (It), R¹¹ and R¹⁵ or R¹³ and R¹⁷ additionally in each case together particularly preferably represent oxygen (═O),
Q particularly preferably represents a heteroaromatic 9-membered fused bicyclic ring system from the group consisting of Q1, Q2, Q3, Q5, Q6, Q8, Q9, Q10, Q12, Q15, Q20 and Q21,

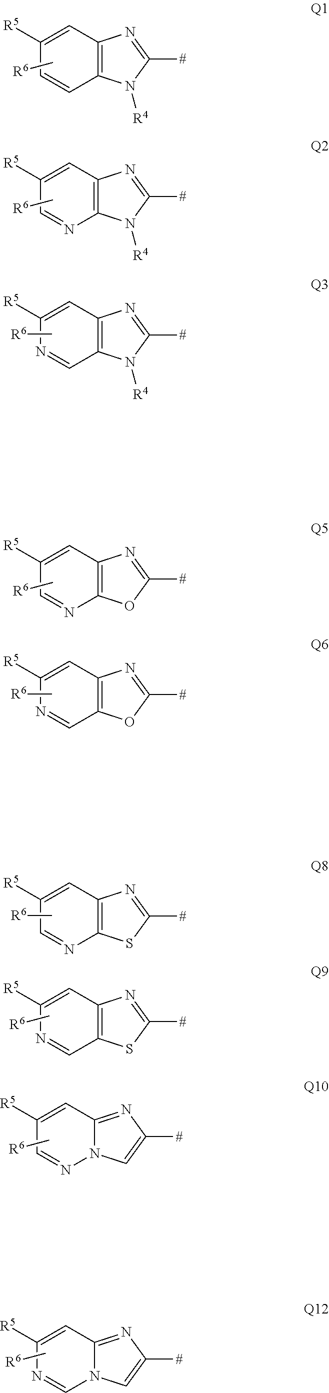

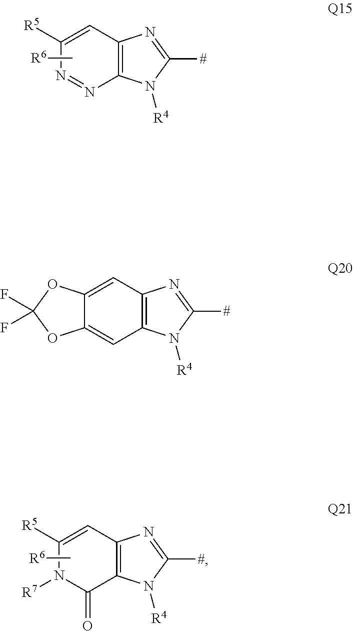

R⁴ particularly preferably represents (C₁-C₄)-alkyl, (C₁-C₄)-haloalkyl, (C₁-C₄)-cyanoalkyl or (C₁-C₄)-alkoxy-(C₁-C₄)-alkyl,
R⁵ particularly preferably represents halogen, (C₁-C₄)-haloalkyl, (C₁-C₄)-haloalkoxy, (C₁-C₄)-haloalkylthio, (C₁-C₄)-haloalkylsulfinyl, (C₁-C₄)-haloalkylsulfonyl or represents optionally monocyano-substituted (C₃-C₆)-cycloalkyl,
R⁶ particularly preferably represents hydrogen, cyano, halogen or (C₁-C₄)-alkyl,
R⁷ particularly preferably represents hydrogen, (C₁-C₄)-alkyl, (C₁-C₄)-haloalkyl, (C₁-C₄)-cyanoalkyl, (C₁-C₄)-hydroxyalkyl, (C₁-C₄)-alkoxy-(C₁-C₄)-alkyl, (C₁-C₄)-haloalkoxy-(C₁-C₄)-alkyl, (C₂-C₄)-alkenyl, (C₂-C₄)-alkenyloxy-((C₁-C₄)-alkyl, (C₂-C₄)-haloalkenyloxy-(C₁-C₄)-alkyl, (C₂-C₄)-haloalkenyl, (C₂-C₄)-cyanoalkenyl, (C₂-C₄)-alkynyl, (C₂-C₄)-haloalkynyl, (C₃-C₆)-cycloalkyl, (C₃-C₆)-cycloalkyl-(C₃-C₆)-cycloalkyl, (C₁-C₄)-alkynyl-(C₃-C₆)-cycloalkyl, halo-(C₃-C₆)-cycloalkyl, (C₁-C₄)-alkylthio-(C₁-C₄)-alkyl, (C₁-C₄)-alkylsulfinyl-(C₁-C₄)-alkyl or (C₁-C₄)-alkylsulfonyl-(C₁-C₄)-alkyl,
   and
n particularly preferably represents 0, 1 or 2.

### Configuration 4-1

Very particular preference is given to compounds of the formulae (Ia), (Ib), (Id), (Ie), (Ii) to (Ik), (In), (Iq) and (Ir)

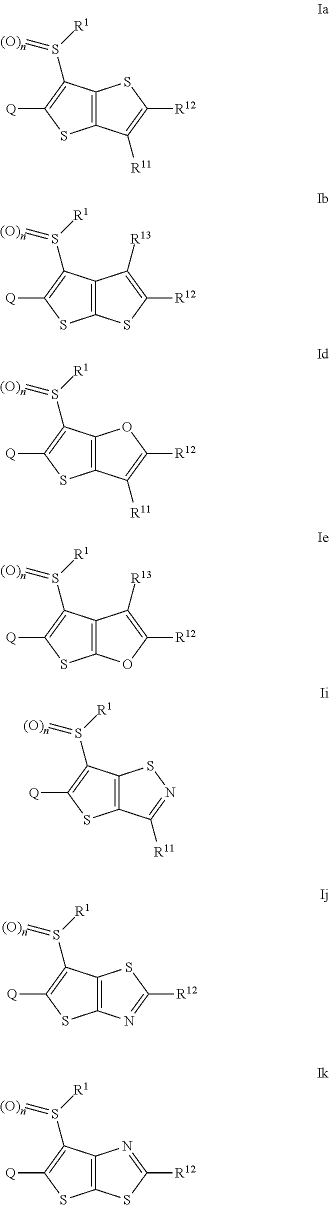

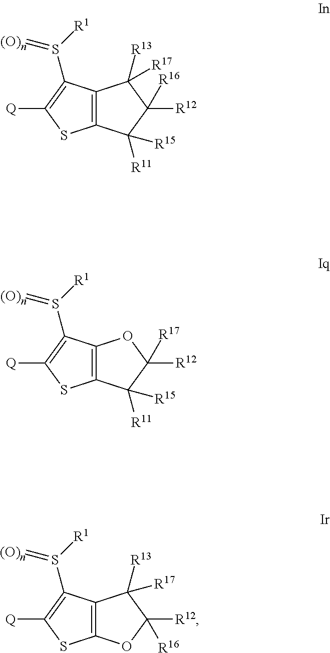

where
R¹ very particularly preferably represents (C₁-C₄)-alkyl, (C₃—C)-cycloalkyl or (C₁-C₄)-haloalkyl,
R¹³, R¹⁵, R¹⁶, R¹⁷ independently of one another very particularly preferably represent hydrogen, halogen, (C₁-C₄)-haloalkyl or (C₁-C₄)-alkyl,
R¹¹, R¹² independently of one another very particularly preferably represent hydrogen, cyano, halogen, nitro, hydroxy, (C₃-C₆)-cycloalkyl, (C₁-C₄)-alkyl-(C₃-C₆)-cycloalkyl, halo-(C₃-C₆)-cycloalkyl, (C₁-C₄)-alkyl, (C₁-C₄)-haloalkyl, (C₁-C₄)-alkoxy-(C₁-C₄)-alkyl, (C₁-C₄)-alkoxy, (C₁-C₄)-haloalkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, aminosulfonyl, (C₁-C₄)-alkylaminosulfonyl, di-(C₁-C₄)-alkylaminosulfonyl, aminocarbonyl, (C₁-C₄)-alkylaminocarbonyl, di-(C₁-C₄)-alkylaminocarbonyl, (C₃-C₆)-cycloalkylaminocarbonyl, (C₁-C₄)-alkylcarbonylamino (—NHCO—(C₁-C₄)-alkyl), (C₃-C₆)-cycloalkylcarbonylamino, (C₁-C₄)-alkylcarbonyl-(C₁-C₄)-alkylamino (—N—(C₁-C₄)-alkyl-CO—(C₁-C₆)-alkyl), ((C₁-C₄)-alkoxycarbonyl)amino (—NHCOO—(C₁-C₄)-alkyl), (N—(C₁-C₄)-alkylcarbamoyl)amino (—NHCONH—(C₁-C₄)-alkyl), (N,N-di-(C₁-C₄)-alkylcarbamoyl)amino (—NHCON—(C₁-C₄)-dialkyl), (N—(C₁-C₄)-alkylcarbamoyl)-(C₁-C₄)-alkylamino (—N(C₁-C₄)-alkyl-CONH—(C₁-C₄)-alkyl), carbamoylamino (—NHCONH₂), (carbamoyl)-N—(C₁-C₄)-alkylamino (—N—(C₁-C₄)-alkyl-CONH₂)
   or very particularly preferably represent phenyl, pyrazole or triazole, each of which is optionally mono- or disubstituted by identical or different substituents, possible substituents being in each case: cyano, halogen, (C₃-C₆)-cycloalkyl, (C₁-C₄)-alkyl-(C₃-C₆)-cycloalkyl, halo-(C₃-C₆)-cycloalkyl, (C₁-C₄)-alkyl, (C₁-C₄)-haloalkyl, (C₁-C₄)-alkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, (C₁-C₄)-alkylsulfonyloxy, aminocarbonyl, (C₁-C₄)-alkylaminocarbonyl, di-(C₁-C₄)-alkylaminocarbonyl, (C₁-C₄)-alkylcarbonylamino,
where at most two of the radicals R¹¹, R¹², R¹³, R¹⁵, R¹⁶, R¹⁷ represent a substituent other than hydrogen,
and, in the case that neither R¹¹ nor R¹⁵, neither R² nor R¹⁶ or neither R¹³ nor R¹⁷ represents hydrogen, R¹⁴, R¹⁵, R¹⁶ and R¹⁷ each independently of one another only represent halogen, (C₁-C₄)-haloalkyl or (C₁-C₄)-alkyl,
or, in the case of formulae of the structures (In), (Iq) and (Ir), R¹¹ and R¹⁵ or R¹³ and R¹⁷ additionally in each case together very particularly preferably represent oxygen (═O),
Q very particularly preferably represents a heteroaromatic 9-membered fused bicyclic ring system from the group consisting of Q1, Q2, Q3, Q5, Q6, Q12, Q10, Q15, Q20 and Q21

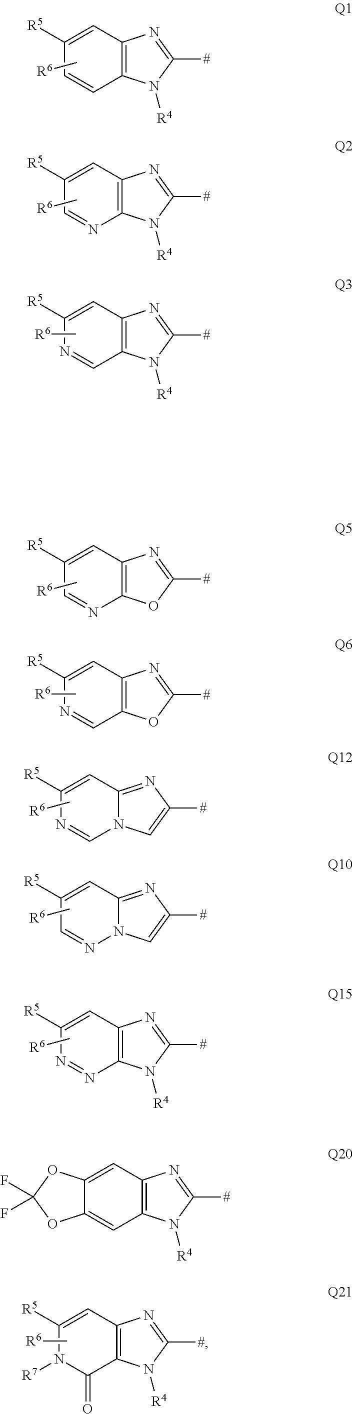

where
R⁴ very particularly preferably represents (C₁-C₄)-alkyl,
R⁵ very particularly preferably represents halogen, (C₁-C₄)-haloalkyl, (C₁-C₄)-haloalkoxy, (C₁-C₄)-haloalkylthio, (C₁-C₄)-haloalkylsulfonyl, (C₁-C₄)-haloalkylsulfinyl or represents optionally monocyano-substituted (C₃-C₆)-cycloalkyl,
R⁶ very particularly preferably represents hydrogen,
R⁷ very particularly preferably represents hydrogen, (C₁-C₄)-alkyl, (C₁-C₄)-haloalkyl, (C₁-C₄)-cyanoalkyl, (C₁-C₄)-alkoxy-(C₁-C₄)-alkyl, (C₁-C₄)-haloalkoxy-(C₁-C₄)-alkyl, (C₂-C₄)-alkenyl, (C₂-C₄)-haloalkenyl, (C₂-C₄)-cyanoalkenyl, (C₂-C₄)-alkynyl, (C₂-C₄)-haloalkynyl, (C₃-C₆)-cycloalkyl, (C₃-C₆)-cycloalkyl-(C₃-C₆)-cycloalkyl, (C₁-C₄)-alkyl-(C₃-C₆)-cycloalkyl, halo-(C₃-C₆)-cycloalkyl, (C₁-C₄)-alkylthio-(C₁-C₄)-alkyl, (C₁-C₄)-alkylsulfinyl-(C₁-C₄)-alkyl or (C₁-C₄)-alkylsulfonyl-(C₁-C₄)-alkyl,
   and
n is even more preferably 0, 1 or 2.

Configuration 5-1

Emphasis is given to compounds of the formulae (Ia), (Id), (Ij) and (In),

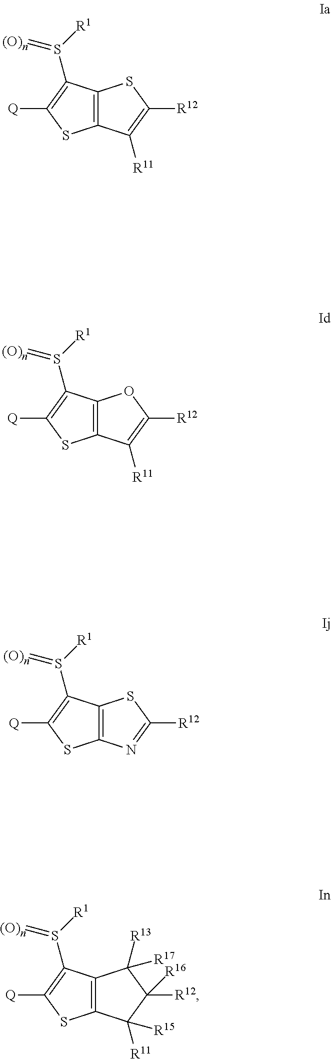

where
R¹ most preferably represents (C₁-C₄)-alkyl,
R¹³ most preferably represents hydrogen or halogen,
R¹⁵, R¹⁶, R¹⁷ most preferably represent hydrogen,
R¹¹, R¹² independently of one another most preferably represent hydrogen, hydroxy, cyano, halogen, (C₁-C₄)-alkyl, (C₃-C₆)-cycloalkyl, (C₁-C₄)-haloalkyl, (C₁-C₄)-haloalkoxy, aminocarbonyl, (C₁-C₄)-alkylaminocarbonyl, di-(C₁-C₄)-alkylaminocarbonyl, (C₃-C₆)-cycloalkylaminocarbonyl, (C₁-C₄)-alkylcarbonylamino (—NHCO—(C₁-C₄)-alkyl), (C₃-C₆)-cycloalkylcarbonylamino, (N—(C₁-C₄)-alkylcarbamoyl)amino (—NHCONH—(C₁-C₄)-alkyl), phenyl,
   or represent pyrazole or triazole, each of which is optionally monosubstituted by (C₁-C₄)-alkyl, halogen or (C₁-C₄)-haloalkyl,
or, in the case of formulae of the structure (In), R¹¹ and R¹⁵ or R¹³ and R¹⁷ additionally in each case together most preferably represent oxygen (═O),
Q most preferably represents a heteroaromatic 9-membered fused bicyclic ring system from the group consisting of Q2, Q3, Q15 and Q21,

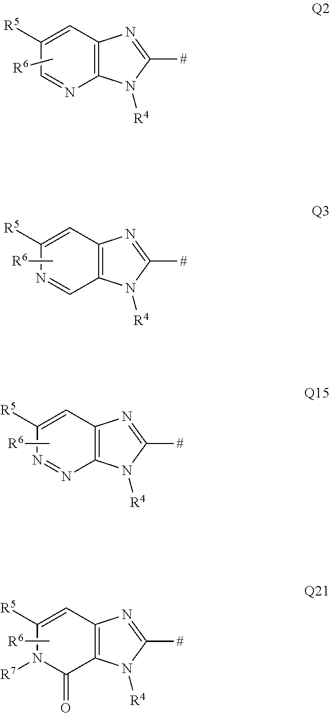

R⁴ most preferably represents (C₁-C₄)-alkyl,
R⁵ most preferably represents (C₁-C₄)-haloalkyl, (C₁-C₄)-haloalkylthio, (C₁-C₄)-haloalkylsulfonyl, (C₁-C₄)-haloalkylsulfinyl or monocyano-substituted cyclopropyl,
R⁶ most preferably represents hydrogen and
R⁷ most preferably represents (C₁-C₄)-alkyl, cyclopropyl, methoxymethyl or methoxyethyl and
n most preferably represents 0 or 2.

Configuration 6-1

Emphasis is given to compounds of the formulae (Ia), (Id), (Ij) and (In),

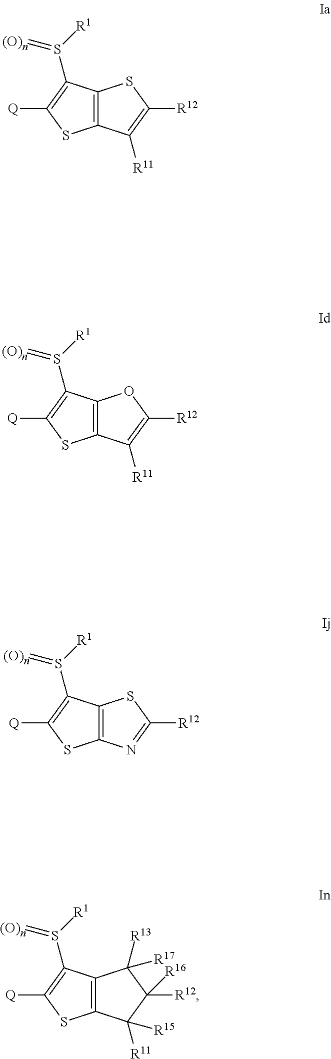

where
R¹ especially represents ethyl,
R¹³ especially represents hydrogen or fluorine,
R¹⁵, R¹⁶, R¹⁷ especially represent hydrogen,
R¹¹ especially represents hydrogen, hydroxy, cyano, cyclopropyl, trifluoromethyl, bromine, iodine, fluorine, aminocarbonyl, methylaminocarbonyl, methylcarbonylamino, cyclopropylcarbonylamino, (N-methylcarbamoyl)amino (—NHCONHMe), phenyl, N-methylpyrazole, trifluoromethylimidazole or chloropyrazole,
R¹² especially represents hydrogen, cyclopropyl, methyl, trifluoromethyl or chlorine, or, in the case of formulae of the structure (In), R¹¹ and R¹⁵ or R¹³ and R¹⁷ additionally in each case together especially represent oxygen (═O),
Q especially represents a heteroaromatic 9-membered fused bicyclic ring system from the group consisting of Q2, Q3, Q15 and Q21,

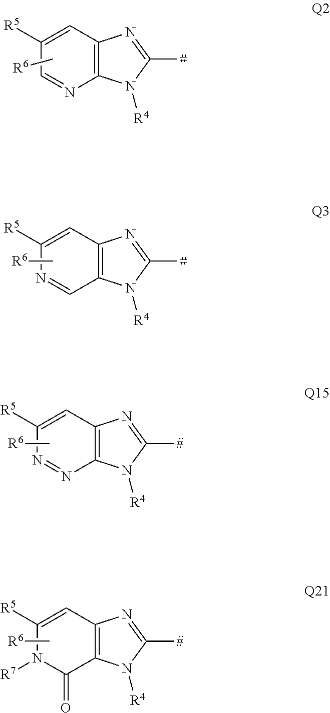

R⁴ especially represents methyl,
R⁵ especially represents trifluoromethyl, trifluoromethylsulfonyl, trifluoromethylthio, trifluoromethylsulfinyl or pentafluoroethyl,
R⁶ especially represents hydrogen,
R⁷ especially represents methyl
   and
n especially represents 2.

Further alternative embodiments of the formula (I) are illustrated below:

Compounds of the formula (I)

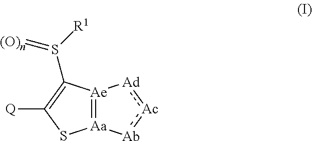

where [symbol] represents two single bonds or one double bond and one single bond,
in which (Configuration 1-2)
if [symbol] represents a double bond and a single bond, and Aa to Ae thus form an aromatic ring,
Aa represents nitrogen or carbon,
Ab represents nitrogen, oxygen, sulfur or C(R¹¹),
Ac represents nitrogen, oxygen, sulfur or C(R¹²),
Ad represents nitrogen, oxygen, sulfur or C(R¹³),
Ae represents nitrogen or carbon,
where at most three of the groups Aa, Ab, Ac, Ad and Ae may represent nitrogen and at most one may represent oxygen or at most one may represent sulfur,
or, in the case that [symbol] represent exclusively single bonds,
Aa represents carbon,
Ab represents sulfur, oxygen or C(R¹¹)(R⁵),
Ac represents sulfur, oxygen or C(R¹²)(R¹⁶) and
Ad represents sulfur, oxygen or C(R¹³)(R¹⁷),
Ae represents carbon,
where at most one of the groups Aa, Ab, Ac, Ad and Ae may represent oxygen or sulfur,
R¹ represents (C₁-C₆)-alkyl, (C₁-C₆)-haloalkyl, (C₁-C₆)-cyanoalkyl, (C₁-C₆)-hydroxyalkyl, (C₁-C₆)-alkoxy-(C₁-C₆)-alkyl, (C₁-C₆)-haloalkoxy-(C₁-C₆)-alkyl, (C₂-C₆)-alkenyl, (C₂-C₆)-alkenyloxy-(C₁-C₆)-alkyl, (C₂-C₆)-haloalkenyloxy-(C₁-C₆)-alkyl, (C₂-C₆)-haloalkenyl, (C₂-C₆)-cyanoalkenyl, (C₂-C₆)-alkynyl, (C₂-C₆)-alkynyloxy-(C₁-C₆)-alkyl, (C₂-C₆)-haloalkynyloxy-(C₁-C₆)-alkyl, (C₂-C₆)-haloalkynyl, (C₂-C₆)-cyanoalkynyl, (C₃-C₈)-cycloalkyl, (C₃-C₈)-cycloalkyl-(C₃-C₈)-cycloalkyl, (C₁-C₆)-alkyl-(C₃-C₈)-cycloalkyl, halo-(C₃-C₈)-cycloalkyl, amino, (C₁-C₆)-alkylamino, di-(C₁-C₆)-alkylamino, (C₃-C₈)-cycloalkylamino, (C₁-C₆)-alkylcarbonylamino, (C₁-C₆)-alkylthio-(C₁-C₆)-alkyl, (C₁-C₆)-haloalkylthio-(C₁-C₆)-alkyl, (C₁-C₆)-alkylsulfinyl-(C₁-C₆)-alkyl, (C₁-C₆)-haloalkylsulfinyl-(C₁-C₆)-alkyl, (C₁-C₆)-alkylsulfonyl-(C₁-C₆)-alkyl, (C₁-C₆)-haloalkylsulfonyl-(C₁-C₆)-alkyl, (C₁-C₆)-alkoxy-(C₁-C₆)-alkylthio-(C₁-C₆)-alkyl, (C₁-C₆)-alkoxy-(C₁-C₆)-alkylsulfinyl-(C₁-C₆)-alkyl, (C₁-C₆)-alkoxy-(C₁-C₆)-alkylsulfonyl-(C₁-C₆)-alkyl, (C₁-C₆)-alkylcarbonyl-(C₁-C₆)-alkyl, (C₁-C₆)-haloalkylcarbonyl-(C₁-C₆)-alkyl, (C₁-C₆)-alkoxycarbonyl-(C₁-C₆)-alkyl, (C₁-C₆)-haloalkoxycarbonyl-(C₁-C₆)-alkyl, (C₁-C₆)-alkylsulfonylamino, aminosulfonyl-(C₁-C₆)-alkyl, (C₁-C₆)-alkylaminosulfonyl-(C₁-C₆)-alkyl, di-(C₁-C₆)-alkylaminosulfonyl-(C₁-C₆)-alkyl,
   or represents (C₁-C₆)-alkyl, (C₁-C₆)-alkoxy, (C₂-C₆)-alkenyl, (C₂-C₆)-alkynyl, (C₃-C₈)-cycloalkyl, each of which is optionally mono- or polysubstituted by identical or different substituents from the group consisting of aryl, hetaryl and heterocyclyl, where aryl, hetaryl or heterocyclyl may each optionally be mono- or polysubstituted by identical or different substituents from the group consisting of halogen, cyano, nitro, hydroxy, amino, carboxy, carbamoyl, aminosulfonyl, (C₁-C₆)-alkyl, (C₃-C₆)-cycloalkyl, (C₁-C₆)-alkoxy, (C₁-C₆)-haloalkyl, (C₁-C₆)-haloalkoxy, (C₁-C₆)-alkylthio, (C₁-C₆)-alkylsulfinyl, (C₁-C₆)-alkylsulfonyl, (C₁-C₆)-alkylsulfimino, (C₁-C₆)-alkylsulfimino-(C₁-C₆)-alkyl, (C₁-C₆)-alkylsulfimino-(C₂-C₆)-alkylcarbonyl, (C₁-C₆)-alkylsulfoximino, (C₁-C₆)-alkylsulfoximino-(C₁-C₆)-alkyl, (C₁-C₆)-alkylsulfoximino-(C₂-C₆)-alkylcarbonyl, (C₁-C₆)-alkoxycarbonyl, (C₁-C₆)-alkylcarbonyl, (C₃-C₆)-trialkylsilyl and benzyl, or
R¹ represents aryl, hetaryl or heterocyclyl, each of which is optionally mono- or polysubstituted by identical or different substituents from the group consisting of halogen, cyano, nitro, hydroxy, amino, carboxyl, carbamoyl, (C₁-C₆)-alkyl, (C₃-C₈)-cycloalkyl, (C₁-C₆)-alkoxy, (C₁-C₆)-haloalkyl, (C₁-C₆)-haloalkoxy, (C₁-C₆)-alkylthio, (C₁-C₆)-alkylsulfinyl, (C₁-C₆)-alkylsulfonyl, (C₁-C₆)-alkylsulfimino, (C₁-C₆)-alkylsulfimino-(C₁-C₆)-alkyl, (C₁-C₆)-alkylsulfimino-(C₂-C₆)-alkylcarbonyl, (C₁-C₆)-alkylsulfoximino, (C₁-C₆)-alkylsulfoximino-(C₁-C₆)-alkyl, (C₁-C₆)-alkylsulfoximino-(C₂-C₆)-alkylcarbonyl, (C₁-C₆)-alkoxycarbonyl, (C₁-C₆)-alkylcarbonyl, (C₃-C₆)-trialkylsilyl, (═O) (only in the case of heterocyclyl) and (═O)₂ (only in the case of heterocyclyl),
R¹¹, R¹², R¹³, R¹⁵, R¹⁶, R¹⁷ independently of one another represent hydrogen, cyano, halogen, nitro, hydroxy, amino, tri-(C₁-C₆)-alkylsilyl, (C₃-C₈)-cycloalkyl, (C₃-C₈)-cycloalkyl-(C₃-C₈)-cycloalkyl, (C₁-C₆)-alkyl-(C₃-C₈)-cycloalkyl, halo-(C₃-C₈)-cycloalkyl, (C₁-C₆)-alkyl, (C₁-C₆)-haloalkyl, (C₁-C₆)-cyanoalkyl, (C₁-C₆)-hydroxyalkyl, hydroxycarbonyl-(C₁-C₆)-alkoxy, (C₁-C₆)-alkoxycarbonyl-(C₁-C₆)-alkyl, (C₁-C₆)-alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)-haloalkyl-(C₁-C₆)-alkoxyimino, (C₁-C₆)-alkylthio, (C₁-C₆)-haloalkylthio, (C₁-C₆)-alkoxy-(C₁-C₆)-alkylthio, (C₁-C₆)-alkylthio-(C₁-C₆)-alkyl, (C₁-C₆)-alkylsulfinyl, (C₁-C₆)-haloalkylsulfinyl, (C₁-C₆)-alkoxy-(C₁-C₆)-alkylsulfinyl, (C₁-C₆)-alkylsulfinyl-(C₁-C₆)-alkyl, (C₁-C₆)-alkylsulfonyl, (C₁-C₆)-haloalkylsulfonyl, (C₁-C₆)-alkoxy-(C₁-C₆)-alkylsulfonyl, (C₁-C₆)-alkylsulfonyl-(C₁-C₆)-alkyl, (C₁-C₆)-alkylsulfonyloxy, (C₁-C₆)-alkylcarbonyl, (C₁-C₆)-alkylthiocarbonyl, (C₁-C₆)-haloalkylcarbonyl, (C₁-C₆)-alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)-alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulfonylamino, (C₁-C₆)-alkylamino, di-(C₁-C₆)-alkylamino, (C₃-C₈)-cycloalkylamino, aminosulfonyl, (C₁-C₆)-alkylaminosulfonyl, di-(C₁-C₆)-alkylaminosulfonyl, (C₁-C₆)-alkylsulfoximino, aminothiocarbonyl, (C₁-C₆)-alkylaminothiocarbonyl, di-(C₁-C₆)-alkylaminothiocarbonyl, (C₁-C₆)-alkylcarbonylamino (—NHCO—(C₁-C₆)-alkyl), (C₃-C₈)-cycloalkylcarbonylamino, (C₁-C₆)-alkylcarbonyl-(C₁-C₆)-alkylamino (—N(C₁-C₆)-alkyl-CO—(C₁-C₆)-alkyl), ((C₁-C₆)-alkoxycarbonyl)amino (—NHCOO—(C₁-C₆)-alkyl), ((C₁-C₆)-alkoxycarbonyl)-(C₁-C₆)-alkylamino (—N—(C₁-C₆)-alkyl-COO—(C₁-C₆)-alkyl), (C₁-C₆)-alkylcarbamoyloxy (—OCONH—(C₁-C₆)-alkyl), di-(C₁-C₆)-alkylcarbamoyloxy (—OCON—(C₁-C₆)-dialkyl), (N—(C₁-C₆)-alkylcarbamoyl)amino (—NHCONH—(C₁-C₆)-alkyl), (N,N-di-(C₁-C₆)-alkylcarbamoyl)amino (—NHCON—(C₁-C₆)-dialkyl), (N—(C₁-C₆)-alkylcarbamoyl)-(C₁-C₆)-alkylamino (—N(C₁-C₆)-alkyl-CONH—(C₁-C₆)-alkyl), (N,N-di-(C₁-C₆)-alkylcarbamoyl)-(C₁-C₆)-alkylamino (—N—(C₁-C₆)-alkyl-CON—(C₁-C₆)-dialkyl), carbamoylamino (—NHCONH₂), (carbamoyl)-N—(C₁-C₆)-alkylamino (—N—(C₁-C₆)-alkyl-CONH₂) or
   represent aryl or hetaryl, each of which is optionally mono- or polysubstituted by identical or different substituents, where (in the case of hetaryl) at least one carbonyl group may optionally be present and where possible substituents are in each case as follows: cyano, carboxyl, halogen, nitro, hydroxy, amino, tri-(C₁-C₆)-alkylsilyl, (C₃-C₈)-cycloalkyl, (C₃-C₈)-cycloalkyl-(C₃-C₈)-cycloalkyl, (C₁-C₆)-alkyl-(C₃-C₈)-cycloalkyl, halo-(C₃-C₈)-cycloalkyl, (C₁-C₆)-alkyl, (C₁-C₆)-haloalkyl, (C₁-C₆)-cyanoalkyl, (C₁-C₆)-hydroxyalkyl, hydroxycarbonyl-(C₁-C₆)-alkoxy, (C₁-C₆)-alkoxycarbonyl-(C₁-C₆)-alkyl, (C₁-C₆)-alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)-haloalkyl-(C₁-C₆)-alkoxyimino, (C₁-C₆)-alkylthio, (C₁-C₆)-haloalkylthio, (C₁-C₆)-alkoxy-(C₁-C₆)-alkylthio, (C₁-C₆)-alkylthio-(C₁-C₆)-alkyl, (C₁-C₆)-alkylsulfinyl, (C₁-C₆)-haloalkylsulfinyl, (C₁-C₆)-alkoxy-(C₁-C₆)-alkylsulfinyl, (C₁-C₆)-alkylsulfinyl-(C₁-C₆)-alkyl, (C₁-C₆)-alkylsulfonyl, (C₁-C₆)-haloalkylsulfonyl, (C₁-C₆)-alkoxy-(C₁-C₆)-alkylsulfonyl, (C₁-C₆)-alkylsulfonyl-(C₁-C₆)-alkyl, (C₁-C₆)-alkylsulfonyloxy, (C₁-C₆)-alkylcarbonyl, (C₁-C₆)-haloalkylcarbonyl, (C₁-C₆)-alkylcarbonyloxy, (C₁-C₆)-alkoxycarbonyl, (C₁-C₆)-haloalkoxycarbonyl, aminocarbonyl, (C₁-C₆)-alkylaminocarbonyl, di-(C₁-C₆)-alkylaminocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulfonylamino, (C₁-C₆)-alkylamino, di-(C₁-C₆)-alkylamino, aminosulfonyl, (C₁-C₆)-alkylaminosulfonyl, di-(C₁-C₆)-alkylaminosulfonyl, (C₁-C₆)-alkylsulfoximino, aminothiocarbonyl, (C₁-C₆)-alkylaminothiocarbonyl, di-(C₁-C₆)-alkylaminothiocarbonyl, (C₃-C₈)-cycloalkylamino or (C₁-C₆)-alkylcarbonylamino,
   where only one or two of the radicals R¹¹, R¹², R¹³, R¹⁵, R¹⁶, R¹⁷ represent a substituent other than hydrogen and, in the case that neither R¹¹ nor R¹⁵, neither R¹² nor R¹⁶ or neither R¹³ nor R¹⁷ represents hydrogen, R¹⁵, R¹⁶ and R¹⁷ each independently of one another only represent cyano, halogen, (C₃-C₆)-cycloalkyl, (C₃-C₆)-cycloalkyl-(C₃-C₆)-cycloalkyl, (C₁-C₄)-alkyl-(C₃-C₆)-cycloalkyl, halo-(C₃-C₆)-cycloalkyl, (C₁-C₄)-alkyl, (C₁-C₄)-haloalkyl or (C₁-C₄)-cyanoalkyl,
Q represents a partly saturated or saturated heterocyclic or heteroaromatic 8-, 9-, 10-, 11- or 12-membered fused bicyclic or tricyclic ring system which may optionally contain at least one carbonyl group and/or where the ring system is optionally mono- or polysubstituted by identical or different substituents, where the substituents independently of one another may be selected from cyano, halogen, nitro, acetyl, hydroxy, amino, SCN, tri-(C₁-C₆)-alkylsilyl, (C₃-C₈)-cycloalkyl, (C₃-C₈)-cycloalkyl-(C₃-C₈)-cycloalkyl, (C₁-C₆)-alkyl-(C₃-C₈)-cycloalkyl, halo-(C₃-C₈)-cycloalkyl, (C₁-C₆)-alkyl, (C₁-C₆)-haloalkyl, (C₁-C₆)-cyanoalkyl, (C₁-C₆)-hydroxyalkyl, hydroxycarbonyl-(C₁-C₆)-alkoxy, (C₁-C₆)-alkoxycarbonyl-(C₁-C₆)-alkyl, (C₁-C₆)-alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-alkynyloxy-(C₁-C₄)-alkyl, (C₂-C₆)-haloalkynyl, (C₂-C₆)-cyanoalkynyl, (C₁-C₆)-alkoxy, (C₁-C₆)-haloalkoxy, (C₁-C₆)-haloalkoxy-(C₁-C₆)-alkyl, (C₂-C₆)-alkenyloxy-(C₁-C₆)-alkyl, (C₂-C₆)-haloalkenyloxy-(C₁-C₆)-alkyl, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)-haloalkyl-(C₁-C₆)-alkoxyimino, (C₁-C₆)-alkylthio, (C₁-C₆)-haloalkylthio, (C₁-C₆)-alkoxy-(C₁-C₆)-alkylthio, (C₁-C₆)-alkylthio-(C₁-C₆)-alkyl, (C₁-C₆)-alkylsulfinyl, (C₁-C₆)-haloalkylsulfinyl, (C₁-C₆)-alkoxy-(C₁-C₆)-alkylsulfinyl, (C₁-C₆)-alkylsulfinyl-(C₁-C₆)-alkyl, (C₁-C₆)-alkylsulfonyl, (C₁-C₆)-haloalkylsulfonyl, (C₁-C₆)-alkoxy-(C₁-C₆)-alkylsulfonyl, (C₁-C₆)-alkylsulfonyl-(C₁-C₆)-alkyl, (C₁-C₆)-alkylsulfonyloxy, (C₁-C₆)-alkylcarbonyl, (C₁-C₆)-alkylcarbonyl-(C₁-C₆)-alkyl, (C₁-C₆)-alkylthiocarbonyl, (C₁-C₆)-haloalkylcarbonyl, (C₁-C₆)-alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)-alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulfonylamino, (C₁-C₆)-alkylamino, di-(C₁-C₆)-alkylamino, aminosulfonyl, (C₁-C₆)-alkylaminosulfonyl, di-(C₁-C₆)-alkylaminosulfonyl, (C₁-C₆)-alkylsulfoximino, aminothiocarbonyl, (C₁-C₆)-alkylaminothiocarbonyl, di-(C₁-C₆)-alkylaminothiocarbonyl, (C₃-C₈)-cycloalkylamino, NHCO—(C₁-C₆)-alkyl ((C₁-C₆)-alkylcarbonylamino),
   or where the substituents independently of one another may be selected from phenyl and a 5- or 6-membered heteroaromatic ring, where phenyl and the ring may optionally be mono- or polysubstituted by identical or different substituents selected from the group consisting of C₁-C₆-alkyl, C₂-C₆-alkenyl, C₂-C₆-alkynyl, C₃-C₆-cycloalkyl, C₁-C₆-haloalkyl, C₂-C₆-haloalkenyl, C₂-C₆-haloalkynyl, C₃-C₆-halocycloalkyl, halogen, CN, NO₂, C₁-C₄-alkoxy, C₁-C₄-haloalkoxy, and
n represents 0, 1 or 2.

Configuration 2-2

Compounds of the formula (I) in which preferably

in the case that [symbol] represents a single bond and a double bond, and Aa to Ae thus form an aromatic ring,
Aa represents nitrogen or carbon,
Ab represents nitrogen, oxygen, sulfur or C(R¹¹),
Ac represents nitrogen, oxygen, sulfur or C(R¹²),
Ad represents nitrogen, oxygen, sulfur or C(R¹³),
Ae represents nitrogen or carbon,
where at most three of the groups Aa, Ab, Ac, Ad and Ae may represent nitrogen and at most one may represent oxygen or at most one may represent sulfur,
or, in the case that [symbol] represent exclusively single bonds,
Aa represents carbon,
Ab represents sulfur, oxygen or C(R¹¹)(R¹⁵),
Ac represents sulfur, oxygen or C(R¹²)(R¹⁶) and
Ad represents sulfur, oxygen or C(R¹³)(R¹⁷),
Ae represents carbon,
where at most one of the groups Aa, Ab, Ac, Ad and Ae may represent oxygen or sulfur,
R¹ preferably represents (C₁-C₄)-alkyl, (C₁-C₄)-hydroxyalkyl, (C₁-C₄)-haloalkyl, (C₂-C₄)-alkenyl, (C₂-C₄)-haloalkenyl, (C₂-C₄)-alkynyl, (C₂-C₄)-haloalkynyl, (C₃-C₆)-cycloalkyl, (C₁-C₄)-alkylthio-(C₁-C₄)-alkyl, (C₁-C₄)-haloalkylthio-(C₁-C₄)-alkyl, (C₁-C₄)-alkylsulfinyl-(C₁-C₄)-alkyl, (C₁-C₄)-alkylsulfonyl-(C₁-C₄)-alkyl,
   or represents (C₁-C₄)-alkyl, (C₂-C₄)-alkenyl, (C₂-C₄)-alkynyl, (C₃-C₆)-cycloalkyl, each of which is optionally mono- or disubstituted by identical or different aryl or heterocyclyl substituents, where aryl or heterocyclyl may in each case optionally be mono- or disubstituted by identical or different halogen, (C₁-C₄)-alkyl, (C₁-C₄)-haloalkyl, (C₃-C₄)-cycloalkyl, (C₁-C₄)-alkoxy or (C₁-C₄)-alkylthio substituents,
R¹¹, R¹², R¹³, R¹⁵, R¹⁶, R¹⁷ independently of one another preferably represent hydrogen, cyano, halogen, nitro, hydroxy, amino, tri-(C₁-C₄)-alkylsilyl, (C₃-C₆)-cycloalkyl, (C₃-C₆)-cycloalkyl-(C₃-C₆)-cycloalkyl, (C₁-C₄)-alkyl-(C₃-C₆)-cycloalkyl, halo-(C₃-C₆)-cycloalkyl, (C₁-C₄)-alkyl, (C₁-C₄)-haloalkyl, (C₁-C₄)-cyanoalkyl, (C₁-C₄)-hydroxyalkyl, (C₁-C₄)-alkoxy-(C₁-C₄)-alkyl, (C₂-C₄)-alkenyl, (C₂-C₄)-haloalkenyl, (C₂-C₄)-cyanoalkenyl, (C₂-C₄)-alkynyl, (C₂-C₄)-haloalkynyl, (C₂-C₄)-cyanoalkynyl, (C₁-C₄)-alkoxy, (C₁-C₄)-haloalkoxy, (C₁-C₄)-cyanoalkoxy, (C₁-C₄)-alkoxy-(C₁-C₄)-alkoxy, (C₁-C₄)-alkylhydroxyimino, (C₁-C₄)-alkoxyimino, (C₁-C₄)-alkyl-(C₁-C₄)-alkoxyimino, (C₁-C₄)-haloalkyl-(C₁-C₄)-alkoxyimino, (C₁-C₄)-alkylthio, (C₁-C₄)-haloalkylthio, (C₁-C₄)-alkylthio-(C₁-C₄)-alkyl, (C₁-C₄)-alkylsulfinyl, (C₁-C₄)-haloalkylsulfinyl, (C₁-C₄)-alkylsulfinyl-(C₁-C₄)-alkyl, (C₁-C₄)-alkylsulfonyl, (C₁-C₄)-haloalkylsulfonyl, (C₁-C₄)-alkylsulfonyl-(C₁-C₄)-alkyl, (C₁-C₄)-alkylsulfonyloxy, (C₁-C₄)-alkylcarbonyl, (C₁-C₄)-haloalkylcarbonyl, aminocarbonyl, aminothiocarbonyl, (C₁-C₄)-alkylaminocarbonyl, di-(C₁-C₄)-alkylaminocarbonyl, (C₃-C₆)-cycloalkylaminocarbonyl, (C₁-C₄)-alkylsulfonylamino, (C₁-C₄)-alkylamino, di-(C₁-C₄)-alkylamino, aminosulfonyl, (C₁-C₄)-alkylaminosulfonyl, di-(C₁-C₄)-alkylaminosulfonyl, aminothiocarbonyl, (C₁-C₄)-alkylcarbonylamino, (C₁-C₄)-alkylcarbonylamino (—NHCO—(C₁-C₄)-alkyl), (C₃-C₆)-cycloalkylcarbonylamino, (C₁-C₄)-alkylcarbonyl-(C₁-C₄)-alkylamino (—N—(C₁-C₄)-alkyl-CO—(C₁-C₆)-alkyl), ((C₁-C₄)-alkoxycarbonyl)amino (—NHCOO—(C₁-C₄)-alkyl), ((C₁-C₄)-alkoxycarbonyl)-(C₁-C₄)-alkylamino (—N—(C₁-C₄)-alkyl-COO—(C₁-C₆)-alkyl), (C₁-C₄)-alkylcarbamoyloxy (—OCONH—(C₁-C₄)-alkyl), di-(C₁-C₄)-alkylcarbamoyloxy (—OCON—(C₁-C₄)-dialkyl), (N—(C₁-C₄)-alkylcarbamoyl)amino (—NHCONH—(C₁-C₄)-alkyl), (N,N-di-(C₁-C₄)-alkylcarbamoyl)amino (—NHCON—(C₁-C₄)-dialkyl), (N—(C₁-C₄)-alkylcarbamoyl)-(C₁-C₄)-alkylamino (—N—(C₁-C₄)-alkyl-CONH—(C₁-C₄)-alkyl), (N,N-di-(C₁-C₄)-alkylcarbamoyl)-(C₁-C₄)-alkylamino (—N—(C₁-C₄)-alkyl-CON—(C₁-C₄)-dialkyl), carbamoylamino (—NHCONH₂), (carbamoyl)-N—(C₁-C₄)-alkylamino (—N(C₁-C₄)-alkyl-CONH₂) or preferably represent phenyl or 5- or 6-membered hetaryl, each of which is optionally mono- or disubstituted by identical or different substituents, where (in the case of hetaryl) optionally at least one carbonyl group may be present and the heteroatoms are selected from N, S and O, and where possible substituents are in each case as follows: cyano, halogen, acetyl, (C₃-C₆)-cycloalkyl, (C₃-C₆)-cycloalkyl-(C₃-C₆)-cycloalkyl, (C₁-C₄)-alkyl-(C₃-C₆)-cycloalkyl, halo-(C₃-C₆)-cycloalkyl, (C₁-C₄)-alkyl, (C₁-C₄)-haloalkyl, (C₁-C₄)-cyanoalkyl, (C₁-C₄)-alkoxy-(C₁-C₄)-alkyl, (C₂-C₄)-alkenyl, (C₂-C₄)-alkynyl, (C₂— (C₁-C₄)-alkoxy, (C₁-C₄)-alkylthio, (C₁-C₄)-haloalkylthio, (C₁-C₄)-alkylthio-(C₁-C₄)-alkyl, (C₁-C₄)-alkylsulfinyl, (C₁-C₄)-haloalkylsulfinyl, (C₁-C₄)-alkylsulfinyl-(C₁-C₄)-alkyl, (C₁-C₄)-alkylsulfonyl, (C₁-C₄)-haloalkylsulfonyl, (C₁-C₄)-alkylsulfonyl-(C₁-C₄)-alkyl, (C₁-C₄)-alkylsulfonyloxy, (C₁-C₄)-alkylcarbonyl, aminocarbonyl, (C₁-C₄)-alkylaminocarbonyl, di-(C₁-C₄)-alkylaminocarbonyl, (C₁-C₄)-alkylsulfonylamino, aminosulfonyl, (C₁-C₄)-alkylaminosulfonyl, di-(C₁-C₄)-alkylaminosulfonyl or (C₁-C₄)-alkylcarbonylamino,
   where only one or two of the radicals R¹¹, R¹², R¹³, R¹⁵, R¹⁶, R¹⁷ represent a substituent other than hydrogen,
and, in the case that neither R¹¹ nor R¹⁵, neither R¹² nor R¹⁶ or neither R¹³ nor R¹⁷ represents hydrogen, R¹⁵, R¹⁶ and R¹⁷ each independently of one another only represent cyano, halogen, (C₃-C₆)-cycloalkyl, (C₃-C₆)-cycloalkyl-(C₃-C₆)-cycloalkyl, (C₁-C₄)-alkyl-(C₃-C₆)-cycloalkyl, halo-(C₃-C₆)-cycloalkyl, (C₁-C₄)-alkyl, (C₁-C₄)-haloalkyl or (C₁-C₄)-cyanoalkyl,
Q preferably represents a heteroaromatic 9-membered or 12-membered fused bicyclic or tricyclic ring system from the group consisting of Q1 to Q13 and Q15 to Q20

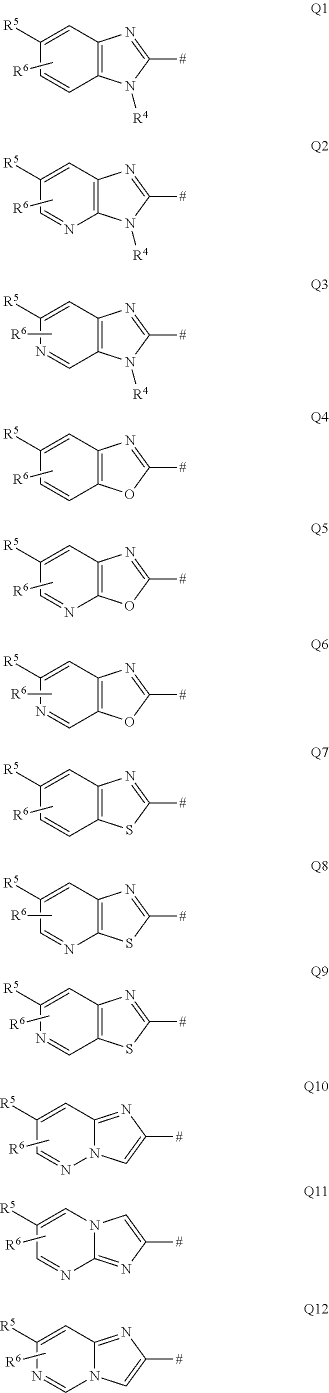

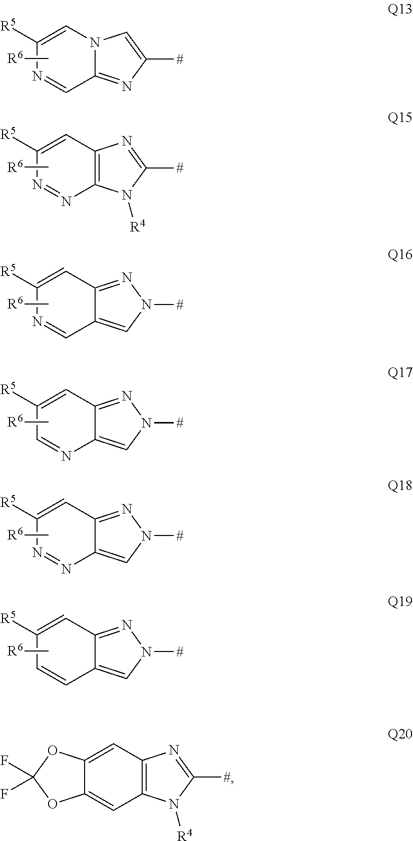

R⁴ preferably represents (C₁-C₄)-alkyl, (C₁-C₄)-haloalkyl, (C₁-C₄)-cyanoalkyl, (C₁-C₄)-hydroxyalkyl, (C₁-C₄)-alkoxy-(C₁-C₄)-alkyl, (C₁-C₄)-haloalkoxy-(C₁-C₄)-alkyl, (C₂-C₄)-alkenyl, (C₂-C₄)-alkenyloxy-(C₁-C₄)-alkyl, (C₂-C₄)-haloalkenyloxy-(C₁-C₄)-alkyl, (C₂-C₄)-haloalkenyl, (C₂-C₄)-cyanoalkenyl, (C₂-C₄)-alkynyl, (C₂-C₄)-alkynyloxy-(C₁-C₄)-alkyl, (C₂-C₄)-haloalkynyl, (C₃-C₆)-cycloalkyl, (C₃-C₆)-cycloalkyl-(C₃-C₆)-cycloalkyl, (C₁-C₄)-alkyl-(C₃-C₆)-cycloalkyl, halo-(C₃-C₆)-cycloalkyl, (C₁-C₄)-alkylthio-(C₁-C₄)-alkyl, (C₁-C₄)-alkylsulfinyl-(C₁-C₄)-alkyl, (C₁-C₄)-alkylsulfonyl-(C₁-C₄)-alkyl or (C₁-C₄)-alkylcarbonyl-(C₁-C₄)-alkyl,
R⁵, R⁶ independently of one another preferably represent hydrogen, cyano, halogen, (C₁-C₄)-alkyl, (C₁-C₄)-haloalkyl, (C₂-C₄)-alkenyl, (C₂-C₄)-haloalkenyl, (C₂-C₄)-alkynyl, (C₂-C₄)-haloalkynyl, (C₃-C₆)-cycloalkyl, (C₃-C₆)-cycloalkyl-(C₃-C₆)-cycloalkyl, (C₁-C₄)-alkyl-(C₃-C₆)-cycloalkyl, (C₁-C₄)-alkoxy, (C₁-C₄)-haloalkoxy, (C₁-C₄)-alkoxyimino, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, (C₁-C₄)-alkylsulfonyloxy, (C₁-C₄)-alkylcarbonyl, (C₁-C₄)-haloalkylcarbonyl, aminocarbonyl, (C₁-C₄)-alkylaminocarbonyl, di-(C₁-C₄)-alkylaminocarbonyl, (C₁-C₄)-alkylsulfonylamino, (C₁-C₄)-alkylamino, di-(C₁-C₄)-alkylamino, aminosulfonyl, (C₁-C₄)-alkylaminosulfonyl or di-(C₁-C₄)-alkylaminosulfonyl,
   and
n preferably represents 0, 1 or 2.

Configuration 3-2

Compounds of the formulae (Ia), (Ib), (Id), (Ie), (Ig) to (Ik) and (Im) to (It),

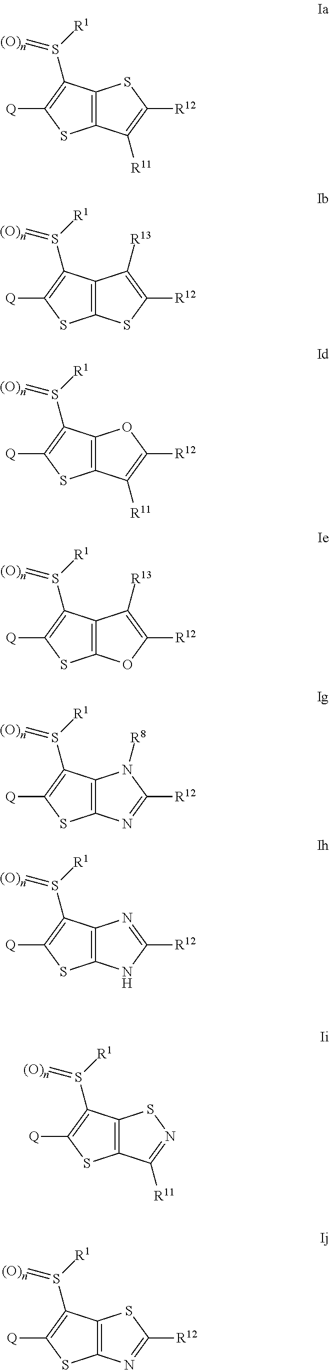

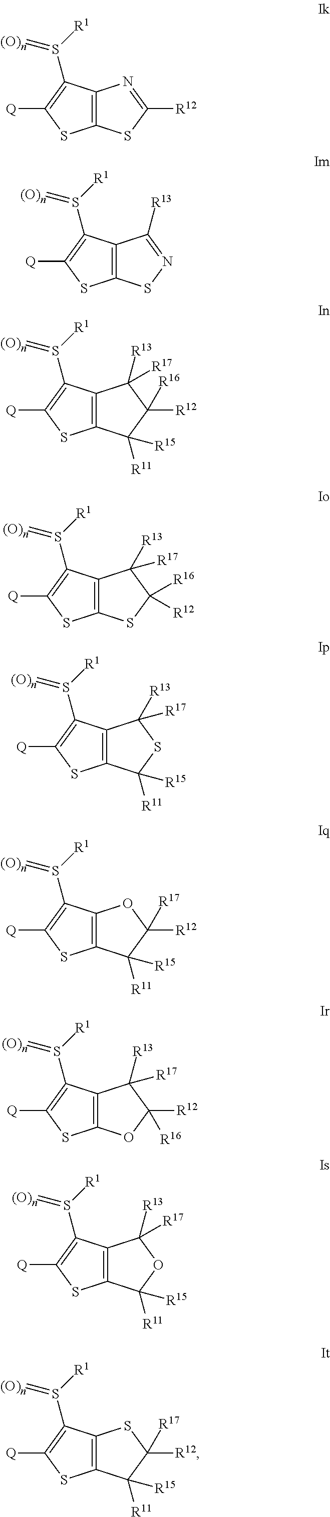

R¹ particularly preferably represents (C₁-C₄)-alkyl-, (C₁-C₄)-haloalkyl or (C₃-C₆)-cycloalkyl,
R¹¹, R¹², R¹³, R¹⁵, R¹⁶, R¹⁷ independently of one another particularly preferably represent hydrogen, cyano, halogen, nitro, hydroxy, (C₃-C₆)-cycloalkyl, (C₁-C₄)-alkyl-(C₃-C₆)-cycloalkyl, halo-(C₃-C₆)-cycloalkyl, (C₁-C₄)-alkyl, (C₁-C₄)-haloalkyl, (C₁-C₄)-alkoxy-(C₁-C₄)-alkyl, (C₁-C₄)-alkoxy, (C₁-C₄)-haloalkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, aminosulfonyl, (C₁-C₄)-alkylaminosulfonyl, di-(C₁-C₄)-alkylaminosulfonyl, aminocarbonyl, (C₁-C₄)-alkylaminocarbonyl, di-(C₁-C₄)-alkylaminocarbonyl, (C₃-C₆)-cycloalkylaminocarbonyl, (C₁-C₄)-alkylcarbonylamino (—NHCO(C₁-C₄)-alkyl), (C₃-C₆)-cycloalkylcarbonylamino, (C₁-C₄)-alkylcarbonyl-(C₁-C₄)-alkylamino (—N—(C₁-C₄)-alkyl-CO—(C₁-C₆)-alkyl), ((C₁-C₄)-alkoxycarbonyl)amino (—NHCOO—(C₁-C₄)-alkyl), (N—(C₁-C₄)-alkylcarbamoyl)amino (—NHCONH—(C₁-C₄)-alkyl), (N,N-di-(C₁-C₄)-alkylcarbamoyl)amino (—NHCON—(C₁-C₄)-dialkyl), (N—(C₁-C₄)-alkylcarbamoyl)-(C₁-C₄)-alkylamino (—N—(C₁-C₄)-alkyl-CONH—(C₁-C₄)-alkyl), (N,N-di-(C₁-C₄)-alkylcarbamoyl)-(C₁-C₄)-alkylamino (—N—(C₁-C₄)-alkyl-CON—(C₁-C₄)-dialkyl), carbamoylamino (—NHCONH₂), (carbamoyl)-N—(C₁-C₄)-alkylamino (—N—(C₁-C₄)-alkyl-CONH₂) or particularly preferably represent phenyl, triazole or pyrazole, each of which is optionally mono- or disubstituted by identical or different substituents, possible substituents being in each case: cyano, halogen, (C₃-C₆)-cycloalkyl, (C₃-C₆)-cycloalkyl-(C₃-C₆)-cycloalkyl, (C₁-C₄)-alkyl-(C₃-C₆)-cycloalkyl, halo-(C₃-C₆)-cycloalkyl, (C₁-C₄)-alkyl, (C₁-C₄)-haloalkyl, (C₁-C₄)-cyanoalkyl, (C₂-C₄)-alkenyl, (C₂-C₄)-alkynyl, (C₁-C₄)-alkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, (C₁-C₄)-alkylsulfonyloxy, (C₁-C₄)-alkylcarbonyl, aminocarbonyl, (C₁-C₄)-alkylaminocarbonyl, di-(C₁-C₄)-alkylaminocarbonyl, (C₁-C₄)-alkylsulfonylamino, aminosulfonyl, (C₁-C₄)-alkylaminosulfonyl, di-(C₁-C₄)-alkylaminosulfonyl, (C₁-C₄)-alkylcarbonylamino,
   where only one or two of the radicals R¹¹, R¹², R¹³, R¹⁵, R¹⁶, R¹⁷ represent a substituent other than hydrogen,
   and, in the case that neither R¹¹ nor R¹⁵, neither R¹² nor R¹⁶ or neither R¹³ nor R¹⁷ represents hydrogen, R¹⁴, R¹⁵, R¹⁶ and R¹⁷ each independently of one another only represent cyano, halogen, (C₃-C₆)-cycloalkyl, (C₃-C₆)-cycloalkyl-(C₃-C₆)-cycloalkyl, (C₁-C₄)-alkyl-(C₃-C₆)-cycloalkyl, halo-(C₃-C₆)-cycloalkyl, (C₁-C₄)-alkyl or (C₁-C₄)-haloalkyl,
Q particularly preferably represents a heteroaromatic 9-membered fused bicyclic ring system from the group consisting of Q1, Q2, Q3, Q5, Q6, Q8, Q9, Q10, Q12, Q15 and Q20,

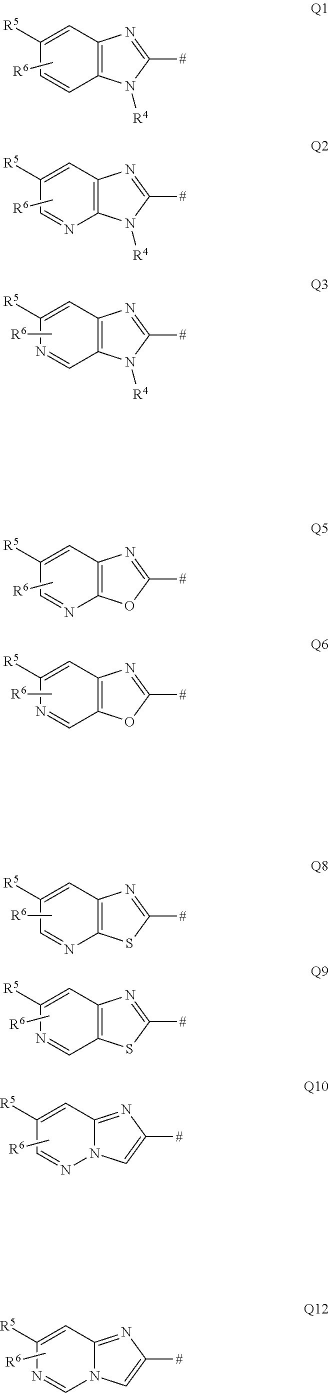

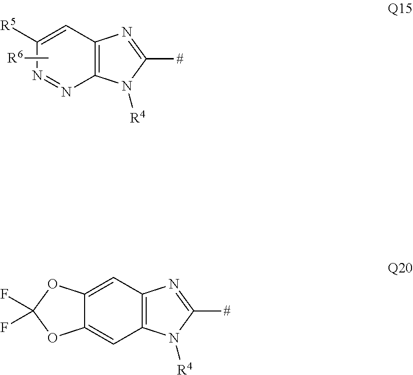

R⁴ particularly preferably represents (C₁-C₄)-alkyl, (C₁-C₄)-haloalkyl, (C₁-C₄)-cyanoalkyl or (C₁-C₄)-alkoxy-(C₁-C₄)-alkyl,
R⁵ particularly preferably represents halogen, (C₁-C₄)-haloalkyl, (C₁-C₄)-haloalkoxy, (C₁-C₄)-haloalkylthio, (C₁-C₄)-haloalkylsulfinyl or (C₁-C₄)-haloalkylsulfonyl,
R⁶ particularly preferably represents hydrogen, cyano, halogen or (C₁-C₄)-alkyl and
n particularly preferably represents 0, 1 or 2.

Configuration 4-2

Very particular preference is given to compounds of the formulae (Ia), (Ib), (Id), (Ie), (Ii) to (Ik) and (In) to (It)

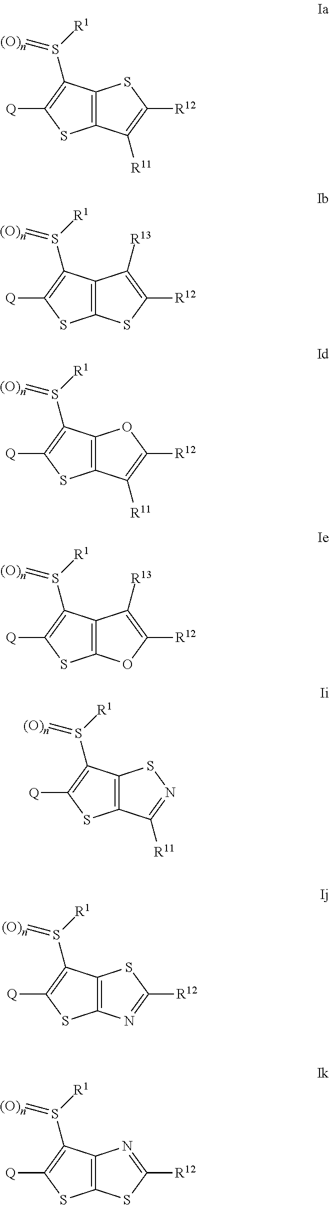

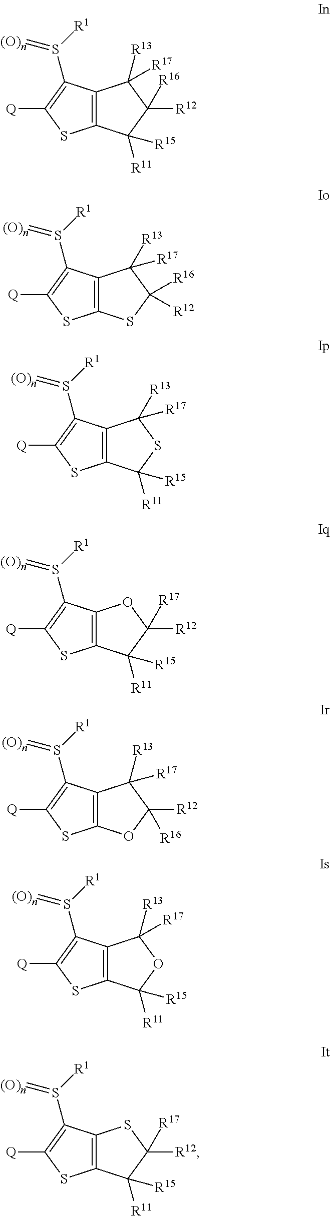

R¹ very particularly preferably represents (C₁-C₄)-alkyl, (C₃-C₆)-cycloalkyl or (C₁-C₄)-haloalkyl,
R¹¹, R¹³, R¹⁵, R¹⁶, R¹⁷ independently of one another very particularly preferably represent hydrogen, halogen, (C₁-C₄)-haloalkyl or (C₁-C₄)-alkyl,
R¹² very particularly preferably represents hydrogen, cyano, halogen, nitro, hydroxy, (C₃-C₆)-cycloalkyl, (C₁-C₄)-alkyl-(C₃-C₆)-cycloalkyl, halo-(C₃-C₆)-cycloalkyl, (C₁-C₄)-alkyl, (C₁-C₄)-haloalkyl, (C₁-C₄)-alkoxy-(C₁-C₄)-alkyl, (C₁-C₄)-alkoxy, (C₁-C₄)-haloalkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, aminosulfonyl, (C₁-C₄)-alkylaminosulfonyl, di-(C₁-C₄)-alkylaminosulfonyl, aminocarbonyl, (C₁-C₄)-alkylaminocarbonyl, di-(C₁-C₄)-alkyl-aminocarbonyl, (C₃-C₆)-cycloalkylaminocarbonyl, (C₁-C₄)-alkylcarbonylamino (—NHCO—(C₁-C₄)-alkyl), (C₃-C₆)-cycloalkylcarbonylamino, (C₁-C₄)-alkylcarbonyl-(C₁-C₄)-alkylamino (—N—(C₁-C₄)-alkyl-CO—(C₁-C₆)-alkyl), ((C₁-C₄)-alkoxycarbonyl)amino (—NHCOO—(C₁-C₄)-alkyl), (N—(C₁-C₄)-alkylcarbamoyl)amino (—NHCONH—(C₁-C₄)-alkyl), (N,N-di-(C₁-C₄)-alkylcarbamoyl)amino (—NHCON—(C₁-C₄)-dialkyl), (N—(C₁-C₄)-alkylcarbamoyl)-(C₁-C₄)-alkylamino (—N—(C₁-C₄)-alkyl-CONH—(C₁-C₄)-alkyl), carbamoylamino (—NHCONH₂), (carbamoyl)-N—(C₁-C₄)-alkylamino (—N—(C₁-C₄)-alkyl-CONH₂)
   or very particularly preferably represents phenyl, pyrazole or triazole, each of which is optionally mono- or disubstituted by identical or different substituents, possible substituents being in each case: cyano, halogen, (C₃-C₆)-cycloalkyl, (C₁-C₄)-alkyl-(C₃-C₆)-cycloalkyl, halo-(C₃-C₆)-cycloalkyl, (C₁-C₄)-alkyl, (C₁-C₄)-haloalkyl, (C₁-C₄)-alkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, (C₁-C₄)-alkylsulfonyloxy, aminocarbonyl, (C₁-C₄)-alkylaminocarbonyl, di-(C₁-C₄)-alkylaminocarbonyl, (C₁-C₄)-alkylcarbonylamino,
where only one or two of the radicals R¹¹, R¹², R¹³, R¹⁵, R¹⁶, R¹⁷ represent a substituent other than hydrogen,
   and, in the case that neither R¹¹ nor R¹⁵, neither R¹² nor R¹⁶ or neither R¹³ nor R¹⁷ represents hydrogen, R¹⁴, R¹⁵, R¹⁶ and R¹⁷ each independently of one another only represent halogen, (C₁-C₄)-haloalkyl or (C₁-C₄)-alkyl,
Q very particularly preferably represents a heteroaromatic 9-membered fused bicyclic ring system from the group consisting of Q1, Q2, Q3, Q5, Q6, Q12, Q10, Q15 and Q20

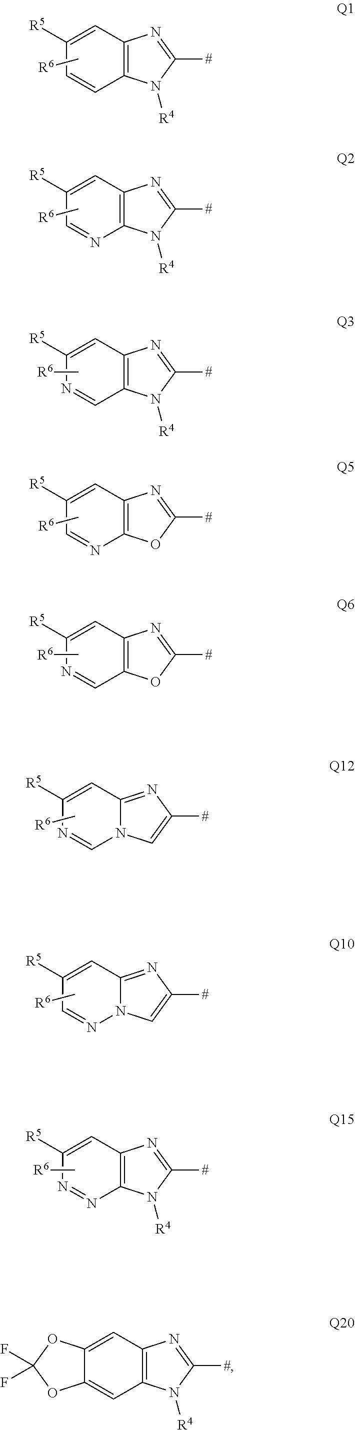

where
R⁴ very particularly preferably represents (C₁-C₄)-alkyl,
R⁵ very particularly preferably represents halogen, (C₁-C₄)-haloalkyl, (C₁-C₄)-haloalkoxy or (C₁-C₄)-haloalkylthio,
R⁶ very particularly preferably represents hydrogen and
n very particularly preferably represents 0, 1 or 2.

Configuration 5-2

Emphasis is given to compounds of the formulae (Ia), (Ib), (Id), (Ii), (Ij), (Ik) and (In),

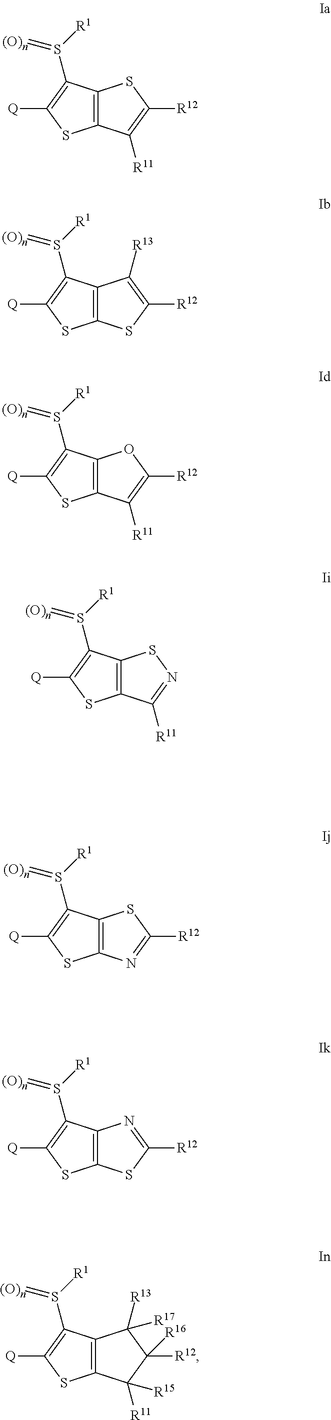

where
R¹ most preferably represents (C₁-C₄)-alkyl,
R¹³, R¹⁵, R¹⁶, R¹⁷ independently of one another most preferably represent hydrogen,
R¹¹ most preferably represents hydrogen, (C₁-C₄)-alkyl or halogen,
R¹² most preferably represents hydrogen, halogen, (C₁-C₄)-alkyl, cyclopropyl, (C₁-C₄)-haloalkyl, (C₁-C₄)-haloalkoxy, aminocarbonyl, (C₁-C₄)-alkylaminocarbonyl, di-(C₁-C₄)-alkylaminocarbonyl, (C₁-C₄)-alkylcarbonylamino, (C₃-C₆)-cycloalkylaminocarbonyl, (C₁-C₄)-alkylcarbonylamino (—NHCO—(C₁-C₄)-alkyl), cyclopropylcarbonylamino, (C₁-C₄)-alkylcarbonyl-(C₁-C₄)-alkylamino (—N—(C₁-C₄)-alkyl-CO—(C₁-C₆)-alkyl), ((C₁-C₄)-alkoxycarbonyl)amino (—NHCOO—(C₁-C₄)-alkyl), (N—(C₁-C₄)-alkylcarbamoyl)amino (—NHCONH—(C₁-C₄)-alkyl), carbamoylamino (—NHCONH₂), (carbamoyl)-N—(C₁-C₄)-alkylamino (—N—(C₁-C₄)-alkyl-CONH₂)
   or represents phenyl, pyrazole or triazole, each of which is optionally mono- or disubstituted by identical or different halogens,
Q most preferably represents a heteroaromatic 9-membered fused bicyclic ring system from the group consisting of Q2, Q3 and Q15,

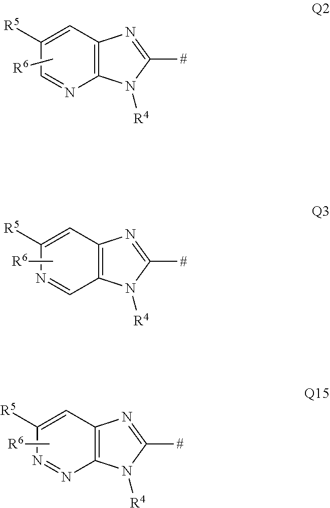

R⁴ most preferably represents (C₁-C₄)-alkyl,
R⁵ most preferably represents (C₁-C₄)-haloalkyl,
R⁶ most preferably represents hydrogen and
n most preferably represents 0 or 2.

Configuration 6-2

Special preference is given to compounds of the formulae (Ia), (Id), (In)

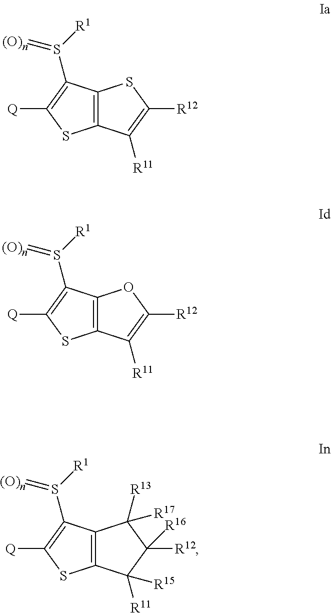

where
R¹ especially represents ethyl,
R¹³, R¹⁵, R¹⁶, R¹⁷ especially represent hydrogen,
R¹² especially represents hydrogen or trifluoromethyl,
R¹¹ especially represents hydrogen,
Q especially represents Q3,
R⁴ especially represents methyl,
R⁵ especially represents trifluoromethyl,
R⁶ especially represents hydrogen and
n especially represents 2.

Hereinbelow, the statements concerning the compounds of the formula (I) do, of course, also apply to the compounds of the formulae (Ia), (Ib), (Id), (Ie), (Ig) to (Ik) and (Im) to (It) which are embraced by formula (I).

The expression Configuration x hereinafter is synonymous with Configuration x-1 or Configuration x-2 where x represents 1, 2, 3, 4, 5 or 6.

In a preferred embodiment, the invention relates to compounds of the formula (I) where

R¹ represents (C₁-C₄)-alkyl, (C₁-C₄)-haloalkyl or (C₃-C₆)-cycloalkyl and
Aa, Ab, Ac, Ad, R⁴, R⁵, R⁶, R⁷, R⁸, R¹¹, R¹², R¹³, R¹⁵, R¹⁶, R¹⁷, Q and n have the meanings described in Configuration (1) or Configuration (2) or Configuration (5) or Configuration (6).

In a particularly preferred embodiment, the invention relates to compounds of the formula (I) where

R¹ represents (C₁-C₄)-alkyl and

Aa, Ab, Ac, Ad, R⁴, R⁵, R⁶, R⁷, R⁸, R¹¹, R¹², R¹³, R¹⁵, R¹⁶, R¹⁷, Q and n have the meanings described in Configuration (1) or Configuration (2) or Configuration (3) or Configuration (4) or Configuration (6).

In a very particularly preferred embodiment, the invention relates to compounds of the formula (I) where

R¹ represents ethyl and
Aa, Ab, Ac, Ad, R⁴, R⁵, R⁶, R⁷, R⁸, R¹¹, R¹², R¹³, R¹⁵, R¹⁶, R¹⁷, Q and n have the meanings described in Configuration (1) or Configuration (2) or Configuration (3) or Configuration (4) or Configuration (5).

In a preferred embodiment, the invention relates to compounds of the formula (I) where
Q represents a heteroaromatic 9-membered or 12-membered fused bicyclic or tricyclic ring system from the group consisting of Q1 to Q13 and Q15 to Q21

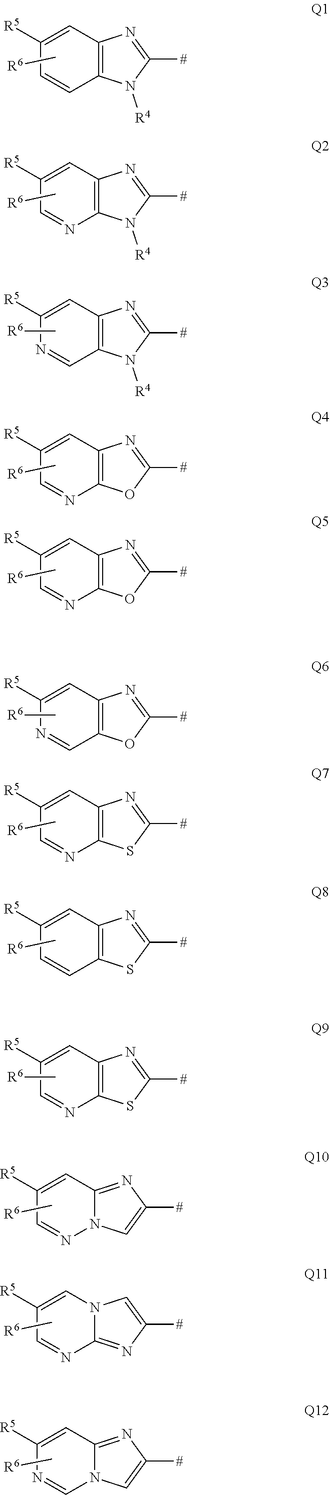

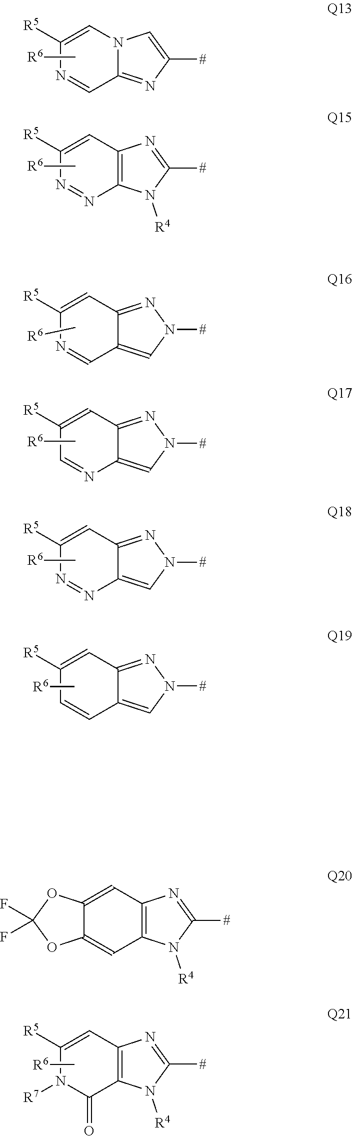

and Aa, Ab, Ac, Ad, R⁴, R⁵, R⁶, R⁷, R⁸, R¹¹, R¹², R¹³, R¹⁵, R¹⁶, R¹⁷, R¹ and n have the meanings described in Configuration (1) or Configuration (3) or Configuration (4) or Configuration (5) or Configuration (6).

In a particularly preferred embodiment, the invention relates to compounds of the formula (I) where
Q represents a heteroaromatic 9-membered or 12-membered fused bicyclic or tricyclic ring system from the group consisting of Q1, Q2, Q3, Q5, Q6, Q8, Q9, Q10, Q12, Q15, Q20 and Q21
   and Aa, Ab, Ac, Ad, R⁴, R⁵, R⁶, R⁷, R⁸, R¹¹, R¹², R¹³, R¹⁵, R¹⁶, R¹⁷, R¹ and n have the meanings described in Configuration (1) or Configuration (2) or Configuration (4) or Configuration (5) or Configuration (6).

In a very particularly preferred embodiment, the invention relates to compounds of the formula (I) where
Q represents a heteroaromatic 9-membered or 12-membered fused bicyclic or tricyclic ring system from the group consisting of Q1, Q2, Q3, Q5, Q6, Q12, Q10, Q15, Q20 and Q21
   and Aa, Ab, Ac, Ad, R⁴, R⁵, R⁶, R⁷, R⁸, R¹¹, R¹², R¹³, R¹⁵, R¹⁶, R¹⁷, R¹ and n have the meanings described in Configuration (1) or Configuration (2) or Configuration (3) or Configuration (5) or Configuration (6).

In a most preferred embodiment, the invention relates to compounds of the formula (I) where
Q represents a heteroaromatic 9-membered fused bicyclic ring system from the group consisting of Q2, Q3, Q15 and Q21
   and Aa, Ab, Ac, Ad, R⁴, R⁵, R⁶, R⁷, R⁸, R¹¹, R¹², R¹³, R¹⁵, R¹⁶, R¹⁷, R¹ and n have the meanings described in Configuration (1) or Configuration (2) or Configuration (3) or Configuration (4).

In a preferred embodiment, the invention relates to compounds of the formula (I) where
R⁴ represents (C₁-C₄)-alkyl, (C₁-C₄)-haloalkyl, (C₁-C₄)-cyanoalkyl or (C₁-C₄)-alkoxy-(C₁-C₄)-alkyl and
   Aa, Ab, Ac, Ad, R¹, R⁵, R⁶, R⁷, R⁸, R¹¹, R¹², R¹³, R¹⁵, R¹⁶, R¹⁷, Q and n have the meanings described in Configuration (1) or Configuration (2) or Configuration (4) or Configuration (5) or Configuration (6).

In a particularly preferred embodiment, the invention relates to compounds of the formula (I) where

R⁴ represents (C₁-C₄)-alkyl and
Aa, Ab, Ac, Ad, R¹, R⁵, R⁶, R⁷, R⁸, R¹¹, R¹², R¹³, R¹⁵, R¹⁶, R¹⁷, Q and n have the meanings described in Configuration (1) or Configuration (2) or Configuration (3) or Configuration (6).

In a very particularly preferred embodiment, the invention relates to compounds of the formula (I) where

R⁴ represents methyl and
Aa, Ab, Ac, Ad, R¹, R⁵, R⁶, R⁷, R⁸, R¹¹, R¹², R¹³, R¹⁵, R¹⁶, R¹⁷, Q and n have the meanings described in Configuration (1) or Configuration (2) or Configuration (3) or Configuration (4) or Configuration (5).

In a preferred embodiment, the invention relates to compounds of the formula (I) where
R⁵ represents halogen, (C₁-C₄)-haloalkyl, (C₁-C₄)-haloalkoxy, (C₁-C₄)-haloalkylthio, (C₁-C₄)-haloalkylsulfinyl, (C₁-C₄)-haloalkylsulfonyl or represents optionally monocyano-substituted (C₃-C₆)-cycloalkyl and
   Aa, Ab, Ac, Ad, R¹, R⁴, R⁶, R⁷, R⁸, R¹¹, R¹², R¹³, R¹⁵, R¹⁶, R¹⁷, Q and n have the meanings described in Configuration (1) or Configuration (2) or Configuration (4) or Configuration (5) or Configuration (6).

In a particularly preferred embodiment, the invention relates to compounds of the formula (I) where
R⁵ represents halogen, (C₁-C₄)-haloalkyl, (C₁-C₄)-haloalkoxy, (C₁-C₄)-haloalkylthio, (C₁-C₄)-haloalkylsulfinyl, (C₁-C₄)-haloalkylsulfonyl or represents optionally monocyano-substituted (C₃-C₆)-cycloalkyl and
   Aa, Ab, Ac, Ad, R¹, R⁴, R⁶, R⁷, R⁸, R¹¹, R¹², R¹³, R¹⁵, R¹⁶, R¹⁷, Q and n have the meanings described in Configuration (1) or Configuration (2) or Configuration (3) or Configuration (5) or Configuration (6).

In a very particularly preferred embodiment, the invention relates to compounds of the formula (I) where
R⁵ represents (C₁-C₄)-haloalkyl, (C₁-C₄)-haloalkylthio, (C₁-C₄)-haloalkylsulfonyl, (C₁-C₄)-haloalkylsulfinyl or monocyano-substituted cyclopropyl and
   Aa, Ab, Ac, Ad, R¹, R⁴, R⁶, R⁷, R⁸, R¹¹, R¹², R¹³, R¹⁵, R¹⁶, R¹⁷, Q and n have the meanings described in Configuration (1) or Configuration (2) or Configuration (3) or Configuration (4) or Configuration (6).

In a specially preferred embodiment, the invention relates to compounds of the formula (I) where
R⁵ represents (C₁-C₄)-haloalkyl, (C₁-C₄)-haloalkylthio, (C₁-C₄)-haloalkylsulfonyl or (C₁-C₄)-haloalkylsulfinyl and Aa, Ab, Ac, Ad, R¹, R⁴, R⁶, R⁷, R⁸, R¹¹, R¹², R¹³, R¹⁵, R¹⁶, R¹⁷, Q and n have the meanings described in Configuration (1) or Configuration (2) or Configuration (3) or Configuration (4) or Configuration (5) or Configuration (6).

In a further specially preferred embodiment, the invention relates to compounds of the formula (I) where
R⁵ represents trifluoromethylsulfonyl, trifluoromethylsulfinyl, trifluoromethylthio, trifluoromethyl or pentafluoroethyl and
   Aa, Ab, Ac, Ad, R¹, R⁴, R⁶, R⁷, R⁸, R¹¹, R¹², R¹³, R¹⁵, R¹⁶, R¹⁷, Q and n have the meanings described in Configuration (1) or Configuration (2) or Configuration (3) or Configuration (4) or Configuration (5).

In a preferred embodiment, the invention relates to compounds of the formula (I) where
Q represents a heteroaromatic 9-membered fused bicyclic ring system from the group consisting of Q2, Q3, Q15 and Q21,
R⁴ represents methyl,
R⁵ represents trifluoromethylsulfonyl, trifluoromethylsulfinyl, trifluoromethylthio, trifluoromethyl or pentafluoroethyl,
R⁶ represents hydrogen,
R⁷ represents methyl and
   Aa, Ab, Ac, Ad, R¹, R⁸, R¹¹, R¹², R¹³, R¹⁵, R¹⁶, R¹⁷ and n have the meanings described in Configuration (1) or Configuration (2) or Configuration (3) or Configuration (4) or Configuration (5).

Especially preferably, the following structures (Ia), (Ib), (Id), (Ie), (Ig) to (Ik) and (Im) to (It) result for the formulae (I)

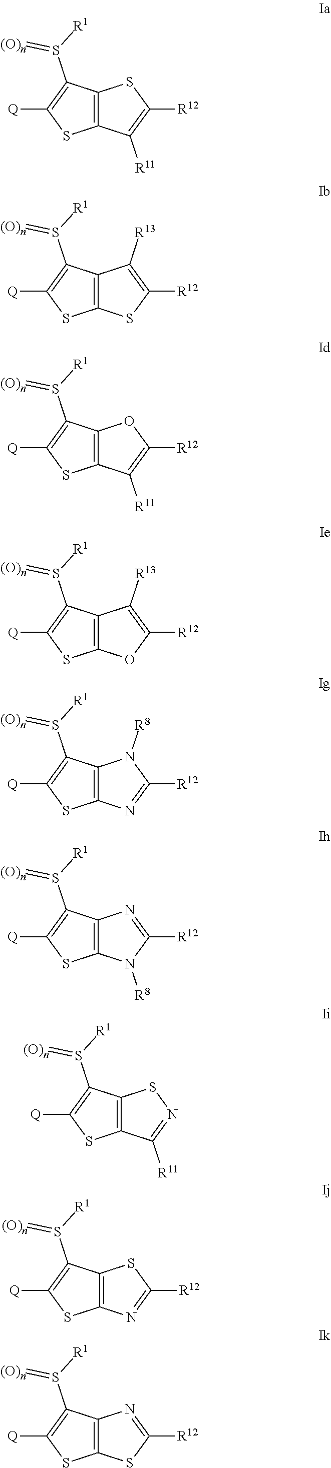

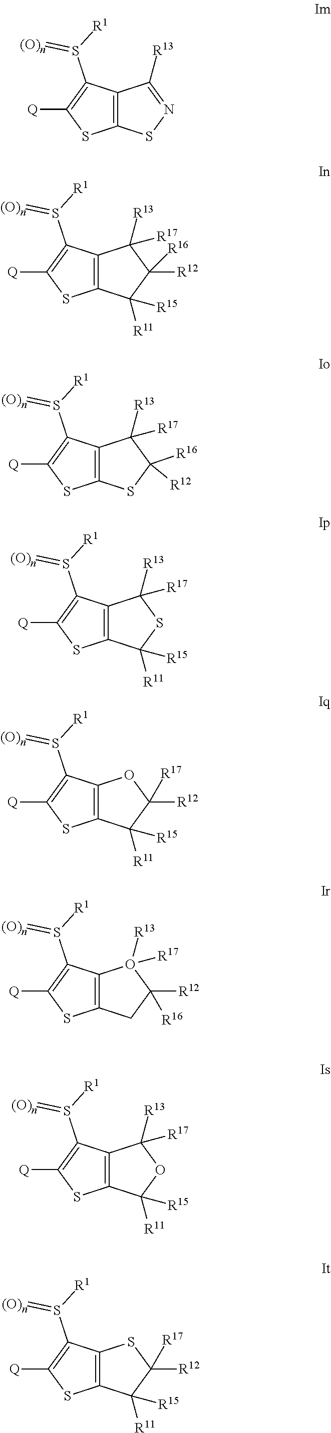

where R¹, R⁴, R⁵, R⁶, R⁷, R⁸, R¹¹, R¹², R¹³, R¹⁵, R¹⁶, R¹⁷, Q and n have the meanings described in Configuration (1) or Configuration (2) or Configuration (4) or Configuration (5) or Configuration (6).

Especially preferably, the following structures (Ia), (Ib), (Id), (Ie), (Ii) to (Ik), (In), (Iq) and (Ir) result for the formulae (I)

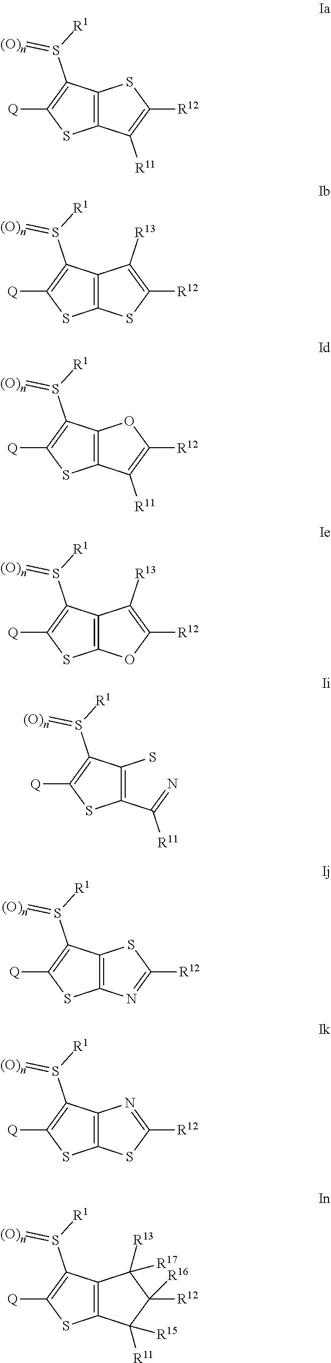

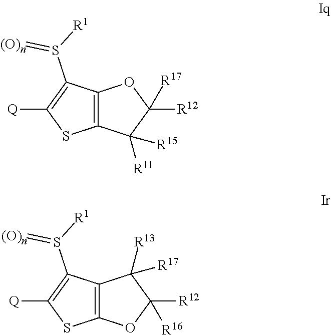

where R¹, R⁴, R⁵, R⁶, R⁷, R¹¹, R¹², R¹³, R¹⁵, R¹⁶, R¹⁷, Q and n have the meanings described in Configuration (1) or Configuration (2) or Configuration (3) or Configuration (5) or Configuration (6).

Especially particularly preferably, the following structures result for the formulae (I) (Ia), (Id), (Ij) and (In),

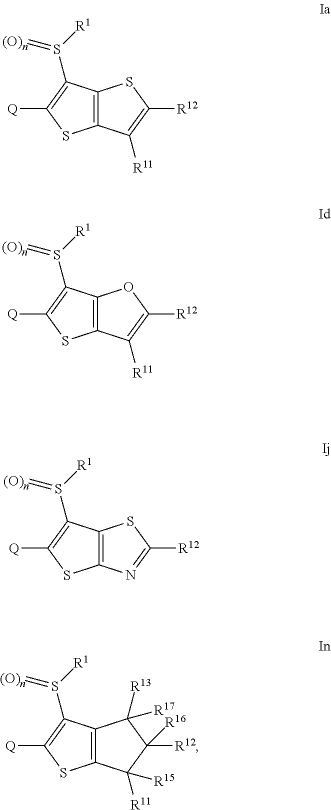

where R¹, R⁴, R⁵, R⁶, R⁷, R¹¹, R¹², R¹³, R¹⁵, R¹⁶, R¹⁷, Q and n have the meanings described in Configuration (1) or Configuration (2) or Configuration (3) or Configuration (4).

In a preferred embodiment, the invention relates to compounds of the formulae (Ia), (Ib), (Id), (Ie), (Ig) to (Ik) and (Im) to (It) where
R¹³, R¹⁵, R¹⁶, R¹⁷ independently of one another represent hydrogen, halogen, (C₁-C₄)-haloalkyl or (C₁-C₄)-alkyl,
R¹¹, R¹² independently of one another represent hydrogen, cyano, halogen, nitro, hydroxy, (C₃-C₆)-cycloalkyl, (C₁-C₄)-alkyl-(C₃-C₆)-cycloalkyl, halo-(C₃-C₆)-cycloalkyl, (C₁-C₄)-alkyl, (C₁-C₄)-haloalkyl, (C₁-C₄)-alkoxy-(C₁-C₄)-alkyl, (C₁-C₄)-alkoxy, (C₁-C₄)-haloalkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, aminosulfonyl, (C₁-C₄)-alkylaminosulfonyl, di-(C₁-C₄)-alkylaminosulfonyl, aminocarbonyl, (C₁-C₄)-alkylaminocarbonyl, di-(C₁-C₄)-alkylaminocarbonyl, (C₃-C₆)-cycloalkylaminocarbonyl, (C₁-C₄)-alkylcarbonylamino (—NHCO—(C₁-C₄)-alkyl), (C₃-C₆)-cycloalkylcarbonylamino, (C₁-C₄)-alkylcarbonyl-(C₁-C₄)-alkylamino (—N—(C₁-C₄)-alkyl-CO—(C₁-C₆)-alkyl), ((C₁-C₄)-alkoxycarbonyl)amino (—NHCOO—(C₁-C₄)-alkyl), (N—(C₁-C₄)-alkylcarbamoyl)amino (—NHCONH—(C₁-C₄)-alkyl), (N,N-di-(C₁-C₄)-alkylcarbamoyl)amino (—NHCON—(C₁-C₄)-dialkyl), (N—(C₁-C₄)-alkylcarbamoyl)-(C₁-C₄)-alkylamino (—N—(C₁-C₄)-alkyl-CONH—(C₁-C₄)-alkyl), carbamoylamino (—NHCONH₂), (carbamoyl)-N—(C₁-C₄)-alkylamino (—N(C₁-C₄)-alkyl-CONH₂)
   or very particularly preferably represent phenyl, pyrazole or triazole, each of which is optionally mono- or disubstituted by identical or different substituents, possible substituents being in each case: cyano, halogen, (C₃-C₆)-cycloalkyl, (C₁-C₄)-alkyl-(C₃-C₆)-cycloalkyl, halo-(C₃-C₆)-cycloalkyl, (C₁-C₄)-alkyl, (C₁-C₄)-haloalkyl, (C₁-C₄)-alkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, (C₁-C₄)-alkylsulfonyloxy, aminocarbonyl, (C₁-C₄)-alkylaminocarbonyl, di-(C₁-C₄)-alkylaminocarbonyl, (C₁-C₄)-alkylcarbonylamino,
where at most two of the radicals R¹¹, R¹², R¹³, R¹⁵, R¹⁶, R¹⁷ represent a substituent other than hydrogen,
and, in the case that neither R¹¹ nor R¹⁵, neither R¹² nor R¹⁶ or neither R¹³ nor R¹⁷ represents hydrogen, R¹⁴, R¹⁵, R¹⁶ and R¹⁷ each independently of one another only represent halogen, (C₁-C₄)-haloalkyl or (C₁-C₄)-alkyl, or,
in the case of formulae of the structures (In) to (It), R¹¹ and R¹⁵ or R¹³ and R¹⁷ additionally in each case together very particularly preferably represent oxygen (═O),
   and where Aa, Ab, Ac, Ad, R¹, R⁴, R⁵, R⁶, R⁷, Q and n have the meanings described in Configuration (1) or Configuration (2) or Configuration (3) or Configuration (5) or Configuration (6).

In a particularly preferred embodiment, the invention relates to compounds of the formulae (Ia), (Ib), (Id), (Ie), (Ig) to (Ik) and (Im) to (It) where
R¹³ represents hydrogen or halogen,
R¹⁵, R¹⁶, R¹⁷ represent hydrogen,
R¹¹, R¹² independently of one another represent hydrogen, hydroxy, cyano, halogen, (C₁-C₄)-alkyl, (C₃-C₆)-cycloalkyl, (C₁-C₄)-haloalkyl, (C₁-C₄)-haloalkoxy, aminocarbonyl, (C₁-C₄)-alkylaminocarbonyl, di-(C₁-C₄)-alkylaminocarbonyl, (C₃-C₆)-cycloalkylaminocarbonyl, (C₁-C₄)-alkylcarbonylamino (—NHCO—(C₁-C₄)-alkyl), (C₃-C₆)-cycloalkylcarbonylamino, (N—(C₁-C₄)-alkylcarbamoyl)amino (—NHCONH—(C₁-C₄)-alkyl), phenyl,
   or represent pyrazole or triazole, each of which is optionally monosubstituted by (C₁-C₄)-alkyl, halogen or (C₁-C₄)-haloalkyl, or,
in the case of formulae of the structures (In) to (It), R¹¹ and R¹⁵ or R¹³ and R¹⁷ additionally in each case together represent oxygen (═O),
   where Aa, Ab, Ac, Ad, R¹, R⁴, R⁵, R⁶, R⁷, Q and n have the meanings described in Configuration (1) or Configuration (2) or Configuration (3) or Configuration (4) or Configuration (6).

In a very particularly preferred embodiment, the invention relates to compounds of the formulae (Ia), (Ib), (Id), (Ie), (Ig) to (Ik) and (Im) to (It) where
R¹³ represents hydrogen or halogen,
R⁵, R¹⁶, R⁷ represent hydrogen,
R¹¹ represents hydrogen, hydroxy, cyano, halogen, (C₁-C₄)-alkyl, (C₃-C₆)-cycloalkyl, (C₁-C₄)-haloalkyl, aminocarbonyl, (C₁-C₄)-alkylaminocarbonyl, (C₃-C₆)-cycloalkylaminocarbonyl, (C₁-C₄)-alkylcarbonylamino (—NHCO—(C₁-C₄)-alkyl), (C₃-C₆)-cycloalkylcarbonylamino, (N—(C₁-C₄)-alkylcarbamoyl)amino (—NHCONH—(C₁-C₄)-alkyl), phenyl,
   or represents pyrazole or triazole, each of which is optionally monosubstituted by (C₁-C₄)-alkyl, halogen or (C₁-C₄)-haloalkyl,
R¹² represents hydrogen, halogen, (C₁-C₄)-alkyl, (C₃-C₆)-cycloalkyl or (C₁-C₄)-haloalkyl, or, in the case of formulae of the structures (In) to (It), R¹¹ and R¹⁵ or R¹³ and R¹⁷ additionally in each case together represent oxygen (═O),
   where Aa, Ab, Ac, Ad, R¹, R⁴, R⁵, R⁶, R⁷, Q and n have the meanings described in Configuration (1) or Configuration (2) or Configuration (3) or Configuration (4) or Configuration (5) or Configuration (6).

In a likewise very particularly preferred embodiment, the invention relates to compounds of the formulae (Ia), (Ib), (Id), (Ie), (Ig) to (Ik) and (Im) to (It) where
R¹³ represents hydrogen or fluorine,
R¹⁵, R¹⁶, R¹⁷ represent hydrogen,
R¹¹ represents hydrogen, hydroxy, cyano, cyclopropyl, trifluoromethyl, bromine, fluorine, iodine, aminocarbonyl, methylaminocarbonyl, methylcarbonylamino, cyclopropylcarbonylamino, (N-methylcarbamoyl)amino (—NHCONHMe), phenyl, N-methylpyrazole, trifluoromethylimidazole or chloropyrazole,
R¹² represents hydrogen, cyclopropyl, methyl, trifluoromethyl or chlorine,
or, in the case of formulae of the structures (In) to (It), R¹¹ and R¹⁵ or R¹³ and R¹⁷ additionally in each case together represent oxygen (═O),
   where Aa, Ab, Ac, Ad, R¹, R⁴, R⁵, R⁶, R⁷, Q and n have the meanings described in Configuration (1) or Configuration (2) or Configuration (3) or Configuration (4) or Configuration (5).

In a preferred embodiment of the invention, in the compounds of the formula (Ia), (Ib), (Id), (Ie), (Ig), (Ih), (Ij) or (Ik) at least one of the radicals R¹¹ or R¹² represents a radical different from hydrogen.

Preference is furthermore given to compounds of the formulae I(a), I(d) and I(j), where R¹¹ and/or R¹² represent a radical according to Configuration (1) or Configuration (2) or Configuration (3) or Configuration (4) or Configuration (5) or Configuration (6) but does not represent hydrogen, and where R¹, R⁴, R⁵, R⁶, R⁷, R¹¹, R¹³, R¹⁵, R¹⁶, R¹⁷, Q and n have the meanings described in Configuration (1) or Configuration (2) or Configuration (3) or Configuration (4) or Configuration (5) or Configuration (6).

Preference is furthermore given to compounds of the formula I(a)

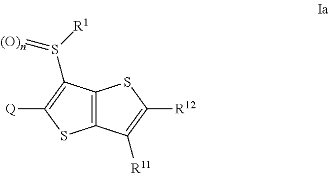

where R¹, R⁴, R⁵, R⁶, R⁷, R¹¹, R¹², Q and n have the meanings described in Configuration (1) or Configuration (2) or Configuration (3) or Configuration (4) or Configuration (5) or Configuration (6).

Particular preference is given to compounds of the formula I(a)

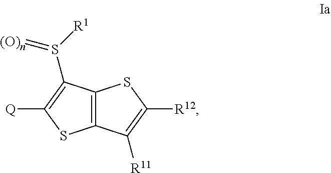

where
R¹ represents (C₁-C₄)-alkyl,
R¹² represents hydrogen, (C₁-C₄)-alkyl, halogen, (C₃-C₆)-cycloalkyl or (C₁-C₄)-haloalkyl,
R¹¹ represents hydrogen, cyano, (C₃-C₆)-cycloalkyl, (C₁-C₄)-haloalkyl, halogen, aminocarbonyl, (C₁-C₄)-alkylaminocarbonyl, (C₁-C₄)-alkylcarbonylamino, (C₃-C₆)-cycloalkylcarbonylamino, (N—(C₁-C₄)-alkylcarbamoyl), phenyl, N—(C₁-C₄)-alkylpyrazole, (C₁-C₄)-haloalkylimidazole or halopyrazole,
Q represents Q3, Q15 or Q21,
R⁴ represents (C₁-C₄)-alkyl,
R⁵ represents (C₁-C₄)-haloalkylsulfonyl, (C₁-C₄)-haloalkylsulfinyl, (C₁-C₄)-haloalkylthio, (C₁-C₄)-haloalkyl or monocyano-substituted cyclopropyl,
R⁶ represents hydrogen,
R⁷ represents (C₁-C₄)-alkyl, cyclopropyl, methoxymethyl or methoxyethyl,
   and
n represents 2.

Very particular preference is given to compounds of the formula I(a)

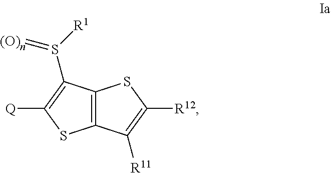

where
R¹ represents ethyl,
R¹² represents hydrogen, methyl, chlorine, cyclopropyl or trifluoromethyl,
R¹¹ represents hydrogen, cyano, cyclopropyl, trifluoromethyl, bromine, iodine, aminocarbonyl, methylaminocarbonyl, methylcarbonylamino, cyclopropylcarbonylamino, (N-methylcarbamoyl)amino (—NHCONHMe), phenyl, N-methylpyrazole, trifluoromethylimidazole or chloropyrazole,
Q represents Q3, Q15 or Q21,
R⁴ represents methyl,
R⁵ represents trifluoromethylsulfonyl, trifluoromethylsulfinyl, trifluoromethylthio, trifluoromethyl or pentafluoroethyl,
R⁶ represents hydrogen,
R⁷ represents methyl,
   and
n represents 2.

Preference is furthermore given to compounds of the formula I(d)

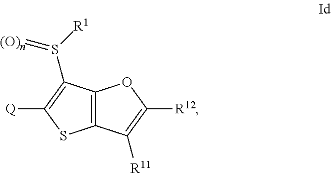

where R¹, R⁴, R⁵, R⁶, R¹¹, R¹², Q and n have the meanings described in Configuration (1) or Configuration (2) or Configuration (3) or Configuration (4) or Configuration (5) or Configuration (6).

Particular preference is furthermore given to compounds of the formula I(d)

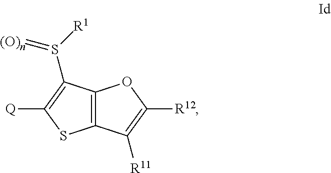

where
R¹ represents (C₁-C₄)-alkyl,
R¹² represents hydrogen or (C₁-C₄)-haloalkyl, preferably (C₁-C₄)-haloalkyl,
R¹¹ represents hydrogen,
Q represents Q3,
R⁴ represents (C₁-C₄)-alkyl,
R⁵ represents (C₁-C₄)-haloalkyl,
R⁶ represents hydrogen and
n represents 2.

Very particular preference is furthermore given to compounds of the formula I(d)

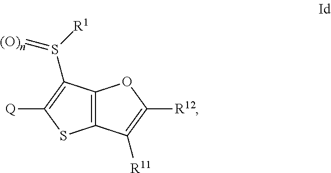

where
R¹ represents ethyl,
R¹² represents hydrogen or trifluoromethyl, preferably trifluoromethyl,
R¹¹ represents hydrogen,
Q represents Q3,
R⁴ represents methyl,
R⁵ represents trifluoromethyl,
R⁶ represents hydrogen and
n represents 2.

Preference is furthermore given to compounds of the formula I(j)

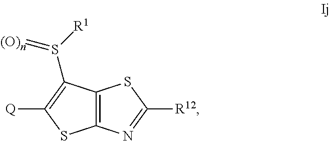

where R¹, R⁴, R⁵, R⁶, R¹², Q and n have the meanings described in Configuration (1) or Configuration (2) or Configuration (3) or Configuration (4) or Configuration (5) or Configuration (6).

Particular preference is furthermore given to compounds of the formula I(j)

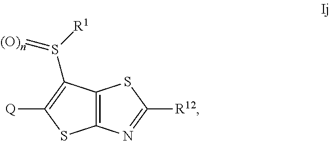

where
R¹ represents (C₁-C₄)-alkyl,
R¹² represents hydrogen or (C₁-C₄)-haloalkyl, preferably (C₁-C₄)-haloalkyl,
Q represents Q2, Q3 or Q15,
R⁴ represents (C₁-C₄)-alkyl,
R⁵ represents (C₁-C₄)-haloalkyl,
R⁶ represents hydrogen and
n represents 2.

Very particular preference is furthermore given to compounds of the formula I(j)

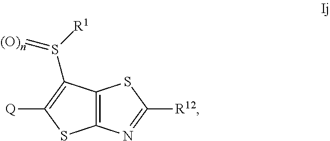

where
R¹ represents ethyl,
R¹² represents hydrogen or trifluoromethyl, preferably trifluoromethyl,
Q represents Q2, Q3 or Q15,
R⁴ represents methyl,
R⁵ represents trifluoromethyl or pentafluoroethyl,
R⁶ represents hydrogen and
n represents 2.

Preference is furthermore given to compounds of the formula I(n)

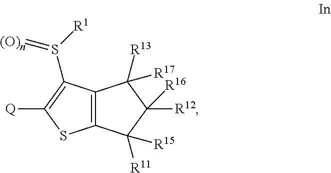

where R¹, R⁴, R⁵, R⁶, R¹¹, R¹², R¹², R¹⁵, R¹⁶, R¹⁷, Q and n have the meanings described in Configuration (1) or Configuration (2) or Configuration (3) or Configuration (4) or Configuration (5) or Configuration (6).

Particular preference is furthermore given to compounds of the formula I(n)

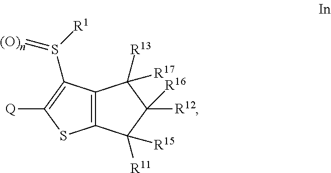

where
R¹ represents (C₁-C₄)-alkyl,
R¹², R¹⁵, R¹⁶, R¹⁷ represent hydrogen,
R¹¹, R¹³ independently of one another represent hydrogen, hydroxy or halogen,
or R¹¹ and R¹⁵ or R¹³ and R¹⁷ in each case together represent ═O,
Q represents Q3,
R⁴ represents (C₁-C₄)-alkyl,
R) represents (C₁-C₄)-haloalkyl,
R⁶ represents hydrogen and
n represents 2.

Very particular preference is furthermore given to compounds of the formula I(n)

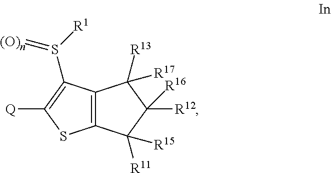

where
R¹ represents ethyl,
R¹², R¹⁵, R¹⁶, R¹⁷ represent hydrogen, R¹¹, R¹³ independently of one another represent hydrogen, hydroxy or fluorine,
or R¹¹ and R¹⁵ or R¹³ and R¹⁷ in each case together represent ═O,
Q represents Q3,
R⁴ represents methyl,
R⁵ represents trifluoromethyl,
R⁶ represents hydrogen and
n represents 2.

By definition, unless stated otherwise, halogen is selected from the group of fluorine, chlorine, bromine and iodine, preferably in turn from the group of fluorine, chlorine and bromine.

In the context of the present invention, unless defined differently elsewhere, the term “alkyl”, either on its own or else in combination with further terms, for example haloalkyl, is understood to mean a radical of a saturated, aliphatic hydrocarbon group which has 1 to 12 carbon atoms and may be branched or unbranched. Examples of C₁-C₁₂-alkyl radicals are methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1-ethylpropyl, 1,2-dimethylpropyl, hexyl, n-heptyl, n-octyl, n-nonyl, n-decyl, n-undecyl and n-dodecyl.

According to the invention, unless defined differently elsewhere, the term “alkenyl”, either on its own or else in combination with further terms, is understood to mean a straight-chain or branched C₂-C₁₂-alkenyl radical which has at least one double bond, for example vinyl, allyl, 1-propenyl, isopropenyl, 1-butenyl, 2-butenyl, 3-butenyl, 1,3-butadienyl, 1-pentenyl, 2-pentenyl, 3-pentenyl, 4-pentenyl, 1,3-pentadienyl, 1-hexenyl, 2-hexenyl, 3-hexenyl, 4-hexenyl, 5-hexenyl and 1,4-hexadienyl.

According to the invention, unless defined differently elsewhere, the term “alkynyl”, either on its own or else in combination with further terms, is understood to mean a straight-chain or branched C₂-C₁₂-alkynyl radical which has at least one triple bond, for example ethynyl, 1-propynyl and propargyl. The alkynyl radical may also contain at least one double bond.

According to the invention, unless defined differently elsewhere, the term “cycloalkyl”, either on its own or else in combination with further terms, is understood to mean a C₃-C₈-cycloalkyl radical, for example cyclopropyl, cyclobutyl, cyclopentyl, cyclohexyl, cycloheptyl and cyclooctyl.

The term “alkoxy”, either on its own or else in combination with further terms, for example haloalkoxy, is understood in the present case to mean an O-alkyl radical, where the term “alkyl” is as defined above.

Halogen-substituted radicals, for example haloalkyl, are mono- or polyhalogenated, up to the maximum number of possible substituents. In the case of polyhalogenation, the halogen atoms may be identical or different. Unless stated otherwise, optionally substituted radicals may be mono- or polysubstituted, where the substituents in the case of polysubstitutions may be the same or different.

According to the invention, unless defined differently elsewhere, the term “aryl” is understood to mean an aromatic radical having 6 to 14 carbon atoms, preferably phenyl, naphthyl, anthryl or phenanthrenyl, more preferably phenyl.

Unless defined differently elsewhere, the term “arylalkyl” is understood to mean a combination of the radicals “aryl” and “alkyl” defined according to the invention, where the radical is generally attached via the alkyl group. Examples of these are benzyl, phenylethyl or α-methylbenzyl, benzyl being particularly preferred.

Unless defined differently elsewhere, “hetaryl” denotes a mono-, bi- or tricyclic heterocyclic group of carbon atoms and at least one heteroatom, where at least one cycle is aromatic. Preferably, the hetaryl group contains 3, 4, 5, 6, 7 or 8 carbon atoms and is selected from the group consisting of furyl, thienyl, pyrrolyl, pyrazolyl, imidazolyl, 1,2,3-triazolyl, 1,2,4-triazolyl, oxazolyl, isoxazolyl, thiazolyl, isothiazolyl, 1,2,3-oxadiazolyl, 1,2,4-oxadiazolyl, 1,3,4-oxadiazolyl, 1,2,5-oxadiazolyl, 1,2,3-thiadiazolyl, 1,2,4-thiadiazolyl, 1,3,4-thiadiazolyl, 1,2,5-thiadiazolyl, pyridyl, pyrimidinyl, pyridazinyl, pyrazinyl, 1,2,3-triazinyl, 1,2,4-triazinyl, 1,3,5-triazinyl, benzofuryl, benzisofuryl, benzothienyl, benzisothienyl, indolyl, isoindolyl, indazolyl, benzothiazolyl, benzisothiazolyl, benzoxazolyl, benzisoxazolyl, benzimidazolyl, 2,1,3-benzoxadiazole, quinolinyl, isoquinolinyl, cinnolinyl, phthalazinyl, quinazolinyl, quinoxalinyl, naphthyridinyl, benzotriazinyl, purinyl, pteridinyl, imidazopyridinyl and indolizinyl.

Unless defined differently elsewhere, “heterocyclyl” denotes a monocyclic, saturated or partially saturated 4-, 5-, 6- or 7-membered ring of carbon atoms and at least one heteroatom in the ring. Preferably, the heterocyclyl group contains 3, 4, 5 or 6 carbon atoms and 1 or 2 heteroatoms from the group consisting of oxygen, sulfur and nitrogen. Examples of heterocyclyl are azetidinyl, azolidinyl, azinanyl, oxetanyl, oxolanyl, oxanyl, dioxanyl, thietanyl, thiolanyl, thianyl and tetrahydrofuryl.

Depending on the nature of the substituents, the compounds of the formula (I) may take the form of geometric and/or optically active isomers or corresponding isomer mixtures in different compositions. These stereoisomers are, for example, enantiomers, diastereomers, atropisomers or geometric isomers. The invention therefore encompasses pure stereoisomers and any desired mixtures of these isomers.

The compounds of the formula (I) can also be present as salts, in particular acid addition salts and metal salt complexes. The compounds of the formula (I) and their acid addition salts and metal salt complexes have good efficacy, especially for control of animal pests.

Suitable salts of the compounds of the general formula (I) include customary nontoxic salts, i.e. salts with appropriate bases and salts with added acids. Preference is given to salts with inorganic bases, such as alkali metal salts, for example sodium, potassium or caesium salts, alkaline earth metal salts, for example calcium or magnesium salts, ammonium salts, salts with organic bases and with inorganic amines, for example triethylammonium, dicyclohexylammonium, N,N′-dibenzylethylenediammonium, pyridinium, picolinium or ethanolammonium salts, salts with inorganic acids, for example hydrochlorides, hydrobromides, dihydrosulfates, trihydrosulfates, or phosphates, salts with organic carboxylic acids or organic sulfonic acids, for example formates, acetates, trifluoroacetates, maleates, tartrates, methanesulfonates, benzenesulfonates or para-toluenesulfonates, salts with basic amino acids, for example arginates, aspartates or glutamates, and the like.

The radical definitions or illustrations given above in general terms or listed within ranges of preference apply correspondingly to the end products and to the starting materials and intermediates. These radical definitions can be combined with one another as desired, i.e. including combinations between the respective preferred ranges.

Preference according to the invention is given to using compounds of the formula (I) which contain a combination of the meanings listed above as being preferred.

Particular preference according to the invention is given to using compounds of the formula (I) which contain a combination of the meanings listed above as being particularly preferred.

Very particular preference according to the invention is given to using compounds of the formula (I) which contain a combination of the definitions listed above as being very particularly preferred.

Most preference according to the invention is given to using compounds of the formula (I) which contain a combination of the meanings listed above as being most preferable.

Especially used according to the invention are compounds of the formula (I) which contain a combination of the meanings listed above as being especially emphasized.

The inventive compounds of the formula (I) can be obtained by the processes shown in the following schemes:

### Process A

The radicals Aa, Ab, Ac, Ad, Ae have the meanings described above. X represents halogen.

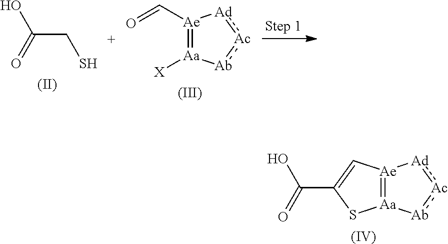

### Step 1:

Compounds of the formula (IV) are commercially available or can generally be prepared from derivatives of mercaptoacetic acid (II) and aldehydes of the formula (III). Solvents suitable for the reaction are, for example, polar solvents such as DMF or dimethyl sulfoxide, or alcohols such as methanol or ethanol. The reaction can be accelerated by addition of bases such as amine bases, for example triethylamine, or inorganic bases such as, for example, potassium carbonate or sodium methoxide, or be accelerated by further additives such as 18-crown-6 or copper salts.

In the case that Aa represents carbon, Ab represents C(R¹¹), Ac represents C(R¹²), Ad represents sulfur, Ae represents carbon, the preparation is carried out, for example, analogously to WO2008/106019 A2, 2008.

In the case that Aa represents carbon, Ab represents sulfur, Ac represents C(R¹²), Ad represents C(R¹³), Ae represents carbon, the preparation is carried out, for example, analogously to Journal of Medicinal Chemistry, 1991, vol. 34, 1805-1818.

In the case that Aa represents carbon, Ab represents C(R¹¹), Ac represents sulfur, Ad represents C(R¹³), Ae represents carbon, the preparation is carried out, for example, analogously to Chemistry—A European Journal, 2016, vol. 22, 694-703.

In the case that Aa represents carbon, Ab represents C(R¹¹), Ac represents C(R¹²), Ad represents oxygen, Ae represents carbon, the preparation is carried out, for example, analogously to Collection of Czechoslovak Chemical Communications, 1997, vol. 62, 1468-1480.

In the case that Aa represents carbon, Ab represents oxygen, Ac represents C(R¹²), Ad represents C(R¹³), Ae represents carbon, the preparation is carried out, for example, analogously to US2003/236263 A1, 2003.

In the case that Aa represents carbon, Ab represents nitrogen, Ac represents C(R¹²), Ad represents sulfur, Ae represents carbon, the preparation is carried out, for example, analogously to US2014/200215 A1, 2014 or WO2015/193506 A1, 2015.

In the case that Aa represents carbon, Ab represents nitrogen, Ac represents sulfur, Ad represents nitrogen, Ae represents carbon, the preparation is carried out, for example, analogously to Journal of Medicinal Chemistry, 1998, vol. 41, 109-116.

In the case that Aa represents carbon, Ab represents sulfur, Ac represents nitrogen, Ad represents C(R¹³), Ae represents carbon, the preparation is carried out, for example, analogously to Australian Journal of Chemistry, 1982, vol. 35, 393-404.

In the case that Aa represents carbon, Ab represents nitrogen, Ac represents C(R¹²), Ad represents nitrogen, Ae represents carbon, the preparation is carried out, for example, analogously to WO2011/119870 A1, 2011.

In the case that [symbol] represent exclusively single bonds and Aa represents carbon, Ab represents sulfur, oxygen or C(R¹¹)(R¹⁵), Ac represents sulfur, oxygen or C(R¹²)(R¹⁶) and Ad represents sulfur, oxygen or C(R¹³)(R¹⁷), Ae represents carbon, the preparation is carried out, for example, analogously to Synthesis, 1992, 526-528.

### Process B

In the case that Aa represents carbon, Ab represents C(R¹¹), Ac represents nitrogen, Ad represents sulfur, Ae represents carbon, the preparation is carried out, for example, in five steps such as described comprehensively in Arkivoc, 2013, #3 p. 245-265.

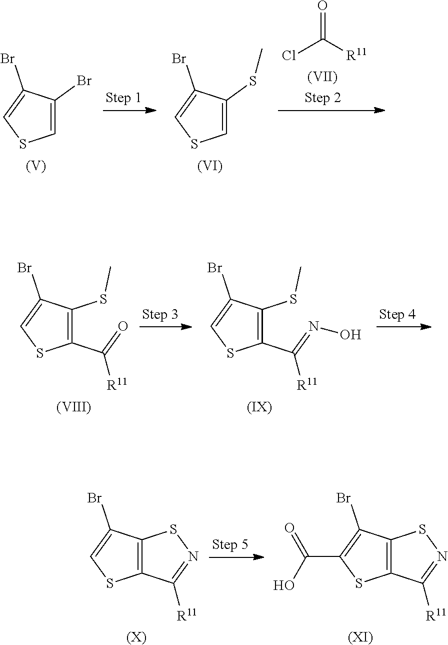

### Step 1

Dibromothiophene (V) is deprotonated with a suitable base, for example n-butyllithium, and then scavenged with a sulfur nucleophile such as dimethyl disulfide.

### Step 2

The carbonylation of thiophene (VIII) is carried out under Friedel-Crafts conditions by reaction with an acid chloride (VII) and a strong Lewis acid such as aluminium trichloride. Alternatively, this reaction can also take place in two steps by initially brominating thiophene (V) with N-bromosuccinimide and then adding a lithium base. Following transmetalation, the lithiated intermediate can be scavenged with a suitable nucleophile such as acid chloride (VII), giving compound (VIII).

### Step 3

The ketone or aldehyde (VIII) can be converted under standard conditions into the oxime (IX). Suitable reaction conditions for this reaction are, for example, addition of hydroxylamine and sodium acetate in an alcoholic solvent such as methanol.

### Step 4

The intramolecular cyclization to thienoisothiazole (X) is carried out after conversion of the hydroxyl function of the oxime into a good leaving group by reaction with mesyl chloride in the presence of an amine base such as triethylamine.

### Step 5

The conversion into acid (XI) can be effected by deprotonation of the thienoisothiazole (X) with LDA and scavenging with CO₂.

### Process C

In the case that Aa represents carbon, Ab represents C(R¹¹), Ac represents nitrogen, Ad represents C(R¹³), Ae represents nitrogen, the required precursor (XIII) is prepared, for example, analogously to EP1908766 A1, 2008 by cyclization of (XII) in the presence of phosphorus oxychloride (Step 1).

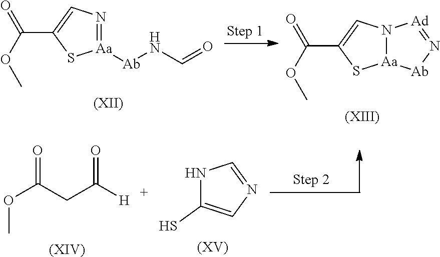

Alternatively (Step 2), the reaction can also be carried out analogously to WO2011/37780 A1, 2011 by reacting an imidazole (XV) with a (3-carbonyl ester (XIV).

### Process D

The general process for the preparation of compounds of the formula (XVII) is described below by way of example. X represents halogens such as fluorine, chlorine, bromine or iodine.

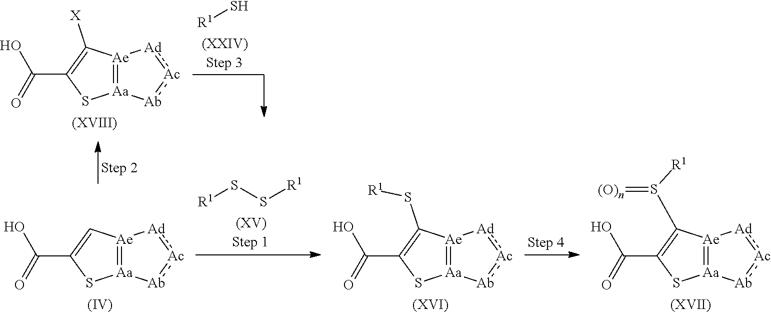

### Step 1

The direct conversion of (IV) into the sulfide (XVI) can be carried out by directed ortholithiation using a suitable base and then scavenging the lithiated species using the appropriate disulfide (XV). Particularly suitable bases for this type of reaction are, for example, lithium 2,2,6,6-tetramethylpiperidin-1-ide or the combination of sec-butyllithium and N,N,N′,N′-tetramethyl-1,2-diaminoethane. This type of reaction is described in a general manner in Organic Letters, 2006, vol. 8, #4 p. 765-768 or US2010/48531 A1, 2010, for example.

### Steps 2 and 3

Alternatively, the conversion into the sulfide (XVI) can take place in two steps starting with (IV). To this end, initially compound (IV) is halogenated under standard conditions in the presence of a suitable halogenating agent to give (XVIII). Suitable halogenating agents are, for example, N-bromosuccinimide (see e.g. Molecules, 2016, 1227) or N-chlorosuccinimide.

The compounds of the formula (XVI) can then be prepared by reacting the compounds of the formula (XXVIII) with the compounds of the formula (XXIV) in the presence of a base.

Mercaptan derivatives of the formula (XXIV), for example methyl mercaptan, ethyl mercaptan or isopropyl mercaptan, are either commercially available or can be prepared by known methods, for example analogously to the processes described in US2006/25633, US2006/111591, U.S. Pat. No. 2,820,062, Chemical Communications, 13 (2000), 1163-1164 or Journal of the American Chemical Society, 44 (1922), p. 1329.

The conversion to the compound of the formula (XVI) can be carried out neat or in a solvent, preference being given to conducting the reaction in a solvent selected from customary solvents that are inert under the prevailing reaction conditions. Preference is given to ethers, for example diisopropyl ether, dioxane, tetrahydrofuran, 1,2-dimethoxyethane, tert-butyl methyl ether; nitriles, for example acetonitrile or propionitrile; aromatic hydrocarbons, for example toluene or xylene; aprotic polar solvents, for example N,N-dimethylformamide, N-methylpyrrolidone or dimethyl sulfoxide.

Examples of suitable bases are inorganic bases from the group consisting of acetates, phosphates and carbonates of alkali metals or alkaline earth metals. Preference is given here to caesium carbonate, sodium carbonate and potassium carbonate. Further suitable bases are alkali metal hydrides, for example sodium hydride.

The reaction can be conducted under reduced pressure, at standard pressure or under elevated pressure, and at temperatures of 0° C. to 200° C.

### Step 4

The compounds of the formula (XVII), where n represents 2, can also be prepared in a one-step process by oxidizing the compounds of the formula (XVI). The oxidation is generally carried out in a solvent.

Preference is given to halogenated hydrocarbons, for example dichloromethane, chloroform, carbon tetrachloride, 1,2-dichloroethane or chlorobenzene; alcohols such as methanol or ethanol; formic acid, acetic acid, propionic acid or water.

Examples of suitable oxidizing agents are hydrogen peroxide and meta-chloroperbenzoic acid.

The reaction can be conducted under reduced pressure, at standard pressure or under elevated pressure, and at temperatures of

### −20° C. to 120° C.

### Process E

The general method for the preparation of compounds of the formula (I) in which Q represents Q1, Q2, Q3, Q4, Q5, Q6, Q7, Q8, Q9, Q15, Q20 or Q21 is described hereinafter by way of example with reference to compounds of the formula (I) in which Q represents Q2. In the case of Q1, Q2, Q3, Q15, Q20 and Q21, Y represents —NHR₄. In the case of Q4 and Q5, Y represents chlorine, or in the case of Q7 and Q8, Y represents SH.

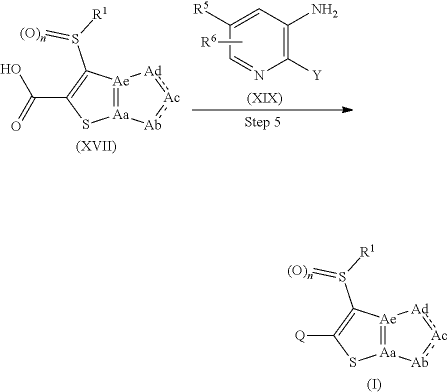

The compounds of the formula (I) can be prepared in analogy to the process described in U.S. Pat. No. 5,576,335 by the reaction of compounds of the formula (XIX) with carboxylic acids of the formula (XVII) in the presence of a condensing agent or a base.

Compounds of the formula (XIX) are either commercially available or can be prepared by known methods, for example analogously to the processes described in US2003/69257, WO2006/65703, WO2009/131237, WO2010/125985, WO2011/043404, WO2011/040629, WO2012/086848, WO2013/018928 or WO2015/000715.

Carboxylic acids of the formula (XVII) are either commercially available or can be prepared by known methods. Possible preparation routes are described in processes A, B, C and D.

The reaction of the compounds of the formula (XIX) with carboxylic acids of the formula (XVII) can be carried out neat or in a solvent, preference being given to conducting the reaction in a solvent selected from customary solvents that are inert under the prevailing reaction conditions. Preference is given to ethers, for example diisopropyl ether, dioxane, tetrahydrofuran, 1,2-dimethoxyethane; halogenated hydrocarbons, for example dichloromethane, chloroform, carbon tetrachloride, 1,2-dichloroethane or chlorobenzene; nitriles, for example acetonitrile or propionitrile; aromatic hydrocarbons, for example toluene or xylene; aprotic polar solvents, for example N,N-dimethylformamide or N-methylpyrrolidone, or nitrogen compounds, for example pyridine.

Suitable condensing agents are, for example, carbodiimides such as 1-(3-dimethylaminopropyl)-3-ethylcarbodiimide hydrochloride (EDCI) or 1,3-dicyclohexylcarbodiimide.

Suitable bases are inorganic bases which are typically used in such reactions. Preference is given to using bases selected by way of example from the group consisting of acetates, phosphates, carbonates and bicarbonates of alkali metals or alkaline earth metals. Particular preference is given here to sodium acetate, sodium phosphate, potassium phosphate, caesium carbonate, sodium carbonate, potassium carbonate, sodium bicarbonate, potassium bicarbonate.

The reaction can be effected under reduced pressure, at standard pressure or under elevated pressure and at temperatures of 0° C. to 180° C.; with preference, the reaction is carried out at standard pressure and temperatures of 20 to 140° C.

As an alternative to the reaction of the acid (XVII) with a condensing agent, the acid can be converted by reaction with oxalyl chloride or phosphoryl chloride into the corresponding acid chloride and then be reacted with (XIX).

### Process F

The general method for the preparation of compounds of the formula (I) in which Q represents Q10, Q11, Q12 or Q13 is described hereinafter by way of example with reference to compounds of the formula (I) in which Q is Q12.

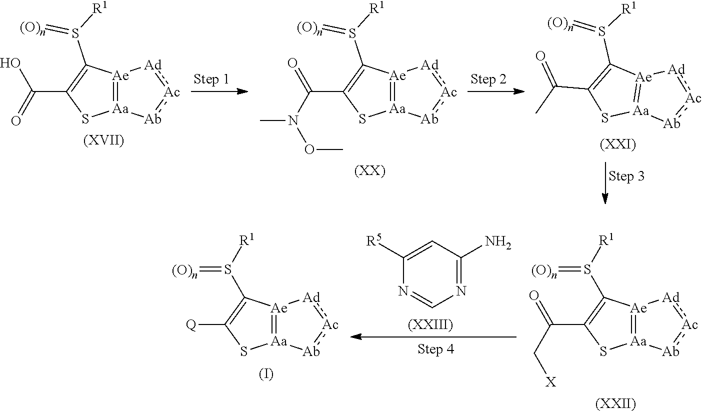

### Step 1

Carboxylic acids of the formula (XVII) are converted in analogy to the process described in WO2015/107117, WO2011/75643 or EP2671582 in the presence of O,N-dimethylhydroxylamine hydrochloride to Weinreb amides of the formula (XX).

Carboxylic acids of the formula (XVII) are either commercially available or can be prepared by known methods. Possible preparation routes are described in processes A, B, C and D.

### Steps 2, 3

Compounds of the formula (XX) can then be converted by known methods, for example analogously to the process described in WO2011/75643, using a Grignard reagent such as, for example, methylmagnesium bromide into ketones of the formula (XXI). Compounds of the formula (XXII) are obtainable by subsequent halogenation analogously to the known method described, for example, in US2012/302573.

### Step 4

The compounds of the formula (I) can be prepared by cyclizing the compounds of the formula (XXII) with amines of the formula (XXIII). The cyclization is carried out, for example, in ethanol, acetonitrile or N,N-dimethylformamide according to known methods analogously, for example, to the processes described in WO2005/66177, WO2012/88411, WO2013/3298, US2009/203705, US2012/258951, WO2012/168733, WO2014/187762 or J. Med. Chem. 31 (1988) 1590-1595.

The compounds of the formula (XXIII) are commercially available.

### Process G

The general method for the preparation of compounds of the formula (I) in which Q represents Q1, Q2, Q3, Q4, Q5, Q6, Q7, Q8, Q9, Q15, Q20 or Q21 is described hereinafter by way of example with reference to compounds of the formula (I) in which Q represents Q2. In the case of Q1, Q2, Q3, Q15, Q20 and Q21, Y represents —NHR₄. In the case of Q4 and Q5, Y represents chlorine, or in the case of Q7 and Q8, Y represents SH.

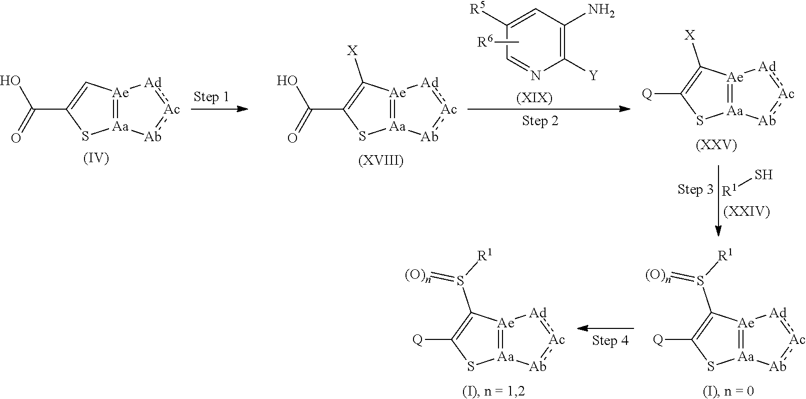

### Step 1

The compounds of the formula XVIII can preferably be prepared by selective metallation of starting material (IV) using suitable bases, followed by reaction with a halogenating agent.

A suitable base is in particular 2,2,6,6-tetramethylpiperidinylmagnesium chloride/lithium chloride complex, as described in Chem. Eur. J. 2011, 17, 866-872. In principle, other sterically hindered amine bases such as diisopropyldiethylamine, having other counterions such as zinc or lithium, for example, are also suitable for the reaction. Also suitable in principle are alkyllithium bases such as n-butyllithium. In this regard, see, for example, WO2005/12311 A1, 2005.

Suitable halogenating agents are, for example, iodine, bromine, N-bromosuccinimide, N-iodosuccinimide and others.

The conversion can be carried out neat or in a solvent, preference being given to conducting the reaction in a solvent selected from customary solvents that are inert under the prevailing reaction conditions.

Preference is given to ethers such as diethyl ether, diisopropyl ether, dioxane, tetrahydrofuran, 1,2-dimethoxyethane; aromatic hydrocarbons such as toluene or xylene.

The reaction can be conducted under reduced pressure, at standard pressure or under elevated pressure and at temperatures of −78 to 100° C.; the reaction is preferably effected at standard pressure and temperatures of −78° C. to 60° C.

Alternatively to the process described above, a compound of the formula (XVII) can also be converted by direct reaction with a halogenating agent such as, for example, bromine or N-bromosuccinimide into (XVIII).

### Step 2

The compounds of the formula (XXIV) can be prepared in analogy to the process described in U.S. Pat. No. 5,576,335 by the reaction of compounds of the formula (XIX) with carboxylic acids of the formula (XVIII) in the presence of a condensing agent or a base.

Compounds of the formula (XIX) are either commercially available or can be prepared by known methods, for example analogously to the processes described in US2003/69257, WO2006/65703, WO2009/131237, WO2010/125985, WO2011/043404, WO2011/040629, WO2012/086848, WO2013/018928 or WO2015/000715.

Carboxylic acids of the formula (XVIII) are either commercially available or can be prepared by known methods. Possible preparation routes are described in processes A, B, C and D.

The reaction of the compounds of the formula (XIX) with carboxylic acids of the formula (XVIII) can be carried out neat or in a solvent, preference being given to conducting the reaction in a solvent selected from customary solvents that are inert under the prevailing reaction conditions. Preference is given to ethers, for example diisopropyl ether, dioxane, tetrahydrofuran, 1,2-dimethoxyethane; halogenated hydrocarbons, for example dichloromethane, chloroform, carbon tetrachloride, 1,2-dichloroethane or chlorobenzene; nitriles, for example acetonitrile or propionitrile; aromatic hydrocarbons, for example toluene or xylene; aprotic polar solvents, for example N,N-dimethylformamide or N-methylpyrrolidone, or nitrogen compounds, for example pyridine.

Suitable condensing agents are, for example, carbodiimides such as 1-(3-dimethylaminopropyl)-3-ethylcarbodiimide hydrochloride (EDCI) or 1,3-dicyclohexylcarbodiimide. As an alternative to the reaction of the acid (XVIII) with a condensing agent, the acid can be converted by reaction with oxalyl chloride or phosphoryl chloride into the corresponding acid chloride and then be reacted with (XIX).

Suitable bases are inorganic bases which are typically used in such reactions. Preference is given to using bases selected by way of example from the group consisting of acetates, phosphates, carbonates and hydrogencarbonates of alkali metals or alkaline earth metals. Particular preference is given here to sodium acetate, sodium phosphate, potassium phosphate, caesium carbonate, sodium carbonate, potassium carbonate, sodium hydrogencarbonate, potassium hydrogencarbonate.

The reaction can be effected under reduced pressure, at standard pressure or under elevated pressure and at temperatures of 0° C. to 180° C.; with preference, the reaction is carried out at standard pressure and temperatures of 20 to 140° C.

### Step 3

Compounds of the formula (I) where n=0 can be prepared by coupling (XXIV) with an appropriate mercaptan (XXIV). Coupling is carried out using a catalyst composed of a metal salt or metal complex and optionally a suitable ligand, optionally with addition of a base.

Suitable combinations of metal salts, metal complexes and ligands are, for example, tris-(dibenzylideneacetone)dipalladium(0), (5-diphenylphosphanyl-9,9-dimethylxanthen-4-yl)diphenylphosphane (see US2009/156642 A1) or copper iodide (see Australian Journal of Chemistry, 1985, vol. 38, 899).

The reaction can be effected neat or in a solvent, preference being given to conducting the reaction in a solvent selected from customary solvents that are inert under the prevailing reaction conditions.

Preference is given to ethers, for example diisopropyl ether, dioxane, tetrahydrofuran, 1,2-dimethoxyethane; halogenated hydrocarbons, for example dichloromethane, chloroform, carbon tetrachloride, 1,2-dichloroethane or chlorobenzene; nitriles, for example acetonitrile or propionitrile; aromatic hydrocarbons, for example toluene or xylene; aprotic polar solvents, for example N,N-dimethylformamide or N-methylpyrrolidone, or nitrogen compounds, for example pyridine.

Suitable bases are inorganic bases which are typically used in such reactions. Preference is given to using bases selected by way of example from the group consisting of acetates, phosphates, carbonates and hydrogencarbonates of alkali metals or alkaline earth metals.

### Step 4:

The compounds of the formula (I) where n is 1 or 2 can be prepared by oxidizing the compounds of the formula (I) where n=0. The oxidation is generally carried out in a solvent. Preference is given to halogenated hydrocarbons, for example dichloromethane, chloroform, carbon tetrachloride, 1,2-dichloroethane or chlorobenzene; alcohols such as methanol or ethanol; formic acid, acetic acid, propionic acid or water.

Examples of suitable oxidizing agents are hydrogen peroxide and meta-chloroperbenzoic acid.

The reaction can be conducted under reduced pressure, at standard pressure or under elevated pressure, and at temperatures of

### −20° C. to 120° C.

### Process H:

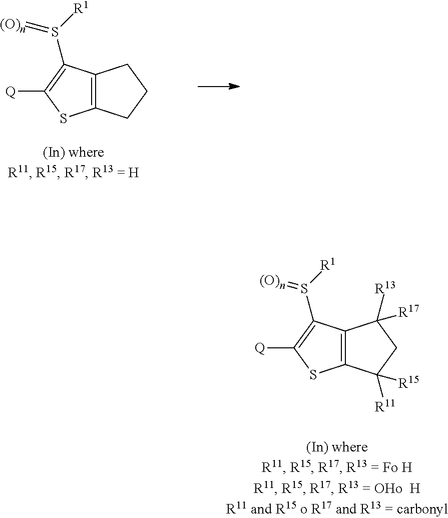

Compounds of the formula (In) in which R¹¹, R¹⁵, R¹³ and R¹⁷ may represent H, OH or F, and compounds in which R¹¹ and R¹⁵ or R¹³ and R¹⁷ may together represent a carbonyl group can all be obtained from compounds of the formula (I) where R¹¹, R¹⁵, R¹³ and R¹⁷═H by reaction with a suitable fluorinating agent and an oxidizing agent.

Suitable fluorinating agents are, for example, Selectfluor, NFPy, Accufluor, NFSI, as described in Chem. Rev. 2015, 115, 9073. A suitable oxidizing agent is potassium peroxodisulfate. Metal-catalytic and photocatalytic processes are likewise possible and are described, for example, in Chem. Rev. 2015, 115, 9073.

### Methods and Uses

The invention also relates to methods for controlling animal pests where compounds of the formula (I) are allowed to act on animal pests and/or their habitat. The control of the animal pests is preferably carried out in agriculture and forestry, and in material protection. This preferably excludes methods for surgical or therapeutic treatment of the human or animal body and diagnostic methods carried out on the human or animal body.

The invention further relates to the use of the compounds of the formula (I) as pesticides, especially crop protection compositions.

In the context of the present application, the term “pesticide” in each case also always encompasses the term “plant protection composition”.

The compounds of the formula (I), given good plant tolerance, favourable homeotherm toxicity and good environmental compatibility, are suitable for protecting plants and plant organs against biotic and abiotic stress factors, for increasing harvest yields, for improving the quality of the harvested material and for controlling animal pests, especially insects, arachnids, helminths, especially nematodes and molluscs, which are encountered in agriculture, in horticulture, in animal husbandry, in aquatic cultures, in forests, in gardens and leisure facilities, in the protection of stored products and of materials, and in the hygiene sector.

In the context of the present patent application, the term “hygiene” should be understood to mean any and all measures, provisions and procedures which have the aim of preventing diseases, especially infection diseases, and which serve to protect the health of humans and animals and/or protect the environment and/or maintain cleanliness. According to the invention, this especially includes measures for cleaning, disinfection and sterilization, for example of textiles or hard surfaces, especially surfaces made of glass, wood, cement, porcelain, ceramic, plastic or else metal(s), in order to ensure that these are free of hygiene pests and/or their secretions. The scope of protection of the invention in this regard preferably excludes surgical or therapeutic treatment procedures to be applied to the human body or the bodies of animals, and diagnostic procedures which are carried out on the human body or the bodies of animals.

The term “hygiene sector” covers all areas, technical fields and industrial applications in which these hygiene measures, provisions and procedures are important, for example with regard to hygiene in kitchens, bakeries, airports, bathrooms, swimming pools, department stores, hotels, hospitals, stables, animal keeping, etc.

The term “hygiene pest” should therefore be understood to mean one or more animal pests whose presence in the hygiene sector is problematic, especially for reasons of health. A main aim is therefore that of avoiding, or limiting to a minimum, the presence of hygiene pests and/or the exposure to these in the hygiene sector. This can especially be achieved through the use of a pesticide which can be used both for prevention of infestation and for prevention of an existing infestation. It is also possible to use formulations which prevent or reduce exposure to pests. Hygiene pests include, for example, the organisms mentioned below.

The term “hygiene protection” thus covers all acts by which these hygiene measures, provisions and procedures are maintained and/or improved.

The compounds of the formula (I) can preferably be used as pesticides. They are active against normally sensitive and resistant species and also against all or specific stages of development. The aforementioned pests include:

pests from the phylum of the Arthropoda, in particular from the class of the Arachnida, for example Acarus spp., e.g. Acarus siro, Aceria kuko, Aceria sheldoni, Aculops spp., Aculus spp., e.g. Aculus fockeui, Aculus schlechtendali, Amblyomma spp., Amphitetranychus viennensis, Argas spp., Boophilus spp., Brevipalpus spp., e.g. Brevipalpus phoenicis, Bryobia graminum, Bryobia praetiosa, Centruroides spp., Chorioptes spp., Dermanyssus gallinae, Dermatophagoides pteronyssinus, Dermatophagoides farinae, Dermacentor spp., Eotetranychus spp., e.g. Eotetranychus hicoriae, Epitrimerus pyri, Eutetranychus spp., e.g. Eutetranychus banksi, Eriophyes spp., e.g. Eriophyes pyri, Glycyphagus domesticus, Halotydeus destructor, Hemitarsonemus spp., e.g. Hemitarsonemus latus (=Polyphagotarsonemus latus), Hyalomma spp., Ixodes spp., Latrodectus spp., Loxosceles spp., Neutrombicula autumnalis, Nuphersa spp., Oligonychus spp., e.g. Oligonychus coffeae, Oligonychus coniferarum, Oligonychus ilicis, Oligonychus indicus, Oligonychus mangiferus, Oligonychus pratensis, Oligonychus punicae, Oligonychus yothersi, Ornithodorus spp., Ornithonyssus spp., Panonychus spp., e.g. Panonychus citri (=Metatetranychus citri), Panonychus ulmi (=Metatetranychus ulmi), Phyllocoptruta oleivora, Platytetranychus multidigituli, Polyphagotarsonemus latus, Psoroptes spp., Rhipicephalus spp., Rhizoglyphus spp., Sarcoptes spp., Scorpio maurus, Steneotarsonemus spp., Steneotarsonemus spinki, Tarsonemus spp., e.g. Tarsonemus confusus, Tarsonemus pallidus, Tetranychus spp., e.g. Tetranychus canadensis, Tetranychus cinnabarinus, Tetranychus turkestani, Tetranychus urticae, Trombicula alfreddugesi, Vaejovis spp., Vasates lycopersici;
from the class of the Chilopoda, for example Geophilus spp., Scutigera spp.;
from the order or the class of the Collembola, for example Onychiurus armatus; Sminthurus viridis;
from the class of the Diplopoda, for example Blaniulus guttulatus;
from the class of the Insecta, for example from the order of the Blattodea, e.g. Blatta orientalis, Blattella asahinai, Blattella germanica, Leucophaea maderae, Loboptera decipiens, Neostylopyga rhombifolia, Panchlora spp., Parcoblatta spp., Periplaneta spp., e.g. Periplaneta americana, Periplaneta australasiae, Pycnoscelus surinamensis, Supella longipalpa;
from the order of the Coleoptera, for example Acalymma vittatum, Acanthoscelides obtectus, Adoretus spp., Aethina tumida, Agelastica alni, Agrilus spp., e.g. Agrilus planipennis, Agrilus coxalis, Agrilus bilineatus, Agrilus anxius, Agriotes spp., e.g. Agriotes linneatus, Agriotes mancus, Alphitobius diaperinus, Amphimallon solstitialis, Anobium punctatum, Anoplophora spp., e.g. Anoplophora glabripennis, Anthonomus spp., e.g. Anthonomus grandis, Anthrenus spp., Apion spp., Apogonia spp., Atomaria spp., e.g. Atomaria linearis, Attagenus spp., Baris caerulescens, Bruchidius obtectus, Bruchus spp., e.g. Bruchus pisorum, Bruchus rufimanus, Cassida spp., Cerotoma trifurcata, Ceutorrhynchus spp., e.g. Ceutorrhynchus assimilis, Ceutorrhynchus quadridens, Ceutorrhynchus rapae, Chaetocnema spp., e.g. Chaetocnema confinis, Chaetocnema denticulata, Chaetocnema ectypa, Cleonus mendicus, Conoderus spp., Cosmopolites spp., e.g. Cosmopolites sordidus, Costelytra zealandica, Ctenicera spp., Curculio spp., e.g. Curculio caryae, Curculio caryatrypes, Curculio obtusus, Curculio sayi, Cryptolestes ferrugineus, Cryptolestes pusillus, Cryptorhynchus lapathi, Cryptorhynchus mangiferae, Cylindrocopturus spp., Cylindrocopturus adspersus, Cylindrocopturus fumissi, Dendroctonus spp., e.g. Dendroctonus ponderosae, Dermestes spp., Diabrotica spp., e.g. Diabrotica balteata, Diabrotica barberi, Diabrotica undecimpunctata howardi, Diabrotica undecimpunctata undecimpunctata, Diabrotica virgifera virgifera, Diabrotica virgifera zeae, Dichocrocis spp., Dicladispa armigera, Diloboderus spp., Epicaerus spp., Epilachna spp., e.g. Epilachna borealis, Epilachna varivestis, Epitrix spp., e.g. Epitrix cucumeris, Epitrix fuscula, Epitrix hirtipennis, Epitrix subcrinita, Epitrix tuberis, Faustinus spp., Gibbium psylloides, Gnathocerus comutus, Hellula undalis, Heteronychus arator, Heteronyx spp., Hylamorpha elegans, Hylotrupes bajulus, Hypera postica, Hypomeces squamosus, Hypothenemus spp., e.g. Hypothenemus hampei, Hypothenemus obscurus, Hypothenemus pubescens, Lachnosterna consanguinea, Lasioderma serricorne, Latheticus oryzae, Lathridius spp., Lema spp., Leptinotarsa decemlineata, Leucoptera spp., e.g. Leucoptera coffeella, Limonius ectypus, Lissorhoptrus oryzophilus, Listronotus (=Hyperodes) spp., Lixus spp., Luperodes spp., Luperomorpha xanthodera, Lyctus spp., Megacyllene spp., e.g. Megacyllene robiniae, Megascelis spp., Melanotus spp., e.g. Melanotus longulus oregonensis, Meligethes aeneus, Melolontha spp., e.g. Melolontha melolontha, Migdolus spp., Monochamus spp., Naupactus xanthographus, Necrobia spp., Neogalerucella spp., Niptus hololeucus, Oryctes rhinoceros, Oryzaephilus surinamensis, Oryzaphagus oryzae, Otiorhynchus spp., e.g. Otiorhynchus cribricollis, Otiorhynchus ligustici, Otiorhynchus ovatus, Otiorhynchus rugosostriarus, Otiorhynchus sulcatus, Oulema spp., e.g. Oulema melanopus, Oulema oryzae, Oxycetonia jucunda, Phaedon cochleariae, Phyllophaga spp., Phyllophaga helleri, Phyllotreta spp., e.g. Phyllotreta armoraciae, Phyllotreta pusilla, Phyllotreta ramosa, Phyllotreta striolata, Popillia japonica, Premnotrypes spp., Prostephanus truncatus, Psylliodes spp., e.g. Psylliodes affinis, Psylliodes chrysocephala, Psylliodes punctulata, Ptinus spp., Rhizobius ventralis, Rhizopertha dominica, Rhynchophorus spp., Rhynchophorus ferrugineus, Rhynchophorus palmarum, Scolytus spp., e.g. Scolytus multistriatus, Sinoxylon perforans, Sitophilus spp., e.g. Sitophilus granarius, Sitophilus linearis, Sitophilus oryzae, Sitophilus zeamais, Sphenophorus spp., Stegobium paniceum, Stemechus spp., e.g. Sternechus paludatus, Symphyletes spp., Tanymecus spp., e.g. Tanymecus dilaticollis, Tanymecus indicus, Tanymecus palliatus, Tenebrio molitor, Tenebrioides mauretanicus, Tribolium spp., e.g. Tribolium audax, Tribolium castaneum, Tribolium confusum, Trogoderma spp., Tychius spp., Xylotrechus spp., Zabrus spp., e.g. Zabrus tenebrioides;
from the order of the Dermaptera, for example Anisolabis maritime, Forficula auricularia, Labidura riparia;
from the order of the Diptera, for example Aedes spp., e.g. Aedes aegypti, Aedes albopictus, Aedes sticticus, Aedes vexans, Agromyza spp., e.g. Agromyza frontella, Agromyza parvicornis, Anastrepha spp., Anopheles spp., e.g. Anopheles quadrimaculatus, Anopheles gambiae, Asphondylia spp., Bactrocera spp., e.g. Bactrocera cucurbitae, Bactrocera dorsalis, Bactrocera oleae, Bibio hortulanus, Calliphora erythrocephala, Calliphora vicina, Ceratitis capitata, Chironomus spp., Chrysomya spp., Chrysops spp., Chrysozona pluvialis, Cochliomya spp., Contarinia spp., e.g. Contarinia johnsoni, Contarinia nasturtii, Contarinia pyrivora, Contarinia schulzi, Contarinia sorghicola, Contarinia tritici, Cordylobia anthropophaga, Cricotopus sylvestris, Culex spp., e.g. Culex pipiens, Culex quinquefasciatus, Culicoides spp., Culiseta spp., Cuterebra spp., Dacus oleae, Dasineura spp., e.g. Dasineura brassicae, Delia spp., e.g. Delia antiqua, Delia coarctata, Delia florilega, Delia platura, Delia radicum, Dermatobia hominis, Drosophila spp., e.g. Drosphila melanogaster, Drosophila suzukii, Echinocnemus spp., Euleia heraclei, Fannia spp., Gasterophilus spp., Glossina spp., Haematopota spp., Hydrellia spp., Hydrellia griseola, Hylemya spp., Hippobosca spp., Hypoderma spp., Liriomyza spp., e.g. Liriomyza brassicae, Liriomyza huidobrensis, Liriomyza sativae, Lucilia spp., e.g. Lucilia cuprina, Lutzomyia spp., Mansonia spp., Musca spp., e.g. Musca domestica, Musca domestica vicina, Oestrus spp., Oscinella frit, Paratanytarsus spp., Paralauterborniella subcincta, Pegomya oder Pegomyia spp., e.g. Pegomya betae, Pegomya hyoscyami, Pegomya rubivora, Phlebotomus spp., Phorbia spp., Phormia spp., Piophila casei, Platyparea poeciloptera, Prodiplosis spp., Psila rosae, Rhagoletis spp., e.g. Rhagoletis cingulata, Rhagoletis completa, Rhagoletis fausta, Rhagoletis indifferens, Rhagoletis mendax, Rhagoletis pomonella, Sarcophaga spp., Simulium spp., e.g. Simulium meridionale, Stomoxys spp., Tabanus spp., Tetanops spp., Tipula spp., e.g. Tipula paludosa, Tipula simplex, Toxotrypana curvicauda;
from the order of the Hemiptera, for example Acizzia acaciaebaileyanae, Acizzia dodonaeae, Acizzia uncatoides, Acrida turrita, Acyrthosipon spp., e.g. Acyrthosiphon pisum, Acrogonia spp., Aeneolamia spp., Agonoscena spp., Aleurocanthus spp., Aleyrodes proletella, Aleurolobus barodensis, Aleurothrixus floccosus, Allocaridara malayensis, Amrasca spp., e.g. Amrasca bigutulla, Amrasca devastans, Anuraphis cardui, Aonidiella spp., e.g. Aonidiella aurantii, Aonidiella citrina, Aonidiella inornata, Aphanostigma piri, Aphis spp., e.g. Aphis citricola, Aphis craccivora, Aphis fabae, Aphis forbesi, Aphis glycines, Aphis gossypii, Aphis hederae, Aphis illinoisensis, Aphis middletoni, Aphis nasturtii, Aphis nerii, Aphis pomi, Aphis spiraecola, Aphis viburniphila, Arboridia apicalis, Arytainilla spp., Aspidiella spp., Aspidiotus spp., e.g. Aspidiotus nerii, Atanus spp., Aulacorthum solani, Bemisia tabaci, Blastopsylla occidentalis, Boreioglycaspis melaleucae, Brachycaudus helichrysi, Brachycolus spp., Brevicoryne brassicae, Cacopsylla spp., e.g. Cacopsylla pyricola, Calligypona marginata, Capulinia spp., Carneocephala fulgida, Ceratovacuna lanigera, Cercopidae, Ceroplastes spp., Chaetosiphon fragaefolii, Chionaspis tegalensis, Chlorita onukii, Chondracris rosea, Chromaphis juglandicola, Chrysomphalus aonidum, Chrysomphalus ficus, Cicadulina mbila, Coccomytilus halli, Coccus spp., e.g. Coccus hesperidum, Coccus longulus, Coccus pseudomagnoliarum, Coccus viridis, Cryptomyzus ribis, Cryptoneossa spp., Ctenarytaina spp., Dalbulus spp., Dialeurodes chittendeni, Dialeurodes citri, Diaphorina citri, Diaspis spp., Diuraphis spp., Doralis spp., Drosicha spp., Dysaphis spp., e.g. Dysaphis apiifolia, Dysaphis plantaginea, Dysaphis tulipae, Dysmicoccus spp., Empoasca spp., e.g. Empoasca abrupta, Empoasca fabae, Empoasca maligna, Empoasca solana, Empoasca stevensi, Eriosoma spp., e.g. Eriosoma americanum, Eriosoma lanigerum, Eriosoma pyricola, Erythroneura spp., Eucalyptolyma spp., Euphyllura spp., Euscelis bilobatus, Ferrisia spp., Fiorinia spp., Furcaspis oceanica, Geococcus coffeae, Glycaspis spp., Heteropsylla cubana, Heteropsylla spinulosa, Homalodisca coagulata, Hyalopterus arundinis, Hyalopterus pruni, Icerya spp., e.g. Icerya purchasi, Idiocerus spp., Idioscopus spp., Laodelphax striatellus, Lecanium spp., e.g. Lecanium comi (=Parthenolecanium corni), Lepidosaphes spp., e.g. Lepidosaphes ulmi, Lipaphis erysimi, Lopholeucaspis japonica, Lycorma delicatula, Macrosiphum spp., e.g. Macrosiphum euphorbiae, Macrosiphum lilii, Macrosiphum rosae, Macrosteles facifrons, Mahanarva spp., Melanaphis sacchari, Metcalfiella spp., Metcalfa pruinosa, Metopolophium dirhodum, Monellia costalis, Monelliopsis pecanis, Myzus spp., e.g. Myzus ascalonicus, Myzus cerasi, Myzus ligustri, Myzus ornatus, Myzus persicae, Myzus nicotianae, Nasonovia ribisnigri, Neomaskellia spp., Nephotettix spp., e.g. Nephotettix cincticeps, Nephotettix nigropictus, Nettigoniclla spectra, Nilaparvata lugens, Oncometopia spp., Orthezia praelonga, Oxya chinensis, Pachypsylla spp., Parabemisia myricae, Paratrioza spp., e.g. Paratrioza cockerelli, Parlatoria spp., Pemphigus spp., e.g. Pemphigus bursarius, Pemphigus populivenae, Peregrinus maidis, Perkinsiella spp., Phenacoccus spp., e.g. Phenacoccus madeirensis, Phloeomyzus passerinii, Phorodon humuli, Phylloxera spp., e.g. Phylloxera devastatrix, Phylloxera notabilis, Pinnaspis aspidistrae, Planococcus spp., e.g. Planococcus citri, Prosopidopsylla flava, Protopulvinaria pyriformis, Pseudaulacaspis pentagona, Pseudococcus spp., e.g. Pseudococcus calceolariae, Pseudococcus comstocki, Pseudococcus longispinus, Pseudococcus maritimus, Pseudococcus vibumi, Psyllopsis spp., Psylla spp., e.g. Psylla buxi, Psylla mali, Psylla pyri, Pteromalus spp., Pulvinaria spp., Pyrilla spp., Quadraspidiotus spp., e.g. Quadraspidiotus juglansregiae, Quadraspidiotus ostreaeformis, Quadraspidiotus perniciosus, Quesada gigas, Rastrococcus spp., Rhopalosiphum spp., e.g. Rhopalosiphum maidis, Rhopalosiphum oxyacanthae, Rhopalosiphum padi, Rhopalosiphum rufiabdominale, Saissetia spp., e.g. Saissetia coffeae, Saissetia miranda, Saissetia neglecta, Saissetia oleae, Scaphoideus titanus, Schizaphis graminum, Selenaspidus articulatus, Sipha flava, Sitobion avenae, Sogata spp., Sogatella furcifera, Sogatodes spp., Stictocephala festina, Siphoninus phillyreae, Tenalaphara malayensis, Tetragonocephela spp., Tinocallis caryaefoliae, Tomaspis spp., Toxoptera spp., e.g. Toxoptera aurantii, Toxoptera citricidus, Trialeurodes vaporariorum, Trioza spp., e.g. Trioza diospyri, Typhlocyba spp., Unaspis spp., Viteus vitifolii, Zygina spp.;
from the suborder of the Heteroptera, for example, Aelia spp., Anasa tristis, Antestiopsis spp., Boisea spp., Blissus spp., Calocoris spp., Campylomma livida, Cavelerius spp., Cimex spp., e.g. Cimex adjunctus, Cimex hemipterus, Cimex lectularius, Cimex pilosellus, Collaria spp., Creontiades dilutus, Dasynus piperis, Dichelops furcatus, Diconocoris hewetti, Dysdercus spp., Euschistus spp., e.g. Euschistus heros, Euschistus servus, Euschistus tristigmus, Euschistus variolarius, Eurydema spp., Eurygaster spp., Halyomorpha halys, Heliopeltis spp., Horcias nobilellus, Leptocorisa spp., Leptocorisa varicornis, Leptoglossus occidentalis, Leptoglossus phyllopus, Lygocoris spp., e.g. Lygocoris pabulinus, Lygus spp., e.g. Lygus elisus, Lygus hesperus, Lygus lineolaris, Macropes excavatus, Megacopta cribraria, Miridae, Monalonion atratum, Nezara spp., e.g. Nezara viridula, Nysius spp., Oebalus spp., Pentomidae, Piesma quadrata, Piezodorus spp., e.g. Piezodorus guildinii, Psallus spp., Pseudacysta persea, Rhodnius spp., Sahlbergella singularis, Scaptocoris castanea, Scotinophora spp., Stephanitis nashi, Tibraca spp., Triatoma spp.;
from the order of the Hymenoptera, for example, Acromyrmex spp., Athalia spp., e.g. Athalia rosae, Atta spp., Camponotus spp., Dolichovespula spp., Diprion spp., e.g. Diprion similis, Hoplocampa spp., e.g. Hoplocampa cookei, Hoplocampa testudinea, Lasius spp., Linepithema (Iridiomyrmex) humile, Monomorium pharaonis, Paratrechina spp., Paravespula spp., Plagiolepis spp., Sirex spp., e.g. Sirex noctilio, Solenopsis invicta, Tapinoma spp., Technomyrmex albipes, Urocerus spp., Vespa spp., e.g. Vespa crabro, Wasmannia auropunctata, Xeris spp.;
from the order of the Isopoda, for example Armadillidium vulgare, Oniscus asellus, Porcellio scaber;
from the order of the Isoptera, for example, Coptotermes spp., e.g. Coptotermes formosanus, Cornitermes cumulans, Cryptotermes spp., Incisitermes spp., Kalotermes spp., Microtermes obesi, Nasutitermis spp., Odontotermes spp., Porotermes spp., Reticulitermes spp., e.g. Reticulitermes flavipes, Reticulitermes hesperus;
from the order of the Lepidoptera, for example Achroia grisella, Acronicta major, Adoxophyes spp., e.g. Adoxophyes orana, Aedia leucomelas, Agrotis spp., e.g. Agrotis segetum, Agrotis ipsilon, Alabama spp., e.g. Alabama argillacea, Amyelois transitella, Anarsia spp., Anticarsia spp., e.g. Anticarsia gemmatalis, Argyroploce spp., Autographa spp., Barathra brassicae, Blastodacna atra, Borbo cinnara, Bucculatrix thurberiella, Bupalus piniarius, Busseola spp., Cacoecia spp., Caloptilia theivora, Capua reticulana, Carpocapsa pomonella, Carposina niponensis, Cheimatobia brumata, Chilo spp., e.g. Chilo plejadellus, Chilo suppressalis, Choreutis pariana, Choristoneura spp., Chrysodeixis chalcites, Clysia ambiguella, Cnaphalocerus spp., Cnaphalocrocis medinalis, Cnephasia spp., Conopomorpha spp., Conotrachelus spp., Copitarsia spp., Cydia spp., e.g. Cydia nigricana, Cydia pomonella, Dalaca noctuides, Diaphania spp., Diparopsis spp., Diatraea saccharalis, Dioryctria spp., e.g. Dioryctria zimmermani, Earias spp., Ecdytolopha aurantium, Elasmopalpus lignosellus, Eldana saccharina, Ephestia spp., e.g. Ephestia elutella, Ephestia kuehniella, Epinotia spp., Epiphyas postvittana, Erannis spp., Erschoviella musculana, Etiella spp., Eudocima spp., Eulia spp., Eupoecilia ambiguella, Euproctis spp., e.g. Euproctis chrysorrhoea, Euxoa spp., Feltia spp., Galleria mellonella, Gracillaria spp., Grapholitha spp., e.g. Grapholita molesta, Grapholita prunivora, Hedylepta spp., Helicoverpa spp., e.g. Helicoverpa armigera, Helicoverpa zea, Heliothis spp., e.g. Heliothis virescens, Hofmannophila pseudospretella, Homoeosoma spp., Homona spp., Hyponomeuta padella, Kakivoria flavofasciata, Lampides spp., Laphygma spp., Laspeyresia molesta, Leucinodes orbonalis, Leucoptera spp., e.g. Leucoptera coffeella, Lithocolletis spp., e.g. Lithocolletis blancardella, Lithophane antennata, Lobesia spp., e.g. Lobesia botrana, Loxagrotis albicosta, Lymantria spp., e.g. Lymantria dispar, Lyonetia spp., e.g. Lyonetia clerkella, Malacosoma neustria, Maruca testulalis, Mamestra brassicae, Melanitis leda, Mocis spp., Monopis obviella, Mythimna separata, Nemapogon cloacellus, Nymphula spp., Oiketicus spp., Omphisa spp., Operophtera spp., Oria spp., Orthaga spp., Ostrinia spp., e.g. Ostrinia nubilalis, Panolis flammea, Parnara spp., Pectinophora spp., e.g. Pectinophora gossypiella, Perileucoptera spp., Phthorimaea spp., e.g. Phthorimaea operculella, Phyllocnistis citrella, Phyllonorycter spp., e.g. Phyllonorycter blancardella, Phyllonorycter crataegella, Pieris spp., e.g. Pieris rapae, Platynota stultana, Plodia interpunctella, Plusia spp., Plutella xylostella (=Plutella maculipennis), Podesia spp., e.g. Podesia syringae, Prays spp., Prodenia spp., Protoparce spp., Pseudaletia spp., e.g. Pseudaletia unipuncta, Pseudoplusia includens, Pyrausta nubilalis, Rachiplusia nu, Schoenobius spp., e.g. Schoenobius bipunctifer, Scirpophaga spp., e.g. Scirpophaga innotata, Scotia segetum, Sesamia spp., e.g. Sesamia inferens, Sparganothis spp., Spodoptera spp., e.g. Spodoptera eradiana, Spodoptera exigua, Spodoptera frugiperda, Spodoptera praefica, Stathmopoda spp., Stenoma spp., Stomopteryx subsecivella, Synanthedon spp., Tecia solanivora, Thaumetopoea spp., Thermesia gemmatalis, Tinea cloacella, Tinea pellionella, Tineola bisselliella, Tortrix spp., Trichophaga tapetzella, Trichoplusia spp., e.g. Trichoplusia ni, Tryporyza incertulas, Tuta absoluta, Virachola spp.;
from the order of the Orthoptera or Saltatoria, for example Acheta domesticus, Dichroplus spp., Gryllotalpa spp., e.g. Gryllotalpa gryllotalpa, Hieroglyphus spp., Locusta spp., e.g. Locusta migratoria, Melanoplus spp., e.g. Melanoplus devastator, Paratlanticus ussuriensis, Schistocerca gregaria;

from the order of the Phthiraptera, for example Damalinia spp., Haematopinus spp., Linognathus spp., Pediculus spp., Phylloxera vastatrix, Phthirus pubis, Trichodectes spp.;

from the order of the Psocoptera, for example Lepinotus spp., Liposcelis spp.;
from the order of the Siphonaptera, for example Ceratophyllus spp., Ctenocephalides spp., e.g. Ctenocephalides canis, Ctenocephalides felis, Pulex irritans, Tunga penetrans, Xenopsylla cheopis;
from the order of the Thysanoptera, for example Anaphothrips obscurus, Baliothrips biformis, Chaetanaphothrips leeuweni, Drepanothrips reuteri, Enneothrips flavens, Frankliniella spp., e.g. Frankliniella fusca, Frankliniella occidentalis, Frankliniella schultzei, Frankliniella tritici, Frankliniella vaccinii, Frankliniella williamsi, Haplothrips spp., Heliothrips spp., Hercinothrips femoralis, Kakothrips spp., Rhipiphorothrips cruentatus, Scirtothrips spp., Taeniothrips cardamomi, Thrips spp., e.g. Thrips palmi, Thrips tabaci;
from the order of the Zygentoma (=Thysanura), for example Ctenolepisma spp., Lepisma saccharina, Lepismodes inquilinus, Thermobia domestica;
from the class of the Symphyla, for example Scutigerella spp., e.g. Scutigerella immaculata;
pests from the phylum of the Mollusca, for example from the class of the Bivalvia, e.g. Dreissena spp.;
and from the class of the Gastropoda, for example Arion spp., e.g. Arion ater rufus, Biomphalaria spp., Bulinus spp., Deroceras spp., e.g. Deroceras laeve, Galba spp., Lymnaea spp., Oncomelania spp., Pomacea spp., Succinea spp.;
plant pests from the phylum of the Nematoda, i.e. phytoparasitic nematodes, in particular Aglenchus spp., e.g. Aglenchus agricola, Anguina spp., e.g. Anguina tritici, Aphelenchoides spp., e.g. Aphelenchoides arachidis, Aphelenchoides fragariae, Belonolaimus spp., e.g. Belonolaimus gracilis, Belonolaimus longicaudatus, Belonolaimus nortoni, Bursaphelenchus spp., e.g. Bursaphelenchus cocophilus, Bursaphelenchus eremus, Bursaphelenchus xylophilus, Cacopaurus spp., e.g. Cacopaurus pestis, Criconemella spp., e.g. Criconemella curvata, Criconemella onoensis, Criconemella ornata, Criconemella rusium, Criconemella xenoplax (=Mesocriconema xenoplax), Criconemoides spp., e.g. Criconemoides ferniae, Criconemoides onoense, Criconemoides ornatum, Ditylenchus spp., e.g. Ditylenchus dipsaci, Dolichodorus spp., Globodera spp., e.g. Globodera pallida, Globodera rostochiensis, Helicotylenchus spp., e.g. Helicotylenchus dihystera, Hemicriconemoides spp., Hemicycliophora spp., Heterodera spp., e.g. Heterodera avenae, Heterodera glycines, Heterodera schachtii, Hirschmaniella spp., Hoplolaimus spp., Longidorus spp., e.g. Longidorus africanus, Meloidogyne spp., e.g. Meloidogyne chitwoodi, Meloidogyne fallax, Meloidogyne hapla, Meloidogyne incognita, Meloinema spp., Nacobbus spp., Neotylenchus spp., Paralongidorus spp., Paraphelenchus spp., Paratrichodorus spp., e.g. Paratrichodorus minor, Paratylenchus spp., Pratylenchus spp., e.g. Pratylenchus penetrans, Pseudohalenchus spp., Psilenchus spp., Punctodera spp., Quinisulcius spp., Radopholus spp., e.g. Radopholus citrophilus, Radopholus similis, Rotylenchulus spp., Rotylenchus spp., Scutellonema spp., Subanguina spp., Trichodorus spp., e.g. Trichodorus obtusus, Trichodorus primitivus, Tylenchorhynchus spp., e.g. Tylenchorhynchus annulatus, Tylenchulus spp., e.g. Tylenchulus semipenetrans, Xiphinema spp., e.g. Xiphinema index.

The compounds of the formula (I) can, as the case may be, at certain concentrations or application rates, also be used as herbicides, safeners, growth regulators or agents to improve plant properties, as microbicides or gametocides, for example as fungicides, antimycotics, bactericides, virucides (including agents against viroids) or as agents against MLO (mycoplasma-like organisms) and RLO (rickettsia-like organisms). They can, as the case may be, also be used as intermediates or precursors for the synthesis of other active compounds.

### Formulations

The present invention further relates to formulations and application forms prepared therefrom as pesticides, for example drench, drip and spray liquors, comprising at least one compound of the formula (I). Optionally, the application forms comprise further pesticides and/or adjuvants which improve action, such as penetrants, e.g. vegetable oils, for example rapeseed oil, sunflower oil, mineral oils, for example paraffin oils, alkyl esters of vegetable fatty acids, for example rapeseed oil methyl ester or soya oil methyl ester, or alkanol alkoxylates and/or spreaders, for example alkylsiloxanes and/or salts, for example organic or inorganic ammonium or phosphonium salts, for example ammonium sulfate or diammonium hydrogenphosphate and/or retention promoters, for example dioctyl sulfosuccinate or hydroxypropylguar polymers and/or humectants, for example glycerol and/or fertilizers, for example ammonium-, potassium- or phosphorus-containing fertilizers.

Customary formulations are, for example, water-soluble liquids (SL), emulsion concentrates (EC), emulsions in water (EW), suspension concentrates (SC, SE, FS, OD), water-dispersible granules (WG), granules (GR) and capsule concentrates (CS); these and further possible formulation types are described, for example, by Crop Life International and in Pesticide Specifications, Manual on development and use of FAO and WHO specifications for pesticides, FAO Plant Production and Protection Papers—173, prepared by the FAO/WHO Joint Meeting on Pesticide Specifications, 2004, ISBN: 9251048576. The formulations, in addition to one or more compounds of the formula (I), optionally comprise further agrochemical active compounds.

Preference is given to formulations or application forms comprising auxiliaries, for example extenders, solvents, spontaneity promoters, carriers, emulsifiers, dispersants, frost protection agents, biocides, thickeners and/or further auxiliaries, for example adjuvants. An adjuvant in this context is a component which enhances the biological effect of the formulation, without the component itself having any biological effect. Examples of adjuvants are agents which promote retention, spreading, attachment to the leaf surface or penetration.

These formulations are produced in a known manner, for example by mixing the compounds of the formula (I) with auxiliaries, for example extenders, solvents and/or solid carriers and/or other auxiliaries, for example surfactants. The formulations are produced either in suitable facilities or else before or during application.

The auxiliaries used may be substances suitable for imparting special properties, such as certain physical, technical and/or biological properties, to the formulation of the compounds of the formula (I), or to the application forms prepared from these formulations (for example ready-to-use pesticides such as spray liquors or seed-dressing products).

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the simple and substituted amines, amides, lactams (such as N-alkylpyrrolidones) and lactones, the sulfones and sulfoxides (such as dimethyl sulfoxide).

If the extender used is water, it is also possible to use, for example, organic solvents as auxiliary solvents. Useful liquid solvents are essentially: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulfoxide, and also water.

In principle, it is possible to use all suitable solvents. Examples of suitable solvents are aromatic hydrocarbons, for example xylene, toluene or alkylnaphthalenes, chlorinated aromatic or chlorinated aliphatic hydrocarbons, for example chlorobenzene, chloroethylene or methylene chloride, aliphatic hydrocarbons, for example cyclohexane, paraffins, petroleum fractions, mineral and vegetable oils, alcohols, for example methanol, ethanol, isopropanol, butanol or glycol and their ethers and esters, ketones, for example acetone, methyl ethyl ketone, methyl isobutyl ketone or cyclohexanone, strongly polar solvents, for example dimethyl sulfoxide, and water.

In principle, it is possible to use all suitable carriers. Suitable carriers include more particularly the following: e.g. ammonium salts and natural, finely ground rocks, such as kaolins, aluminas, talc, chalk, quartz, attapulgite, montmorillonite or diatomaceous earth, and synthetic, finely ground rocks, such as highly disperse silica, aluminium oxide and natural or synthetic silicates, resins, waxes and/or solid fertilizers. It is likewise possible to use mixtures of such carriers. Useful carriers for granules include: for example crushed and fractionated natural rocks such as calcite, marble, pumice, sepiolite, dolomite, and synthetic granules of inorganic and organic flours, and also granules of organic material such as sawdust, paper, coconut shells, maize cobs and tobacco stalks.

It is also possible to use liquefied gaseous extenders or solvents. Especially suitable extenders or carriers are those which are gaseous at standard temperature and under atmospheric pressure, for example aerosol propellants such as halogenated hydrocarbons, and also butane, propane, nitrogen and carbon dioxide.

Examples of emulsifiers and/or foam formers, dispersants or wetting agents having ionic or nonionic properties or mixtures of these surface-active substances are salts of polyacrylic acid, salts of lignosulfonic acid, salts of phenolsulfonic acid or naphthalenesulfonic acid, polycondensates of ethylene oxide with fatty alcohols or with fatty acids or with fatty amines, with substituted phenols (preferably alkylphenols or arylphenols), salts of sulfosuccinic esters, taurine derivatives (preferably alkyl taurates), phosphoric esters of polyethoxylated alcohols or phenols, fatty acid esters of polyols, and derivatives of the compounds containing sulfates, sulfonates and phosphates, for example alkylaryl polyglycol ethers, alkylsulfonates, alkyl sulfates, arylsulfonates, protein hydrolysates, lignosulfite waste liquors and methylcellulose. The presence of a surfactant is advantageous if one of the compounds of the formula (I) and/or one of the inert carriers is insoluble in water and if the application takes place in water.

Further auxiliaries which may be present in the formulations and the application forms derived therefrom include dyes such as inorganic pigments, for example iron oxide, titanium oxide and Prussian Blue, and organic dyes such as alizarin dyes, azo dyes and metal phthalocyanine dyes, and nutrients and trace nutrients such as salts of iron, manganese, boron, copper, cobalt, molybdenum and zinc.

Additional components which may be present are stabilizers, such as cold stabilizers, preservatives, antioxidants, light stabilizers, or other agents which improve chemical and/or physical stability. Foam generators or antifoams may also be present.

In addition, the formulations and application forms derived therefrom may also comprise, as additional auxiliaries, stickers such as carboxymethylcellulose and natural and synthetic polymers in the form of powders, granules or latices, such as gum arabic, polyvinyl alcohol and polyvinyl acetate, and natural phospholipids such as cephalins and lecithins and synthetic phospholipids. Further auxiliaries may be mineral and vegetable oils.

It is possible if appropriate for still further auxiliaries to be present in the formulations and the application forms derived therefrom. Examples of such additives are fragrances, protective colloids, binders, adhesives, thickeners, thixotropic agents, penetrants, retention promoters, stabilizers, sequestrants, complexing compositions, humectants, spreaders. In general, the compounds of the formula (I) can be combined with any solid or liquid additive commonly used for formulation purposes.

Useful retention promoters include all those substances which reduce dynamic surface tension, for example dioctyl sulfosuccinate, or increase viscoelasticity, for example hydroxypropylguar polymers.

Useful penetrants in the present context are all those substances which are typically used to improve the penetration of agrochemical active compounds into plants. Penetrants are defined in this context by their ability to penetrate from the (generally aqueous) application liquor and/or from the spray coating into the cuticle of the plant and hence to increase the mobility of the active compounds in the cuticle. The method described in the literature (Baur et al., 1997, Pesticide Science 51, 131-152) can be used for determining this property. Examples include alcohol alkoxylates such as coconut fatty ethoxylate (10) or isotridecyl ethoxylate (12), fatty acid esters, for example rapeseed oil methyl ester or soya oil methyl ester, fatty amine alkoxylates, for example tallowamine ethoxylate (15), or ammonium and/or phosphonium salts, for example ammonium sulfate or diammonium hydrogenphosphate.

The formulations preferably comprise between 0.00000001% and 98% by weight of the compound of the formula (I), more preferably between 0.01% and 95% by weight of the compound of the formula (I), most preferably between 0.5% and 90% by weight of the compound of the formula (I), based on the weight of the formulation.

The content of the compound of the formula (I) in the application forms prepared from the formulations (in particular pesticides) may vary within wide ranges. The concentration of the compound of the formula (I) in the application forms may typically be between 0.00000001% and 95% by weight of the compound of the formula (I), preferably between 0.00001% and 1% by weight, based on the weight of the application form. Application is accomplished in a customary manner appropriate for the application forms.

### Mixtures

The compounds of the formula (I) can also be used in a mixture with one or more suitable fungicides, bactericides, acaricides, molluscicides, nematicides, insecticides, microbiological agents, beneficial organisms, herbicides, fertilizers, bird repellents, phytotonics, sterilants, safeners, semiochemicals and/or plant growth regulators, in order thus, for example, to broaden the spectrum of action, prolong the period of action, enhance the rate of action, prevent repellency or prevent evolution of resistance. In addition, active compound combinations of this kind can improve plant growth and/or tolerance to abiotic factors, for example high or low temperatures, to drought or to elevated levels of water or soil salinity. It is also possible to improve flowering and fruiting performance, optimize germination capacity and root development, facilitate harvesting and improve harvest yields, influence maturation, improve the quality and/or the nutritional value of the harvested products, prolong storage life and/or improve the processability of the harvested products.

In addition, the compounds of the formula (I) may be present in a mixture with other active compounds or semiochemicals such as attractants and/or bird repellents and/or plant activators and/or growth regulators and/or fertilizers. Likewise, the compounds of the formula (I) can be used to improve plant properties, for example growth, yield and quality of the harvested material.

In a particular embodiment according to the invention, the compounds of the formula (I) are present in formulations or in the application forms prepared from these formulations in a mixture with further compounds, preferably those as described below.

If one of the compounds mentioned below can occur in different tautomeric forms, these forms are also included even if not explicitly mentioned in each case. All the mixing components mentioned, as the case may be, may also form salts with suitable bases or acids if they are capable of doing so on the basis of their functional groups.

### Insecticides/Acaricides/Nematicides

The active compounds specified here by their common names are known and are described for example in “The Pesticide Manual” (16th ed., British Crop Protection Council 2012) or can be searched for on the Internet (e.g. http://www.alanwood.net/pesticides). The classification is based on the IRAC Mode of Action Classification Scheme applicable at the time of filing of this patent application.

(1) Acetylcholinesterase (AChE) inhibitors, for example carbamates, e.g. alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC and xylylcarb; or organophosphates, e.g. acephate, azamethiphos, azinphos-ethyl, azinphos-methyl, cadusafos, chlorethoxyfos, chlorfenvinphos, chlormephos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothiophosphoryl) salicylate, isoxathion, malathion, mecarbam, methamidophos, methidathion, mevinphos, monocrotophos, naled, omethoate, oxydemeton-methyl, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, triclorfon and vamidothion.
(2) GABA-gated chloride channel blockers, for example cyclodiene-organochlorines, e.g. chlordane and endosulfan or phenylpyrazoles (fiproles), e.g. ethiprole and fipronil.
(3) Sodium channel modulators, for example pyrethroids, e.g. acrinathrin, allethrin, d-cis-trans allethrin, d-trans allethrin, bifenthrin, bioallethrin, bioallethrin S-cyclopentenyl isomer, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin [(1R)-trans isomer], deltamethrin, empenthrin [(EZ)-(1R) isomer], esfenvalerate, etofenprox, fenpropathrin, fenvalerate, flucythrinate, flumethrin, tau-fluvalinate, halfenprox, imiprothrin, kadethrin, momfluorothrin, permethrin, phenothrin [(1R)-trans isomer], prallethrin, pyrethrins (pyrethrum), resmethrin, silafluofen, tefluthrin, tetramethrin, tetramethrin [(1R) isomer], tralomethrin and transfluthrin or DDT or methoxychlor.
(4) Nicotinic acetylcholine receptor (nAChR) competitive modulators, for example neonicotinoids, e.g. acetamiprid, clothianidin, dinotefuran, imidacloprid, nitenpyram, thiacloprid and thiamethoxam or nicotine or sulfoxaflor or flupyradifurone.
(5) Nicotinic acetylcholine receptor (nAChR) allosteric modulators, for example spinosyns, e.g. spinetoram and spinosad.
(6) Glutamate-gated chloride channel (GluCl) allosteric modulators, for example avermectins/milbemycins, e.g. abamectin, emamectin benzoate, lepimectin and milbemectin.
(7) Juvenile hormone mimetics, for example juvenile hormone analogues, e.g. hydroprene, kinoprene and methoprene or fenoxycarb or pyriproxyfen.
(8) Miscellaneous non-specific (multisite) inhibitors, for example alkyl halides, e.g. methyl bromide and other alkyl halides; or chloropicrin or sulfuryl fluoride or borax or tartar emetic or methyl isocyanate generator, e.g. diazomet and metam.
(9) Chordotonal organ modulators, e.g. pymetrozine or flonicamide.
(10) Mite growth inhibitors, for example clofentezine, hexythiazox and diflovidazin or etoxazole.
(11) Microbial disruptors of the insect gut membrane, for example Bacillus thuringiensis subspecies israelensis, Bacillus sphaericus, Bacillus thuringiensis subspecies aizawai, Bacillus thuringiensis subspecies kurstaki, Bacillus thuringiensis subspecies tenebrionis and B.t. plant proteins: Cry1Ab, Cry1Ac, Cry1Fa, Cry1A.105, Cry2Ab, VIP3A, mCry3A, Cry3Ab, Cry3Bb, Cry34Ab1/35Ab1.
(12) Inhibitors of mitochondrial ATP synthase, such as ATP disruptors, for example diafenthiuron or organotin compounds, e.g. azocyclotin, cyhexatin and fenbutatin oxide or propargite or tetradifon.
(13) Uncouplers of oxidative phosphorylation via disruption of the proton gradient, for example chlorfenapyr, DNOC and sulfluramid.
(14) Nicotinic acetylcholine receptor channel blockers, for example bensultap, cartap hydrochloride, thiocyclam, and thiosultap-sodium.
(15) Inhibitors of chitin biosynthesis, type 0, for example bistrifluron, chlorfluazuron, diflubenzuron, flucycloxuron, flufenoxuron, hexaflumuron, lufenuron, novaluron, noviflumuron, teflubenzuron and triflumuron.
(16) Inhibitors of chitin biosynthesis, type 1, for example buprofezin.
(17) Moulting disruptors (especially in the case of Diptera), for example cyromazine.
(18) Ecdysone receptor agonists, for example chromafenozide, halofenozide, methoxyfenozide and tebufenozide.
(19) Octopamine receptor agonists, for example amitraz.
(20) Mitochondrial complex III electron transport inhibitors, for example hydramethylnon or acequinocyl or fluacrypyrim.
(21) Mitochondrial complex I electron transport inhibitors, for example METI acaricides, e.g. fenazaquin, fenpyroximate, pyrimidifen, pyridaben, tebufenpyrad and tolfenpyrad or rotenone (Derris).
(22) Voltage-dependent sodium channel blockers, for example indoxacarb or metaflumizone.
(23) Inhibitors of acetyl CoA carboxylase, for example tetronic and tetramic acid derivatives, e.g. spirodiclofen, spiromesifen and spirotetramat.
(24) Mitochondrial complex IV electron transport inhibitors, for example phosphines, e.g. aluminium phosphide, calcium phosphide, phosphine and zinc phosphide, or cyanides, calcium cyanide, potassium cyanide and sodium cyanide.
(25) Mitochondrial complex II electron transport inhibitors, for example beta-keto nitrile derivatives, e.g. cyenopyrafen and cyflumetofen and carboxanilides, for example pyflubumide.
(28) Ryanodine receptor modulators, for example diamides, e.g. chlorantraniliprole, cyantraniliprole and flubendiamide,
further active compounds, for example afidopyropen, afoxolaner, azadirachtin, benclothiaz, benzoximate, bifenazate, broflanilide, bromopropylate, chinomethionat, chloroprallethrin, cryolite, cyclaniliprole, cycloxaprid, cyhalodiamide, dicloromezotiaz, dicofol, epsilon metofluthrin, epsilon momfluthrin, flometoquin, fluazaindolizine, fluensulfone, flufenerim, flufenoxystrobin, flufiprole, fluhexafon, fluopyram, fluralaner, fluxametamide, fufenozide, guadipyr, heptafluthrin, imidaclothiz, iprodione, kappa bifenthrin, kappa tefluthrin, lotilaner, meperfluthrin, paichongding, pyridalyl, pyrifluquinazon, pyriminostrobin, spirobudiclofen, tetramethylfluthrin, tetraniliprole, tetrachlorantraniliprole, tioxazafen, thiofluoximate, triflumezopyrim and iodomethane; additionally preparations based on Bacillus firmus (I-1582, BioNeem, Votivo), and the following compounds: 1-{2-fluoro-4-methyl-5-[(2,2,2-trifluoroethyl)sulfinyl]phenyl}-3-(trifluoromethyl)-1H-1,2,4-triazole-5-amine (known from WO2006/043635) (CAS 885026-50-6), {1′-[(2E)-3-(4-chlorophenyl)prop-2-en-1-yl]-5-fluorospiro[indole-3,4′-piperidine]-1(2H)-yl}(2-chloropyridin-4-yl)methanone (known from WO2003/106457) (CAS 637360-23-7), 2-chloro-N-[2-{1-[(2E)-3-(4-chlorophenyl)prop-2-en-1-yl]piperidin-4-yl}-4-(trifluoromethyl)phenyl]isonicotinamide (known from WO2006/003494) (CAS 872999-66-1), 3-(4-chloro-2,6-dimethylphenyl)-4-hydroxy-8-methoxy-1,8-diazaspiro[4.5]dec-3-en-2-one (known from WO 2010052161) (CAS 1225292-17-0), 3-(4-chloro-2,6-dimethylphenyl)-8-methoxy-2-oxo-1,8-diazaspiro[4.5]dec-3-en-4-yl ethylcarbonate (known from EP 2647626) (CAS-1440516-42-6), 4-(but-2-yn-1-yloxy)-6-(3,5-dimethylpiperidin-1-yl)-5-fluoropyrimidine (known from WO2004/099160) (CAS 792914-58-0), PF1364 (known from JP2010/018586) (CAS Reg. No. 1204776-60-2), N-[(2E)-1-[(6-chloropyridin-3-yl)methyl]pyridin-2(1H)-ylidene]-2,2,2-trifluoroacetamide (known from WO2012/029672) (CAS 1363400-41-2), (3E)-3-[1-[(6-chloro-3-pyridyl)methyl]-2-pyridylidene]-1,1,1-trifluoropropan-2-one (known from WO2013/144213) (CAS 1461743-15-6), N-[3-(benzylcarbamoyl)-4-chlorophenyl]-1-methyl-3-(pentafluoroethyl)-4-(trifluoromethyl)-1H-pyrazole-5-carboxamide (known from WO2010/051926) (CAS 1226889-14-0), 5-bromo-4-chloro-N-[4-chloro-2-methyl-6-(methylcarbamoyl)phenyl]-2-(3-chloro-2-pyridyl)pyrazole-3-carboxamide (known from CN103232431) (CAS 1449220-44-3), 4-[5-(3,5-dichlorophenyl)-4,5-dihydro-5-(trifluoromethyl)-3-isoxazolyl]-2-methyl-N-(cis-1-oxido-3-thietanyl)benzamide, 4-[5-(3,5-dichlorophenyl)-4,5-dihydro-5-(trifluoromethyl)-3-isoxazolyl]-2-methyl-N-(trans-1-oxido-3-thietanyl)benzamide and 4-[(5S)-5-(3,5-dichlorophenyl)-4,5-dihydro-5-(trifluoromethyl)-3-isoxazolyl]-2-methyl-N-(cis-1-oxido-3-thietanyl)benzamide (known from WO 2013/050317 A1) (CAS 1332628-83-7), N-[3-chloro-1-(3-pyridinyl)-1H-pyrazol-4-yl]-N-ethyl-3-[(3,3,3-trifluoropropyl)sulfinyl]propanamide, (+)-N-[3-chloro-1-(3-pyridinyl)-1H-pyrazol-4-yl]-N-ethyl-3-[(3,3,3-trifluoropropyl)sulfinyl]propanamide and (−)-N-[3-chloro-1-(3-pyridinyl)-1H-pyrazol-4-yl]-N-ethyl-3-[(3,3,3-trifluoropropyl)sulfinyl]propanamide (known from WO 2013/162715 A2, WO 2013/162716 A2, US 2014/0213448 A1) (CAS 1477923-37-7), 5-[[(2E)-3-chloro-2-propen-1-yl]amino]-1-[2,6-dichloro-4-(trifluoromethyl)phenyl]-4-[(trifluoromethyl)sulfinyl]-1H-pyrazole-3-carbonitrile (known from CN 101337937 A) (CAS 1105672-77-2), 3-bromo-N-[4-chloro-2-methyl-6-[(methylamino)thioxomethyl]phenyl]-1-(3-chloro-2-pyridinyl)-1H-pyrazole-5-carboxamide, (liudaibenjiaxuanan, known from CN 103109816 A) (CAS 1232543-85-9); N-[4-chloro-2-[[(1,1-dimethylethyl)amino]carbonyl]-6-methylphenyl]-1-(3-chloro-2-pyridinyl)-3-(fluoromethoxy)-1H-pyrazole-5-carboxamide (known from WO 2012/034403 A1) (CAS 1268277-22-0), N-[2-(5-amino-1,3,4-thiadiazol-2-yl)-4-chloro-6-methylphenyl]-3-bromo-1-(3-chloro-2-pyridinyl)-1H-pyrazole-5-carboxamide (known from WO 2011/085575 A1) (CAS 1233882-22-8), 4-[3-[2,6-dichloro-4-[(3,3-dichloro-2-propen-1-yl)oxy]phenoxy]propoxy]-2-methoxy-6-(trifluoromethyl)pyrimidine (known from CN 101337940 A) (CAS 1108184-52-6); (2E)- and 2(Z)-2-[2-(4-cyanophenyl)-1-[3-(trifluoromethyl)phenyl]ethylidene]-N-[4-(difluoromethoxy)phenyl]hydrazinecarboxamide (known from CN 101715774 A) (CAS 1232543-85-9); 3-(2,2-dichloroethenyl)-2,2-dimethyl-4-(1H-benzimidazol-2-yl)phenyl cyclopropanecarboxylate (known from CN 103524422 A) (CAS 1542271-46-4); methyl (4aS)-7-chloro-2,5-dihydro-2-[[(methoxycarbonyl) [4-[(trifluoromethyl)thio]phenyl]amino]carbonyl]indeno[1,2-e][1,3,4]oxadiazine-4a(3H)-carboxylate (known from CN 102391261 A) (CAS 1370358-69-2); 6-deoxy-3-O-ethyl-2,4-di-O-methyl-1-[N-[4-[1-[4-(1,1,2,2,2-pentafluoroethoxy)phenyl]-1H-1,2,4-triazol-3-yl]phenyl]carbamate]-α-L-mannopyranose (known from US 2014/0275503 A1) (CAS 1181213-14-8); 8-(2-cyclopropylmethoxy-4-trifluoromethylphenoxy)-3-(6-trifluoromethylpyridazin-3-yl)-3-azabicyclo[3.2.1]octane (CAS 1253850-56-4), (8-anti)-8-(2-cyclopropylmethoxy-4-trifluoromethylphenoxy)-3-(6-trifluoromethylpyridazin-3-yl)-3-azabicyclo[3.2.1]octane (CAS 933798-27-7), (8-syn)-8-(2-cyclopropylmethoxy-4-trifluoromethylphenoxy)-3-(6-trifluoromethylpyridazin-3-yl)-3-azabicyclo[3.2.1]octane (known from WO 2007040280 A1, WO 2007040282 A1) (CAS 934001-66-8), N-[3-chloro-1-(3-pyridinyl)-1H-pyrazol-4-yl]-N-ethyl-3-[(3,3,3-trifluoropropyl)thio]propanamide (known from WO 2015/058021 A1, WO 2015/058028 A1) (CAS 1477919-27-9) and N-[4-(aminothioxomethyl)-2-methyl-6-[(methylamino)carbonyl]phenyl]-3-bromo-1-(3-chloro-2-pyridinyl)-1H-pyrazole-5-carboxamide (known from CN 103265527 A) (CAS 1452877-50-7).

### Fungicides

The active compounds specified here by their common names are known and described, for example, in “Pesticide Manual” (16th Ed. British Crop Protection Council) or searchable on the Internet (for example: http://www.alanwood.net/pesticides).

All the mixing components mentioned in classes (1) to (15), as the case may be, may form salts with suitable bases or acids if they are capable of doing so on the basis of their functional groups. All the fungicidal mixing components mentioned in classes (1) to (15), as the case may be, may include tautomeric forms.

1) Ergosterol biosynthesis inhibitors, for example (1.001) cyproconazole, (1.002) difenoconazole, (1.003) epoxiconazole, (1.004) fenhexamid, (1.005) fenpropidine, (1.006) fenpropimorph, (1.007) fenpyrazamine, (1.008) fluquinconazole, (1.009) flutriafol, (1.010) imazalil, (1.011) imazalil sulfate, (1.012) ipconazole, (1.013) metconazole, (1.014) myclobutanil, (1.015) paclobutrazole, (1.016) prochloraz, (1.017) propiconazole, (1.018) prothioconazole, (1.019) pyrisoxazole, (1.020) spiroxamine, (1.021) tebuconazole, (1.022) tetraconazole, (1.023) triadimenol, (1.024) tridemorph, (1.025) triticonazole, (1.026) (1R,2S,5S)-5-(4-chlorobenzyl)-2-(chloromethyl)-2-methyl-1-(1H-1,2,4-triazol-1-ylmethyl)cyclopentanol, (1.027) (1S,2R,5R)-5-(4-chlorobenzyl)-2-(chloromethyl)-2-methyl-1-(1H-1,2,4-triazol-1-ylmethyl)cyclopentanol, (1.028) (2R)-2-(1-chlorocyclopropyl)-4-[(1R)-2,2-dichlorocyclopropyl]-1-(1H-1,2,4-triazol-1-yl)butan-2-ol, (1.029) (2R)-2-(1-chlorocyclopropyl)-4-[(1S)-2,2-dichlorocyclopropyl]-1-(1H-1,2,4-triazol-1-yl)butan-2-ol, (1.030) (2R)-2-[4-(4-chlorophenoxy)-2-(trifluoromethyl)phenyl]-1-(1H-1,2,4-triazol-1-yl)propan-2-ol, (1.031) (2S)-2-(1-chlorocyclopropyl)-4-[(1R)-2,2-dichlorocyclopropyl]-1-(1H-1,2,4-triazol-1-yl)butan-2-ol, (1.032) (2S)-2-(1-chlorocyclopropyl)-4-[(1S)-2,2-dichlorocyclopropyl]-1-(1H-2,4-triazol-1-yl)butan-2-ol, (1.033) (2S)-2-[4-(4-chlorophenoxy)-2-(trifluoromethyl)phenyl]-1-(1H-2,4-triazol-1-yl)propan-2-ol, (1.034) (R)-[3-(4-chloro-2-fluorophenyl)-5-(2,4-difluorophenyl)-1,2-oxazol-4-yl](pyridin-3-yl)methanol, (1.035) (S)-[3-(4-chloro-2-fluorophenyl)-5-(2,4-difluorophenyl)-1,2-xazol-4-yl](pyridin-3-yl)methanol, (1.036) [3-(4-chloro-2-fluorophenyl)-5-(2,4-difluorophenyl)-1,2-oxazol-4-yl](pyridin-3-yl)methanol, (1.037) 1-({(2R,4S)-2-[2-chloro-4-(4-chlorophenoxy)phenyl]-4-methyl-1,3-dioxolan-2-yl}methyl)-1H-1,2,4-triazole, (1.038) 1-({(2S,4S)-2-[2-chloro-4-(4-chlorophenoxy)phenyl]-4-methyl-1,3-dioxolan-2-yl}methyl)-1H-1,2,4-triazole, (1.039) 1-{[3-(2-chlorophenyl)-2-(2,4-difluorophenyl)oxiran-2-yl]methyl}-1H-1,2,4-triazol-5-yl thiocyanate, (1.040) 1-{[rel(2R,3R)-3-(2-chlorophenyl)-2-(2,4-difluorophenyl)oxiran-2-yl]methyl}-1H-1,2,4-triazol-5-yl thiocyanate, (1.041) 1-{[rel(2R,3S)-3-(2-chlorophenyl)-2-(2,4-difluorophenyl)oxiran-2-yl]methyl}-1H-1,2,4-triazol-5-yl thiocyanate, (1.042) 2-[(2R,4R,5R)-1-(2,4-dichlorophenyl)-5-hydroxy-2,6,6-trimethylheptan-4-yl]-2,4-dihydro-3H-1,2,4-triazole-3-thione, (1.043) 2-[(2R,4R,5S)-1-(2,4-dichlorophenyl)-5-hydroxy-2,6,6-trimethylheptan-4-yl]-2,4-dihydro-3H-1,2,4-triazole-3-thione, (1.044) 2-[(2R,4S,5R)-1-(2,4-dichlorophenyl)-5-hydroxy-2,6,6-trimethylheptan-4-yl]-2,4-dihydro-3H-1,2,4-triazole-3-thione, (1.045) 2-[(2R,4S,5S)-1-(2,4-dichlorophenyl)-5-hydroxy-2,6,6-trimethylheptan-4-yl]-2,4-dihydro-3H-1,2,4-triazole-3-thione, (1.046) 2-[(2S,4R,5R)-1-(2,4-dichlorophenyl)-5-hydroxy-2,6,6-trimethylheptan-4-yl]-2,4-dihydro-3H-1,2,4-triazole-3-thione, (1.047) 2-[(2S,4R,5S)-1-(2,4-dichlorophenyl)-5-hydroxy-2,6,6-trimethylheptan-4-yl]-2,4-dihydro-3H-1,2,4-triazole-3-thione, (1.048) 2-[(2S,4S,5R)-1-(2,4-dichlorophenyl)-5-hydroxy-2,6,6-trimethylheptan-4-yl]-2,4-dihydro-3H-1,2,4-triazole-3-thione, (1.049) 2-[(2S,4S,5S)-1-(2,4-dichlorophenyl)-5-hydroxy-2,6,6-trimethylheptan-4-yl]-2,4-dihydro-3H-1,2,4-triazole-3-thione, (1.050) 2-[1-(2,4-dichlorophenyl)-5-hydroxy-2,6,6-trimethylheptan-4-yl]-2,4-dihydro-3H-1,2,4-triazole-3-thione, (1.051) 2-[2-chloro-4-(2,4-dichlorophenoxy)phenyl]-1-(1H-1,2,4-triazol-1-yl)propan-2-ol, (1.052) 2-[2-chloro-4-(4-chlorophenoxy)phenyl]-1-(1H-1,2,4-triazol-1-yl)butan-2-ol, (1.053) 2-[4-(4-chlorophenoxy)-2-(trifluoromethyl)phenyl]-1-(1H-1,2,4-triazol-1-yl)butan-2-ol, (1.054) 2-[4-(4-chlorophenoxy)-2-(trifluoromethyl)phenyl]-1-(1H-1,2,4-triazol-1-yl)pentan-2-ol, (1.055) 2-[4-(4-chlorophenoxy)-2-(trifluoromethyl)phenyl]-1-(1H-1,2,4-triazol-1-yl)propan-2-ol, (1.056) 2-{[3-(2-chlorophenyl)-2-(2,4-difluorophenyl)oxiran-2-yl]methyl}-2,4-dihydro-3H-1,2,4-triazole-3-thione, (1.057) 2-{[rel(2R,3R)-3-(2-chlorophenyl)-2-(2,4-difluorophenyl)oxiran-2-yl]methyl}-2,4-dihydro-3H-1,2,4-triazole-3-thione, (1.058) 2-{[rel(2R,3S)-3-(2-chlorophenyl)-2-(2,4-difluorophenyl)oxiran-2-yl]methyl}-2,4-dihydro-3H-1,2,4-triazole-3-thione, (1.059) 5-(4-chlorobenzyl)-2-(chloromethyl)-2-methyl-1-(1H-1,2,4-triazol-1-ylmethyl)cyclopentanol, (1.060) 5-(allylsulfanyl)-1-{[3-(2-chlorophenyl)-2-(2,4-difluorophenyl)oxiran-2-yl]methyl}-1H-1,2,4-triazole, (1.061) 5-(allylsulfanyl)-1-{[rel(2R,3R)-3-(2-chlorophenyl)-2-(2,4-difluorophenyl)oxiran-2-yl]methyl}-1H-1,2,4-triazole, (1.062) 5-(allylsulfanyl)-1-{[rel(2R,3S)-3-(2-chlorophenyl)-2-(2,4-difluorophenyl)oxiran-2-yl]methyl}-1H-1,2,4-triazole, (1.063) N′-(2,5-dimethyl-4-{[3-(1,1,2,2-tetrafluoroethoxy)phenyl]sulfanyl}phenyl)-N-ethyl-N-methylimidoformamide, (1.064) N′-(2,5-dimethyl-4-{[3-(2,2,2-trifluoroethoxy)phenyl]sulfanyl}phenyl)-N-ethyl-N-methylimidoformamide, (1.065) N′-(2,5-dimethyl-4-{[3-(2,2,3,3-tetrafluoropropoxy)phenyl]sulfanyl}phenyl)-N-ethyl-N-methylimidoformamide, (1.066) N′-(2,5-dimethyl-4-{[3-(pentafluoroethoxy)phenyl]sulfanyl}phenyl)-N-ethyl-N-methylimidoformamide, (1.067) N′-(2,5-dimethyl-4-{3-[(1,1,2,2-tetrafluoroethyl)sulfanyl]phenoxy}phenyl)-N-ethyl-N-methylimidoformamide, (1.068) N′-(2,5-dimethyl-4-{3-[(2,2,2-trifluoroethyl)sulfanyl]phenoxy}phenyl)-N-ethyl-N-methylimidoformamide, (1.069) N′-(2,5-dimethyl-4-{3-[(2,2,3,3-tetrafluoropropyl)sulfanyl]phenoxy}phenyl)-N-ethyl-N-methylimidoformamide, (1.070) N′-(2,5-dimethyl-4-{3-[(pentafluoroethyl)sulfanyl]phenoxy}phenyl)-N-ethyl-N-methylimidoformamide, (1.071) N′-(2,5-dimethyl-4-phenoxyphenyl)-N-ethyl-N-methylimidoformamide, (1.072) N′-(4-{[3-(difluoromethoxy)phenyl]sulfanyl}-2,5-dimethylphenyl)-N-ethyl-N-methylimidoformamide, (1.073) N′-(4-{3-[(difluoromethyl)sulfanyl]phenoxy}-2,5-dimethylphenyl)-N-ethyl-N-methylimidoformamide, (1.074) N′-[5-bromo-6-(2,3-dihydro-1H-inden-2-yloxy)-2-methylpyridin-3-yl]-N-ethyl-N-methylimidoformamide, (1.075) N′-{4-[(4,5-dichloro-1,3-thiazol-2-yl)oxy]-2,5-dimethylphenyl}-N-ethyl-N-methylimidoformamide, (1.076) N′-{5-bromo-6-[(1R)-1-(3,5-difluorophenyl)ethoxy]-2-methylpyridin-3-yl}-N-ethyl-N-methylimidoformamide, (1.077) N′-{5-bromo-6-[(1 S)-1-(3,5-difluorophenyl)ethoxy]-2-methylpyridin-3-yl}-N-ethyl-N-methylimidoformamide, (1.078) N′-{5-bromo-6-[(cis-4-isopropylcyclohexyl)oxy]-2-methylpyridin-3-yl}-N-ethyl-N-methylimidoformamide, (1.079) N′-{5-bromo-6-[(trans-4-isopropylcyclohexyl)oxy]-2-methylpyridin-3-yl}-N-ethyl-N-methylimidoformamide, (1.080) N′-{5-bromo-6-[1-(3,5-difluorophenyl)ethoxy]-2-methylpyridin-3-yl}-N-ethyl-N-methylimidoformamide, (1.081) mefentrifluconazole, (1.082) ipfentrifluconazole.
2) Inhibitors of the respiratory chain in complex I or II, for example (2.001) benzovindiflupyr, (2.002) bixafen, (2.003) boscalid, (2.004) carboxin, (2.005) fluopyram, (2.006) flutolanil, (2.007) fluxapyroxad, (2.008) furametpyr, (2.009) isofetamid, (2.010) isopyrazam (anti-epimeric enantiomer 1R,4S,9S), (2.011) isopyrazam (anti-epimeric enantiomer 1S,4R,9R), (2.012) isopyrazam (anti-epimeric racemate 1RS,4SR,9SR), (2.013) isopyrazam (mixture of the syn-epimeric racemate 1RS,4SR,9RS and the anti-epimeric racemate 1RS,4SR,9SR), (2.014) isopyrazam (syn-epimeric enantiomer 1R,4S,9R), (2.015) isopyrazam (syn-epimeric enantiomer 1S,4R,9S), (2.016) isopyrazam (syn-epimeric racemate 1RS,4SR,9RS), (2.017) penflufen, (2.018) penthiopyrad, (2.019) pydiflumetofen, (2.020) pyraziflumid, (2.021) sedaxane, (2.022) 1,3-dimethyl-N-(1,1,3-trimethyl-2,3-dihydro-1H-inden-4-yl)-1H-pyrazole-4-carboxamide, (2.023) 1,3-dimethyl-N-[(3R)-1,1,3-trimethyl-2,3-dihydro-1H-inden-4-yl]-1H-pyrazole-4-carboxamide, (2.024) 1,3-dimethyl-N-[(3S)-1,1,3-trimethyl-2,3-dihydro-1H-inden-4-yl]-1H-pyrazole-4-carboxamide, (2.025) 1-methyl-3-(trifluoromethyl)-N-[2′-(trifluoromethyl)biphenyl-2-yl]-1H-pyrazole-4-carboxamide, (2.026) 2-fluoro-6-(trifluoromethyl)-N-(1,1,3-trimethyl-2,3-dihydro-1H-inden-4-yl)benzamide, (2.027) 3-(difluoromethyl)-1-methyl-N-(1,1,3-trimethyl-2,3-dihydro-1H-inden-4-yl)-1H-pyrazole-4-carboxamide, (2.028) 3-(difluoromethyl)-1-methyl-N-[(3R)-1,1,3-trimethyl-2,3-dihydro-1H-inden-4-yl]-1H-pyrazole-4-carboxamide, (2.029) 3-(difluoromethyl)-1-methyl-N-[(3S)-1,1,3-trimethyl-2,3-dihydro-1H-inden-4-yl]-1H-pyrazole-4-carboxamide, (2.030) 3-(difluoromethyl)-N-(7-fluoro-1,1,3-trimethyl-2,3-dihydro-1H-inden-4-yl)-1-methyl-1H-pyrazole-4-carboxamide, (2.031) 3-(difluoromethyl)-N-[(3R)-7-fluoro-1,1,3-trimethyl-2,3-dihydro-1H-inden-4-yl]-1-methyl-1H-pyrazole-4-carboxamide, (2.032) 3-(difluoromethyl)-N-[(3S)-7-fluoro-1,1,3-trimethyl-2,3-dihydro-1H-inden-4-yl]-1-methyl-1H-pyrazole-4-carboxamide, (2.033) 5,8-difluoro-N-[2-(2-fluoro-4-{[4-(trifluoromethyl)pyridin-2-yl]oxy}phenyl)ethyl]quinazoline-4-amine, (2.034) N-(2-cyclopentyl-5-fluorobenzyl)-N-cyclopropyl-3-(difluoromethyl)-5-fluoro-1-methyl-1H-pyrazole-4-carboxamide, (2.035) N-(2-tert-butyl-5-methylbenzyl)-N-cyclopropyl-3-(difluoromethyl)-5-fluoro-1-methyl-1H-pyrazole-4-carboxamide, (2.036) N-(2-tert-butylbenzyl)-N-cyclopropyl-3-(difluoromethyl)-5-fluoro-1-methyl-1H-pyrazole-4-carboxamide, (2.037) N-(5-chloro-2-ethylbenzyl)-N-cyclopropyl-3-(difluoromethyl)-5-fluoro-1-methyl-1H-pyrazole-4-carboxamide, (2.038) N-(5-chloro-2-isopropylbenzyl)-N-cyclopropyl-3-(difluoromethyl)-5-fluoro-1-methyl-1H-pyrazole-4-carboxamide, (2.039) N-[(1R,4S)-9-(dichloromethylene)-1,2,3,4-tetrahydro-1,4-methanonaphthalin-5-yl]-3-(difluoromethyl)-1-methyl-1H-pyrazole-4-carboxamide, (2.040) N-[(1S,4R)-9-(dichloromethylene)-1,2,3,4-tetrahydro-1,4-methanonaphthalin-5-yl]-3-(difluoromethyl)-1-methyl-1H-pyrazole-4-carboxamide, (2.041) N-[1-(2,4-dichlorophenyl)-1-methoxypropan-2-yl]-3-(difluoromethyl)-1-methyl-1H-pyrazole-4-carboxamide, (2.042) N-[2-chloro-6-(trifluoromethyl)benzyl]-N-cyclopropyl-3-(difluoromethyl)-5-fluoro-1-methyl-1H-pyrazole-4-carboxamide, (2.043) N-[3-chloro-2-fluoro-6-(trifluoromethyl)benzyl]-N-cyclopropyl-3-(difluoromethyl)-5-fluoro-1-methyl-1H-pyrazole-4-carboxamide, (2.044) N-[5-chloro-2-(trifluoromethyl)benzyl]-N-cyclopropyl-3-(difluoromethyl)-5-fluoro-1-methyl-1H-pyrazole-4-carboxamide, (2.045) N-cyclopropyl-3-(difluoromethyl)-5-fluoro-1-methyl-N-[5-methyl-2-(trifluoromethyl)benzyl]-1H-pyrazole-4-carboxamide, (2.046) N-cyclopropyl-3-(difluoromethyl)-5-fluoro-N-(2-fluoro-6-isopropylbenzyl)-1-methyl-1H-pyrazole-4-carboxamide, (2.047) N-cyclopropyl-3-(difluoromethyl)-5-fluoro-N-(2-isopropyl-5-methylbenzyl)-1-methyl-1H-pyrazole-4-carboxamide, (2.048) N-cyclopropyl-3-(difluoromethyl)-5-fluoro-N-(2-isopropylbenzyl)-1-methyl-1H-pyrazole-4-carbothioamide, (2.049) N-cyclopropyl-3-(difluoromethyl)-5-fluoro-N-(2-isopropylbenzyl)-1-methyl-1H-pyrazole-4-carboxamide, (2.050) N-cyclopropyl-3-(difluoromethyl)-5-fluoro-N-(5-fluoro-2-isopropylbenzyl)-1-methyl-1H-pyrazole-4-carboxamide, (2.051) N-cyclopropyl-3-(difluoromethyl)-N-(2-ethyl-4,5-dimethylbenzyl)-5-fluoro-1-methyl-1H-pyrazole-4-carboxamide, (2.052) N-cyclopropyl-3-(difluoromethyl)-N-(2-ethyl-5-fluorobenzyl)-5-fluoro-1-methyl-1H-pyrazole-4-carboxamide, (2.053) N-cyclopropyl-3-(difluoromethyl)-N-(2-ethyl-5-methylbenzyl)-5-fluoro-1-methyl-1H-pyrazole-4-carboxamide, (2.054) N-cyclopropyl-N-(2-cyclopropyl-5-fluorobenzyl)-3-(difluoromethyl)-5-fluoro-1-methyl-1H-pyrazole-4-carboxamide, (2.055) N-cyclopropyl-N-(2-cyclopropyl-5-methylbenzyl)-3-(difluoromethyl)-5-fluoro-1-methyl-1H-pyrazole-4-carboxamide, (2.056) N-cyclopropyl-N-(2-cyclopropylbenzyl)-3-(difluoromethyl)-5-fluoro-1-methyl-1H-pyrazole-4-carboxamide.
3) Inhibitors of the respiratory chain in complex III, for example (3.001) ametoctradin, (3.002) amisulbrom, (3.003) azoxystrobin, (3.004) coumethoxystrobin, (3.005) coumoxystrobin, (3.006) cyazofamid, (3.007) dimoxystrobin, (3.008) enoxastrobin, (3.009) famoxadon, (3.010) fenamidon, (3.011) flufenoxystrobin, (3.012) fluoxastrobin, (3.013) kresoxim-methyl, (3.014) metominostrobin, (3.015) orysastrobin, (3.016) picoxystrobin, (3.017) pyraclostrobin, (3.018) pyrametostrobin, (3.019) pyraoxystrobin, (3.020) trifloxystrobin, (3.021) (2E)-2-{2-[({[(1E)-1-(3-{[(E)-1-fluoro-2-phenylvinyl]oxy}phenyl)ethylidene]amino}oxy)methyl]phenyl}-2-(methoxyimino)-N-methylacetamide, (3.022) (2E,3Z)-5-{[1-(4-chlorophenyl)-1H-pyrazol-3-yl]oxy}-2-(methoxyimino)-N,3-dimethylpent-3-enamide, (3.023) (2R)-2-{2-[(2,5-dimethylphenoxy)methyl]phenyl}-2-methoxy-N-methylacetamide, (3.024) (2S)-2-{2-[(2,5-dimethylphenoxy)methyl]phenyl}-2-methoxy-N-methylacetamide, (3.025) (3S,6S,7R,8R)-8-benzyl-3-[({3-[(isobutyryloxy)methoxy]-4-methoxypyridin-2-yl}carbonyl)amino]-6-methyl-4,9-dioxo-1,5-dioxonan-7-yl 2-methylpropanoate, (3.026) 2-{2-[(2,5-dimethylphenoxy)methyl]phenyl}-2-methoxy-N-methylacetamide, (3.027) N-(3-ethyl-3,5,5-trimethylcyclohexyl)-3-formamido-2-hydroxybenzamide, (3.028) (2E,3Z)-5-{[1-(4-chloro-2-fluorophenyl)-1H-pyrazol-3-yl]oxy}-2-(methoxyimino)-N,3-dimethylpent-3-enamide, (3.029) methyl {5-[3-(2,4-dimethylphenyl)-1H-pyrazol-1-yl]-2-methylbenzyl}carbamate.
4) Mitosis and cell division inhibitors, for example (4.001) carbendazim, (4.002) diethofencarb, (4.003) ethaboxam, (4.004) fluopicolid, (4.005) pencycuron, (4.006) thiabendazole, (4.007) thiophanate-methyl, (4.008) zoxamide, (4.009) 3-chloro-4-(2,6-difluorophenyl)-6-methyl-5-phenylpyridazine, (4.010) 3-chloro-5-(4-chlorophenyl)-4-(2,6-difluorophenyl)-6-methylpyridazine, (4.011) 3-chloro-5-(6-chloropyridin-3-yl)-6-methyl-4-(2,4,6-trifluorophenyl)pyridazine, (4.012) 4-(2-bromo-4-fluorophenyl)-N-(2,6-difluorophenyl)-1,3-dimethyl-1H-pyrazol-5-amine, (4.013) 4-(2-bromo-4-fluorophenyl)-N-(2-bromo-6-fluorophenyl)-1,3-dimethyl-1H-pyrazol-5-amine, (4.014) 4-(2-bromo-4-fluorophenyl)-N-(2-bromophenyl)-1,3-dimethyl-1H-pyrazol-5-amine, (4.015) 4-(2-bromo-4-fluorophenyl)-N-(2-chloro-6-fluorophenyl)-1,3-dimethyl-1H-pyrazol-5-amine, (4.016) 4-(2-bromo-4-fluorophenyl)-N-(2-chlorophenyl)-1,3-dimethyl-1H-pyrazol-5-amine, (4.017) 4-(2-bromo-4-fluorophenyl)-N-(2-fluorophenyl)-1,3-dimethyl-1H-pyrazol-5-amine, (4.018) 4-(2-chloro-4-fluorophenyl)-N-(2,6-difluorophenyl)-1,3-dimethyl-1H-pyrazol-5-amine, (4.019) 4-(2-chloro-4-fluorophenyl)-N-(2-chloro-6-fluorophenyl)-1,3-dimethyl-1H-pyrazol-5-amine, (4.020) 4-(2-chloro-4-fluorophenyl)-N-(2-chlorophenyl)-1,3-dimethyl-1H-pyrazol-5-amine, (4.021) 4-(2-chloro-4-fluorophenyl)-N-(2-fluorophenyl)-1,3-dimethyl-1H-pyrazol-5-amine, (4.022) 4-(4-chlorophenyl)-5-(2,6-difluorophenyl)-3,6-dimethylpyridazine, (4.023) N-(2-bromo-6-fluorophenyl)-4-(2-chloro-4-fluorophenyl)-1,3-dimethyl-1H-pyrazol-5-amine, (4.024) N-(2-bromophenyl)-4-(2-chloro-4-fluorophenyl)-1,3-dimethyl-1H-pyrazol-5-amine, (4.025) N-(4-chloro-2,6-difluorophenyl)-4-(2-chloro-4-fluorophenyl)-1,3-dimethyl-1H-pyrazol-5-amine.
5) Compounds having capacity for multisite activity, for example (5.001) Bordeaux mixture, (5.002) captafol, (5.003) captan, (5.004) chlorothalonil, (5.005) copper hydroxide, (5.006) copper naphthenate, (5.007) copper oxide, (5.008) copper oxychloride, (5.009) copper(2+) sulfate, (5.010) dithianon, (5.011) dodin, (5.012) folpet, (5.013) mancozeb, (5.014) maneb, (5.015) metiram, (5.016) zinc metiram, (5.017) copper oxine, (5.018) propineb, (5.019) sulfur and sulfur preparations including calcium polysulfide, (5.020) thiram, (5.021) zineb, (5.022) ziram, (5.023) 6-ethyl-5,7-dioxo-6,7-dihydro-5H-pyrrolo[3′,4′:5,6][1,4]dithiino[2,3-c][1,2]thiazole-3-carbonitrile.
6) Compounds capable of triggering host defense, for example (6.001) acibenzolar-S-methyl, (6.002) isotianil, (6.003) probenazole, (6.004) tiadinil.
7) Amino acid and/or protein biosynthesis inhibitors, for example (7.001) cyprodinil, (7.002) kasugamycin, (7.003) kasugamycin hydrochloride hydrate, (7.004) oxytetracycline, (7.005) pyrimethanil, (7.006) 3-(5-fluoro-3,3,4,4-tetramethyl-3,4-dihydroisoquinolin-1-yl)quinoline.
(8) ATP production inhibitors, for example (8.001) silthiofam.
9) Cell wall synthesis inhibitors, for example (9.001) benthiavalicarb, (9.002) dimethomorph, (9.003) flumorph, (9.004) iprovalicarb, (9.005) mandipropamid, (9.006) pyrimorph, (9.007) valifenalate, (9.008) (2E)-3-(4-tert-butylphenyl)-3-(2-chloropyridin-4-yl)-1-(morpholin-4-yl)prop-2-en-1-one, (9.009) (2Z)-3-(4-tert-butylphenyl)-3-(2-chloropyridin-4-yl)-1-(morpholin-4-yl)prop-2-en-1-one.
10) Lipid and membrane synthesis inhibitors, for example (10.001) propamocarb, (10.002) propamocarb hydrochloride, (10.003) tolclofos-methyl.
11) Melanin biosynthesis inhibitors, for example (11.001) tricyclazole, (11.002) 2,2,2-trifluoroethyl {3-methyl-1-[(4-methylbenzoyl)amino]butan-2-yl}carbamate.
12) Nucleic acid synthesis inhibitors, for example (12.001) benalaxyl, (12.002) benalaxyl-M (kiralaxyl), (12.003) metalaxyl, (12.004) metalaxyl-M (mefenoxam).
13) Signal transduction inhibitors, for example (13.001) fludioxonil, (13.002) iprodione, (13.003) procymidone, (13.004) proquinazid, (13.005) quinoxyfen, (13.006) vinclozolin.
14) Compounds that can act as uncouplers, for example (14.001) fluazinam, (14.002) meptyldinocap.
15) Further compounds, for example (15.001) abscisic acid, (15.002) benthiazole, (15.003) bethoxazine, (15.004) capsimycin, (15.005) carvone, (15.006) chinomethionat, (15.007) cufraneb, (15.008) cyflufenamid, (15.009) cymoxanil, (15.010) cyprosulfamide, (15.011) flutianil, (15.012) fosetyl-aluminium, (15.013) fosetyl-calcium, (15.014) fosetyl-sodium, (15.015) methyl isothiocyanate, (15.016) metrafenone, (15.017) mildiomycin, (15.018) natamycin, (15.019) nickel dimethyldithiocarbamate, (15.020) nitrothal-isopropyl, (15.021) oxamocarb, (15.022) oxathiapiproline, (15.023) oxyfenthiine, (15.024) pentachlorophenol and salts, (15.025) phosphonic acid and its salts, (15.026) propamocarb-fosetylate, (15.027) pyriofenone (chlazafenone), (15.028) tebufloquin, (15.029) tecloftalam, (15.030) tolnifanide, (15.031) 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.032) 1-(4-{4-[(5S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.033) 2-(6-benzylpyridin-2-yl)quinazoline, (15.034) 2,6-dimethyl-1H,5H-[1,4]dithiino[2,3-c:5,6-c′]dipyrrole-1,3,5,7(2H,6H)-tetrone, (15.035) 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)piperidin-1-yl]ethanone, (15.036) 2-[3,5-bis(difluoromethyl)-1H-pyrazol-1-yl]-1-[4-(4-{5-[2-chloro-6-(prop-2-yn-1-yloxy)phenyl]-4,5-dihydro-1,2-oxazol-3-yl}-1,3-thiazol-2-yl)piperidin-1-yl]ethanone, (15.037) 2-[3,5-bis(difluoromethyl)-1H-pyrazol-1-yl]-1-[4-(4-{5-[2-fluoro-6-(prop-2-yn-1-yloxy)phenyl]-4,5-dihydro-1,2-oxazol-3-yl}-1,3-thiazol-2-yl)piperidin-1-yl]ethanone, (15.038) 2-[6-(3-fluoro-4-methoxyphenyl)-5-methylpyridin-2-yl]quinazoline, (15.039) 2-{(5R)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.040) 2-{(5 S)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.041) 2-{2-[(7,8-difluoro-2-methylquinolin-3-yl)oxy]-6-fluorophenyl}propan-2-ol, (15.042) 2-{2-fluoro-6-[(8-fluoro-2-methylquinolin-3-yl)oxy]phenyl}propan-2-ol, (15.043) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulfonate, (15.044) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}phenyl methanesulfonate, (15.045) 2-phenylphenol and its salts, (15.046) 3-(4,4,5-trifluoro-3,3-dimethyl-3,4-dihydroisoquinolin-1-yl)quinoline, (15.047) 3-(4,4-difluoro-3,3-dimethyl-3,4-dihydroisoquinolin-1-yl)quinoline, (15.048) 4-amino-5-fluoropyrimidin-2-ol (tautomeric form: 4-amino-5-fluoropyrimidin-2(1H)-one), (15.049) 4-oxo-4-[(2-phenylethyl)amino]butyric acid, (15.050) 5-amino-1,3,4-thiadiazole-2-thiol, (15.051) 5-chloro-N′-phenyl-N′-(prop-2-yn-1-yl)thiophene 2-sulfonohydrazide, (15.052) 5-fluoro-2-[(4-fluorobenzyl)oxy]pyrimidin-4-amine, (15.053) 5-fluoro-2-[(4-methylbenzyl)oxy]pyrimidin-4-amine, (15.054) 9-fluoro-2,2-dimethyl-5-(quinolin-3-yl)-2,3-dihydro-1,4-benzoxazepine, (15.055) but-3-yn-1-yl {6-[({[(Z)-(1-methyl-1H-tetrazol-5-yl)(phenyl)methylene]amino}oxy)methyl]pyridin-2-yl}carbamate, (15.056) ethyl (2Z)-3-amino-2-cyano-3-phenylacrylate, (15.057) phenazine-1-carboxylic acid, (15.058) propyl 3,4,5-trihydroxybenzoate, (15.059) quinolin-8-ol, (15.060) quinolin-8-ol sulfate (2:1), (15.061) tert-butyl {6-[({[(1-methyl-1H-tetrazol-5-yl)(phenyl)methylene]amino}oxy)methyl]pyridin-2-yl}carbamate, (15.062) 5-fluoro-4-imino-3-methyl-1-[(4-methylphenyl)sulfonyl]-3,4-dihydropyrimidin-2(1H)-one.

### Biological Pesticides as Mixing Components

The compounds of the formula (I) can be combined with biological pesticides.

Biological pesticides especially include bacteria, fungi, yeasts, plant extracts and products formed by microorganisms, including proteins and secondary metabolites.

Biological pesticides include bacteria such as spore-forming bacteria, root-colonizing bacteria and bacteria which act as biological insecticides, fungicides or nematicides.

Examples of such bacteria which are used or can be used as biological pesticides are:

Bacillus amyloliquefaciens, strain FZB42 (DSM 231179), or Bacillus cereus, especially B. cereus strain CNCM I-1562, or Bacillus firmus, strain I-1582 (Accession number CNCM I-1582), or Bacillus pumilus, especially strain GB34 (Accession No. ATCC 700814) and strain QST2808 (Accession No. NRRL B-30087), or Bacillus subtilis, especially strain GB03 (Accession No. ATCC SD-1397), or Bacillus subtilis strain QST713 (Accession No. NRRL B-21661) or Bacillus subtilis strain OST 30002 (Accession No. NRRL B-50421), Bacillus thuringiensis, especially B. thuringiensis subspecies israelensis (serotype H-14), strain AM65-52 (Accession No. ATCC 1276), or B. thuringiensis subsp. aizawai, especially strain ABTS-1857 (SD-1372), or B. thuringiensis subsp. kurstaki strain HD-1, or B. thuringiensis subsp. tenebrionis strain NB 176 (SD-5428), Pasteuria penetrans, Pasteuria spp. (Rotylenchulus reniformis nematode)-PR3 (Accession Number ATCC SD-5834), Streptomyces microflavus strain AQ6121 (=QRD 31.013, NRRL B-50550), Streptomyces galbus strain AQ 6047 (Accession Number NRRL 30232).

Examples of fungi and yeasts which are used or can be used as biological pesticides are:

Beauveria bassiana, in particular strain ATCC 74040, Coniothyrium minitans, in particular strain CON/M/91-8 (Accession No. DSM-9660), Lecanicillium spp., in particular strain HRO LEC 12, Lecanicillium lecanii (formerly known as Verticillium lecanii), in particular strain KV01, Metarhizium anisopliae, in particular strain F52 (DSM3884/ATCC 90448), Metschnikowia fructicola, in particular strain NRRL Y-30752, Paecilomyces fumosoroseus (new: Isariafumosorosea), in particular strain IFPC 200613, or strain Apopka 97 (Accession No. ATCC 20874), Paecilomyces lilacinus, in particular P. lilacinus strain 251 (AGAL 89/030550), Talaromyces flavus, in particular strain V117b, Trichoderma atroviride, in particular strain SC1 (Accession Number CBS 122089), Trichoderma harzianum, in particular T. harzianum rifai T39 (Accession number CNCM I-952).

Examples of viruses which are used or can be used as biological pesticides are:

Adoxophyes orana (summer fruit tortrix) granulosis virus (GV), Cydia pomonella (codling moth) granulosis virus (GV), Helicoverpa armigera (cotton bollworm) nuclear polyhedrosis virus (NPV), Spodoptera exigua (beet armyworm) mNPV, Spodoptera frugiperda (fall armyworm) mNPV, Spodoptera littoralis (African cotton leafworm) NPV.

Also included are bacteria and fungi which are added as ‘inoculant’ to plants or plant parts or plant organs and which, by virtue of their particular properties, promote plant growth and plant health. Examples include:

Agrobacterium spp., Azorhizobium caulinodans, Azospirillum spp., Azotobacter spp., Bradyrhizobium spp., Burkholderia spp., especially Burkholderia cepacia (formerly known as Pseudomonas cepacia), Gigaspora spp., or Gigaspora monosporum, Glomus spp., Laccaria spp., Lactobacillus buchneri, Paraglomus spp., Pisolithus tinctorus, Pseudomonas spp., Rhizobium spp., especially Rhizobium trifolii, Rhizopogon spp., Scleroderma spp., Suillus spp., Streptomyces spp.

Examples of plant extracts and products formed by microorganisms, including proteins and secondary metabolites, which are used or can be used as biological pesticides are:

Allium sativum, Artemisia absinthium, azadirachtin, Biokeeper WP, Cassia nigricans, Celastrus angulatus, Chenopodium anthelminticum, chitin, Armour-Zen, Dryopteris filix-mas, Equisetum arvense, Fortune Aza, Fungastop, Heads Up (Chenopodium quinoa saponin extract), pyrethrum/pyrethrins, Quassia amara, Quercus, Quillaja, Regalia, “Requiem™ Insecticide”, rotenone, ryania/ryanodine, Symphytum officinale, Tanacetum vulgare, thymol, Triact 70, TriCon, Tropaeulum majus, Urtica dioica, Veratrin, Viscum album, Brassicaceae extract, especially oilseed rape powder or mustard powder.

### Safener as Mixing Components

The compounds of the formula (I) can be combined with safeners, for example benoxacor, cloquintocet (-mexyl), cyometrinil, cyprosulfamide, dichlormid, fenchlorazole (-ethyl), fenclorim, flurazole, fluxofenim, furilazole, isoxadifen (-ethyl), mefenpyr (-diethyl), naphthalic anhydride, oxabetrinil, 2-methoxy-N-({4-[(methylcarbamoyl)amino]phenyl}sulfonyl)benzamide (CAS 129531-12-0), 4-(dichloroacetyl)-1-oxa-4-azaspiro[4.5]decane (CAS 71526-07-3), 2,2,5-trimethyl-3-(dichloroacetyl)-1,3-oxazolidine (CAS 52836-31-4).

### Plants and Plant Parts

All plants and plant parts can be treated in accordance with the invention. Plants are understood here to mean all plants and populations of plants, such as desirable and undesirable wild plants or crop plants (including naturally occurring crop plants), for example cereals (wheat, rice, triticale, barley, rye, oats), maize, soya beans, potatoes, sugar beet, sugar cane, tomatoes, bell peppers, cucumbers, melons, carrots, water melons, onions, lettuce, spinach, leeks, beans, Brassica oleracea (e.g. cabbage) and other vegetable species, cotton, tobacco, oilseed rape, and also fruit plants (the fruits being apples, pears, citrus fruits and grapes). Crop plants may be plants which can be obtained by conventional breeding and optimization methods or by biotechnological and genetic engineering methods or combinations of these methods, including the transgenic plants and including the plant cultivars which are protectable or non-protectable by plant breeders' rights. Plants shall be understood to mean all development stages such as seed, seedlings, young (immature) plants, up to and including mature plants. Plant parts shall be understood to mean all parts and organs of the plants above and below ground, such as shoot, leaf, flower and root, examples given being leaves, needles, stalks, stems, flowers, fruit bodies, fruits and seeds, and also roots, tubers and rhizomes. Plant parts also include harvested plants or harvested plant parts and vegetative and generative propagation material, for example cuttings, tubers, rhizomes, slips and seeds.

The inventive treatment of the plants and parts of plants with the compounds of the formula (I) is effected directly or by allowing the compounds to act on the surroundings, the habitat or the storage space thereof by the customary treatment methods, for example by dipping, spraying, evaporating, fogging, scattering, painting on, injecting, and, in the case of propagation material, especially in the case of seeds, also by applying one or more coats.

As already mentioned above, it is possible to treat all plants and their parts in accordance with the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (genetically modified organisms), and parts thereof are treated. The term “parts” or “parts of plants” or “plant parts” has been explained above. Particular preference is given in accordance with the invention to treating plants of the respective commercially customary plant cultivars or those that are in use. Plant cultivars are understood to mean plants having novel properties (“traits”) and which have been obtained by conventional breeding, by mutagenesis or by recombinant DNA techniques. They may be cultivars, varieties, biotypes or genotypes.

### Transgenic Plants, Seed Treatment and Integration Events

The preferred transgenic plants or plant cultivars (those obtained by genetic engineering) which are to be treated in accordance with the invention include all plants which, through the genetic modification, received genetic material which imparts particular advantageous useful properties (“traits”) to these plants. Examples of such properties are better plant growth, increased tolerance to high or low temperatures, increased tolerance to drought or to levels of water or soil salinity, enhanced flowering performance, easier harvesting, accelerated ripening, higher harvest yields, higher quality and/or higher nutritional value of the harvested products, better capability for storage and/or processability of the harvested products. Further and particularly emphasized examples of such properties are increased resistance of the plants to animal and microbial pests, such as insects, arachnids, nematodes, mites, slugs and snails, owing, for example, to toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof), and also increased resistance of the plants to phytopathogenic fungi, bacteria and/or viruses caused, for example, by systemic acquired resistance (SAR), systemin, phytoalexins, elicitors and resistance genes and correspondingly expressed proteins and toxins, and also increased tolerance of the plants to certain herbicidal active compounds, for example imidazolinones, sulfonylureas, glyphosate or phosphinothricin (for example the “PAT” gene). The genes which impart the desired properties (“traits”) in question may also be present in combinations with one another in the transgenic plants. Examples of transgenic plants mentioned include the important crop plants, such as cereals (wheat, rice, triticale, barley, rye, oats), maize, soya beans, potatoes, sugar beet, sugar cane, tomatoes, peas and other vegetable species, cotton, tobacco, oilseed rape, and also fruit plants (the fruits being apples, pears, citrus fruits and grapes), particular emphasis being given to maize, soya beans, wheat, rice, potatoes, cotton, sugar cane, tobacco and oilseed rape. Properties (“traits”) which are particularly emphasized are the increased resistance of the plants to insects, arachnids, nematodes and slugs and snails.

### Plant Protection—Types of Treatment

The plants and plant parts are treated with the compounds of the formula (I) directly or by action on their surroundings, habitat or storage space using customary treatment methods, for example by dipping, spraying, atomizing, irrigating, evaporating, dusting, fogging, broadcasting, foaming, painting, spreading-on, injecting, watering (drenching), drip irrigating and, in the case of propagation material, in particular in the case of seed, additionally by dry seed treatment, liquid seed treatment, slurry treatment, by incrusting, by coating with one or more coats, etc. It is furthermore possible to apply the compounds of the formula (I) by the ultra-low volume method or to inject the application form or the compound of the formula (I) itself into the soil.

A preferred direct treatment of the plants is foliar application, meaning that the compounds of the formula (I) are applied to the foliage, in which case the treatment frequency and the application rate should be adjusted according to the level of infestation with the pest in question.

In the case of systemically active compounds, the compounds of the formula (I) also access the plants via the root system. The plants are then treated by the action of the compounds of the formula (I) on the habitat of the plant. This can be accomplished, for example, by drenching, or by mixing into the soil or the nutrient solution, meaning that the locus of the plant (e.g. soil or hydroponic systems) is impregnated with a liquid form of the compounds of the formula (I), or by soil application, meaning that the compounds of the formula (I) according to the invention are introduced in solid form (e.g. in the form of granules) into the locus of the plants. In the case of paddy rice crops, this can also be accomplished by metering the compound of the formula (I) in a solid application form (for example as granules) into a flooded paddy field.

### Seed Treatment

The control of animal pests by the treatment of the seed of plants has long been known and is the subject of constant improvements. Nevertheless, the treatment of seed entails a series of problems which cannot always be solved in a satisfactory manner. Thus, it is desirable to develop methods for protecting the seed and the germinating plant which dispense with, or at least reduce considerably, the additional application of pesticides during storage, after sowing or after emergence of the plants. It is additionally desirable to optimize the amount of active compound used so as to provide optimum protection for the seed and the germinating plant from infestation by animal pests, but without damage to the plant itself by the active compound used. In particular, methods for the treatment of seed should also take account of the intrinsic insecticidal or nematicidal properties of pest-resistant or -tolerant transgenic plants in order to achieve optimal protection of the seed and also the germinating plant with a minimum expenditure on pesticides.

The present invention therefore in particular also relates to a method for the protection of seed and germinating plants from infestation by pests, by treating the seed with one of the compounds of the formula (I). The method according to the invention for protecting seed and germinating plants against infestation by pests further comprises a method in which the seed is treated simultaneously in one operation or sequentially with a compound of the formula (I) and a mixing component. It further also comprises a method where the seed is treated at different times with a compound of the formula (I) and a mixing component.

The invention likewise relates to the use of the compounds of the formula (I) for the treatment of seed for protecting the seed and the resulting plant from animal pests.

The invention further relates to seed which has been treated with a compound of the formula (I) according to the invention for protection from animal pests. The invention also relates to seed which has been treated simultaneously with a compound of the formula (I) and a mixing component. The invention further relates to seed which has been treated at different times with a compound of the formula (I) and a mixing component. In the case of seed which has been treated at different times with a compound of the formula (I) and a mixing component, the individual substances may be present on the seed in different layers. In this case, the layers comprising a compound of the formula (I) and mixing components may optionally be separated by an intermediate layer. The invention also relates to seed in which a compound of the formula (I) and a mixing component have been applied as part of a coating or as a further layer or further layers in addition to a coating.

The invention further relates to seed which, after the treatment with a compound of the formula (I), is subjected to a film-coating process to prevent dust abrasion on the seed.

One of the advantages that occur when a compound of the formula (I) acts systemically is that the treatment of the seed protects not only the seed itself but also the plants resulting therefrom, after emergence, from animal pests. In this way, the immediate treatment of the crop at the time of sowing or shortly thereafter can be dispensed with.

A further advantage is that the treatment of the seed with a compound of the formula (I) can enhance germination and emergence of the treated seed.

It is likewise considered to be advantageous that compounds of the formula (I) can especially also be used for transgenic seed.

Compounds of the formula (I) can also be used in combination with signalling technology compositions, leading to better colonization by symbionts, for example rhizobia, mycorrhizae and/or endophytic bacteria or fungi, and/or to optimized nitrogen fixation.

The compounds of the formula (I) are suitable for the protection of seed of any plant variety which is used in agriculture, in greenhouses, in forests or in horticulture. More particularly, this is the seed of cereals (for example wheat, barley, rye, millet and oats), maize, cotton, soya beans, rice, potatoes, sunflowers, coffee, tobacco, canola, oilseed rape, beet (for example sugar beet and fodder beet), peanuts, vegetables (for example tomatoes, cucumbers, beans, cruciferous vegetables, onions and lettuce), fruit plants, lawns and ornamental plants. Of particular significance is the treatment of the seed of cereals (such as wheat, barley, rye and oats), maize, soya beans, cotton, canola, oilseed rape, vegetables and rice.

As already mentioned above, the treatment of transgenic seed with a compound of the formula (I) is also of particular importance. This involves the seed of plants which generally contain at least one heterologous gene which controls the expression of a polypeptide having insecticidal and/or nematicidal properties in particular. The heterologous genes in transgenic seed may originate from microorganisms such as Bacillus, Rhizobium, Pseudomonas, Serratia, Trichoderma, Clavibacter, Glomus or Gliocladium. The present invention is particularly suitable for treatment of transgenic seed which comprises at least one heterologous gene originating from Bacillus sp. The heterologous gene is more preferably derived from Bacillus thuringiensis.

In the context of the present invention, the compound of the formula (I) is applied to the seed. The seed is preferably treated in a state in which it is sufficiently stable for no damage to occur in the course of treatment. In general, the seed can be treated at any time between harvest and sowing. It is customary to use seed which has been separated from the plant and freed from cobs, shells, stalks, coats, hairs or the flesh of the fruits. For example, it is possible to use seed which has been harvested, cleaned and dried down to a moisture content which allows storage. Alternatively, it is also possible to use seed which, after drying, has been treated with, for example, water and then dried again, for example priming. In the case of rice seed, it is also possible to use seed which has been soaked, for example in water, until it reaches a certain stage of the rice embryo (“pigeon breast stage”) which results in stimulation of germination and more uniform emergence.

When treating the seed, care must generally be taken that the amount of the compound of the formula (I) applied to the seed and/or the amount of further additives is chosen in such a way that the germination of the seed is not adversely affected, or that the resulting plant is not damaged. This has to be ensured particularly in the case of active compounds which can exhibit phytotoxic effects at certain application rates.

In general, the compounds of the formula (I) are applied to the seed in the form of a suitable formulation. Suitable formulations and methods for seed treatment are known to the person skilled in the art.

The compounds of the formula (I) can be converted to the customary seed-dressing formulations, such as solutions, emulsions, suspensions, powders, foams, slurries or other coating compositions for seed, and also ULV formulations.

These formulations are prepared in a known manner, by mixing the compounds of the formula (I) with customary additives, for example customary extenders and solvents or diluents, dyes, wetting agents, dispersants, emulsifiers, antifoams, preservatives, secondary thickeners, adhesives, gibberellins, and also water.

Suitable dyes which may be present in the seed-dressing formulations usable in accordance with the invention are all dyes which are customary for such purposes. It is possible to use either pigments, which are sparingly soluble in water, or dyes, which are soluble in water. Examples include the dyes known by the names Rhodamine B, C.I. Pigment Red 112 and C.I. Solvent Red 1.

Useful wetting compositions which may be present in the seed-dressing formulations usable in accordance with the invention are all substances which promote wetting and which are customary for the formulation of agrochemically active compounds. Usable with preference are alkyl naphthalenesulfonates, such as diisopropyl or diisobutyl naphthalenesulfonates.

Suitable dispersants and/or emulsifiers which may be present in the seed-dressing formulations usable in accordance with the invention are all nonionic, anionic and cationic dispersants customary for the formulation of agrochemically active compounds. Nonionic or anionic dispersants or mixtures of nonionic or anionic dispersants can be used with preference. Suitable nonionic dispersants especially include ethylene oxide/propylene oxide block polymers, alkylphenol polyglycol ethers and tristyrylphenol polyglycol ethers, and the phosphated or sulfated derivatives thereof. Suitable anionic dispersants are especially lignosulfonates, polyacrylic acid salts and arylsulfonate-formaldehyde condensates.

Antifoams which may be present in the seed-dressing formulations usable in accordance with the invention are all foam-inhibiting substances customary for the formulation of agrochemically active compounds. Silicone antifoams and magnesium stearate can be used with preference.

Preservatives which may be present in the seed-dressing formulations usable in accordance with the invention are all substances usable for such purposes in agrochemical compositions. Examples include dichlorophene and benzyl alcohol hemiformal.

Useful secondary thickeners which may be present in the seed-dressing formulations usable in accordance with the invention are all substances which can be used for such purposes in agrochemical compositions. Preferred examples include cellulose derivatives, acrylic acid derivatives, xanthan, modified clays and finely divided silica.

Useful stickers which may be present in the seed-dressing formulations usable in accordance with the invention are all customary binders usable in seed-dressing products. Preferred examples include polyvinylpyrrolidone, polyvinyl acetate, polyvinyl alcohol and tylose.

Useful gibberellins which may be present in the seed-dressing formulations usable in accordance with the invention are preferably the gibberellins A1, A3 (=gibberellic acid), A4 and A7; particular preference is given to using gibberellic acid. The gibberellins are known (cf. R. Wegler “Chemie der Pflanzenschutz-und Schädlingsbekampfungsmittel”, vol. 2, Springer Verlag, 1970, pp. 401-412).

The seed-dressing formulations usable in accordance with the invention can be used to treat a wide variety of different kinds of seed, either directly or after prior dilution with water. For instance, the concentrates or the preparations obtainable therefrom by dilution with water can be used to dress the seed of cereals, such as wheat, barley, rye, oats and triticale, and also the seed of maize, rice, oilseed rape, peas, beans, cotton, sunflowers, soya beans and beets, or else a wide variety of different vegetable seed. The seed-dressing formulations usable in accordance with the invention, or the dilute application forms thereof, can also be used to dress seed of transgenic plants.

For the treatment of seed with the seed-dressing formulations usable in accordance with the invention, or the application forms prepared therefrom through the addition of water, all mixing units usable customarily for the seed dressing are useful. Specifically, the procedure in seed dressing is to place the seed into a mixer in batchwise or continuous operation, to add the particular desired amount of seed-dressing formulations, either as such or after prior dilution with water, and to mix until the formulation is distributed homogeneously on the seed. If appropriate, this is followed by a drying operation.

The application rate of the seed-dressing formulations usable in accordance with the invention can be varied within a relatively wide range. It is guided by the particular content of the compounds of the formula (I) in the formulations and by the seed. The application rates of the compound of the formula (I) are generally between 0.001 and 50 g per kilogram of seed, preferably between 0.01 and 15 g per kilogram of seed.

### Animal Health

In the animal health field, i.e. the field of veterinary medicine, the compounds of the formula (I) are active against animal parasites, in particular ectoparasites or endoparasites. The term “endoparasite” includes especially helminths and protozoa, such as coccidia. Ectoparasites are typically and preferably arthropods, especially insects or acarids.

In the field of veterinary medicine, the compounds of the formula (I) having favourable endotherm toxicity are suitable for controlling parasites which occur in animal husbandry and animal keeping in livestock, breeding animals, zoo animals, laboratory animals, experimental animals and domestic animals. They are active against all or specific stages of development of the parasites.

Agricultural livestock include, for example, mammals, such as sheep, goats, horses, donkeys, camels, buffalo, rabbits, reindeer, fallow deer and especially cattle and pigs; or poultry such as turkeys, ducks, geese and especially chickens; or fish or crustaceans, for example in aquaculture; or, as the case may be, insects such as bees.

Domestic animals include, for example, mammals, such as hamsters, guinea pigs, rats, mice, chinchillas, ferrets, and particularly dogs, cats, caged birds; reptiles, amphibians or aquarium fish.

In a specific embodiment, the compounds of the formula (I) are administered to mammals.

In another specific embodiment, the compounds of the formula (I) are administered to birds, namely caged birds or particularly poultry.

Use of the compounds of the formula (I) for the control of animal parasites is intended to reduce or prevent illness, cases of death and reductions in performance (in the case of meat, milk, wool, hides, eggs, honey and the like), such that more economical and simpler animal keeping is enabled and better animal well-being is achievable.

In relation to the field of animal health, the term “control” or “controlling” in the present context means that the compounds of the formula (I) are effective in reducing the incidence of the particular parasite in an animal infected with such parasites to an innocuous degree. More specifically, “controlling” in the present context means that the compounds of the formula (I) kill the respective parasite, inhibit its growth, or inhibit its proliferation.

The arthropods include, for example, but are not limited to,

from the order of Anoplurida, for example Haematopinus spp., Linognathus spp., Pediculus spp., Phtirus spp. and Solenopotes spp.;
from the order of Mallophagida and the suborders Amblycerina and Ischnocerina, for example, Bovicola spp., Damalina spp., Felicola spp.; Lepikentron spp., Menopon spp., Trichodectes spp., Trimenopon spp., Trinoton spp., Werneckiella spp;
from the order of Diptera and the suborders Nematocerina and Brachycerina, for example Aedes spp., Anopheles spp., Atylotus spp., Braula spp., Calliphora spp., Chrysomyia spp., Chrysops spp., Culex spp., Culicoides spp., Eusimulium spp., Fannia spp., Gasterophilus spp., Glossina spp., Haematobia spp., Haematopota spp., Hippobosca spp., Hybomitra spp., Hydrotaea spp., Hypoderma spp., Lipoptena spp., Lucilia spp., Lutzomyia spp., Melophagus spp., Morellia spp., Musca spp., Odagmia spp., Oestrus spp., Philipomyia spp., Phlebotomus spp., Rhinoestrus spp., Sarcophaga spp., Simulium spp., Stomoxys spp., Tabanus spp., Tipula spp., Wilhelmia spp., Wohlfahrtia spp.;
from the order of Siphonapterida, for example Ceratophyllus spp., Ctenocephalides spp., Pulex spp., Tunga spp., Xenopsylla spp.;
from the order of Heteropterida, for example Cimex spp., Panstrongylus spp., Rhodnius spp., Triatoma spp.; and also nuisance and hygiene pests from the order Blattarida.

In addition, in the case of the arthropods, mention should be made by way of example, without limitation, of the following Acari:

from the subclass of Acari (Acarina) and the order of Metastigmata, for example from the family of Argasidae such as Argas spp., Ornithodorus spp., Otobius spp., from the family of Ixodidae such as Amblyomma spp., Dermacentor spp., Haemaphysalis spp., Hyalomma spp., Ixodes spp., Rhipicephalus (Boophilus) spp., Rhipicephalus spp. (the original genus of multi-host ticks); from the order of Mesostigmata such as Dermanyssus spp., Ornithonyssus spp., Pneumonyssus spp., Raillietia spp., Sternostoma spp., Tropilaelaps spp., Varroa spp.; from the order of the Actinedida (Prostigmata), for example, Acarapis spp., Cheyletiella spp., Demodex spp., Listrophorus spp., Myobia spp., Neotrombicula spp., Ornithocheyletia spp., Psorergates spp., Trombicula spp.; and from the order of the Acaridida (Astigmata), for example, Acarus spp., Caloglyphus spp., Chorioptes spp., Cytodites spp., Hypodectes spp., Knemidocoptes spp., Laminosioptes spp., Notoedres spp., Otodectes spp., Psoroptes spp., Pterolichus spp., Sarcoptes spp., Trixacarus spp., Tyrophagus spp.

Examples of parasitic protozoa include, but are not limited to:

Mastigophora (Flagellata), such as:
Metamonada: from the order of Diplomonadida, for example Giardia spp., Spironucleus spp.
Parabasala: from the order of Trichomonadida, for example Histomonas spp., Pentatrichomonas spp., Tetratrichomonas spp., Trichomonas spp., Tritrichomonas spp.
Euglenozoa: from the order of Trypanosomatida, for example Leishmania spp., Trypanosoma spp.
Sarcomastigophora (Rhizopoda) such as Entamoebidae, for example Entamoeba spp., Centramoebidae, for example Acanthamoeba sp., Euamoebidae, e.g. Hartmanella sp.
Alveolata such as Apicomplexa (Sporozoa): e.g. Cryptosporidium spp.; from the order of Eimeriida, for example, Besnoitia spp., Cystoisospora spp., Eimeria spp., Hammondia spp., Isospora spp., Neospora spp., Sarcocystis spp., Toxoplasma spp.; from the order of Adeleida, for example, Hepatozoon spp., Klossiella spp.; from the order of Haemosporida, for example, Leucocytozoon spp., Plasmodium spp.; from the order of Piroplasmida, for example, Babesia spp., Ciliophora spp., Echinozoon spp., Theileria spp.; from the order of Vesibuliferida, for example, Balantidium spp., Buxtonella spp.
Microspora such as Encephalitozoon spp., Enterocytozoon spp., Globidium spp., Nosema spp., and also, for example, Myxozoa spp.

The helminths that are pathogenic to humans or animals include, for example, Acanthocephala, nematodes, Pentastoma and Platyhelminthes (e.g. Monogenea, cestodes and trematodes).

Exemplary helminths include, but are not limited to:

### Monogenea: e.g.: Dactylogyrus spp., Gyrodactylus spp., Microbothrium spp., Polystoma spp., Troglecephalus spp.;

Cestodes: from the order of Pseudophyllidea, for example: Bothridium spp., Diphyllobothrium spp., Diplogonoporus spp., Ichthyobothrium spp., Ligula spp., Schistocephalus spp., Spirometra spp.
From the order of Cyclophyllida, for example: Andyra spp., Anoplocephala spp., Avitellina spp., Bertiella spp., Cittotaenia spp., Davainea spp., Diorchis spp., Diplopylidium spp., Dipylidium spp., Echinococcus spp., Echinocotyle spp., Echinolepis spp., Hydatigera spp., Hymenolepis spp., Joyeuxiella spp., Mesocestoides spp., Moniezia spp., Paranoplocephala spp., Raillietina spp., Stilesia spp., Taenia spp., Thysaniezia spp., Thysanosoma spp.
Trematodes: from the class of Digenea, for example: Austrobilharzia spp., Brachylaima spp., Calicophoron spp., Catatropis spp., Clonorchis spp. Collyriclum spp., Cotylophoron spp., Cyclocoelum spp., Dicrocoelium spp., Diplostomum spp., Echinochasmus spp., Echinoparyphium spp., Echinostoma spp., Eurytrema spp., Fasciola spp., Fasciolides spp., Fasciolopsis spp., Fischoederius spp., Gastrothylacus spp., Gigantobilharzia spp., Gigantocotyle spp., Heterophyes spp., Hypoderaeum spp., Leucochloridium spp., Metagonimus spp., Metorchis spp., Nanophyetus spp., Notocotylus spp., Opisthorchis spp., Omithobilharzia spp., Paragonimus spp., Paramphistomum spp., Plagiorchis spp., Posthodiplostomum spp., Prosthogonimus spp., Schistosoma spp., Trichobilharzia spp., Troglotrema spp., Typhlocoelum spp.
Nematodes: from the order of Trichinellida, for example: Capillaria spp., Trichinella spp., Trichomosoides spp., Trichuris spp.
From the order of Tylenchida, for example: Micronema spp., Parastrangyloides spp., Strongyloides spp.
From the order of Rhabditina, for example: Aelurostrongylus spp., Amidostomum spp., Ancylostoma spp., Angiostrongylus spp., Bronchonema spp., Bunostomum spp., Chabertia spp., Cooperia spp., Cooperioides spp., Crenosoma spp., Cyathostomum spp., Cyclococercus spp., Cyclodontostomum spp., Cylicocyclus spp., Cylicostephanus spp., Cylindropharynx spp., Cystocaulus spp., Dictyocaulus spp., Elaphostrongylus spp., Filaroides spp., Globocephalus spp., Graphidium spp., Gyalocephalus spp., Haemonchus spp., Heligmosomoides spp., Hyostrongylus spp., Marshallagia spp., Metastrongylus spp., Muellerius spp., Necator spp., Nematodirus spp., Neostrongylus spp., Nippostrongylus spp., Obeliscoides spp., Oesophagodontus spp., Oesophagostomum spp., Ollulanus spp.; Omithostrongylus spp., Oslerus spp., Ostertagia spp., Paracooperia spp., Paracrenosoma spp., Parafilaroides spp., Parelaphostrongylus spp., Pneumocaulus spp., Pneumostrongylus spp., Poteriostomum spp., Protostrongylus spp., Spicocaulus spp., Stephanurus spp., Strongylus spp., Syngamus spp., Teladorsagia spp., Trichonema spp., Trichostrongylus spp., Triodontophorus spp., Troglostrongylus spp., Uncinaria spp.
From the order of Spirurida, for example: Acanthocheilonema spp., Anisakis spp., Ascaridia spp.; Ascaris spp., Ascarops spp., Aspiculuris spp., Baylisascaris spp., Brugia spp., Cercopithifilaria spp., Crassicauda spp., Dipetalonema spp., Dirofilaria spp., Dracunculus spp.; Draschia spp., Enterobius spp., Filaria spp., Gnathostoma spp., Gongylonema spp., Habronema spp., Heterakis spp.; Litomosoides spp., Loa spp., Onchocerca spp., Oxyuris spp., Parabronema spp., Parafilaria spp., Parascaris spp., Passalurus spp., Physaloptera spp., Probstmayria spp., Pseudofilaria spp., Setaria spp., Skjrabinema spp., Spirocerca spp., Stephanofilaria spp., Strongyluris spp., Syphacia spp., Thelazia spp., Toxascaris spp., Toxocara spp., Wuchereria spp.
Acanthocephala: from the order of Oligacanthorhynchida, for example: Macracanthorhynchus spp., Prosthenorchis spp.; from the order of Moniliformida, for example: Moniliformis spp.
From the order of Polymorphida, for example: Filicollis spp.; from the order of Echinorhynchida, for example Acanthocephalus spp., Echinorhynchus spp., Leptorhynchoides spp.
Pentastoma: from the order of Porocephalida, for example, Linguatula spp.

In the veterinary field and in animal keeping, the compounds of the formula (I) are administered by methods generally known in the art, such as via the enteral, parenteral, dermal or nasal route in the form of suitable preparations. Administration may be prophylactic, metaphylactic or therapeutic.

Thus, one embodiment of the present invention relates to the compounds of the formula (I) for use as a medicament.

A further aspect relates to the compounds of the formula (I) for use as an antiendoparasitic agent.

A further specific aspect of the invention relates to the compounds of the formula (I) for use as an antihelminthic agent, especially for use as a nematicide, platyhelminthicide, acanthocephalicide or pentastomicide.

A further specific aspect of the invention relates to the compounds of the formula (I) for use as an antiprotozoic agent.

A further aspect relates to the compounds of the formula (I) for use as an antiectoparasitic agent, especially an arthropodicide, very particularly an insecticide or an acaricide.

Further aspects of the invention are veterinary medicine formulations comprising an effective amount of at least one compound of the formula (I) and at least one of the following: a pharmaceutically acceptable excipient (e.g. solid or liquid diluents), a pharmaceutically acceptable auxiliary (e.g. surfactants), especially a pharmaceutically acceptable excipient used conventionally in veterinary medicine formulations and/or a pharmaceutically acceptable auxiliary conventionally used in veterinary medicine formulations.

A related aspect of the invention is a method for production of a veterinary medicine formulation as described here, which comprises the step of mixing at least one compound of the formula (I) with pharmaceutically acceptable excipients and/or auxiliaries, especially with pharmaceutically acceptable excipients used conventionally in veterinary medicine formulations and/or auxiliaries used conventionally in veterinary medicine formulations.

Another specific aspect of the invention is veterinary medicine formulations selected from the group of ectoparasiticidal and endoparasiticidal formulations, especially selected from the group of anthelmintic, antiprotozoic and arthropodicidal formulations, very particularly selected from the group of nematicidal, platyhelminthicidal, acanthocephalicidal, pentastomicidal, insecticidal and acaricidal formulations, according to the aspects mentioned, and methods for production thereof.

Another aspect relates to a method for treatment of a parasitic infection, especially an infection caused by a parasite selected from the group of the ectoparasites and endoparasites mentioned here, by use of an effective amount of a compound of the formula (I) in an animal, especially a nonhuman animal, having a need therefor.

Another aspect relates to a method for treatment of a parasitic infection, especially an infection caused by a parasite selected from the group of the ectoparasites and endoparasites mentioned here, by use of a veterinary medicine formulation as defined here in an animal, especially a nonhuman animal, having a need therefor.

Another aspect relates to the use of the compounds of the formula (I) in the treatment of a parasite infection, especially an infection caused by a parasite selected from the group of the ectoparasites and endoparasites mentioned here, in an animal, especially a nonhuman animal.

In the present context of animal health or veterinary medicine, the term “treatment” includes prophylactic, metaphylactic and therapeutic treatment.

In a particular embodiment, in this way, mixtures of at least one compound of the formula (I) with other active compounds, especially with endo- and ectoparasiticides, are provided for the field of veterinary medicine.

In the field of animal health, “mixture” means not just that two (or more) different active compounds are formulated in a common formulation and are correspondingly employed together, but also relates to products comprising formulations separated for each active compound. Accordingly, when more than two active compounds are to be employed, all active compounds can be formulated in a common formulation or all active compounds can be formulated in separate formulations; likewise conceivable are mixed forms in which some of the active compounds are formulated together and some of the active compounds are formulated separately. Separate formulations allow the separate or successive application of the active compounds in question.

The active compounds specified here by their common names are known and are described, for example, in the “Pesticide Manual” (see above) or can be searched for on the Internet (e.g.: http://www.alanwood.net/pesticides).

Illustrative active compounds from the group of the ectoparasiticides as mixing components include, without any intention that this should constitute a restriction, the insecticides and acaricides listed in detail above. Further usable active compounds are listed below in accordance with the abovementioned classification based on the current IRAC Mode of Action Classification Scheme: (1) acetylcholinesterase (AChE) inhibitors; (2) GABA-gated chloride channel blockers; (3) sodium channel modulators; (4) nicotinic acetylcholine receptor (nAChR) competitive modulators; (5) nicotinic acetylcholine receptor (nAChR) allosteric modulators; (6) glutamate-gated chloride channel (GluCl) allosteric modulators; (7) juvenile hormone mimetics; (8) miscellaneous non-specific (multisite) inhibitors; (9) chordotonal organ modulators; (10) mite growth inhibitors; (12) inhibitors of mitochondrial ATP synthase, such as ATP disruptors; (13) uncouplers of oxidative phosphorylation via disruption of the proton gradient; (14) nicotinic acetylcholine receptor channel blockers; (15) inhibitors of chitin biosynthesis, type 0; (16) inhibitors of chitin biosynthesis, type 1; (17) moulting disruptors (especially in the case of Diptera); (18) ecdysone receptor agonists; (19) octopamine receptor agonists; (21) mitochondrial complex I electron transport inhibitors; (25) mitochondrial complex II electron transport inhibitors; (20) mitochondrial complex III electron transport inhibitors; (22) voltage-dependent sodium channel blockers; (23) inhibitors of acetyl CoA carboxylase; (28) ryanodine receptor modulators;

active compounds having unknown or non-specific mechanisms of action, e.g. fentrifanil, fenoxacrim, cycloprene, chlorobenzilate, chlordimeform, flubenzimin, dicyclanil, amidoflumet, quinomethionat, triarathene, clothiazoben, tetrasul, potassium oleate, petroleum, metoxadiazone, gossyplur, flutenzine, brompropylate, cryolite;
compounds from other classes, for example butacarb, dimetilan, cloethocarb, phosphocarb, pirimiphos(-ethyl), parathion(-ethyl), methacrifos, isopropyl o-salicylate, trichlorfon, sulprofos, propaphos, sebufos, pyridathion, prothoate, dichlofenthion, demeton-S-methyl sulfone, isazofos, cyanofenphos, dialifos, carbophenothion, autathiofos, aromfenvinfos(-methyl), azinphos(-ethyl), chlorpyrifos(-ethyl), fosmethilan, iodofenphos, dioxabenzofos, formothion, fonofos, flupyrazofos, fensulfothion, etrimfos; organochlorine compounds, for example camphechlor, lindane, heptachlor; or phenylpyrazoles, e.g. acetoprole, pyrafluprole, pyriprole, vaniliprole, sisapronil; or isoxazolines, e.g. sarolaner, afoxolaner, lotilaner, fluralaner;
pyrethroids, e.g. (cis-, trans-)metofluthrin, profluthrin, flufenprox, flubrocythrinate, fubfenprox, fenfluthrin, protrifenbut, pyresmethrin, RU15525, terallethrin, cis-resmethrin, heptafluthrin, bioethanomethrin, biopermethrin, fenpyrithrin, cis-cypermethrin, cis-permethrin, clocythrin, cyhalothrin (lambda-), chlovaporthrin, or halogenated hydrocarbon compounds (HCHs),
neonicotinoids, e.g. nithiazine
dicloromezotiaz, triflumezopyrim
macrocyclic lactones, e.g. nemadectin, ivermectin, latidectin, moxidectin, selamectin, eprinomectin, doramectin, emamectin benzoate; milbemycin oxime
triprene, epofenonane, diofenolan;
biologicals, hormones or pheromones, for example natural products, e.g. thuringiensin, codlemone or neem components
dinitrophenols, e.g. dinocap, dinobuton, binapacryl;
benzoylureas, e.g. fluazuron, penfluron,
amidine derivatives, e.g. chlormebuform, cymiazole, demiditraz
beehive Varroa acaricides, for example organic acids, e.g. formic acid, oxalic acid.

Illustrative active compounds from the group of the endoparasiticides, as mixing components, include, but are not limited to, anthelmintically active compounds and antiprotozoic active compounds.

The anthelmintic active ingredients include but are not limited to the following nematicidally, trematicidally and/or cestocidally active ingredients:

from the class of the macrocyclic lactones, for example: eprinomectin, abamectin, nemadectin, moxidectin, doramectin, selamectin, lepimectin, latidectin, milbemectin, ivermectin, emamectin, milbemycin;
from the class of the benzimidazoles and probenzimidazoles, for example: oxibendazole, mebendazole, triclabendazole, thiophanate, parbendazole, oxfendazole, netobimin, fenbendazole, febantel, thiabendazole, cyclobendazole, cambendazole, albendazole sulfoxide, albendazole, flubendazole; from the class of the depsipeptides, preferably cyclic depsipeptides, especially 24-membered cyclic depsipeptides, for example: emodepside, PF1022A;
from the class of the tetrahydropyrimidines, for example: morantel, pyrantel, oxantel; from the class of the imidazothiazoles, for example: butamisole, levamisole, tetramisole; from the class of the aminophenylamidines, for example: amidantel, deacylated amidantel (dAMD), tribendimidine;
from the class of the aminoacetonitriles, for example: monepantel;
from the class of the paraherquamides, for example: paraherquamide, derquantel;
from the class of the salicylanilides, for example: tribromsalan, bromoxanide, brotianide, clioxanide, closantel, niclosamide, oxyclozanide, rafoxanide;
from the class of the substituted phenols, for example: nitroxynil, bithionol, disophenol, hexachlorophene, niclofolan, meniclopholan;
from the class of the organophosphates, for example: trichlorfon, naphthalofos, dichlorvos/DDVP, crufomate, coumaphos, haloxon;
from the class of the piperazinones/quinolines, for example: praziquantel, epsiprantel;
from the class of the piperazines, for example: piperazine, hydroxyzine;
from the class of the tetracyclines, for example: tetracycline, chlorotetracycline, doxycycline, oxytetracycline, rolitetracycline;
from various other classes, for example: bunamidine, niridazole, resorantel, omphalotin, oltipraz, nitroscanate, nitroxynil, oxamniquin, mirasan, miracil, lucanthon, hycanthon, hetolin, emetin, diethylcarbamazine, dichlorophen, diamfenetide, clonazepam, bephenium, amoscanate, clorsulon.

Antiprotozoic active compounds include, but are not limited to, the following active compounds:

from the class of the triazines, for example: diclazuril, ponazuril, letrazuril, toltrazuril;
from the class of polyether ionophores, for example: monensin, salinomycin, maduramicin, narasin;
from the class of the macrocyclic lactones, for example: milbemycin, erythromycin;
from the class of the quinolones, for example: enrofloxacin, pradofloxacin;
from the class of the quinines, for example: chloroquine;
from the class of the pyrimidines, for example: pyrimethamine;
from the class of the sulfonamides, for example: sulfaquinoxaline, trimethoprim, sulfaclozin;
from the class of the thiamines, for example: amprolium;
from the class of the lincosamides, for example: clindamycin;
from the class of the carbanilides, for example: imidocarb;
from the class of the nitrofurans, for example: nifurtimox;
from the class of the quinazolinone alkaloids, for example: halofuginone;
from various other classes, for example: oxamniquin, paromomycin;
from the class of the vaccines or antigens from microorganisms, for example: Babesia canis rossi, Eimeria tenella, Eimeria praecox, Eimeria necatrix, Eimeria mitis, Eimeria maxima, Eimeria brunetti, Eimeria acervulina, Babesia canis vogeli, Leishmania infantum, Babesia canis canis, Dictyocaulus viviparus.

All the mixing components mentioned, as the case may be, may also form salts with suitable bases or acids if they are capable of doing so on the basis of their functional groups.

### Vector Control

The compounds of the formula (I) can also be used in vector control. In the context of the present invention, a vector is an arthropod, especially an insect or arachnid, capable of transmitting pathogens, for example viruses, worms, single-cell organisms and bacteria, from a reservoir (plant, animal, human, etc.) to a host. The pathogens can be transmitted either mechanically (for example trachoma by non-stinging flies) onto a host or after injection into a host (for example malaria parasites by mosquitoes).

Examples of vectors and the diseases or pathogens they transmit are:

### 1) Mosquitoes

Anopheles: malaria, filariasis;
Culex: Japanese encephalitis, filariasis, other viral diseases, transmission of other worms;
Aedes: yellow fever, dengue fever, other viral diseases, filariasis;
Simuliidae: transmission of worms, especially Onchocerca volvulus;
Psychodidae: transmission of leishmaniasis
   2) Lice: skin infections, epidemic typhus;
   3) Fleas: plague, endemic typhus, tapeworms;
   4) Flies: sleeping sickness (trypanosomiasis); cholera, other bacterial diseases;
   5) Mites: acariosis, epidemic typhus, rickettsialpox, tularaemia, Saint Louis encephalitis, tick-borne encephalitis (TBE), Crimean-Congo haemorrhagic fever, borreliosis;
   6) Ticks: borelliosis such as Borrelia bungdorferi sensu lato., Borrelia duttoni, tick-borne encephalitis, Q fever (Coxiella burnetii), babesia (Babesia canis canis), ehrlichiosis.

Examples of vectors in the context of the present invention are insects, for example aphids, flies, leafhoppers or thrips, which can transmit plant viruses to plants. Other vectors capable of transmitting plant viruses are spider mites, lice, beetles and nematodes.

Further examples of vectors in the context of the present invention are insects and arachnids such as mosquitoes, especially of the genera Aedes, Anopheles, for example A. gambiae, A. arabiensis, A. funestus, A. dirus (malaria) and Culex, Psychodidae such as Phlebotomus, Lutzomyia, lice, fleas, flies, mites and ticks, which can transmit pathogens to animals and/or humans.

Vector control is also possible if the compounds of the formula (I) are resistance-breaking.

Compounds of the formula (I) are suitable for use in the prevention of diseases and/or pathogens transmitted by vectors. Thus, a further aspect of the present invention is the use of compounds of the formula (I) for vector control, for example in agriculture, in horticulture, in forests, in gardens and in leisure facilities, and also in the protection of materials and stored products.

### Protection of Industrial Materials

The compounds of the formula (I) are suitable for protecting industrial materials against infestation or destruction by insects, for example from the orders of Coleoptera, Hymenoptera, Isoptera, Lepidoptera, Psocoptera and Zygentoma.

Industrial materials in the present context are understood to mean inanimate materials, such as preferably plastics, adhesives, glues, papers and cards, leather, wood, processed wood products and coating compositions. The use of the invention for protection of wood is particularly preferred.

In a further embodiment, the compounds of the formula (I) are used together with at least one further insecticide and/or at least one fungicide.

In a further embodiment, the compounds of the formula (I) take the form of a ready-to-use pesticide, meaning that they can be applied to the material in question without further modifications. Useful further insecticides or fungicides especially include those mentioned above.

Surprisingly, it has also been found that the compounds of the formula (I) can be employed for protecting objects which come into contact with saltwater or brackish water, in particular hulls, screens, nets, buildings, moorings and signalling systems, against fouling. It is equally possible to use the compounds of the formula (I), alone or in combinations with other active compounds, as antifouling compositions.

### Control of Animal Pests in the Hygiene Sector

The compounds of the formula (I) are suitable for controlling animal pests in the hygiene sector. More particularly, the invention can be used in the domestic protection sector, in the hygiene protection sector and in the protection of stored products, particularly for control of insects, arachnids, ticks and mites encountered in enclosed spaces, for example dwellings, factory halls, offices, vehicle cabins, animal breeding facilities. For controlling animal pests, the compounds of the formula (I) are used alone or in combination with other active compounds and/or auxiliaries. They are preferably used in domestic insecticide products. The compounds of the formula (I) are effective against sensitive and resistant species, and against all developmental stages.

These pests include, for example, pests from the class Arachnida, from the orders Scorpiones, Araneae and Opiliones, from the classes Chilopoda and Diplopoda, from the class Insecta the order Blattodea, from the orders Coleoptera, Dermaptera, Diptera, Heteroptera, Hymenoptera, Isoptera, Lepidoptera, Phthiraptera, Psocoptera, Saltatoria or Orthoptera, Siphonaptera and Zygentoma and from the class Malacostraca the order Isopoda.

Application is effected, for example, in aerosols, unpressurized spray products, for example pump and atomizer sprays, automatic fogging systems, foggers, foams, gels, evaporator products with evaporator tablets made of cellulose or plastic, liquid evaporators, gel and membrane evaporators, propeller-driven evaporators, energy-free, or passive, evaporation systems, moth papers, moth bags and moth gels, as granules or dusts, in baits for spreading or bait stations.

### PREPARATION EXAMPLES

### 2-[6-(Ethylsulfonyl)thieno[3,2-b]furan-5-yl]-3-methyl-6-(trifluoromethyl)-3H-imidazo[4,5-c]pyridine (Ex. I-2)

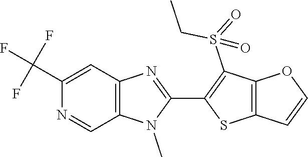 [SMILES: CCS(=O)(=O)c1c(-c2nc3cc(C(F)(F)F)ncc3n2C)sc2ccoc12]

At room temperature, 2-[6-(ethylsulfanyl)thieno[3,2-b]furan-5-yl]-3-methyl-6-(trifluoromethyl)-3H-imidazo[4,5-c]pyridine (405 mg, 1.05 mmol) was dissolved in 23 ml of DCM. Formic acid (243 mg, 5.28 mmol) and hydrogen peroxide solution (aq. 35% strength; 719 mg, 7.39 mmol) were added and the mixture was stirred for 12 h. 40% strength sodium bisulfite solution was added slowly to the reaction solution and the mixture was stirred for 1 h. The organic phase was removed, washed with saturated sodium bicarbonate solution, dried over sodium sulfate and freed of the solvent on a rotary evaporator.

The residue was purified by column chromatography on silica gel with a cyclohexane/ethyl acetate gradient as mobile phase.

MH⁺:416; ¹H-NMR (400 MHz, d-DMSO) δ ppm=9.27 (s, 1H), 8.29 (s, 1H), 8.23 (d, 1H), 7.25 (d, 1H), 3.92 (s, 3H), 3.53-3.47 (q, 2H), 1.19-1.16 (t, 3H).

### 2-[6-(Ethylsulfanyl)thieno[3,2-b]furan-5-yl]-3-methyl-6-(trifluoromethyl)-3H-imidazo[4,5-c]pyridine (Ex. I-2)

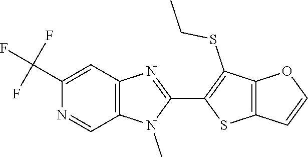 [SMILES: CCSc1c(-c2nc3cc(C(F)(F)F)ncc3n2C)sc2ccoc12]

6-(Ethylsulfanyl)thieno[3,2-b]furan-5-carboxylic acid (770 mg, 2.69 mmol) was dissolved in 4 ml of dichloromethane and 0.02 ml of DMF and cooled to 0° C. Oxalyl chloride (321 mg, 26.9 mmol) was added dropwise and the mixture was then stirred at room temperature for 1 h. All volatile constituents were removed on a rotary evaporator. The crude product was dissolved in 10 ml of 1,4-dioxane and slowly added dropwise to a solution of N3-methyl-6-(trifluoromethyl)pyridine-3,4-diamine (516 mg, 2.69 mmol) in 10 ml of dioxane. The reaction solution was heated at 90° C. for 12 h. After cooling to room temperature, silica gel was added to the solution and the solvent was removed on a rotary evaporator. The residue was purified by column chromatography on silica gel with a cyclohexane/ethyl acetate gradient as mobile phase.

MH⁺:384; ¹H-NMR (400 MHz, d-DMSO) δ ppm: 9.21 (s, 1H), 8.25 (s, 1H), 7.18 (d, 1H), 4.01 (s, 3H), 3.14-3.08 (q, 2H), 1.17-1.13 (t, 3H).

### 6-(Ethylsulfanyl)thieno[3,2-b]furan-5-carboxylic acid

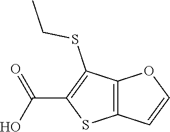

Under an atmosphere of argon, 2,2,6,6-tetramethylpiperidine (1.84 g, 13 mmol) was dissolved in 50 ml of dry THF and cooled to −25° C. n-Butyllithium (2.5 M in hexane, 13 mmol) was slowly added dropwise and the mixture was stirred for 30 min. Thieno[3,2-b]furan-5-carboxylic acid (1.00 g, 5.94 mmol; commercially available) dissolved in 5 ml of THF was added dropwise to the reaction solution. After 30 min of stirring at −25° C., diethyl disulfide (1.82 g, 14.8 mmol) was added dropwise and the mixture was stirred for a further 30 min. The mixture was slowly warmed to room temperature and then concentrated on a rotary evaporator. The residue was taken up in cyclohexane and the resulting precipitate was isolated by filtration. The organic phase was washed twice with 1N hydrochloric acid solution, dried over sodium sulfate and freed of the solvent. The crude product was used in the subsequent reaction without further purification.

MH⁺: 229; ¹H-NMR (400 MHz, d-DMSO) δ ppm: 8.12-8.09 (m, 1H), 7.07 (m, 1H), 3.33 (m, 2H), 1.29 (t, 3H).

### 2-[3-(Ethylsulfonyl)-5-(trifluoromethyl)thieno[3,2-b]thiophen-2-yl]-3-methyl-6-(trifluoromethyl)-3H-imidazo[4,5-c]pyridine (Ex. I-3)

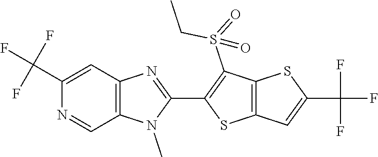 [SMILES: CCS(=O)(=O)c1c(-c2nc3cc(C(F)(F)F)ncc3n2C)sc2cc(C(F)(F)F)sc12]

At room temperature, 2-[3-(ethylsulfanyl)-5-(trifluoromethyl)thieno[3,2-b]thiophen-2-yl]-3-methyl-6-(trifluoromethyl)-3H-imidazo[4,5-c]pyridine (55 mg, 0.09 mmol) was dissolved in 3 ml of DCM. Formic acid (22 mg, 0.48 mmol) and hydrogen peroxide solution (aq. 35% strength; 65 mg, 0.67 mmol) were added and the mixture was stirred for 12 h. 40% strength sodium bisulfite solution was added slowly to the reaction solution and the mixture was stirred for 1 h. The organic phase was removed, washed with saturated sodium bicarbonate solution, dried over sodium sulfate and freed of the solvent on a rotary evaporator. The residue was purified by column chromatography on silica gel with a cyclohexane/ethyl acetate gradient as mobile phase.

MH⁺:501; ¹H-NMR (400 MHz, d-DMSO) δ ppm: 9.32 (s, 1H), 8.37 (m, 1H), 8.34 (m, 1H), 3.95 (s, 3H), 3.62-3.57 (q, 2H), 1.20-1.16 (t, 3H).

### 2-[3-(Ethylsulfanyl)-5-(trifluoromethyl)thieno[3,2-b]thiophen-2-yl]-3-methyl-6-(trifluoromethyl)-3H-imidazo[4,5-c]pyridine (Ex. I-3)

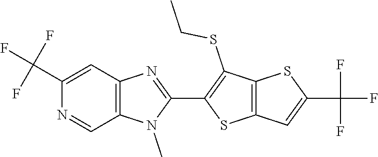 [SMILES: CCSc1c(-c2nc3cc(C(F)(F)F)ncc3n2C)sc2cc(C(F)(F)F)sc12]

3-(Ethylsulfanyl)-5-(trifluoromethyl)thieno[3,2-b]thiophene-2-carboxylic acid (768 mg, 2.45 mmol) was dissolved in 4 ml of dichloromethane and 0.09 ml of DMF and cooled to 0° C. Oxalyl chloride (3120 mg, 24.5 mmol) was added dropwise and the mixture was then stirred at room temperature for 1 h. All volatile constituents were removed on a rotary evaporator. The crude product was dissolved in 5 ml of 1,4-dioxane and slowly added dropwise to a solution of N3-methyl-6-(trifluoromethyl)pyridine-3,4-diamine (417 mg, 2.45 mmol) in 17 ml of dioxane. The reaction solution was heated at 90° C. for 12 h. After cooling to room temperature, the solvent was removed on a rotary evaporator. The residue was taken up in ethyl acetate and washed twice with water. The organic phase was dried over sodium sulfate and concentrated. The residue was purified by column chromatography via preparative HPLC with a water/acetonitrile gradient as mobile phase.

MH⁺:468; ¹H-NMR (400 MHz, d-DMSO) δ ppm: 9.27 (s, 1H), 8.35 (m, 1H), 8.31 (m, 1H), 4.01 (s, 3H), 2.93-2.88 (q, 2H), 1.06-1.03 (t, 3H).

### 3-(Ethylsulfanyl)-5-(trifluoromethyl)thieno[3,2-b]thiophene-2-carboxylic acid

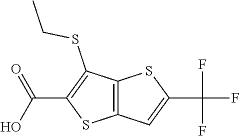 [SMILES: CCSc1c(C(=O)O)sc2cc(C(F)(F)F)sc12]

Under an atmosphere of argon, 2,2,6,6-tetramethylpiperidine (1.127 g, 8.0 mmol) was dissolved in 95 ml of dry THF and cooled to −25° C. n-Butyllithium (2.5 M in hexane, 8.0 mmol) was slowly added dropwise and the mixture was stirred for 30 min. 5-(Trifluoromethyl)thieno[3,2-b]thiophene-2-carboxylic acid (0.915 g, 3.62 mmol) dissolved in 5 ml of THF was added dropwise to the reaction solution. After 30 min of stirring at −25° C., diethyl disulfide (1.109 g, 9.1 mmol) was added dropwise and the mixture was stirred for a further 30 min. The mixture was slowly warmed to room temperature and then concentrated on a rotary evaporator. The residue was taken up in cyclohexane and the resulting precipitate was isolated by filtration. The organic phase was washed twice with 1N hydrochloric acid solution, dried over sodium sulfate and freed of the solvent. The crude product was used in the subsequent reaction without further purification.

### 5-(Trifluoromethyl)thieno[3,2-b]thiophene-2-carboxylic acid

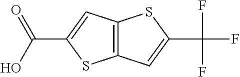 [SMILES: O=C(O)c1cc2sc(C(F)(F)F)cc2s1]

Methyl 5-(trifluoromethyl)thieno[3,2-b]thiophene-2-carboxylate (1.0 g, 3.75 mmol) was dissolved in 10 ml of THF. LiOH (179 mg, 7.51 mmol) dissolved in 2 ml of water was added. The reaction solution was stirred at room temperature for 12 h. 1N hydrochloric acid solution was added and the organic solvent was removed on a rotary evaporator. In the aqueous phase, a precipitate formed which was isolated by filtration and air-dried.

M (neg): 251; ¹H-NMR (400 MHz, d-DMSO) δ ppm: 13.62 (b-s, 1H), 8.23 (s, 1H), 8.21 (s, 1H).

### Methyl 5-(trifluoromethyl)thieno[3,2-b]thiophene-2-carboxylate

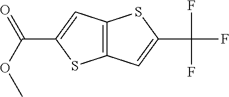 [SMILES: COC(=O)c1cc2sc(C(F)(F)F)cc2s1]

3-Bromo-5-(trifluoromethyl)thiophene-2-carbaldehyde (3.37 g, 13.0 mmol) and potassium carbonate (2.76 g, 20.0 mmol) were initially charged in 50 ml of DMF and heated to 60° C. Ethyl mercaptoacetate (1.4 ml, 13.0 mmol) and a catalytic amount of 18-crown-6 ether were added dropwise to this mixture. The reaction mixture was stirred for 12 h and then poured into 500 ml of water. The mixture was extracted three times with ethyl acetate. The combined organic extracts were washed twice with saturated sodium chloride solution and once with water, dried over magnesium sulfate and concentrated. The residue was purified by column chromatography on silica gel with a cyclohexane/ethyl acetate gradient as mobile phase.

MH⁺:266; ¹H-NMR (400 MHz, CDCl₃) δ ppm: 8.00 (s, 1H), 7.65 (s, 1H), 3.90 (s, 3H).

### 3-Bromo-5-(trifluoromethyl)thiophene-2-carbaldehyde

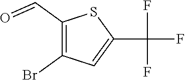 [SMILES: O=Cc1sc(C(F)(F)F)cc1Br]

Under argon, N,N-diisopropylamine (6.00 ml, 42.3 mmol) was dissolved in 24 ml of dry THF. At −78° C., n-butyllithium was added dropwise and the mixture was stirred for 45 min. 4-Bromo-2-(trifluoromethyl)thiophene (7.50 g, 32.5 mmol) dissolved in 24 ml of THF was then added dropwise and the mixture was stirred for 1 h. DMF (3.20 ml, 41.5 mmol) in 30 ml of THF was added and the reaction mixture was stirred for 1 h and slowly warmed to room temperature during this time. Water was added and the mixture was extracted three times with ethyl acetate. The combined organic extracts were dried over sodium sulfate and concentrated. The residue was purified by column chromatography on silica gel with a cyclohexane/ethyl acetate gradient as mobile phase.

### 2-[3-(Ethylsulfonyl)-5,6-dihydro-4H-cyclopenta[b]thiophen-2-yl]-3-methyl-6-(trifluoromethyl)-3H-imidazo[4,5-c]pyridine (Ex. I-4)

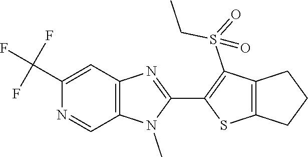 [SMILES: CCS(=O)(=O)c1c(-c2nc3cc(C(F)(F)F)ncc3n2C)sc2c1CCC2]

At room temperature, 2-[3-(ethylsulfanyl)-5-(trifluoromethyl)thieno[3,2-b]thiophen-2-yl]-3-methyl-6-(trifluoromethyl)-3H-imidazo[4,5-c]pyridine (175 mg, 0.41 mmol) was dissolved in 12 ml of DCM. Formic acid (94 mg, 2.05 mmol) and hydrogen peroxide solution (aq. 35% strength; 279 mg, 2.87 mmol) were added and the mixture was stirred for 12 h. 40% strength sodium bisulfite solution was added slowly to the reaction solution and the mixture was stirred for 1 h. The organic phase was removed, washed with saturated sodium bicarbonate solution, dried over sodium sulfate and freed of the solvent on a rotary evaporator. The residue was purified by column chromatography on silica gel with a cyclohexane/ethyl acetate gradient as mobile phase.

MH⁺:416; ¹H-NMR (400 MHz, d-DMSO) δ ppm: 9.24 (s, 1H), 8.26 (s, 1H), 3.86 (s, 3H), 3.35-3.30 (m, 4H), 3.06-2.95 (m, 4H), 1.15-1.11 (t, 3H).

### 2-[3-(Ethylsulfanyl)-5,6-dihydro-4H-cyclopenta[b]thiophen-2-yl]-3-methyl-6-(trifluoromethyl)-3H-imidazo[4,5-c]pyridine (Ex. I-4)

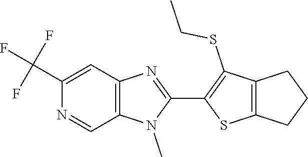 [SMILES: CCSc1c(-c2nc3cc(C(F)(F)F)ncc3n2C)sc2c1CCC2]

3-(Ethylsulfanyl)-5,6-dihydro-4H-cyclopenta[b]thiophene-2-carboxylic acid (465 mg, 1.56 mmol) was dissolved in 4 ml of dichloromethane and 0.02 ml of DMF and cooled to 0° C. Oxalyl chloride (1992 mg, 15.6 mmol) was added dropwise and the mixture was then stirred at room temperature for 1 h. All volatile constituents were removed on a rotary evaporator. The crude product was dissolved in 10 ml of 1,4-dioxane and slowly added dropwise to a solution of N3-methyl-6-(trifluoromethyl)pyridine-3,4-diamine (300 mg, 1.56 mmol) in 10 ml of dioxane. The reaction solution was heated at 90° C. for 12 h. After cooling to room temperature, the solvent was removed on a rotary evaporator. The residue was taken up in ethyl acetate and washed twice with water. The organic phase was dried over sodium sulfate and concentrated. The residue was purified by column chromatography via preparative HPLC using a water/acetonitrile gradient as mobile phase.

MH⁺:384; ¹H-NMR (400 MHz, CDCl₃) δ ppm: 9.18 (s, 1H), 8.22 (s, 1H), 3.94 (s, 3H), 3.32 (m, 2H), 3.02-2.99 (m, 2H), 2.83-2.80 (m, 2H), 2.77-2.71 (m, 2H), 1.05-1.01 (t, 3H).

### 3-(Ethylsulfanyl)-5,6-dihydro-4H-cyclopenta[b]thiophene-2-carboxylic acid

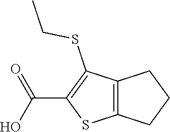

Under an atmosphere of argon, 2,2,6,6-tetramethylpiperidine (1.847 g, 13.0 mmol) was dissolved in 20 ml of dry THF and cooled to −25° C. n-Butyllithium (2.5 M in hexane, 13.0 mmol) was slowly added dropwise and the mixture was stirred for 30 min. 5,6-Dihydro-4H-cyclopenta[b]thiophene-2-carboxylic acid (1.00 g, 5.94 mmol) dissolved in 5 ml of THF was added dropwise to the reaction solution. After 30 min of stirring at −25° C., diethyl disulfide (1.816 g, 14.8 mmol) was added dropwise and the mixture was stirred for a further 30 min. The mixture was slowly warmed to room temperature and then concentrated on a rotary evaporator. The residue was taken up in cyclohexane and the resulting precipitate was isolated by filtration. The organic phase was washed twice with 1N hydrochloric acid solution, dried over sodium sulfate and freed of the solvent. The crude product was used in the subsequent reaction without further purification.

MH⁺:229; ¹H-NMR (400 MHz, CDCl₃) δ ppm: 3.06-3.01 (q, 2H), 2.90-2.68 (m, 6H), 1.17-1.14 (t, 3H).

### 2-[6-Bromo-3-(ethylsulfonyl)thieno[3,2-b]thiophen-2-yl]-3-methyl-6-(trifluoromethyl)-3H-imidazo[4,5-c]pyridine (Ex. I-8)

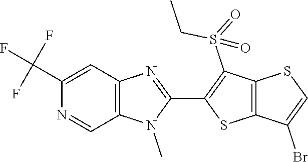 [SMILES: CCS(=O)(=O)c1c(-c2nc3cc(C(F)(F)F)ncc3n2C)sc2c(Br)csc12]

At room temperature, 2-[6-bromo-3-(ethylsulfanyl)thieno[3,2-b]thiophen-2-yl]-3-methyl-6-(trifluoromethyl)-3H-imidazo[4,5-c]pyridine (435 mg, 0.81 mmol) was dissolved in 23 ml of DCM. Formic acid (188 mg, 4.09 mmol) and hydrogen peroxide solution (aq. 35% strength; 556 mg, 5.72 mmol) were added and the mixture was stirred for 12 h. 40% strength sodium bisulfite solution was added slowly to the reaction solution and the mixture was stirred for 1 h. The organic phase was removed, washed with saturated sodium bicarbonate solution, dried over sodium sulfate and freed of the solvent on a rotary evaporator. The residue was purified by column chromatography on silica gel with a cyclohexane/ethyl acetate gradient as mobile phase.

MH⁺: 511; ¹H-NMR (400 MHz, CDCl₃) δ ppm: 9.29 (s, 1H), 8.32 (s, 1H), 7.91 (s, 1H), 3.93 (s, 3H), 3.58-3.51 (q, 2H), 1.19-1.14 (t, 3H).

### 2-[6-Bromo-3-(ethylsulfanyl)thieno[3,2-b]thiophen-2-yl]-3-methyl-6-(trifluoromethyl)-3H-imidazo[4,5-c]pyridine (Ex. I-8)

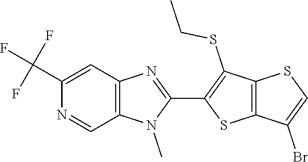 [SMILES: CCSc1c(-c2nc3cc(C(F)(F)F)ncc3n2C)sc2c(Br)csc12]

2-[3-(Ethylsulfanyl)thieno[3,2-b]thiophen-2-yl]-3-methyl-6-(trifluoromethyl)-3H-imidazo[4,5-c]pyridine (1.18 g, 2.95 mmol) was dissolved in 35 ml of tetrahydrofuran. With exclusion of light, N-bromosuccinimide (788.6 mg, 4.43 mmol) was added and the mixture was stirred for 12 h. The reaction mixture was diluted with ethyl acetate and washed with 1N sodium thiosulfate solution. The organic phase was removed, dried over sodium sulfate and freed of the solvent on a rotary evaporator. The crude product was purified by column chromatography (SiO₂, cyclohexane, ethyl acetate).

MH⁺: 480; ¹H-NMR (400 MHz, CDCl₃) δ ppm: 9.25 (s, 1H), 8.28 (s, 1H), 7.87 (s, 1H), 4.00 (s, 3H), 2.91-2.85 (q, 2H), 1.06-1.02 (t, 3H).

### 2-[3-(Ethylsulfanyl)thieno[3,2-b]thiophen-2-yl]-3-methyl-6-(trifluoromethyl)-3H-imidazo[4,5-c]pyridine

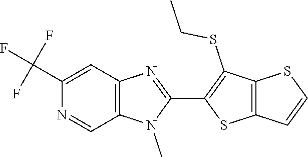 [SMILES: CCSc1c(-c2nc3cc(C(F)(F)F)ncc3n2C)sc2ccsc12]

2-(3-Iodothieno[3,2-b]thiophen-2-yl)-3-methyl-6-(trifluoromethyl)-3H-imidazo[4,5-c]pyridine (1.80 g, 3.86 mmol), ethanethiol (480.8 mg, 7.73 mmol), tris(dibenzylideneacetone)dipalladium(0) (177 mg, 0.19 mmol), Xantphos (223.9 mg, 0.38 mmol) and diisopropylethylamine (0.85 mg, 6.57 mmol) were dissolved in dry dioxane and, under an atmosphere of argon, heated at 80° C. for 3 h. After cooling to room temperature, silica gel was added to the reaction mixture and the solvent was removed on a rotary evaporator. The crude product on silica gel was purified by column chromatography (SiO₂, cyclohexane/ethyl acetate).

MH⁺: 400; ¹H-NMR (400 MHz, CDCl₃) δ ppm: 9.24 (s, 1H), 8.28 (s, 1H), 7.96-7.795 (d, 1H), 7.65-7.64 (d, 1H), 4.00 (s, 3H), 2.95-2.89 (q, 2H), 1.07-1.04 (t, 3H).

### 3-Iodothieno[3,2-b]thiophene-2-carboxylic acid

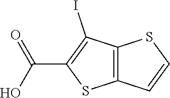 [SMILES: O=C(O)c1sc2ccsc2c1I]

Under an argon atmosphere, methyl thieno[3,2-b]thiophene-2-carboxylate (5.00 g, 25.2 mmol) was dissolved in 20 ml of dry tetrahydrofuran. The solution was cooled to −60° C. and tetramethylpiperidinemagnesium chloride lithium chloride complex (6.11 g, 25.2 mmol, dissolved 1M in toluene/tetrahydrofuran) was slowly added dropwise. After 1 h of stirring at −60° C., iodine (6.40 g, 25.2 mmol, dissolved in 10 ml of tetrahydrofuran) was added dropwise. The mixture was slowly warmed to room temperature and 200 ml of saturated ammonium chloride solution were added. The organic phase was separated off, the solvent was removed on a rotary evaporator and the crude product methyl 3-iodothieno[3,2-b]thiophene-2-carboxylate was used directly for the subsequent reaction.

Methyl 3-iodothieno[3,2-b]thiophene-2-carboxylate (8.1 g, 25 mmol) was dissolved in 100 ml of THF, and lithium hydroxide (2.99 g, 125 mmol) in 20 ml of water was added. The reaction mixture was stirred vigorously at room temperature for 12 h. The mixture was neutralized with 1N hydrochloric acid, and water was added. A precipitate (3-iodothieno[3,2-b]thiophene-2-carboxylic acid) formed, which was isolated by filtration.

### 2-(3-Iodothieno[3,2-b]thiophen-2-yl)-3-methyl-6-(trifluoromethyl)-3H-imidazo[4,5-c]pyridine

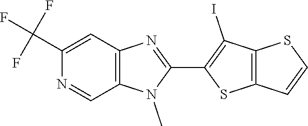 [SMILES: Cn1c(-c2sc3ccsc3c2I)nc2cc(C(F)(F)F)ncc21]

3-Iodothieno[3,2-b]thiophene-2-carboxylic acid (3.24 g, 10.4 mmol) was dissolved in 4 ml of dichloromethane. At 0° C., 0.02 ml of dimethylformamide and oxalyl chloride (13 mg, 104 mmol) were added. The reaction mixture was stirred at room temperature for 1 h and then heated at reflux for 1 h. All volatile constituents were removed on a rotary evaporator and the acid chloride that remained was dissolved in 10 ml of 1,4-dioxane.

N3-Methyl-6-(trifluoromethyl)pyridine-3,4-diamine (2.00 g, 10.4 mmol) was dissolved in 10 ml of dioxane and the acid chloride was slowly added dropwise. The reaction mixture was heated to 90° C. for 12 h. After cooling, crystals formed which were isolated by filtration. The mother liquor was concentrated and the residue was purified by column chromatography (SiO₂, cyclohexane/ethyl acetate).

MH⁺: 465; ¹H-NMR (400 MHz, CDCl₃) δ ppm: 9.27 (s, 1H), 8.31 (s, 1H), 7.99-7.98 (d, 1H), 7.81-7.80 (d, 1H), 4.01 (s, 3H).

### 6-(Ethylsulfonyl)-5-[3-methyl-6-(trifluoromethyl)-3H-imidazo[4,5-c]pyridin-2-yl]thieno[3,2-b]thiophene-3-carbonitrile (Ex. I-5)

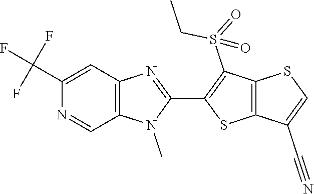 [SMILES: CCS(=O)(=O)c1c(-c2nc3cc(C(F)(F)F)ncc3n2C)sc2c(C#N)csc12]

2-[6-Bromo-3-(ethylsulfonyl)thieno[3,2-b]thiophen-2-yl]-3-methyl-6-(trifluoromethyl)-3H-imidazo[4,5-c]pyridine (50 mg, 0.09 mmol), zinc cyanide (11.5 mg, 0.09 mmol), tris(dibenzylideneacetone)dipalladium(0) (1.8 mg, 0.002 mmol) and 1,1′-bis(diphenylphosphino)ferrocene×DCM (“dppf×DCM”, 1.6 mg, 0.02 mmol) were dissolved in 2 ml of DMF and heated in the microwave at 180° C. for 15 min. The reaction mixture was diluted with MTBE and washed twice with saturated sodium bicarbonate solution. The organic phase was dried over sodium sulfate and freed of the solvent on a rotary evaporator. The residue was purified by column chromatography (SiO₂, cyclohexane/ethyl acetate).

MH⁺: 457; ¹H-NMR (400 MHz, CDCl₃) δ ppm: 9.32 (s, 1H), 8.54 (s, 1H), 8.35 (s, 1H), 3.95 (s, 3H), 3.62-3.57 (q, 2H), 1.19-1.15 (t, 3H).

### N-{6-(Ethylsulfonyl)-5-[3-methyl-6-(trifluoromethyl)-3H-imidazo[4,5-c]pyridin-2-yl]thieno[3,2-b]thiophen-3-yl}acetamide (Ex. I-6)

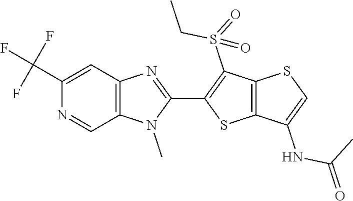 [SMILES: CCS(=O)(=O)c1c(-c2nc3cc(C(F)(F)F)ncc3n2C)sc2c(NC(C)=O)csc12]

2-[6-Bromo-3-(ethylsulfonyl)thieno[3,2-b]thiophen-2-yl]-3-methyl-6-(trifluoromethyl)-3H-imidazo[4,5-c]pyridine (50 mg, 0.09 mmol), acetamide (58 mg, 0.98 mmol), tris(dibenzylideneacetone)dipalladium(0) (9 mg, 0.01 mmol), caesium carbonate (48 mg, 0.15 mmol), trichloromethane (12 mg, 0.1 mmol) and Xantphos (6 mg, 0.01 mmol) were dissolved in 5 ml of dioxane and heated at 100° C. for 4 h. After cooling to room temperature, the reaction solution was purified directly by preparative HPLC.

MH⁺: 489; ¹H-NMR (400 MHz, CDCl₃) δ ppm: 11.72 (s, 1H), 9.27 (s, 1H), 8.29 (s, 1H), 7.19 (s, 1H), 3.92 (s, 3H), 3.54-3.48 (q, 2H), 2.16 (s, 3H), 1.24-1.14 (t, 3H).

### 2-[3-(Ethylsulfonyl)-6-(1-methyl-1H-pyrazol-4-yl)thieno[3,2-b]thiophen-2-yl]-3-methyl-6-(trifluoromethyl)-3H-imidazo[4,5-c]pyridine (Ex. I-11)

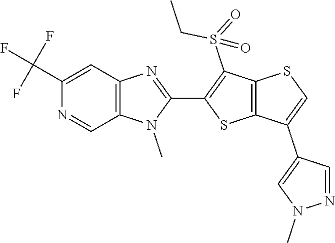 [SMILES: CCS(=O)(=O)c1c(-c2nc3cc(C(F)(F)F)ncc3n2C)sc2c(-c3cnn(C)c3)csc12]

Under an atmosphere of argon, 2-[6-bromo-3-(ethylsulfonyl)thieno[3,2-b]thiophen-2-yl]-3-methyl-6-(trifluoromethyl)-3H-imidazo[4,5-c]pyridine (100 mg, 0.196 mmol), 1-methyl-4-(4,4,5,5-tetramethyl-1,3,2-dioxaborolan-2-yl)-1H-pyrazole (45 mg, 0.216 mmol), tetrakis(triphenylphosphine)palladium(o) (11 mg, 0.01 mmol) and sodium carbonate (83 mg, 0.78 mmol) were dissolved in 3 ml of dioxane and heated at 100° C. for 12 h.

MH⁺: 512; ¹H-NMR (400 MHz, CDCl₃) δ ppm: 9.29 (s, 1H), 8.31 (s, 1H), 8.27 (s, 1H), 7.90 (s, 1H), 7.73 (s, 1H), 5.76 (s, 1H), 3.95 (s, 3H), 3.90 (s, 3H), 3.58-3.53 (q, 2H), 1.20-1.17 (t, 3H).

### 6-(Ethylsulfonyl)-5-[3-methyl-6-(trifluoromethyl)-3H-imidazo[4,5-c]pyridin-2-yl]thieno[3,2-b]thiophene-3-carboxamide (Ex. I-15)

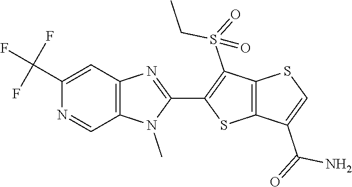 [SMILES: CCS(=O)(=O)c1c(-c2nc3cc(C(F)(F)F)ncc3n2C)sc2c(C(N)=O)csc12]

In a 25 ml autoclave, 2-[6-bromo-3-(ethylsulfonyl)thieno[3,2-b]thiophen-2-yl]-3-methyl-6-(trifluoromethyl)-3H-imidazo[4,5-c]pyridine (200 mg, 0.39 mmol) was dissolved in 5 ml of methanol, and sodium acetate (60 mg, 0.733 mmol) and dichloro[1,1-bis(diphenylphosphino) ferrocene]palladium(II) acetone adduct (“J&M Pd-107”, 60 mg, 0.076 mmol) were added. 3 times, the apparatus was flushed with argon. The mixture was then stirred for 18 h at 40° C. at a carbon monoxide pressure of 5 bar. After removal of all solids by filtration, the organic phase was concentrated and the crude product methyl 6-(ethylsulfonyl)-5-[3-methyl-6-(trifluoromethyl)-3H-imidazo[4,5-c]pyridin-2-yl]thieno[3,2-b]thiophene-3-carboxylate was purified by preparative HPLC. Methyl 6-(ethylsulfonyl)-5-[3-methyl-6-(trifluoromethyl)-3H-imidazo[4,5-c]pyridin-2-yl]thieno[3,2-b]thiophene-3-carboxylate (29 mg, 0.06 mmol) was dissolved in 1 ml of methanol and 1 ml of tetrahydrofuran, and ammonia (152 mg, 2.96 mmol, 33%, aqueous) was added. The mixture was stirred at room temperature for 12 h. After addition of silica gel, all volatile constituents were removed and the crude product on silica gel was purified by column chromatography.

MH⁺: 475; ¹H-NMR (400 MHz, CDCl₃) δ ppm: 9.30 (s, 1H), 8.36 (b-s, 1H), 8.33 (s, 1H), 8.24 (s, 1H), 7.76 (b-s, 1H), 3.94 (s, 3H), 3.57-3.51 (q, 2H), 1.18-1.15 (t, 3H).

### 2-[3-(Ethylsulfonyl)-6-phenylthieno[3,2-b]thiophen-2-yl]-3-methyl-6-(trifluoromethyl)-3H-imidazo[4,5-c]pyridine (Ex. I-16)

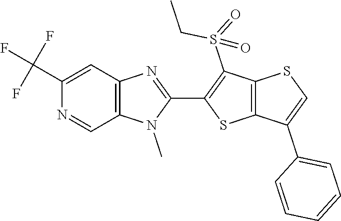 [SMILES: CCS(=O)(=O)c1c(-c2nc3cc(C(F)(F)F)ncc3n2C)sc2c(-c3ccccc3)csc12]

2-[6-Bromo-3-(ethylsulfanyl)thieno[3,2-b]thiophen-2-yl]-3-methyl-6-(trifluoromethyl)-3H-imidazo[4,5-c]pyridine (150 mg, 0.31 mmol), phenylboronic acid (152 mg, 1.25 mmol), palladium acetate (7 mg, 0.031 mmol), S-Phos (26 mg, 0.06 mmol) and potassium phosphate (333 mg, 1.568 mmol) were dissolved in 6 ml of toluene and 3 ml of THF. The mixture was heated at reflux for 7 h. After cooling to room temperature, silica gel was added and all volatile constituents were removed on a rotary evaporator. The residue, 2-[3-(ethylsulfanyl)-6-phenylthieno[3,2-b]thiophen-2-yl]-3-methyl-6-(trifluoromethyl)-3H-imidazo[4,5-c]pyridine on silica gel, was purified by column chromatography.

At room temperature, 2-[3-(ethylsulfanyl)-6-phenylthieno[3,2-b]thiophen-2-yl]-3-methyl-6-(trifluoromethyl)-3H-imidazo[4,5-c]pyridine (77 mg, 0.15 mmol) was dissolved in 23 ml of DCM. Formic acid (37 mg, 0.8 mmol) and hydrogen peroxide solution (aq. 35% strength; 110 mg, 1.13 mmol) were added and the mixture was stirred for 12 h. 40% strength sodium bisulfite solution was added slowly to the reaction solution and the mixture was stirred for 1 h. The organic phase was removed, washed with saturated sodium bicarbonate solution, dried over sodium sulfate and freed of the solvent on a rotary evaporator. The residue was purified by column chromatography on silica gel with a cyclohexane/ethyl acetate gradient as mobile phase.

MH⁺: 508; ¹H-NMR (400 MHz, CDCl₃) δ ppm: 9.30 (s, 1H), 8.33 (s, 1H), 8.11 (s, 1H), 7.81-7.79 (m, 2H), 7.54-7.50 (m, 2H), 7.46-7.43 (m, 1H), 3.96 (s, 3H), 3.60-3.53 (q, 2H), 1.22-1.18 (t, 3H).

### 1-{6-(Ethylsulfonyl)-5-[3-methyl-6-(trifluoromethyl)-3H-imidazo[4,5-c]pyridin-2-yl]thieno[3,2-b]thiophen-3-yl}-3-methylurea (Ex. I-17)

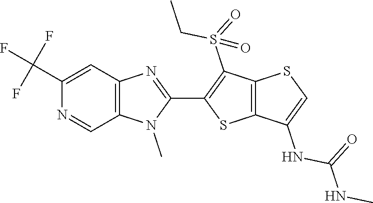 [SMILES: CCS(=O)(=O)c1c(-c2nc3cc(C(F)(F)F)ncc3n2C)sc2c(NC(=O)NC)csc12]

Under an atmosphere of argon, 2-[6-bromo-3-(ethylsulfonyl)thieno[3,2-b]thiophen-2-yl]-3-methyl-6-(trifluoromethyl)-3H-imidazo[4,5-c]pyridine (50 ml, 0.1 mmol), N-methylurea (9 mg, 0.118 mmol), caesium carbonate (48 mg, 0.15 mmol), 4,5-bis(diphenylphosphine)-9,9-dimethylxanthene (6 mg, 0.01 mmol) and tris(dibenzylideneacetone)dipalladium(0) were dissolved in 5 ml of dry degassed dioxane and heated at 80° C. for 4 h. After cooling to room temperature, the mixture was purified by preparative HPLC.

MH⁺: 504; ¹H-NMR (400 MHz, CDCl₃) δ ppm: 9.25 (s, 1H), 8.28 (s, 1H), 6.96 (s, 1H), 3.92 (s, 3H),

### 3.53-3.47 (q, 2H), 2.71-2.67 (m, 3H), 1.18-1.14 (t, 3H). 2-[6-(4-Chloro-1H-pyrazol-1-yl)-3-(ethylsulfonyl)thieno[3,2-b]thiophen-2-yl]-3-methyl-6-(trifluoromethyl)-3H-imidazo[4,5-c]pyridine (Ex. I-20)

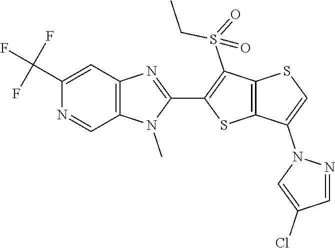 [SMILES: CCS(=O)(=O)c1c(-c2nc3cc(C(F)(F)F)ncc3n2C)sc2c(-n3cc(Cl)cn3)csc12]

In a pressure vessel, 2-[6-bromo-3-(ethylsulfonyl)thieno[3,2-b]thiophen-2-yl]-3-methyl-6-(trifluoromethyl)-3H-imidazo[4,5-c]pyridine (100 mg, 0.19 mmol), 4-chloro-1H-pyrazole (22 mg, 0.216 mmol), copper(I) iodide (4 mg, 0.02 mmol), trans-N,N′-dimethylcyclohexane-1,2-diamine (6 mg, 0.04 mmol), potassium iodide (10 mg, 0.06 mmol) and potassium carbonate (81 mg, 0.6 mmol) were dissolved in 10 ml of dry dioxane and degassed. The reaction vessel was closed and with stirring the mixture was heated at 130° C. for 5 h. After cooling to room temperature, all of the solid constituents were filtered off and the solution was concentrated. The residue was purified by column chromatography (cyclohexane, ethyl acetate).

MH⁺: 533; ¹H-NMR (400 MHz, CDCl₃) δ ppm: 9.30 (s, 1H), 8.95 (s, 1H), 8.32 (s, 1H), 8.00 (s, 1H), 7.92 (s, 1H), 3.96 (s, 3H), 3.61-3.56 (q, 2H), 1.21-1.17 (t, 3H).

### 2-[3-(Ethylsulfonyl)-4,6-difluoro-5,6-dihydro-4H-cyclopenta[b]thiophen-2-yl]-3-methyl-6-(trifluoromethyl)-3H-imidazo[4,5-c]pyridine (Ex. I-29)

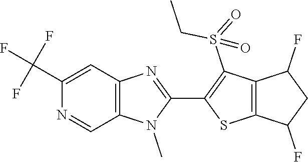 [SMILES: CCS(=O)(=O)c1c(-c2nc3cc(C(F)(F)F)ncc3n2C)sc2c1C(F)CC2F]

In a 10 ml Schlenk vessel under an atmosphere of argon, 2-[3-(ethylsulfonyl)-5,6-dihydro-4H-cyclopenta[b]thiophen-2-yl]-3-methyl-6-(trifluoromethyl)-3H-imidazo[4,5-c]pyridine (50 mg, 0.12 mmol), potassium peroxodisulfate (49 mg, 0.18 mmol) and Selectfluor (85 mg, 0.24 mmol) were dissolved in 1.2 ml of acetonitrile and 1.2 mol of water. The vessel was closed with a septum and the reaction solution was heated at 80° C. for 3 h. The mixture was diluted with dichloromethane and the aqueous phase was removed by filtration over a water-repellent filter. After removal of the solvent, the crude product was purified by preparative HPLC.

In this manner, in addition to Ex. I-33, Example Compounds I-26, I-27, I-28, I-30 were also isolated separately as byproducts.

¹H-NMR (400 MHz, CDCl₃) δ ppm: 9.10 (s, 1H), 8.19 (s, 1H), 6.10-6.09 (m, 1H), 6.01-6.00 (m, 1H), 3.89 (s, 3H), 3.41-3.32 (q, 4H), 1.25-1.22 (t, 3H).

The determination of the M⁺ by LC-MS in the acidic range was carried out at pH 2.7 using the mobile phases 0.1% aqueous formic acid and acetonitrile (contains 0.1% formic acid); linear gradient from 10% acetonitrile to 95% acetonitrile, instrument: Agilent 1100 LC system, Agilent MSD system, HTS PAL.

The determination of the M⁺ by LC-MS in the neutral range was carried out at pH 7.8 using the mobile phases 0.001 molar aqueous ammonium bicarbonate solution and acetonitrile; linear gradient from 10% acetonitrile to 95% acetonitrile.

The NMR data of selected examples are listed either in conventional form (6 values, multiplet splitting, number of hydrogen atoms) or as NMR peak lists.

In each case, the solvent in which the NMR spectrum was recorded is stated.

NMR peak list method

The ¹H NMR data of selected examples are stated in the form of ¹H NMR peak lists. For each signal peak, first the δ value in ppm and then the signal intensity in round brackets are listed. The pairs of δ value-signal intensity numbers for different signal peaks are listed with separation from one another by semicolons.

The peak list for one example therefore takes the form of:

δ₁ (intensity₁); δ₂ (intensity₂); . . . ; δᵢ (intensityᵢ); . . . ; δₙ (intensityₙ)

The intensity of sharp signals correlates with the height of the signals in a printed example of an NMR spectrum in cm and shows the true ratios of the signal intensities. In the case of broad signals, several peaks or the middle of the signal and the relative intensity thereof may be shown in comparison to the most intense signal in the spectrum.

For calibration of the chemical shift of ¹H NMR spectra, we use tetramethylsilane and/or the chemical shift of the solvent, particularly in the case of spectra which are measured in DMSO. Therefore, the tetramethylsilane peak may but need not occur in NMR peak lists.

The lists of the ¹H NMR peaks are similar to the conventional ¹H NMR printouts and thus usually contain all peaks listed in a conventional NMR interpretation.

In addition, like conventional ¹H NMR printouts, they may show solvent signals, signals of stereoisomers of the target compounds which are likewise provided by the invention, and/or peaks of impurities.

In the reporting of compound signals within the delta range of solvents and/or water, our lists of ¹H NMR peaks show the standard solvent peaks, for example peaks of DMSO in DMSO-D₆ and the peak of water, which usually have a high intensity on average.

The peaks of stereoisomers of the target compounds and/or peaks of impurities usually have a lower intensity on average than the peaks of the target compounds (for example with a purity of >90%).

Such stereoisomers and/or impurities may be typical of the particular preparation process. Their peaks can thus help in identifying reproduction of our preparation process with reference to “by-product fingerprints”.

An expert calculating the peaks of the target compounds by known methods (MestreC, ACD simulation, but also with empirically evaluated expected values) can, if required, isolate the peaks of the target compounds, optionally using additional intensity filters. This isolation would be similar to the peak picking in question in conventional ¹H NMR interpretation.

Further details of ¹H NMR peak lists can be found in the Research Disclosure Database Number 564025.

The compounds listed in Table 1 were prepared as described above or analogously thereto.

Table 1

| Ex. | Structure | |
|---|---|---|
| I-1 | 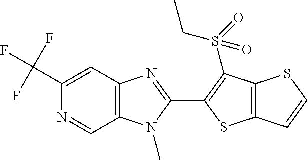 [SMILES: CCS(=O)(=O)c1c(-c2nc3cc(C(F)(F)F)ncc3n2C)sc2ccsc12] | ¹H-NMR (400.0 MHz, d₆-DMSO): δ = 9.2923 (4.0); 8.3186 (4.3); 8.0416 (3.4); 8.0282 (3.7); 7.7090 (3.7); 7.6956 (3.5); 5.7565 (11.0); 3.9335 (16.0); 3.5450 (1.1); 3.5265 (3.6); 3.5081 (3.6); 3.4897 (1.1); 3.3430 (0.6); 3.3254 (15.7); 3.0145 (0.4); 2.9263 (0.4); 2.5077 (26.6); 2.5033 (35.0); 2.4988 (26.3); 1.9899 (0.6); 1.3968 (0.7); 1.2399 (0.5); 1.2232 (0.4); 1.1797 (4.0); 1.1613 (8.6); 1.1428 |
| | | (3.8); 0.0079 (1.2); −0.0002 (27.6); |
| | | −0.0083 (1.2) |
| I-2 | 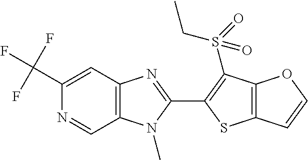 [SMILES: CCS(=O)(=O)c1c(-c2nc3cc(C(F)(F)F)ncc3n2C)sc2ccoc12] | ¹H-NMR (400.0 MHz, d₆-DMSO): δ = 9.2665 (4.4); 8.2922 (4.6); 8.2366 (3.9); 8.2314 (4.0); 7.2544 (4.0); 7.2492 (3.9); 5.7573 (2.7); 3.9144 (16.0); 3.5319 (1.2); 3.5135 (3.8); 3.4950 (3.9); 3.4766 (1.2); 3.3238 (4.6); 2.5079 (16.3); 2.5038 (21.2); 2.4996 (16.2); 1.9905 (0.6); 1.1951 (4.1); 1.1766 (8.7); 1.1582 (4.0); −0.0002 (15.2); −0.0082 (0.7) |
| I-3 | 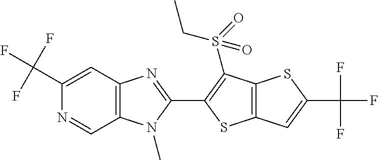 [SMILES: CCS(=O)(=O)c1c(-c2nc3cc(C(F)(F)F)ncc3n2C)sc2cc(C(F)(F)F)sc12] | ¹H-NMR (400.0 MHz, d₆-DMSO): δ = 9.3168 (4.4); 8.3739 (3.2); 8.3721 (3.4); 8.3431 (4.6); 5.7561 (8.2); 3.9551 (16.0); 3.6257 (1.1); 3.6073 (3.6); 3.5889 (3.6); 3.5705 (1.1); 3.3251 (8.3); 2.5093 (16.6); 2.5051 (22.1); 2.5011 (17.0); 1.3966 (0.9); 1.2007 (3.9); 1.1823 (8.4); 1.1639 (3.8); 0.0078 (0.7); −0.0002 (18.0) |
| I-4 | 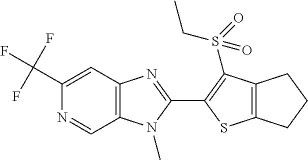 [SMILES: CCS(=O)(=O)c1c(-c2nc3cc(C(F)(F)F)ncc3n2C)sc2c1CCC2] | ¹H-NMR (400.0 MHz, d₆-DMSO): δ = 9.2369 (4.2); 8.2612 (4.4); 5.7567 (7.4); 4.0394 (0.6); 4.0216 (0.6); 3.8595 (16.0); 3.3509 (1.2); 3.3322 (4.8); 3.3259 (12.8);3.3141 (3.9); 3.2956 (1.2); 3.2438 (0.7); 3.0624 (1.3); 3.0444 (2.4); 3.0260 (1.5); 2.9885 (1.4); 2.9704 (2.6); 2.9522 (1.7); 2.5351 (0.7); 2.5080 (16.8); 2.5037 (22.1); 2.4993 (17.7); 2.4804 (1.6); 2.4623 (0.5); 1.9901 (2.5); 1.2349 (0.5); |
| | | 1.2183 (0.4); 1.2148 (0.5); 1.1940 (0.8); |
| | | 1.1761 (1.4); 1.1583 (0.8); 1.1466 (4.1); |
| | | 1.1283 (8.3); 1.1098 (3.7); 0.0078 (0.7); |
| | | −0.0002 (15.7) |
| I-5 | 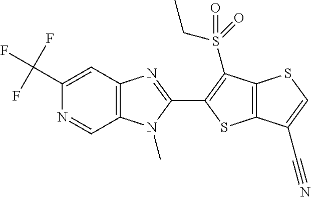 [SMILES: CCS(=O)(=O)c1c(-c2nc3cc(C(F)(F)F)ncc3n2C)sc2c(C#N)csc12] | ¹H-NMR (400.0 MHz, d₆-DMSO): δ = 9.3153 (3.9); 8.5427 (7.0); 8.3465 (4.1); 4.0379 (0.4); 4.0199 (0.5); 3.9489 (16.0); 3.6216 (1.0); 3.6031 (3.4); 3.5846 (3.5); 3.5662 (1.0); 3.3206 (108.3); 2.6750 (0.8); 2.6706 (1.2); 2.6661 (0.9); 2.5238 (3.7); 2.5103 (72.3); 2.5060 (149.8); 2.5015 (200.3); 2.4971 (146.0); 2.4928 (72.4); 2.3327 (0.8); 2.3283 (1.2); 2.3237 (0.8); 1.9885 (2.0); 1.1910 (3.9); 1.1725 (8.5); 1.1540 (3.7); 0.1459 (0.7); 0.0078 (5.6); |
| | | −0.0002 (154.0); −0.0082 (7.0); |
| | | −0.1499 (0.7) |
| I-6 | 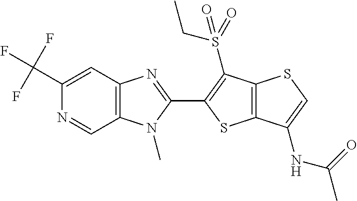 [SMILES: CCS(=O)(=O)c1c(-c2nc3cc(C(F)(F)F)ncc3n2C)sc2c(NC(C)=O)csc12] | ¹H-NMR (400.0 MHz, d₆-DMSO): δ = 11.7220 (3.0); 9.2646 (3.8); 8.3139 (0.6); 8.2912 (4.1); 8.2223 (0.4); 7.3822 (0.3); 7.3348 (0.4); 7.1941 (7.7); 5.7540 (2.5); 3.9231 (16.0); 3.8235 (1.1); 3.5683 (0.6); 3.5379 (1.2); 3.5193 (3.6); 3.5008 (3.6); 3.4828 (1.2); 3.3891 (0.4); 3.3263 (349.8); 2.6758 (1.3); 2.6713 (1.8); 2.6667 (1.3); 2.5244 (5.6); 2.5111 (105.8); 2.5067 (218.4); 2.5022 (290.7); 2.4977 (208.4); 2.4933 (100.2); 2.3335 (1.1); 2.3287 (1.6); 2.3243 (1.3); 2.1581 (15.7); 1.7955 (0.8); |
| | | 1.2350 (1.0); 1.1787 (4.0); 1.1604 (8.7); |
| | | 1.1419 (3.8); 1.0695 (0.4); 0.1463 (0.6); |
| | | 0.0079 (4.9); −0.0002 (137.7); −0.0084 |
| | | (5.6); −0.1496 (0.6) |
| I-7 | 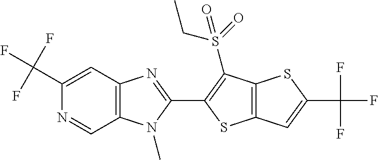 [SMILES: CCS(=O)(=O)c1c(-c2nc3cc(C(F)(F)F)ncc3n2C)sc2cc(C(F)(F)F)sc12] | ¹H-NMR (400.0 MHz, d₆-DMSO): δ = 8.9267 (2.1); 8.9233 (2.2); 8.7334 (2.2); 8.7297 (2.2); 8.3760 (2.9); 8.3730 (2.9); 3.8390 (16.0); 3.6497 (0.9); 3.6313 (3.2); 3.6129 (3.3); 3.5944 (1.0); 3.3234 (112.2); 2.6755 (0.4); 2.6710 (0.6); 2.6665 (0.4); 2.5244 (1.4); 2.5196 (2.2); 2.5109 (36.0); 2.5065 (76.6); 2.5020 (103.0); 2.4974 (73.6); 2.4930 (35.0); 2.3332 (0.4); 2.3287 (0.6); 2.3241 (0.4); 1.3974 (9.0); |
| | | 1.2046 (3.4); 1.1862 (7.9); 1.1677 (3.4); |
| | | −0.0002 (8.8) |
| I-8 | 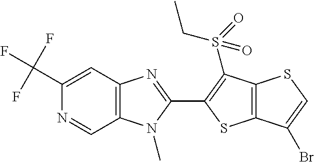 [SMILES: CCS(=O)(=O)c1c(-c2nc3cc(C(F)(F)F)ncc3n2C)sc2c(Br)csc12] | ¹H-NMR (400.0 MHz, d₆-DMSO): δ9.2938 (3.8); 8.3197 (4.1); 7.9110 (8.3); 5.7557 (6.6); 3.9348 (16.0); 3.5834 (1.0); 3.5650 (3.4); 3.5465 (3.5); 3.5279 (1.0); 3.3246 (28.6); 2.5257 (0.5); 2.5120 (12.6); 2.5077 (26.5); 2.5032 (35.6); 2.4987 (25.4); 2.4943 (12.0); 1.3969 (0.4); 1.1819 (3.6); 1.1636 (8.2); 1.1450 (3.5); −0.0002 (7.5) |
| I-9 | 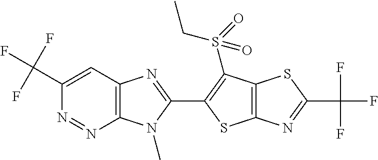 [SMILES: CCS(=O)(=O)c1c(-c2nc3cc(C(F)(F)F)nnc3n2C)sc2nc(C(F)(F)F)sc12] | ¹H-NMR (400.0 MHz, d₆-DMSO): δ = 8.8310 (6.2); 4.0295 (16.0); 3.6745 (1.0); 3.6561 (3.4); 3.6376 (3.5); 3.6192 (1.1); 3.3220 (212.9); 2.6752 (0.9); 2.6707 (1.2); 2.6662 (0.9); 2.5241 (3.2); 2.5104 (75.2); 2.5061 (157.1); 2.5017 (211.6); 2.4972 (154.5); 2.4929 (76.3); 2.3329 (0.8); 2.3286 (1.2); 2.3243 1.3978 (1.3); 1.2199 (3.7); 1.2015 (8.1); 1.1830 (3.6); 0.0079 (1.0); −0.0002 (30.0); −0.0084 (1.4) |
| I-10 | 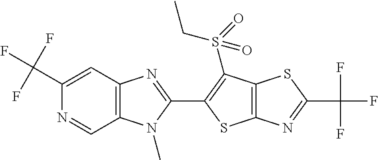 [SMILES: CCS(=O)(=O)c1c(-c2nc3cc(C(F)(F)F)ncc3n2C)sc2nc(C(F)(F)F)sc12] | ¹H-NMR (400.0 MHz, d₆-DMSO): δ = 9.3233 (4.1); 8.3606 (4.4); 3.9749 (16.0); 3.6723 (1.0); 3.6541 (3.5); 3.6356 (3.6); 3.6171 (1.1); 3.3643 (313.8); 3.3568 (283.6); 3.3501 (289.0); 2.6775 (0.7); 2.6728 (1.0); 2.6685 (0.8); 2.5261 (2.3); 2.5126 (63.6); 2.5082 (137.6); 2.5037 (188.1); 2.4992 (135.2); 2.4948 (64.1); 2.3348 (0.7); 2.3303 (1.0); 2.3258 (0.8); 1.3975 (1.3); 1.2339 (0.8); 1.2121 (3.8); 1.1937 (8.4); 1.1753 (3.7); −0.0001 (10.9); −0.0084 (0.5) |
| I-11 | 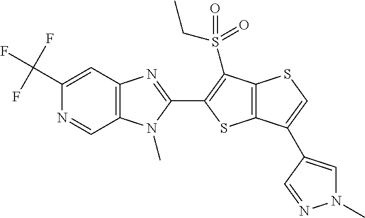 [SMILES: CCS(=O)(=O)c1c(-c2nc3cc(C(F)(F)F)ncc3n2C)sc2c(-c3cnn(C)c3)csc12] | ¹H-NMR (400.0 MHz, d₆-DMSO): δ = 9.2875 (4.0); 8.3139 (4.2); 8.2682 (4.6); 7.9011 (5.0); 7.7291 (7.4); 5.7566 (8.7); 3.9494 (16.0); 3.8996 (14.8); 3.5810 (1.0); 3.5625 (3.4); 3.5441 (3.5); 3.5257 (1.0); 3.3270 (23.6); 2.5259 (0.5); 2.5125 (13.1); 2.5081 (27.5); 2.5037 (36.7); 2.4992 (26.5); 2.4949 (12.8); 1.2035 (3.7); 1.1852 (8.4); 1.1667 (3.6); −0.0002 (6.2) |
| I-12 | 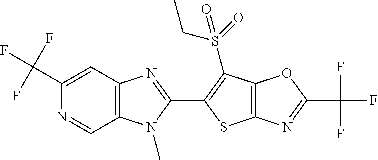 [SMILES: CCS(=O)(=O)c1c(-c2nc3cc(C(F)(F)F)ncc3n2C)sc2nc(C(F)(F)F)oc12] | ¹H-NMR (400.0 MHz, d₆-DMSO): δ = 9.2855 (4.5); 8.3147 (4.8); 7.9349 (3.7); 4.0383 (0.7); 4.0205 (0.7); 3.9186 (16.0); 3.5473 (1.2); 3.5290 (3.9); 3.5105 (3.9); 3.4921 (1.3); 3.3197 (33.6); 2.6712 (0.6); 2.5021 (94.7); 2.3285 (0.6); 1.9889 (2.9); 1.3977 (0.8); 1.2082 (4.2); 1.1899 (9.0); 1.1716 (4.5); 1.1576 (1.0); −0.0002 (31.1) |
| I-13 | 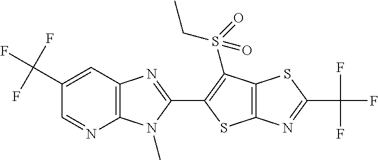 [SMILES: CCS(=O)(=O)c1c(-c2nc3cc(C(F)(F)F)cnc3n2C)sc2nc(C(F)(F)F)sc12] | ¹H-NMR (400.0 MHz, d₆-DMSO): δ = 8.9376 (2.2); 8.9340 (2.4); 8.7501 (2.3); 8.7458 (2.3); 3.8668 (16.0); 3.7054 (1.0); 3.6870 (3.4); 3.6685 (3.4); 3.6501 (1.0); 3.3231 (115.2); 2.6758 (0.5); 2.6712 (0.6); 2.6667 (0.5); 2.5245 (1.6); 2.5110 (38.8); 2.5066 (81.6); 2.5022 (109.6); 2.4977 (79.3); 2.4934 (38.5); 2.3333 (0.4); 2.3289 (0.6); 2.3243 (0.4); 1.3978 (0.4); 1.2232 (3.6); 1.2048 (8.0); 1.1863 (3.4); |
| | | 0.0080 (0.5); −0.0001 (16.6); −0.0083 |
| | | (0.7) |
| I-14 | 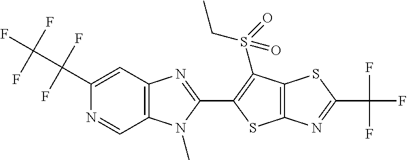 [SMILES: CCS(=O)(=O)c1c(-c2nc3cc(C(F)(F)C(F)(F)F)ncc3n2C)sc2nc(C(F)(F)F)sc12] | ¹H-NMR (400.0 MHz, d₆-DMSO): δ = 9.3480 (4.0); 8.3905 (4.2); 3.9763 (16.0); 3.6813 (1.0); 3.6629 (3.5); 3.6444 (3.5); 3.6260 (1.1); 3.3227 (46.0); 2.6714 (0.3); 2.5246 (0.8); 2.5112 (21.3); 2.5068 (44.8); 2.5023 (60.4); 2.4979 (43.9); 2.4936 (21.6); 2.3292 (0.3); 1.3976 (8.8); 1.2161 (3.7); 1.1976 (8.4); 1.1792 (3.6); −0.0002 (9.1); −0.0083 (0.4) |
| I-15 | 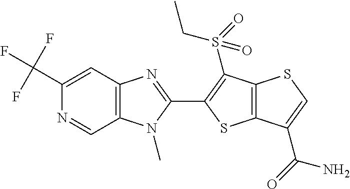 [SMILES: CCS(=O)(=O)c1c(-c2nc3cc(C(F)(F)F)ncc3n2C)sc2c(C(N)=O)csc12] | ¹H-NMR (400.0 MHz, d₆-DMSO): δ = 9.3002 (3.8); 8.3558 (0.7); 8.3283 (4.2); 8.3151 (1.2); 8.2424 (6.6); 7.7570 (0.7); 3.9394 (16.0); 3.5652 (1.0); 3.5470 (3.4); 3.5285 (3.5); 3.5101 (1.1); 3.3209 (120.6); 2.6753 (0.8); 2.6708 (1.1); 2.6662 (0.8); 2.5242 (2.7); 2.5107 (66.1); 2.5063 (138.9); 2.5018 (186.6); 2.4973 (134.2); 2.4929 (64.7); 2.3330 (0.8); 2.3287 (1.1); 2.3241 (0.8); 1.9888 (1.0); 1.3359 (0.4); 1.2985 (0.3); 1.2590 (0.4); 1.2496 (0.6); 1.1927 (0.4); .1835 (3.7); 1.1750 (1.0); |
| | | 1.1651 (8.4); 1.1467 (3.6); 0.1459 (0.5); |
| | | 0.0080 (4.5); −0.0001 (128.5); −0.0084 |
| | | (5.1); −0.1498 (0.5) |
| I-16 | 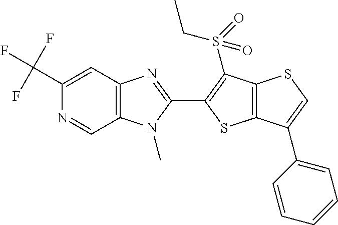 [SMILES: CCS(=O)(=O)c1c(-c2nc3cc(C(F)(F)F)ncc3n2C)sc2c(-c3ccccc3)csc12] | ¹H-NMR (400.0 MHz, d₆-DMSO): δ = 9.2995 (2.6); 8.3256 (2.8); 8.1062 (4.6); 7.8076 (1.4); 7.8043 (2.0); 7.7863 (2.3); 7.5415 (1.0); 7.5234 (2.3); 7.5039 (1.6); 7.4620 (1.0); 7.4437 (1.2); 7.4250 (0.4); 3.9592 (10.6); 3.5979 (0.7); 3.5795 (2.3); 3.5610 (2.3); 3.5426 (0.7); 3.3199 (36.1); 2.6754 (0.5); 2.6709 (0.7); 2.6663 (0.5); 2.5242 (2.0); 2.5193 (3.3); 2.5108 (41.8); 2.5064 (86.0); 2.5019 (113.5); 2.4974 (81.2); 2.4931 (39.4); 2.3332 (0.5); 2.3286 (0.6); 2.3241 (0.5); 1.3977 (16.0); 1.2172 (2.5); 1.1988 (5.6); 1.1804 (2.4); 0.1460 (0.5); 0.0078 (3.9); |
| | | −0.0003 (105.3) −0.0086 (4.1); −0.1497 |
| | | (0.5) |
| I-17 | 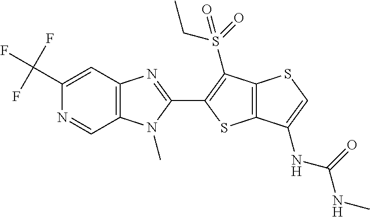 [SMILES: CCS(=O)(=O)c1c(-c2nc3cc(C(F)(F)F)ncc3n2C)sc2c(NC(=O)NC)csc12] | ¹H-NMR (400.0 MHz, d₆-DMSO): δ = 10.3150 (1.0); 9.2518 (1.7); 8.2798 (1.9); 6.9546 (2.0); 6.4475 (0.3); 6.4355 (0.3); 5.7552 (2.1); 3.9189 (7.1); 3.6202 (0.3); 3.6093 (0.4); 3.5275 (0.5); 3.5091 (1.5); 3.4906 (1.5); 3.4722 (0.5); 3.3253 (157.4); 3.1781 (0.4); 3.1517 (0.4); 3.1402 (0.4); 3.1328 (0.4); 3.1217 (0.4); 2.7050 (2.8); 2.6935 (2.8); 2.6755 (0.8); 2.6711 (1.1); 2.6665 (0.8); 2.5243 (3.1); 2.5108 (65.9); 2.5065 (137.1); 2.5020 (183.6); 2.4975 (132.4); 2.4931 (64.2); 2.3333 (0.7); 2.3287 (1.0); 2.3241 (0.8); |
| | | 1.6804 (0.4); 1.6732 (0.4); 1.6629 (0.4); |
| | | 1.5511 (1.2); 1.5363 (1.2); 1.5215 (0.7); |
| | | 1.3371 (16.0); 1.2828 (2.9); 1.2647 (5.2); |
| | | 1.2522 (2.6); 1.2475 (3.5); 1.2344 (1.4); |
| | | 1.1800 (1.8); 1.1616 (3.8); 1.1432 (1.7); |
| | | 0.1458 (0.3); 0.0080 (2.8); −0.0002 |
| | | (81.4) −0.0083 (3.4); −0.1496 (0.4) |
| I-18 | 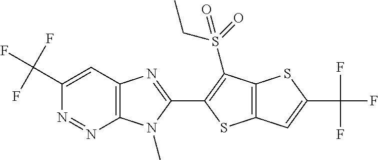 [SMILES: CCS(=O)(=O)c1c(-c2nc3cc(C(F)(F)F)nnc3n2C)sc2cc(C(F)(F)F)sc12] | ¹H-NMR (400.0 MHz, d₆-DMSO): δ = 8.8098 (6.2); 8.3860 (3.0); 8.3841 (3.0); 5.7554 (2.6); 5.7547 (2.5); 4.3521 (0.7); 4.0124 (16.0); 3.6352 (1.0); 3.6168 (3.5); 3.5983 (3.6); 3.5799 (1.1); 3.3489 (4.3); 2.5256 (0.4); 2.5121 (10.6); 2.5080 (21.9); 2.5036 (29.3); 2.4992 (21.2); 2.4950 (10.3); 1.3965 (0.4); 1.3172 (0.4); 1.2084 (3.7); 1.1900 (8.2); 1.1716 (3.7); 0.0071 (0.5); −0.00002 |
| | | (15.8); −0.0083 (0.6) |
| I-19 | 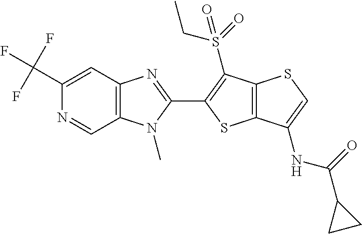 [SMILES: CCS(=O)(=O)c1c(-c2nc3cc(C(F)(F)F)ncc3n2C)sc2c(NC(=O)C3CC3)csc12] | ¹H-NMR (400.0 MHz, d₆-DMSO): δ = 11.9715 (0.8); 10.2040 (0.5); 9.2640 (3.8); 8.2920 (4.0); 7.3838 (0.4); 7.2120 (7.2); 3.9247 (16.0); 3.8247 (0.4); 3.5683 (0.4); 3.5325 (1.0); 3.5233 (0.4); 3.5142 (3.4); 3.4957 (3.4); 3.4772 (1.1); 3.3334 (313.1); 2.6764 (0.7); 2.6720 (1.0); 2.6674 (0.7); 2.5253 (2.6); 2.5205 (4.2); 2.5119 (54.6); 2.5075 (114.8); 2.5029 (154.1); 2.4984 (110.3); 2.4939 (52.8); 2.3342 (0.7); 2.3297 (0.9); 2.3251 (0.6); 2.0739 (0.6); 1.8601 (0.5); 1.8551 (0.6); 1.8424 (1.0); 1.8293 (0.6); 1.8241 (0.6); 1.7555 (0.7); 1.6488 (0.3); 1.1732 (3.8); |
| | | 1.1548 (8.6); 1.1363 (3.7); 0.9367 (2.5); |
| | | 0.9179 (4.8); 0.9078 (3.2); 0.0080 (2.6); |
| | | −0.0002 (78.4); −0.0084 (3.0) |
| I-20 | 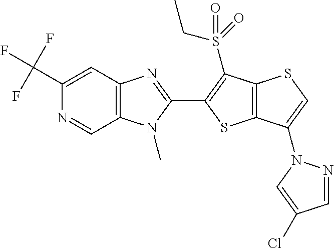 [SMILES: CCS(=O)(=O)c1c(-c2nc3cc(C(F)(F)F)ncc3n2C)sc2c(-n3cc(Cl)cn3)csc12] | ¹H-NMR (400.0 MHz, d₆-DMSO): δ = 9.2965 (3.9); 8.9462 (5.8); 8.3234 (4.2); 8.3150 (0.4); 8.0008 (5.8); 7.9242 (7.2); 5.7553 (0.9); 3.9572 (16.0); 3.6134 (0.9); 3.5951 (3.4); 3.5766 (3.5); 3.5581 (1.0); 3.3225 (141.4); 2.6757 (0.6); 2.6709 (0.9); 2.6665 (0.7); 2.5242 (2.0); 2.5194 (3.3); 2.5108 (55.4); 2.5064 (118.0); 2.5019 (160.2); 2.4974 (115.4); 2.4930 (55.8); 2.3332 (0.6); 2.3286 (0.9); 2.3241 (0.7); 1.2064 (3.6); 1.1880 (8.2); 1.1695 (3.6); 0.1460 (0.4); 0.0078 (3.4); −0.0003 (102.1); −0.0086 (4.1); −0.1497 (0.4) |
| I-21 | 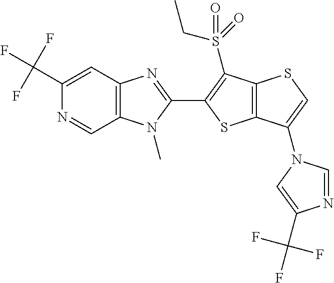 [SMILES: CCS(=O)(=O)c1c(-c2nc3cc(C(F)(F)F)ncc3n2C)sc2c(-n3cnc(C(F)(F)F)c3)csc12] | ¹H-NMR (400.0 MHz, d₆-DMSO): δ = 9.3095 (4.4); 8.5568 (3.2); 8.5211 (3.8); 8.3357 (4.9); 8.3126 (0.4); 8.0353 (6.8); 3.9570 (16.0); 3.6198 (1.2); 3.6024 (3.6); 3.5837 (3.8); 3.5650 (1.2); 3.3832 (0.5); 3.3218 (254.9); 2.6689 (1.5); 2.5321 (0.8); 2.5046 (189.0); 2.5006 (244.7); 2.4968 (180.9); 2.4494 (0.4); 2.3273 (1.3); 2.0726 (0.4); 1.2144 (3.8); 1.1962 (8.2); 1.1779 (3.8); 0.1441 (0.6); 0.0061 (4.3); −0.0014 (113.1); −0.1518 (0.6) |
| I-22 | 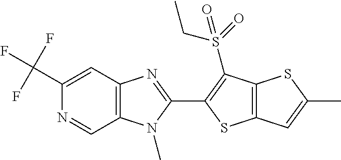 [SMILES: CCS(=O)(=O)c1c(-c2nc3cc(C(F)(F)F)ncc3n2C)sc2cc(C)sc12] | ¹H-NMR (400.0 MHz, d₆-DMSO): δ = 9.2780 (3.7); 8.3141 (0.4); 8.3027 (3.9); 7.4142 (3.1); 7.4113 (3.0); 3.9200 (16.0); 3.5258 (1.0); 3.5073 (3.5); 3.4889 (3.6); 3.4704 (1.1); 3.3185 (76.2); 2.6750 (0.9); 2.6706 (1.2); 2.6660 (0.9); 2.6613 (0.5); 2.6402 (9.4); 2.6380 (9.2); 2.5238 (3.5); 2.5104 (70.1); 2.5060 (139.8); 2.5016 (181.0); 2.4970 (129.6); 2.4926 (62.8); 2.3373 (0.4); 2.3327 (0.8); 2.3282 (1.1); 2.3238 (0.8); 1.3978 (13.8); 1.1709 (3.7); 1.1525 (8.3); 1.1340 (3.6); 0.1459 (0.7); 0.0076 (5.6); −0.0002 (147.1); |
| | | −0.0083 (6.3); −0.1498 (0.7) |
| I-23 | 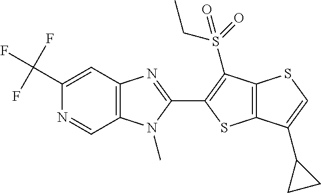 [SMILES: CCS(=O)(=O)c1c(-c2nc3cc(C(F)(F)F)ncc3n2C)sc2c(C3CC3)csc12] | ¹H-NMR (400.0 MHz, d₆-DMSO): δ = 9.2738 (3.7); 8.3004 (4.0); 7.4136 (4.5); 7.4122 (4.9); 3.9306 (0.4); 3.9132 (16.0); 3.5245 (1.0); 3.5061 (3.5); 3.4877 (3.5); 3.4693 (1.1); 3.3199 (67.2); 2.6751 (0.6); 2.6706 (0.9); 2.6661 (0.6); 2.5239 (2.5); 2.5191 (4.1); 2.5105 (57.4); 2.5061 (120.2); 2.5016 (161.1); 2.4971 (117.2); 2.4927 (58.3); 2.3681 (0.4); 2.3556 (0.6); 2.3471 (0.7); 2.3343 (1.6); 2.3282 (1.4); 2.3237 (1.4); 2.3149 (0.7); 2.3018 (0.4); 1.3976 (2.2); 1.3356 (0.4); 1.2981 (0.4); |
| | | 1.2586 (0.6); 1.2494 (0.7); 1.2350 (1.1); |
| | | 1.1674 (4.0); 1.1490 (10.4); 1.1375 (1.6); |
| | | 1.1307 (4.7); 1.1170 (1.0); 0.8655 (1.0); |
| | | 0.8546 (2.7); 0.8494 (2.5); 0.8425 (2.3); |
| | | 0.8371 (2.8); 0.8257 (0.8); 0.1459 (0.7); |
| | | 0.0079 (5.8); −0.0002 (158.2); −0.0084 |
| | | (6.5); −0.1497 (0.7) |
| I-24 | 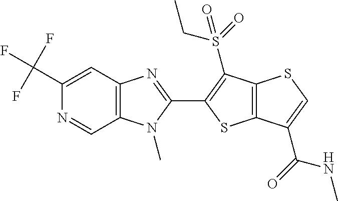 [SMILES: CCS(=O)(=O)c1c(-c2nc3cc(C(F)(F)F)ncc3n2C)sc2c(C(=O)NC)csc12] | ¹H-NMR (400.0 MHz, d₆-DMSO): δ = 9.3012 (3.9); 8.8724 (0.4); 8.8625 (1.2); 8.8509 (1.2); 8.8418 (0.4) 8.3272 (4.1); 8.2057 (6.8); 5.7556 (2.5); 4.0387 (0.8); 4.0209 (0.8); 3.9439 (16.0); 3.5717 (1.0); 3.5532 (3.4); 3.5347 (3.5); 3.5164 (1.0); 3.3257 (42.6); 2.8394 (6.3); 2.8280 (6.3); 2.5255 (0.7); 2.5121 (15.3); 2.5077 (32.0); 2.5032 (43.2); 2.4987 (31.3); 2.4943 (15.3); 1.9895 (3.5); 1.3367 (0.5); 1.2592 (0.5); 1.2500 (0.7); 1.2354 (0.5); 1.1935 (1.2); 1.1865 (3.7); 1.1757 (2.4); |
| | | 1.1681 (8.4); 1.1580 (1.4); 1.1496 (3.6); |
| | | 0.0079 (2.1); −0.0002 (55.2); −0.0084 |
| | | (2.5) |
| I-25 | 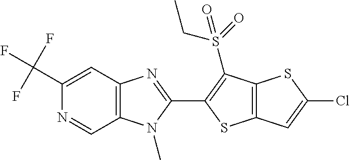 [SMILES: CCS(=O)(=O)c1c(-c2nc3cc(C(F)(F)F)ncc3n2C)sc2cc(Cl)sc12] | ¹H-NMR (400.0 MHz, d₆-DMSO): δ = 9.2952 (3.7); 8.3200 (3.9); 7.8315 (7.7); 5.7553 (3.7); 3.9373 (16.0); 3.5890 (1.0); 3.5706 (3.4); 3.5521 (3.5); 3.5337 (1.0); 3.3215 (9.3); 2.5256 (0.6); 2.5208 (0.9); 2.5121 (12.5); 2.5077 (25.8); 2.5032 (34.0); 2.4987 (24.3); 2.4942 (11.7); 1.9896 (0.6); 1.1856 (3.7); 1.1761 (0.7); 1.1673 (8.3); 1.1585 (0.6); 1.1488 (3.6); 0.0079 (1.2); −0.0002 (35.0); −0.0085 |
| | | (1.2) |
| I-26 | 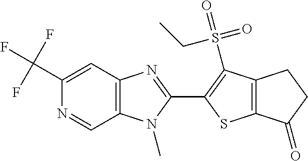 [SMILES: CCS(=O)(=O)c1c(-c2nc3cc(C(F)(F)F)ncc3n2C)sc2c1CCC2=O] | ¹H-NMR (600.1 MHz, CD3CN): δ = 9.1098 (2.8); 8.1952 (2.9); 8.1941 (2.9); 3.9006 (16.0); 3.3766 (1.2); 3.3642 (3.7); 3.3518 (3.8); 3.3395 (1.2); 3.3267 (2.2); 3.3235 (1.2); 3.3218 (1.3); 3.3185 (2.2); 3.3149 (1.3); 3.3132 (1.2); 3.3102 (2.4); 3.0380 (2.5); 3.0349 (1.2); 3.0334 (1.4); 3.0297 (2.2); 3.0263 (1.4); 3.0249 (1.1); 3.0214 (2.2); 2.5335 (0.7); 2.1834 (2.4); 1.9805 (2.1); 1.9764 (4.0); 1.9722 |
| | | (5.6); 1.9681 (3.8); 1.9640 (1.9); 1.2790 |
| | | (3.8); 1.2667 (7.9); 1.2543 (3.7) |
| I-27 | 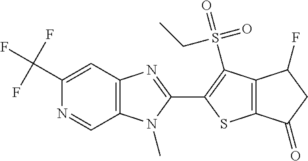 [SMILES: CCS(=O)(=O)c1c(-c2nc3cc(C(F)(F)F)ncc3n2C)sc2c1C(F)CC2=O] | ¹H-NMR (601.6 MHz, CD3CN): δ = 9.1213 (2.8); 8.2075 (3.0); 6.4363 (0.7); 6.4342 (0.7); 6.4260 (0.7); 6.4240 (0.7); 6.3483 (0.7); 6.3463 (0.7); 6.3381 (0.7); 6.3361 (0.7); 3.9045 (16.0); 3.5439 (0.6); 3.5337 (0.6); 3.5219 (0.6); 3.5121 (1.1); 3.5021 (0.7); 3.4904 (0.7); 3.4801 (0.7); 3.4267 (0.7); 3.4194 (0.5); 3.4145 (1.7); 3.4071 (1.6); 3.4022 (1.7); 3.3948 (1.7); 3.3898 (0.6); 3.3827 (0.7); 3.2118 |
| | | (0.8); 3.2097 (0.8); 3.1802 (0.6); 3.1782 |
| | | (0.7); 3.1741 (0.8); 3.1721 (0.8); 3.1426 |
| | | (0.6); 3.1406 (0.6); 2.5308 (6.5); 2.1568 |
| | | (13.5); 1.9932 (0.6); 1.9851 (0.6); 1.9808 |
| | | (0.9); 1.9771 (12.8); 1.9730 (25.0); |
| | | 1.9689 (37.0); 1.9649 (25.2); 1.9607 |
| | | (12.7); 1.2825 (3.2); 1.2701 (6.6); 1.2578 |
| | | (3.2) |
| I-28 | 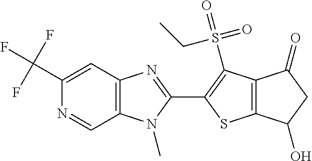 [SMILES: CCS(=O)(=O)c1c(-c2nc3cc(C(F)(F)F)ncc3n2C)sc2c1C(=O)CC2O] | ¹H-NMR (601.6 MHz, CD3CN): δ = 9.0821 (1.9); 8.1842 (2.0); 8.1830 (2.0); 5.5609 (0.3); 5.5541 (0.6); 5.5504 (0.6); 5.5436 (0.3); 4.2584 (0.8); 4.2478 (0.8); 3.8910 (12.1); 3.5839 (0.4); 3.5720 (1.2); 3.5598 (1.6); 3.5478 (1.2); 3.5358 (0.4); 3.4524 (0.8); 3.4417 (0.8); 3.4218 (0.9); 3.4111 (0.9); 2.9434 (0.9); 2.9395 (0.9); 2.9128 (0.8); 2.9089 (0.8); 2.5317 (16.0); 2.1631 (33.4); 1.9929 (0.6); |
| | | 1.9847 (0.6); 1.9805 (0.9); 1.9768 (16.6); |
| | | 1.9727 (32.1); 1.9686 (48.4); 1.9645 |
| | | (33.2); 1.9604 (17.0); 1.2266 (2.7); |
| | | 1.2142 (5.9); 1.2019 (2.7) |
| I-29 | 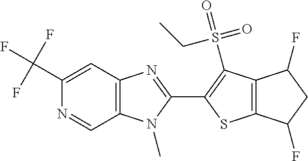 [SMILES: CCS(=O)(=O)c1c(-c2nc3cc(C(F)(F)F)ncc3n2C)sc2c1C(F)CC2F] | ¹H-NMR (601.6 MHz, CD3CN): δ = 9.0965 (1.9); 8.1888 (2.0); 6.1025 (0.7); 6.0920 (0.7); 6.0109 (0.5); 6.0091 (0.5); 6.0009 (0.5); 3.8899 (0.4); 3.8796 (12.1); 3.4062 (0.4); 3.3956 (0.4); 3.3685 (0.5); 3.3589 (0.9); 3.3469 (2.4); 3.3345 (2.4); 3.3221 (0.8); 2.7776 (0.4); 2.7505 (0.4); 2.5312 (16.0); 2.1616 (38.1); 1.9930 (0.6); 1.9849 (0.6); 1.9806 (1.0); 1.9769 (15.3); 1.9729 (28.8); 1.9687 |
| | | (41.7); 1.9646 (29.0); 1.9605 (14.9); |
| | | 1.2478 (2.5); 1.2355 (5.1); 1.2231 (2.5) |
| I-30 | 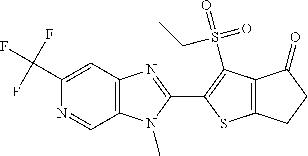 [SMILES: CCS(=O)(=O)c1c(-c2nc3cc(C(F)(F)F)ncc3n2C)sc2c1C(=O)CC2] | ¹H-NMR (601.6 MHz, CD3CN): δ = 9.0828 (0.7); 9.0708 (2.4); 8.1740 (3.3); 3.8843 (16.0); 3.8731 (4.5); 3.5944 (0.8); 3.5820 (2.3); 3.5697 (2.4); 3.5573 (0.8); 3.3538 (2.1); 3.3492 (1.0); 3.3454 (2.1); 3.3412 (1.1); 3.3367 (2.3); 3.3332 (0.7); 3.3319 (0.7); 3.3205 (0.7); 3.0843 (2.2); 3.0801 (1.2); 3.0758 (1.9); 3.0719 (1.1); 3.0673 (2.0); 2.5309 (7.4); 2.1646 (7.8); 1.9808 (0.3); 1.9770 (5.6); |
| | | 1.9729 (10.7); 1.9688 (15.4); 1.9647 |
| | | (10.3); 1.9606 (5.4); 1.2425 (0.9); 1.2302 |
| | | (1.8); 1.2226 (3.7); 1.2179 (1.0); 1.2102 |
| | | (7.8); 1.1979 (3.6) |
| I-31 | 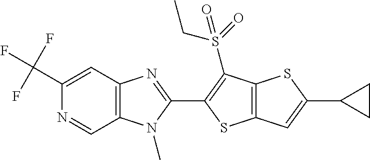 [SMILES: CCS(=O)(=O)c1c(-c2nc3cc(C(F)(F)F)ncc3n2C)sc2cc(C3CC3)sc12] | ¹H-NMR (400.2 MHz, d₆-DMSO): δ = 9.2749 (3.7); 8.3005 (3.9); 7.4135 (4.6); 7.4121 (4.8); 7.2811 (0.4); 7.2794 (0.4); 5.7559 (1.3); 3.9154 (16.0); 3.5262 (1.0); 3.5078 (3.5); 3.4893 (3.6); 3.4709 (1.0); 3.3642 (0.3); 3.3231 (21.0); 2.5253 (1.0); 2.5204 (1.6); 2.5118 (16.9); 2.5074 (33.0); 2.5028 (43.4); 2.4982 (32.5); 2.4938 (16.4); 2.3559 (0.6); 2.3475 |
| | | (0.6); 2.3351 (1.3); 2.3290 (0.6); |
| | | 2.3242 (0.8); 2.3147 (0.6); 1.1692 |
| | | (3.9); 1.1507 (9.6); 1.1381 (1.7); |
| | | 1.1327 (5.2); 1.1283 (3.1); 1.1176 (1.0); |
| | | 0.8664 (0.9); 0.8555 (2.5); 0.8503 (2.4); |
| | | 0.8433 (2.3); 0.8380 (2.8); 0.8267 (0.8); |
| | | 0.0080 (0.3); −0.0002 (9.0); −0.0085 |
| | | (0.3) |
| I-32 | 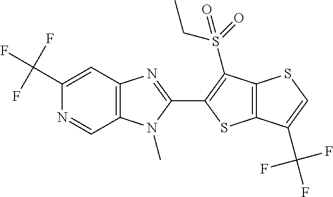 [SMILES: CCS(=O)(=O)c1c(-c2nc3cc(C(F)(F)F)ncc3n2C)sc2c(C(F)(F)F)csc12] | :¹H-NMR (400.2 MHz, d₆-DMSO): δ = 9.3182 (3.5); 8.8420 (2.9); 8.8390 (2.8); 8.3454 (3.8); 8.3436 (3.7); 7.2639 (0.7); 7.2457 (0.7); 7.1889 (0.9); 7.1719 (0.7); 7.1558 (0.4); 4.3404 (0.3); 4.3274 (0.7); 4.3145 (0.4); 3.9585 (16.0); 3.6241 (1.0); 3.6056 (3.4); 3.5871 (3.4); 3.5688 (1.0); 3.3919 (0.4); 3.3759 (0.8); 3.3630 (0.8); 3.3600 (0.5); 3.3466 (0.5); 3.3221 (50.1); 2.6758 (0.4); 2.6712 (0.6); 2.6665 (0.5); 2.5797 (0.5); 2.5607 (0.8); 2.5411 (0.7); 2.5247 |
| | | (2.3); 2.5198 (3.6); 2.5112 (38.8); |
| | | 2.5068 (75.8); 2.5022 (99.3); 2.4976 |
| | | (73.8); 2.4931 (37.1); 2.3337 (0.5); |
| | | 2.3291 (0.6); 2.3246 (0.5); 1.9890 (0.4); |
| | | 1.5783 (0.3); 1.5590 (0.5); 1.5396 (0.4); |
| | | 1.4522 (0.4); 1.4361 (0.4); 1.4160 (0.5); |
| | | 1.3977 (4.8); 1.3127 (0.4); 1.2953 (0.5); |
| | | 1.2046 (3.6); 1.1862 (8.3); 1.1754 (0.7); |
| | | 1.1677 (3.5); 0.0080 (0.7); -0.0002 |
| | | (19.9); −0.0084 (0.7) |
| I-33 | 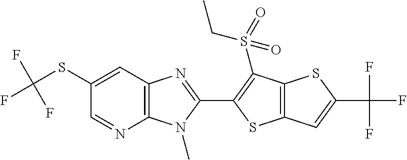 [SMILES: CCS(=O)(=O)c1c(-c2nc3cc(SC(F)(F)F)cnc3n2C)sc2cc(C(F)(F)F)sc12] | 1H-NMR (400.2 MHz, d6-DMSO): δ = 8.7948 (2.6); 8.7899 (2.9); 8.7097 (2.6); 8.7049 (2.2); 8.3724 (1.1); 8.3694 (2.8); 8.3664 (2.7); 3.8180 (16.0); 3.6492 (0.9); 3.6307 (3.1); 3.6123 (3.1); 3.5938 (0.9); 3.3237 (75.8); 2.6757 (0.5); 2.6711 (0.7); 2.6665 (0.5); 2.5247 (2.2); 2.5199 (3.2); 2.5112 (40.8); 2.5067 (82.7); 2.5022 (107.4); 2.4976 (75.6); 2.4930 (35.3); 2.3335 (0.5); 2.3289 (0.7); 2.3244 |
| | | (0.5); 1.2046 (3.4); 1.1862 (8.0); 1.1678 |
| | | (3.3); 0.0079 (1.2); −0.0002 (36.9); |
| | | −0.0086 (1.1) |
| I-34 | 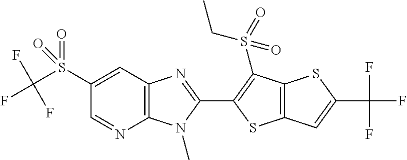 [SMILES: CCS(=O)(=O)c1c(-c2nc3cc(S(=O)(=O)C(F)(F)F)cnc3n2C)sc2cc(C(F)(F)F)sc12] | I1H-NMR (400.2 MHz, d6-DMSO): δ = 9.1931 (2.7); 9.1879 (3.0); 9.0993 (2.5); 9.0943 (2.3); 8.3772 (2.8); 8.3743 (2.8); 3.8724 (16.0); 3.6468 (0.9); 3.6283 (3.2); 3.6099 (3.3); 3.5915 (1.0); 3.3225 (30.4); 2.6716 (0.4); 2.5251 (1.2); 2.5202 (1.9); 2.5114 (25.5); 2.5071 (51.9); 2.5026 (68.1); 2.4980 (49.0); 2.4936 (23.7); 2.3293 (0.4); 2.0750 (1.4); 1.2090 (3.6); 1.1907 (8.3); 1.1722 (3.5); 0.0080 |
| | | (0.6); −0.0002 (19.6); −0.0085 (0.6) |
| I-35 | 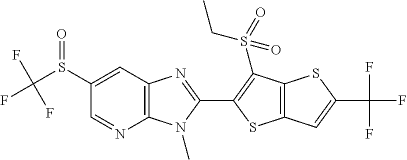 [SMILES: CCS(=O)(=O)c1c(-c2nc3cc(S(=O)C(F)(F)F)cnc3n2C)sc2cc(C(F)(F)F)sc12] | 1H-NMR (400.2 MHz, d6-DMSO): δ = 8.9823 (2.3); 8.9777 (2.4); 8.7830 (2.2); 8.7786 (2.1); 8.3775 (1.1); 8.3746 (2.7); 8.3715 (2.7); 3.8515 (16.0); 3.6583 (0.8); 3.6398 (2.9); 3.6213 (3.0); 3.6028 (0.9); 3.3231 (33.9); 2.6714 (0.4); 2.5249 (1.2); 2.5201 (1.9); 2.5115 (24.8); 2.5070 (51.0); 2.5025 (67.0); 2.4978 (47.6); 2.4933 (22.6); 2.3293 (0.4); 2.0747 (2.7); 1.2108 (3.5); 1.1924 (8.1); 1.1739 (3.4); |
| | | 0.0079 (0.7); −0.0002 (22.8); −0.0086 |
| | | (0.7) |
| I-36 | 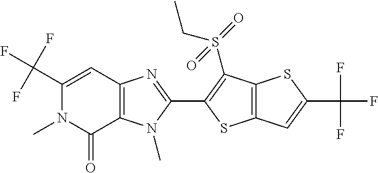 [SMILES: CCS(=O)(=O)c1c(-c2nc3cc(C(F)(F)F)n(C)c(=O)c3n2C)sc2cc(C(F)(F)F)sc12] | I1H-NMR (400.2 MHz, d6-DMSO): δ = 8.3549 (1.2); 8.3519 (3.1); 8.3489 (2.9); 8.3144 (0.4); 7.4718 (4.2); 3.9856 (16.0); 3.62991 (6.8); 3.5531 (1.0); 3.5347 (3.4); 3.5163 (3.4); 3.4979 (1.0); 3.3207 (28.8); 2.6758 (0.5); 2.6712 (0.7); 2.6667 (0.5); 2.5247 (2.3); 2.5199 (3.5); 2.5113 (42.7); 2.5068 (85.8); 2.5023 (111.2); 2.4977 (78.1); 2.4932 (36.6); 2.3335 (0.5); 2.3292 (0.7); 2.3245 (0.5); 2.0742 (1.0); 1.1746 (3.5); 1.1563 (8.0); |
| | | 1.1378 (3.4); −0.0002 (1.4) |
| I-37 | 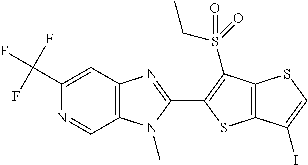 [SMILES: CCS(=O)(=O)c1c(-c2nc3cc(C(F)(F)F)ncc3n2C)sc2c(I)csc12] | 1H-NMR (400.0 MHz, d6-DMSO): δ = 9.2868 (3.5); 8.3145 (4.2); 7.9659 (8.1); 5.7553 (1.2); 3.9243 (16.0); 3.5609 (1.0); 3.5424 (3.4); 3.5240 (3.4); 3.5055 (1.0); 3.3227 (267.8); 3.2995 (0.4); 2.6797 (0.7); 2.6751 (1.5); 2.6706 (2.0); 2.6660 (1.5); 2.6617 (0.7); 2.5241 (6.2); 2.5194 (9.5); 2.5107 (121.5); 2.5062 (247.6); 2.5017 (325.2); 2.4971 |
| | | (231.4); 2.4925 (110.2); 2.3371 (0.6); |
| | | 2.3330 (1.4); 2.3285 (2.0); 2.3239 |
| | | (1.4); 2.3195 (0.6); 1.3508 (0.6); |
| | | 1.2587 (0.3); 1.2347 (1.0); 1.1704 |
| | | (3.6); 1.1520 (8.2); 1.1335 (3.4); |
| | | 0.0081 (0.4); −0.0001 (13.2); −0.0085 |
| | | (0.4) |

### USE EXAMPLES

Ctenocephalides felis—In Vitro Contact Tests with Adult Cat Fleas

For the coating of the test tubes, 9 mg of active compound are first dissolved in 1 ml of acetone p.a. and then diluted to the desired concentration with acetone p.a. 250 μl of the solution are distributed homogeneously on the inner walls and the base of a 25 ml glass tube by turning and rocking on an orbital shaker (rocking rotation at 30 rpm for 2 h). With 900 ppm of active compound solution and internal surface area 44.7 cm², given homogeneous distribution, an area-based dose of 5 μg/cm² is achieved.

After the solvent has evaporated off, the tubes are populated with 5-10 adult cat fleas (Ctenocephalides felis), sealed with a perforated plastic lid and incubated in a horizontal position at room temperature and ambient humidity. After 48 h, efficacy is determined. To this end, the tubes are stood upright and the fleas are knocked to the base of the tube. Fleas which remain motionless at the base or move in an uncoordinated manner are considered to be dead or moribund.

A substance shows good efficacy against Ctenocephalides felis if at least 80% efficacy was achieved in this test at an application rate of 5 μg/cm². 100% efficacy means that all the fleas were dead or moribund. 0% efficacy means that no fleas were harmed.

In this test, for example, the following compounds from the preparation examples show an efficacy of 100% at an application rate of 5 μg/cm² (=500 g of ai/ha): I-10

Ctenocephalides felis—Oral Test

### Solvent: Dimethyl Sulfoxide

To produce a suitable active compound formulation, 10 mg of active compound are mixed with 0.5 ml of dimethyl sulfoxide. Dilution with citrated cattle blood gives the desired concentration.

About 20 unfed adult cat fleas (Ctenocephalides felis) are placed into a chamber which is closed at the top and bottom with gauze. A metal cylinder whose bottom end is closed with parafilm is placed onto the chamber. The cylinder contains the blood/active compound formulation, which can be imbibed by the fleas through the parafilm membrane.

After 2 days, the kill in % is determined. 100% means that all of the fleas have been killed; 0% means that none of the fleas have been killed.

In this test, for example, the following compounds from the preparation examples show an efficacy of 100% at an application rate of 100 ppm: I-18

In this test, for example, the following compounds from the preparation examples show an efficacy of 95% at an application rate of 100 ppm: I-7

In this test, for example, the following compounds from the preparation examples show an efficacy of 80% at an application rate of 100 ppm: I-10, I-12

Lucilia cuprina Test

### Solvent: Dimethyl Sulfoxide

To produce a suitable active compound formulation, 10 mg of active compound are mixed with 0.5 ml of dimethyl sulfoxide, and the concentrate is diluted with water to the desired concentration.

About 20 L1 larvae of the Australian sheep blowfly (Lucilia cuprina) are transferred into a test vessel containing minced horsemeat and the active compound preparation of the desired concentration.

After 2 days, the kill in % is determined. 100% means that all the larvae have been killed; 0% means that no larvae have been killed.

In this test, for example, the following compounds from the preparation examples show an efficacy of 100% at an application rate of 100 ppm: I-1, I-2, I-10

In this test, for example, the following compounds from the preparation examples show an efficacy of 90% at an application rate of 100 ppm: I-4

Musca domestica Test

### Solvent: Dimethyl Sulfoxide

To produce a suitable active compound formulation, 10 mg of active compound are mixed with 0.5 ml of dimethyl sulfoxide, and the concentrate is diluted with water to the desired concentration.

Vessels containing a sponge treated with sugar solution and the desired concentration of active compound formulation are populated with 10 adult houseflies (Musca domestica).

After 2 days, the kill in % is determined. 100% means that all of the flies have been killed; 0% means that none of the flies have been killed.

In this test, for example, the following compounds from the preparation examples show an efficacy of 85% at an application rate of 100 ppm: I-12

In this test, for example, the following compounds from the preparation examples show an efficacy of 80% at an application rate of 100 ppm: I-7, I-10, I-18

Myzus persicae—Oral Test

| | |
|---|---|
| Solvent: | 100 parts by weight of acetone |

To produce a suitable active compound formulation, 1 part by weight of active compound is dissolved with the stated parts by weight of solvent and made up to the desired concentration with water.

50 μl of the active compound formulation are transferred into microtitre plates and made up to a final volume of 200 μl with 150 μl of IPL41 insect medium (33%+15% sugar). Subsequently, the plates are sealed with parafilm, which a mixed population of green peach aphids (Myzus persicae) within a second microtitre plate is able to puncture and imbibe the solution.

After 5 days, the efficacy in % is determined. 100% means that all the aphids have been killed; 0% means that no aphids have been killed.

In this test, for example, the following compounds from the preparation examples show an efficacy of 100% at an application rate of 0.8 ppm: I-19

In this test, for example, the following compounds from the preparation examples show an efficacy of 100% at an application rate of 4 ppm: I-1, I-2, I-3, I-4, I-5, I-8, I-9, I-12, I-15, I-16, I-18, I-20, I-21, I-22, I-23, I-24, II-25

In this test, for example, the following compounds from the preparation examples show an efficacy of 90% at an application rate of 4 ppm: I-10, I-11, I-13, I-14

Myzus persicae—Spray Test

| | |
|---|---|
| Solvent: | 78 parts by weight of acetone |
| | 1.5 parts by weight of dimethylformamide |
| Emulsifier: | alkylaryl polyglycol ether |

To produce a suitable active compound formulation, 1 part by weight of active compound is dissolved with the specified parts by weight of solvent and made up to the desired concentration with water containing an emulsifier concentration of 1000 ppm. To produce further test concentrations, the formulation is diluted with emulsifier-containing water.

Discs of Chinese cabbage leaves (Brassica pekinensis) infested by all stages of the green peach aphid (Myzus persicae) are sprayed with an active compound formulation of the desired concentration.

After 5 days, the efficacy in % is determined. 100% means that all the aphids have been killed; 0% means that no aphids have been killed.

In this test, for example, the following compounds from the preparation examples show an efficacy of 100% at an application rate of 100 g/ha: I-7, I-12, I-18

In this test, for example, the following compounds from the preparation examples show an efficacy of 90% at an application rate of 100 g/ha: I-6, I-11, I-14, I-15, I-16, I-21, I-22, I-24, II-25

Phaedon cochleariae—Spray Test

| | |
|---|---|
| Solvent: | 78.0 parts by weight of acetone |
| | 1.5 parts by weight of dimethylformamide |
| Emulsifier: | alkylaryl polyglycol ether |

To produce a suitable active compound formulation, 1 part by weight of active compound is dissolved with the specified parts by weight of solvent and made up to the desired concentration with water containing an emulsifier concentration of 1000 ppm. To produce further test concentrations, the formulation is diluted with emulsifier-containing water.

Discs of Chinese cabbage leaves (Brassica pekinensis) are sprayed with an active compound formulation of the desired concentration and, after drying, populated with larvae of the mustard beetle (Phaedon cochleariae).

After 7 days, the efficacy in % is determined. 100% means that all the beetle larvae have been killed; 0% means that no beetle larvae have been killed.

In this test, for example, the following compounds from the preparation examples show an efficacy of 100% at an application rate of 100 g/ha: I-3, I-7, II-9, II-10, I-12, I-13, I-14, I-18, I-20, II-21

In this test, for example, the following compounds from the preparation examples show an efficacy of 83% at an application rate of 100 g/ha: I-11

Spodoptera frugiperda—Spray Test

| | |
|---|---|
| Solvent: | 78.0 parts by weight of acetone |
| | 1.5 parts by weight of dimethylformamide |
| Emulsifier: | alkylaryl polyglycol ether |

To produce a suitable active compound formulation, 1 part by weight of active compound is dissolved with the specified parts by weight of solvent and made up to the desired concentration with water containing an emulsifier concentration of 1000 ppm. To produce further test concentrations, the formulation is diluted with emulsifier-containing water.

Leaf discs of maize (Zea mays) are sprayed with an active compound formulation of the desired concentration and, after drying, populated with caterpillars of the fall armyworm (Spodoptera frugiperda).

After 7 days, the efficacy in % is determined. 100% means that all the caterpillars have been killed; 0% means that no caterpillar has been killed.

In this test, for example, the following compounds from the preparation examples show an efficacy of 100% at an application rate of 100 g/ha: I-3, I-7, I-8, I-9, I-10, I-12, I-13, I-14, I-16, I-18, I-20, I-21, I-23, II-25

Tetranychus urticae—Spray Test, OP-Resistant

| | |
|---|---|
| Solvent: | 78.0 parts by weight of acetone |
| | 1.5 parts by weight of dimethylformamide |
| Emulsifier: | alkylaryl polyglycol ether |

To produce a suitable active compound formulation, 1 part by weight of active compound is dissolved with the specified parts by weight of solvent and made up to the desired concentration with water containing an emulsifier concentration of 1000 ppm. To produce further test concentrations, the formulation is diluted with emulsifier-containing water.

Discs of bean leaves (Phaseolus vulgaris) infested with all stages of the greenhouse red spider mite (Tetranychus urticae) are sprayed with an active compound formulation of the desired concentration.

After 6 days, the efficacy in % is determined. 100% means that all the spider mites have been killed; 0% means that no spider mites have been killed.

In this test, for example, the following compounds from the preparation examples shows an efficacy of 90% at an application rate of 100 g/ha: I-7, I-12, I-15, I-30

1. Compound of formula (I) 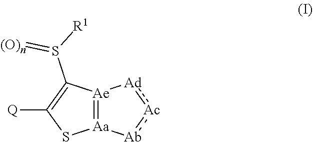
where [symbol] represents two single bonds or one double bond and one single bond,
in which,
if [symbol] represents a double bond and a single bond, and Aa to Ae thus form an aromatic ring,
Aa represents nitrogen or carbon,
Ab represents nitrogen, oxygen, sulfur, N(R⁸) or C(R¹¹),
Ac represents nitrogen, oxygen, sulfur or C(R¹²),
Ad represents nitrogen, oxygen, sulfur, N(R⁸) or C(R¹³),
Ae represents nitrogen or carbon,
where R⁸ represents (C₁-C₄)-alkyl, cyclopropyl or hydrogen and
where at most three of the groups Aa, Ab, Ac, Ad, Ae may represent nitrogen and at most one of the groups Ab, Ac, Ad may represent oxygen or at most one of the groups Ab, Ac, Ad may represent sulfur or at most one of the groups Ab, Ad may represent N(R⁸),
or, in the case that [symbol] represent exclusively single bonds,
Aa represents carbon,
Ab represents sulfur, oxygen or C(R¹¹)(R¹⁵),
Ac represents sulfur, oxygen or C(R¹²)(R¹⁶) and
Ad represents sulfur, oxygen or C(R¹³)(R¹⁷),
Ae represents carbon,
where at most one of the groups Aa, Ab, Ac, Ad, Ae may represent oxygen or sulfur,
R¹ represents (C₁-C₆)-alkyl, (C₁-C₆)-haloalkyl, (C₁-C₆)-cyanoalkyl, (C₁-C₆)-hydroxyalkyl, (C₁-C₆)-alkoxy-(C₁-C₆)-alkyl, (C₁-C₆)-haloalkoxy-(C₁-C₆)-alkyl, (C₂-C₆)-alkenyl, (C₂-C₆)-alkenyloxy-(C₁-C₆)-alkyl, (C₂-C₆)-haloalkenyloxy-(C₁-C₆)-alkyl, (C₂-C₆)-haloalkenyl, (C₂-C₆)-cyanoalkenyl, (C₂-C₆)-alkynyl, (C₂-C₆)-alkynyloxy-(C₁-C₆)-alkyl, (C₂-C₆)-haloalkynyloxy-(C₁-C₆)-alkyl, (C₂-C₆)-haloalkynyl, (C₂-C₆)-cyanoalkynyl, (C₃-C₈)-cycloalkyl, (C₃-C₈)-cycloalkyl-(C₃-C₈)-cycloalkyl, (C₁-C₆)-alkyl-(C₃-C₈)-cycloalkyl, halo-(C₃-C₈)-cycloalkyl, amino, (C₁-C₆)-alkylamino, di-(C₁-C₆)-alkylamino, (C₃-C₈)-cycloalkylamino, (C₁-C₆)-alkylcarbonylamino, (C₁-C₆)-alkylthio-(C₁-C₆)-alkyl, (C₁-C₆)-haloalkylthio-(C₁-C₆)-alkyl, (C₁-C₆)-alkylsulfinyl-(C₁-C₆)-alkyl, (C₁-C₆)-haloalkylsulfinyl-(C₁-C₆)-alkyl, (C₁-C₆)-alkylsulfonyl-(C₁-C₆)-alkyl, (C₁-C₆)-haloalkylsulfonyl-(C₁-C₆)-alkyl, (C₁-C₆)-alkoxy-(C₁-C₆)-alkylthio-(C₁-C₆)-alkyl, (C₁-C₆)-alkoxy-(C₁-C₆)-alkylsulfinyl-(C₁-C₆)-alkyl, (C₁-C₆)-alkoxy-(C₁-C₆)-alkylsulfonyl-(C₁-C₆)-alkyl, (C₁-C₆)-alkylcarbonyl-(C₁-C₆)-alkyl, (C₁-C₆)-haloalkylcarbonyl-(C₁-C₆)-alkyl, (C₁-C₆)-alkoxycarbonyl-(C₁-C₆)-alkyl, (C₁-C₆)-haloalkoxycarbonyl-(C₁-C₆)-alkyl, (C₁-C₆)-alkylsulfonylamino, aminosulfonyl-(C₁-C₆)-alkyl, (C₁-C₆)-alkylaminosulfonyl-(C₁-C₆)-alkyl, di-(C₁-C₆)-alkylaminosulfonyl-(C₁-C₆)-alkyl,
or represents (C₁-C₆)-alkyl, (C₁-C₆)-alkoxy, (C₂-C₆)-alkenyl, (C₂-C₆)-alkynyl, (C₃-C₈)-cycloalkyl, each of which is optionally mono- or polysubstituted by identical or different substituents from the group consisting of aryl, hetaryl and heterocyclyl, where aryl, hetaryl or heterocyclyl may each optionally be mono- or polysubstituted by identical or different substituents from the group consisting of halogen, cyano, nitro, hydroxy, amino, carboxy, carbamoyl, aminosulfonyl, (C₁-C₆)-alkyl, (C₃-C₆)-cycloalkyl, (C₁-C₆)-alkoxy, (C₁-C₆)-haloalkyl, (C₁-C₆)-haloalkoxy, (C₁-C₆)-alkylthio, (C₁-C₆)-alkylsulfinyl, (C₁-C₆)-alkylsulfonyl, (C₁-C₆)-alkylsulfimino, (C₁-C₆)-alkylsulfimino-(C₁-C₆)-alkyl, (C₁-C₆)-alkylsulfimino-(C₂-C₆)-alkylcarbonyl, (C₁-C₆)-alkylsulfoximino, (C₁-C₆)-alkylsulfoximino-(C₁-C₆)-alkyl, (C₁-C₆)-alkylsulfoximino-(C₂-C₆)-alkylcarbonyl, (C₁-C₆)-alkoxycarbonyl, (C₁-C₆)-alkylcarbonyl, (C₃-C₆)-trialkylsilyl and benzyl,
R¹¹, R¹², R¹³, R¹⁵, R¹⁶, R¹⁷ independently of one another represent hydrogen, cyano, halogen, nitro, hydroxy, amino, tri-(C₁-C₆)-alkylsilyl, (C₃-C₈)-cycloalkyl, (C₃-C₈)-cycloalkyl-(C₃-C₈)-cycloalkyl, (C₁-C₆)-alkyl-(C₃-C₈)-cycloalkyl, halo-(C₃-C₈)-cycloalkyl, (C₁-C₆)-alkyl, (C₁-C₆)-haloalkyl, (C₁-C₆)-cyanoalkyl, (C₁-C₆)-hydroxyalkyl, hydroxycarbonyl-(C₁-C₆)-alkoxy, (C₁-C₆)-alkoxycarbonyl-(C₁-C₆)-alkyl, (C₁-C₆)-alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)-haloalkyl-(C₁-C₆)-alkoxyimino, (C₁-C₆)-alkylthio, (C₁-C₆)-haloalkylthio, (C₁-C₆)-alkoxy-(C₁-C₆)-alkylthio, (C₁-C₆)-alkylthio-(C₁-C₆)-alkyl, (C₁-C₆)-alkylsulfinyl, (C₁-C₆)-haloalkylsulfinyl, (C₁-C₆)-alkoxy-(C₁-C₆)-alkylsulfinyl, (C₁-C₆)-alkylsulfinyl-(C₁-C₆)-alkyl, (C₁-C₆)-alkylsulfonyl, (C₁-C₆)-haloalkylsulfonyl, (C₁-C₆)-alkoxy-(C₁-C₆)-alkylsulfonyl, (C₁-C₆)-alkylsulfonyl-(C₁-C₆)-alkyl, (C₁-C₆)-alkylsulfonyloxy, (C₁-C₆)-alkylcarbonyl, (C₁-C₆)-alkylthiocarbonyl, (C₁-C₆)-haloalkylcarbonyl, (C₁-C₆)-alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)-alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulfonylamino, (C₁-C₆)-alkylamino, di-(C₁-C₆)-alkylamino, (C₃-C₈)-cycloalkylamino, aminosulfonyl, (C₁-C₆)-alkylaminosulfonyl, di-(C₁-C₆)-alkylaminosulfonyl, (C₁-C₆)-alkylsulfoximino, aminothiocarbonyl, (C₁-C₆)-alkylaminothiocarbonyl, di-(C₁-C₆)-alkylaminothiocarbonyl, (C₁-C₆)-alkylcarbonylamino (—NHCO—(C₁-C₆)-alkyl), (C₃-C₈)-cycloalkylcarbonylamino, (C₁-C₆)-alkylcarbonyl-(C₁-C₆)-alkylamino (—N—(C₁-C₆)-alkyl-CO—(C₁-C₆)-alkyl), ((C₁-C₆)-alkoxycarbonyl)amino (—NHCOO—(C₁-C₆)-alkyl), ((C₁-C₆)-alkoxycarbonyl)-(C₁-C₆)-alkylamino (—N—(C₁-C₆)-alkyl-COO—(C₁-C₆)-alkyl), (C₁-C₆)-alkylcarbamoyloxy (—OCONH—(C₁-C₆)-alkyl), di-(C₁-C₆)-alkylcarbamoyloxy (—OCON—(C₁-C₆)-dialkyl), (N—(C₁-C₆)-alkylcarbamoyl)amino (—NHCONH—(C₁-C₆)-alkyl), (N,N-di-(C₁-C₆)-alkylcarbamoyl)amino (—NHCON—(C₁-C₆)-dialkyl), (N—(C₁-C₆)-alkylcarbamoyl)-(C₁-C₆)-alkylamino (—N—(C₁-C₆)-alkyl-CONH—(C₁-C₆)-alkyl), (N,N-di-(C₁-C₆)-alkylcarbamoyl)-(C₁-C₆)-alkylamino (—N—(C₁-C₆)-alkyl-CON—(C₁-C₆)-dialkyl), carbamoylamino (—NHCONH₂), (carbamoyl)-N—(C₁-C₆)-alkylamino (—N—(C₁-C₆)-alkyl-CONH₂) or
represent aryl or hetaryl, each of which is optionally mono- or polysubstituted by identical or different substituents, where (in the case of hetaryl) at least one carbonyl group may optionally be present and where possible substituents are in each case as follows: cyano, carboxyl, halogen, nitro, hydroxy, amino, tri-(C₁-C₆)-alkylsilyl, (C₃-C₈)-cycloalkyl, (C₃-C₈)-cycloalkyl-(C₃-C₈)-cycloalkyl, (C₁-C₆)-alkyl-(C₃-C₈)-cycloalkyl, halo-(C₃-C₈)-cycloalkyl, (C₁-C₆)-alkyl, (C₁-C₆)-haloalkyl, (C₁-C₆)-cyanoalkyl, (C₁-C₆)-hydroxyalkyl, hydroxycarbonyl-(C₁-C₆)-alkoxy, (C₁-C₆)-alkoxycarbonyl-(C₁-C₆)-alkyl, (C₁-C₆)-alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-haloalkynyl, (C₂-C₆)-cyanoalkynyl, (C₁-C₆)-alkoxy, (C₁-C₆)-haloalkoxy, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)-haloalkyl-(C₁-C₆)-alkoxyimino, (C₁-C₆)-alkylthio, (C₁-C₆)-haloalkylthio, (C₁-C₆)-alkoxy-(C₁-C₆)-alkylthio, (C₁-C₆)-alkylthio-(C₁-C₆)-alkyl, (C₁-C₆)-alkylsulfinyl, (C₁-C₆)-haloalkylsulfinyl, (C₁-C₆)-alkoxy-(C₁-C₆)-alkylsulfinyl, (C₁-C₆)-alkylsulfinyl-(C₁-C₆)-alkyl, (C₁-C₆)-alkylsulfonyl, (C₁-C₆)-haloalkylsulfonyl, (C₁-C₆)-alkoxy-(C₁-C₆)-alkylsulfonyl, (C₁-C₆)-alkylsulfonyl-(C₁-C₆)-alkyl, (C₁-C₆)-alkylsulfonyloxy, (C₁-C₆)-alkylcarbonyl, (C₁-C₆)-haloalkylcarbonyl, (C₁-C₆)-alkylcarbonyloxy, (C₁-C₆)-alkoxycarbonyl, (C₁-C₆)-haloalkoxycarbonyl, aminocarbonyl, (C₁-C₆)-alkylaminocarbonyl, di-(C₁-C₆)-alkylaminocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulfonylamino, (C₁-C₆)-alkylamino, di-(C₁-C₆)-alkylamino, aminosulfonyl, (C₁-C₆)-alkylaminosulfonyl, di-(C₁-C₆)-alkylaminosulfonyl, (C₁-C₆)-alkylsulfoximino, aminothiocarbonyl, (C₁-C₆)-alkylaminothiocarbonyl, di-(C₁-C₆)-alkylaminothiocarbonyl, (C₃-C₈)-cycloalkylamino or (C₁-C₆)-alkylcarbonylamino,
where at most two of the radicals R¹¹, R¹², R¹³, R¹⁵, R¹⁶, R¹⁷ represent a substituent other than hydrogen,
and, in the case that neither R¹¹ nor R¹⁵, neither R¹² nor R¹⁶ or neither R¹³ nor R¹⁷ represents hydrogen, R¹⁵, R¹⁶ and R¹⁷ each independently of one another only represent cyano, halogen, (C₃-C₆)-cycloalkyl, (C₃-C₆)-cycloalkyl-(C₃-C₆)-cycloalkyl, (C₁-C₄)-alkyl-(C₃-C₆)-cycloalkyl, halo-(C₃-C₆)-cycloalkyl, (C₁-C₄)-alkyl, (C₁-C₄)-haloalkyl or (C₁-C₄)-cyanoalkyl,
or, in the case that [symbol] exclusively represent single bonds, additionally R¹¹ and R¹⁵ and/or R¹³ and R¹⁷ in each case together represent oxygen (═O),
Q represents a partly saturated or saturated heterocyclic or heteroaromatic 8-, 9-, 10-, 11- or 12-membered fused bicyclic or tricyclic ring system which may optionally contain at least one carbonyl group and/or where the ring system is optionally mono- or polysubstituted by identical or different substituents, where the substituents independently of one another may be selected from cyano, halogen, nitro, acetyl, hydroxy, amino, SCN, tri-(C₁-C₆)-alkylsilyl, halo-(C₃-C₈)-cycloalkyl, (C₁-C₆)-alkyl, (C₁-C₆)-haloalkyl, (C₁-C₆)-cyanoalkyl, (C₁-C₆)-hydroxyalkyl, hydroxycarbonyl-(C₁-C₆)-alkoxy, (C₁-C₆)-alkoxycarbonyl-(C₁-C₆)-alkyl, (C₁-C₆)-alkoxy-(C₁-C₆)-alkyl, (C₂-C₆)-alkenyl, (C₂-C₆)-haloalkenyl, (C₂-C₆)-cyanoalkenyl, (C₂-C₆)-alkynyl, (C₂-C₆)-alkynyloxy-(C₁-C₄)-alkyl, (C₂-C₆)-haloalkynyl, (C₂-C₆)-cyanoalkynyl, (C₁-C₆)-alkoxy, (C₁-C₆)-haloalkoxy, (C₁-C₆)-haloalkoxy-(C₁-C₆)-alkyl, (C₂-C₆)-alkenyloxy-(C₁-C₆)-alkyl, (C₂-C₆)-haloalkenyloxy-(C₁-C₆)-alkyl, (C₁-C₆)-cyanoalkoxy, (C₁-C₆)-alkoxycarbonyl-(C₁-C₆)-alkoxy, (C₁-C₆)-alkoxy-(C₁-C₆)-alkoxy, (C₁-C₆)-alkylhydroxyimino, (C₁-C₆)-alkoxyimino, (C₁-C₆)-alkyl-(C₁-C₆)-alkoxyimino, (C₁-C₆)-haloalkyl-(C₁-C₆)-alkoxyimino, (C₁-C₆)-alkylthio, (C₁-C₆)-haloalkylthio, (C₁-C₆)-alkoxy-(C₁-C₆)-alkylthio, (C₁-C₆)-alkylthio-(C₁-C₆)-alkyl, (C₁-C₆)-alkylsulfinyl, (C₁-C₆)-haloalkylsulfinyl, (C₁-C₆)-alkoxy-(C₁-C₆)-alkylsulfinyl, (C₁-C₆)-alkylsulfinyl-(C₁-C₆)-alkyl, (C₁-C₆)-alkylsulfonyl, (C₁-C₆)-haloalkylsulfonyl, (C₁-C₆)-alkoxy-(C₁-C₆)-alkylsulfonyl, (C₁-C₆)-alkylsulfonyl-(C₁-C₆)-alkyl, (C₁-C₆)-alkylsulfonyloxy, (C₁-C₆)-alkylcarbonyl, (C₁-C₆)-alkylcarbonyl-(C₁-C₆)-alkyl, (C₁-C₆)-alkylthiocarbonyl, (C₁-C₆)-haloalkylcarbonyl, (C₁-C₆)-alkylcarbonyloxy, (C₁-C₆)-alkoxycarbonyl, (C₁-C₆)-haloalkoxycarbonyl, aminocarbonyl, (C₁-C₆)-alkylaminocarbonyl, (C₁-C₆)-alkylaminothiocarbonyl, di-(C₁-C₆)-alkylaminocarbonyl, di-(C₁-C₆)-alkylaminothiocarbonyl, (C₂-C₆)-alkenylaminocarbonyl, di-(C₂-C₆)-alkenylaminocarbonyl, (C₃-C₈)-cycloalkylaminocarbonyl, (C₁-C₆)-alkylsulfonylamino, (C₁-C₆)-alkylamino, di-(C₁-C₆)-alkylamino, aminosulfonyl, (C₁-C₆)-alkylaminosulfonyl, di-(C₁-C₆)-alkylaminosulfonyl, (C₁-C₆)-alkylsulfoximino, aminothiocarbonyl, (C₁-C₆)-alkylaminothiocarbonyl, di-(C₁-C₆)-alkylaminothiocarbonyl, (C₃-C₈)-cycloalkylamino, NHCO—(C₁-C₆)-alkyl ((C₁-C₆)-alkylcarbonylamino), or optionally monocyano-substituted (C₃-C₈)-cycloalkyl, (C₃-C₈)-cycloalkyl-(C₃-C₈)-cycloalkyl or (C₁-C₄)-alkyl-(C₃-C₈)-cycloalkyl,
or where the substituents independently of one another may be selected from phenyl and a 5- or 6-membered heteroaromatic ring, where phenyl and the ring may optionally be mono- or polysubstituted by identical or different substituents selected from the group consisting of C₁-C₆-alkyl, C₂-C₆-alkenyl, C₂-C₆-alkynyl, C₃-C₆-cycloalkyl, C₁-C₆-haloalkyl, C₂-C₆-haloalkenyl, C₂-C₆-haloalkynyl, C₃-C₆-halocycloalkyl, halogen, CN, NO₂, C₁-C₄-alkoxy, C₁-C₄-haloalkoxy, and
n represents 0, 1 or 2.

2. Compound of formula (I) according to claim 1, wherein
in the case that [symbol] represents a single bond and a double bond, and Aa to Ae thus form an aromatic ring,
Aa represents nitrogen or carbon,
Ab represents nitrogen, oxygen, sulfur, N(R⁸) or C(R¹¹),
Ac represents nitrogen, oxygen, sulfur or C(R¹²),
Ad represents nitrogen, oxygen, sulfur, N(R⁸) or C(R¹³),
Ae represents nitrogen or carbon,
where R⁸ represents methyl, ethyl, cyclopropyl or hydrogen and
where at most three of the groups Aa, Ab, Ac, Ad, Ae may represent nitrogen and at most one of the groups Ab, Ac, Ad may represent oxygen or at most one of the groups Ab, Ac, Ad may represent sulfur or at most one of the groups Ab, Ad may represent N(R⁸),
or, in the case that [symbol] represent exclusively single bonds,
Aa represents carbon,
Ab represents sulfur, oxygen or C(R¹¹)(R¹⁵),
Ac represents sulfur, oxygen or C(R¹²)(R¹⁶) and
Ad represents sulfur, oxygen or C(R¹³)(R¹⁷),
Ae represents carbon,
where at most one of the groups Aa, Ab, Ac, Ad, Ae may represent oxygen or sulfur,
R¹ represents (C₁-C₄)-alkyl, (C₁-C₄)-hydroxyalkyl, (C₁-C₄)-haloalkyl, (C₂-C₄)-alkenyl, (C₂-C₄)-haloalkenyl, (C₂-C₄)-alkynyl, (C₂-C₄)-haloalkynyl, (C₃-C₆)-cycloalkyl, (C₁-C₄)-alkylthio-(C₁-C₄)-alkyl, (C₁-C₄)-haloalkylthio-(C₁-C₄)-alkyl, (C₁-C₄)-alkylsulfinyl-(C₁-C₄)-alkyl or (C₁-C₄)-alkylsulfonyl-(C₁-C₄)-alkyl,
R¹¹, R¹², R¹³, R¹⁵, R¹⁶, R¹⁷ independently of one another represent hydrogen, cyano, halogen, nitro, hydroxy, amino, tri-(C₁-C₄)-alkylsilyl, (C₃-C₆)-cycloalkyl, (C₃-C₆)-cycloalkyl-(C₃-C₆)-cycloalkyl, (C₁-C₄)-alkyl-(C₃-C₆)-cycloalkyl, halo-(C₃-C₆)-cycloalkyl, (C₁-C₄)-alkyl, (C₁-C₄)-haloalkyl, (C₁-C₄)-cyanoalkyl, (C₁-C₄)-hydroxyalkyl, (C₁-C₄)-alkoxy-(C₁-C₄)-alkyl, (C₂-C₄)-alkenyl, (C₂-C₄)-haloalkenyl, (C₂-C₄)-cyanoalkenyl, (C₂-C₄)-alkynyl, (C₂-C₄)-haloalkynyl, (C₂-C₄)-cyanoalkynyl, (C₁-C₄)-alkoxy, (C₁-C₄)-haloalkoxy, (C₁-C₄)-cyanoalkoxy, (C₁-C₄)-alkoxy-(C₁-C₄)-alkoxy, (C₁-C₄)-alkylhydroxyimino, (C₁-C₄)-alkoxyimino, (C₁-C₄)-alkyl-(C₁-C₄)-alkoxyimino, (C₁-C₄)-haloalkyl-(C₁-C₄)-alkoxyimino, (C₁-C₄)-alkylthio, (C₁-C₄)-haloalkylthio, (C₁-C₄)-alkylthio-(C₁-C₄)-alkyl, (C₁-C₄)-alkylsulfinyl, (C₁-C₄)-haloalkylsulfinyl, (C₁-C₄)-alkylsulfinyl-(C₁-C₄)-alkyl, (C₁-C₄)-alkylsulfonyl, (C₁-C₄)-haloalkylsulfonyl, (C₁-C₄)-alkylsulfonyl-(C₁-C₄)-alkyl, (C₁-C₄)-alkylsulfonyloxy, (C₁-C₄)-alkylcarbonyl, (C₁-C₄)-haloalkylcarbonyl, aminocarbonyl, aminothiocarbonyl, (C₁-C₄)-alkylaminocarbonyl, di-(C₁-C₄)-alkylaminocarbonyl, (C₃-C₆)-cycloalkylaminocarbonyl, (C₁-C₄)-alkylsulfonylamino, (C₁-C₄)-alkylamino, di-(C₁-C₄)-alkylamino, aminosulfonyl, (C₁-C₄)-alkylaminosulfonyl, di-(C₁-C₄)-alkylaminosulfonyl, aminothiocarbonyl, (C₁-C₄)-alkylcarbonylamino, (C₁-C₄)-alkylcarbonylamino (—NHCO—(C₁-C₄)-alkyl), (C₃-C₆)-cycloalkylcarbonylamino, (C₁-C₄)-alkylcarbonyl-(C₁-C₄)-alkylamino (—N—(C₁-C₄)-alkyl-CO—(C₁-C₆)-alkyl), ((C₁-C₄)-alkoxycarbonyl)amino (—NHCOO—(C₁-C₄)-alkyl), ((C₁-C₄)-alkoxycarbonyl)-(C₁-C₄)-alkylamino (—N—(C₁-C₄)-alkyl-COO—(C₁-C₆)-alkyl), (C₁-C₄)-alkylcarbamoyloxy (—OCONH—(C₁-C₄)-alkyl), di-(C₁-C₄)-alkylcarbamoyloxy (—OCON—(C₁-C₄)-dialkyl), (N—(C₁-C₄)-alkylcarbamoyl)amino (—NHCONH—(C₁-C₄)-alkyl), (N,N-di-(C₁-C₄)-alkylcarbamoyl)amino (—NHCON—(C₁-C₄)-dialkyl), (N—(C₁-C₄)-alkylcarbamoyl)-(C₁-C₄)-alkylamino (—N—(C₁-C₄)-alkyl-CONH—(C₁-C₄)-alkyl), (N,N-di-(C₁-C₄)-alkylcarbamoyl)-(C₁-C₄)-alkylamino (—N—(C₁-C₄)-alkyl-CON—(C₁-C₄)-dialkyl), carbamoylamino (—NHCONH₂), (carbamoyl)-N—(C₁-C₄)-alkylamino (—N(C₁-C₄)-alkyl-CONH₂)
or represent phenyl or 5- or 6-membered hetaryl, each of which is optionally mono- or disubstituted by identical or different substituents, where (in the case of hetaryl) optionally at least one carbonyl group may be present and the heteroatoms are selected from N, S and O, and where possible substituents are in each case as follows: cyano, halogen, acetyl, (C₃-C₆)-cycloalkyl, (C₃-C₆)-cycloalkyl-(C₃-C₆)-cycloalkyl, (C₁-C₄)-alkyl-(C₃-C₆)-cycloalkyl, halo-(C₃-C₆)-cycloalkyl, (C₁-C₄)-alkyl, (C₁-C₄)-haloalkyl, (C₁-C₄)-cyanoalkyl, (C₁-C₄)-alkoxy-(C₁-C₄)-alkyl, (C₂-C₄)-alkenyl, (C₂-C₄)-alkynyl, (C₂— (C₁-C₄)-alkoxy, (C₁-C₄)-alkylthio, (C₁-C₄)-haloalkylthio, (C₁-C₄)-alkylthio-(C₁-C₄)-alkyl, (C₁-C₄)-alkylsulfinyl, (C₁-C₄)-haloalkylsulfinyl, (C₁-C₄)-alkylsulfinyl-(C₁-C₄)-alkyl, (C₁-C₄)-alkylsulfonyl, (C₁-C₄)-haloalkylsulfonyl, (C₁-C₄)-alkylsulfonyl-(C₁-C₄)-alkyl, (C₁-C₄)-alkylsulfonyloxy, (C₁-C₄)-alkylcarbonyl, aminocarbonyl, (C₁-C₄)-alkylaminocarbonyl, di-(C₁-C₄)-alkylaminocarbonyl, (C₁-C₄)-alkylsulfonylamino, aminosulfonyl, (C₁-C₄)-alkylaminosulfonyl, di-(C₁-C₄)-alkylaminosulfonyl or (C₁-C₄)-alkylcarbonylamino,
where at most two of the radicals R¹¹, R¹², R¹³, R¹⁵, R¹⁶, R¹⁷ represent a substituent other than hydrogen,
and, in the case that neither R¹¹ nor R¹⁵, neither R¹² nor R¹⁶ or neither R¹³ nor R¹⁷ represents hydrogen, R¹⁵, R¹⁶ and R¹⁷ each independently of one another only represent cyano, halogen, (C₃-C₆)-cycloalkyl, (C₃-C₆)-cycloalkyl-(C₃-C₆)-cycloalkyl, (C₁-C₄)-alkyl-(C₃-C₆)-cycloalkyl, halo-(C₃-C₆)-cycloalkyl, (C₁-C₄)-alkyl, (C₁-C₄)-haloalkyl or (C₁-C₄)-cyanoalkyl,
or, in the case that [symbol] exclusively represent single bonds, additionally R¹¹ and R¹⁵ and/or R¹³ and R¹⁷ in each case together optionally represent oxygen (═O),
Q represents a heteroaromatic 9-membered or 12-membered fused bicyclic or tricyclic ring system from the group consisting of Q1 to Q13 and Q15 to Q21, 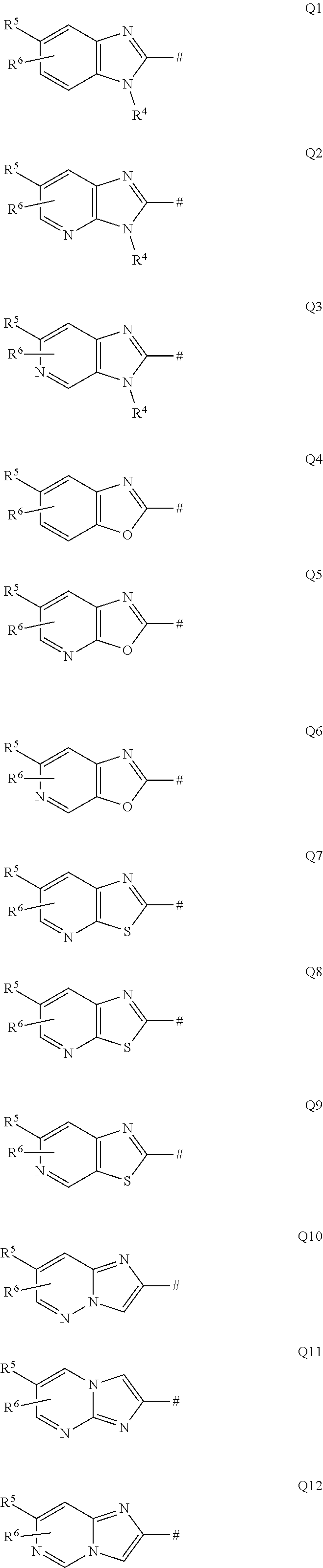 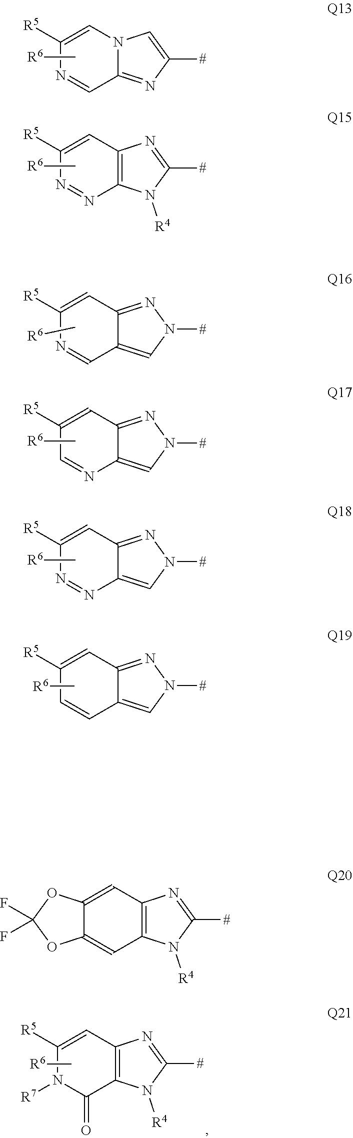
where
R⁴ represents (C₁-C₄)-alkyl, (C₁-C₄)-haloalkyl, (C₁-C₄)-cyanoalkyl, (C₁-C₄)-hydroxyalkyl, (C₁-C₄)-alkoxy-(C₁-C₄)-alkyl, (C₁-C₄)-haloalkoxy-(C₁-C₄)-alkyl, (C₂-C₄)-alkenyl, (C₂-C₄)-alkenyloxy-(C₁-C₄)-alkyl, (C₂-C₄)-haloalkenyloxy-(C₁-C₄)-alkyl, (C₂-C₄)-haloalkenyl, (C₂-C₄)-cyanoalkenyl, (C₂-C₄)-alkynyl, (C₂-C₄)-alkynyloxy-(C₁-C₄)-alkyl, (C₂-C₄)-haloalkynyl, (C₃-C₆)-cycloalkyl, (C₃-C₆)-cycloalkyl-(C₃-C₆)-cycloalkyl, (C₁-C₄)-alkyl-(C₃-C₆)-cycloalkyl, halo-(C₃-C₆)-cycloalkyl, (C₁-C₄)-alkylthio-(C₁-C₄)-alkyl, (C₁-C₄)-alkylsulfinyl-(C₁-C₄)-alkyl, (C₁-C₄)-alkylsulfonyl-(C₁-C₄)-alkyl or (C₁-C₄)-alkylcarbonyl-(C₁-C₄)-alkyl,
R⁵, R⁶ independently of one another represent hydrogen, cyano, halogen, (C₁-C₄)-alkyl, (C₁-C₄)-haloalkyl, (C₂-C₄)-alkenyl, (C₂-C₄)-haloalkenyl, (C₂-C₄)-alkynyl, (C₂-C₄)-haloalkynyl, (C₁-C₄)-alkoxy, (C₁-C₄)-haloalkoxy, (C₁-C₄)-alkoxyimino, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, (C₁-C₄)-alkylsulfonyloxy, (C₁-C₄)-alkylcarbonyl, (C₁-C₄)-haloalkylcarbonyl, aminocarbonyl, (C₁-C₄)-alkylaminocarbonyl, di-(C₁-C₄)-alkyl-aminocarbonyl, (C₁-C₄)-alkylsulfonylamino, (C₁-C₄)-alkylamino, di-(C₁-C₄)-alkylamino, aminosulfonyl, (C₁-C₄)-alkylaminosulfonyl, di-(C₁-C₄)-alkylaminosulfonyl or optionally monocyano-substituted (C₃-C₆)-cycloalkyl, (C₃-C₆)-cycloalkyl-(C₃-C₆)-cycloalkyl or (C₁-C₄)-alkyl-(C₃-C₆)-cycloalkyl,
R⁷ represents hydrogen, (C₉-C₄)-alkyl, (C₁-C₄)-haloalkyl, (C₁-C₄)-cyanoalkyl, (C₁-C₄)-hydroxyalkyl, (C₁-C₄)-alkoxy-(C₁-C₄)-alkyl, (C₁-C₄)-haloalkoxy-(C₁-C₄)-alkyl, (C₂-C₄)-alkenyl, (C₂-C₄)-alkenyloxy-(C₁-C₄)-alkyl, (C₂-C₄)-haloalkenyloxy-(C₁-C₄)-alkyl, (C₂-C₄)-haloalkenyl, (C₂-C₄)-cyanoalkenyl, (C₂-C₄)-alkynyl, (C₂-C₄)-haloalkynyl, (C₃-C₆)-cycloalkyl, (C₃-C₈)-cycloalkyl-(C₃-C₆)-cycloalkyl, (C₁-C₄)-alkyl-(C₃-C₆)-cycloalkyl, halo-(C₃-C₆)-cycloalkyl, (C₁-C₄)-alkylthio-(C₁-C₄)-alkyl, (C₁-C₄)-alkylsulfinyl-(C₁-C₄)-alkyl or (C₁-C₄)-alkylsulfonyl-(C₁-C₄)-alkyl,
and
n represents 0, 1 or 2.

3. Compound according to claim 1, corresponding to at least one of formula (Ia), (Ib), (Id), (Ie), (Ig) to (Ik) and (Im) to (It), 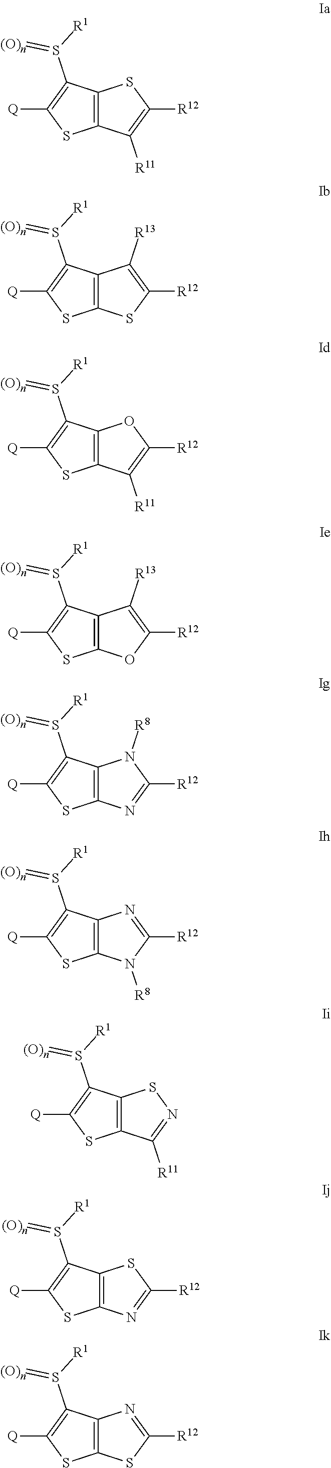 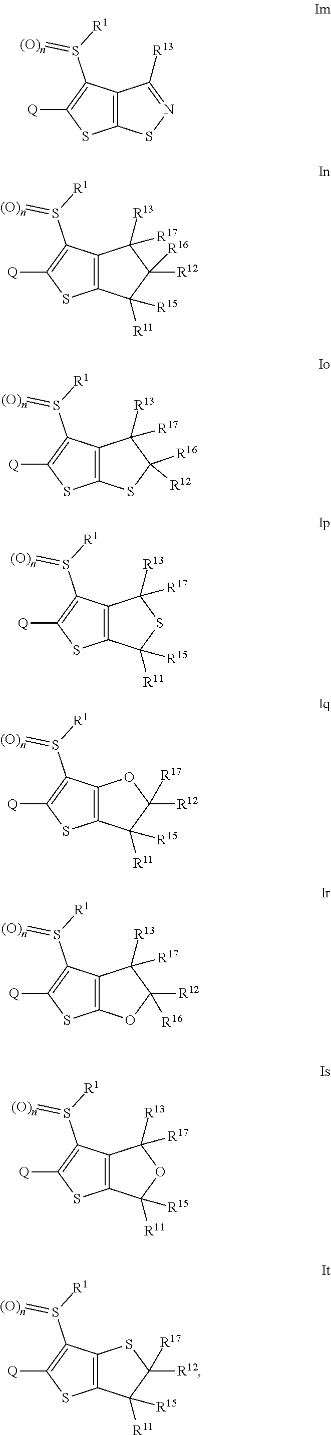
where
R¹ represents (C₁-C₄)-alkyl, (C₁-C₄)-haloalkyl or (C₃-C₆)-cycloalkoxy,
R⁸ represents methyl, ethyl, cyclopropyl or hydrogen,
R¹¹, R¹², R¹³, R¹⁵, R¹⁶, R¹⁷ independently of one another represent hydrogen, cyano, halogen, nitro, hydroxy, (C₃-C₆)-cycloalkyl, (C₁-C₄)-alkyl-(C₃-C₆)-cycloalkyl, halo-(C₃-C₆)-cycloalkyl, (C₁-C₄)-alkyl, (C₁-C₄)-haloalkyl, (C₁-C₄)-alkoxy-(C₁-C₄)-alkyl, (C₁-C₄)-alkoxy, (C₁-C₄)-haloalkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, aminosulfonyl, (C₁-C₄)-alkylaminosulfonyl, di-(C₁-C₄)-alkylaminosulfonyl, aminocarbonyl, (C₁-C₄)-alkylaminocarbonyl, di-(C₁-C₄)-alkylaminocarbonyl, (C₃-C₆)-cycloalkylaminocarbonyl, (C₁-C₄)-alkylcarbonylamino (—NHCO—(C₁-C₄)-alkyl), (C₃-C₆)-cycloalkylcarbonylamino, (C₁-C₄)-alkylcarbonyl-(C₁-C₄)-alkylamino (—N—(C₁-C₄)-alkyl-CO—(C₁-C₆)-alkyl), ((C₁-C₄)-alkoxycarbonyl)amino (—NHCOO—(C₁-C₄)-alkyl), (N—(C₁-C₄)-alkylcarbamoyl)amino (—NHCONH—(C₁-C₄)-alkyl), (N,N-di-(C₁-C₄)-alkylcarbamoyl)amino (—NHCON—(C₁-C₄)-dialkyl), (N—(C₁-C₄)-alkylcarbamoyl)-(C₁-C₄)-alkylamino (—N(C₁-C₄)-alkyl-CONH—(C₁-C₄)-alkyl), (N,N-di-(C₁-C₄)-alkylcarbamoyl)-(C₁-C₄)-alkylamino (—N(C₁-C₄)-alkyl-CON—(C₁-C₄)-dialkyl), carbamoylamino (—NHCONH₂), (carbamoyl)-N—(C₁-C₄)-alkylamino (—N—(C₁-C₄)-alkyl-CONH₂)
Or optionally represents phenyl, triazole or pyrazole, each of which is optionally be mono- or disubstituted by identical or different substituents, possible substituents being in each case: cyano, halogen, (C₃-C₆)-cycloalkyl, (C₃-C₆)-cycloalkyl-(C₃-C₆)-cycloalkyl, (C₁-C₄)-alkyl-(C₃-C₆)-cycloalkyl, halo-(C₃-C₆)-cycloalkyl, (C₁-C₄)-alkyl, (C₁-C₄)-haloalkyl, (C₁-C₄)-cyanoalkyl, (C₂-C₄)-alkenyl, (C₂-C₄)-alkynyl, (C₁-C₄)-alkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, (C₁-C₄)-alkylsulfonyloxy, (C₁-C₄)-alkylcarbonyl, aminocarbonyl, (C₁-C₄)-alkylaminocarbonyl, di-(C₁-C₄)-alkylaminocarbonyl, (C₁-C₄)-alkylsulfonylamino, aminosulfonyl, (C₁-C₄)-alkylaminosulfonyl, di-(C₁-C₄)-alkylaminosulfonyl or (C₁-C₄)-alkylcarbonylamino,
where at most or two of the radicals R¹¹, R¹², R¹³, R¹⁵, R¹⁶, R¹⁷ represent a substituent other than hydrogen,
and, in the case that neither R¹¹ nor R¹⁵, neither R¹² nor R¹⁶ or neither R¹³ nor R¹⁷ represents hydrogen, R¹⁴, R¹⁵, R¹⁶ and R¹⁷ each independently of one another only represent cyano, halogen, (C₃-C₆)-cycloalkyl, (C₃-C₆)-cycloalkyl-(C₃-C₆)-cycloalkyl, (C₁-C₄)-alkyl-(C₃-C₆)-cycloalkyl, halo-(C₃-C₆)-cycloalkyl, (C₁-C₄)-alkyl or (C₁-C₄)-haloalkyl,
or, in the case of formulae of the structures (In) to (It), R¹¹ and R¹⁵ or R¹³ and R¹⁷ additionally in each case together represent oxygen (═O),
Q represents a heteroaromatic 9-membered fused bicyclic ring system from the group consisting of Q1, Q2, Q3, Q5, Q6, Q8, Q9, Q10, Q12, Q15, Q20 and Q21, 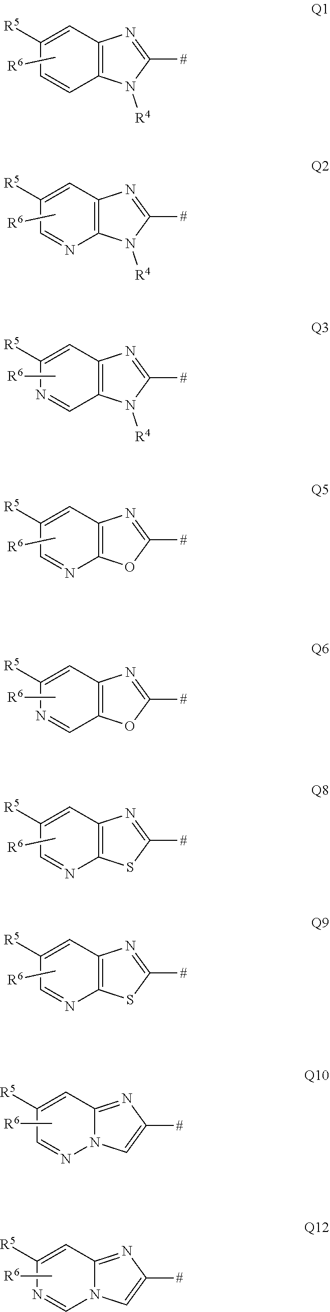 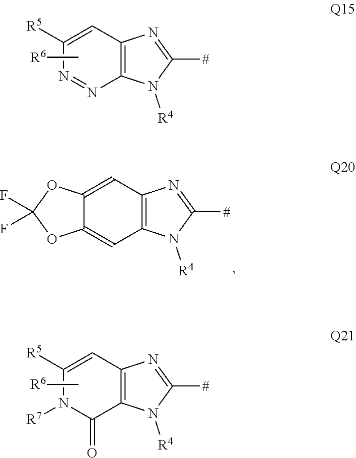
R⁴ represents (C₁-C₄)-alkyl, (C₁-C₄)-haloalkyl, (C₁-C₄)-cyanoalkyl or (C₁-C₄)-alkoxy-(C₁-C₄)-alkyl,
R⁵ represents halogen, (C₁-C₄)-haloalkyl, (C₁-C₄)-haloalkoxy, (C₁-C₄)-haloalkylthio, (C₁-C₄)-haloalkylsulfinyl, (C₁-C₄)-haloalkylsulfonyl or represents optionally monocyano-substituted (C₃-C₆)-cycloalkyl,
R⁶ represents hydrogen, cyano, halogen or (C₁-C₄)-alkyl,
R⁷ represents hydrogen, (C₁-C₄)-alkyl, (C₁-C₄)-haloalkyl, (C₁-C₄)-cyanoalkyl, (C₁-C₄)-hydroxyalkyl, (C₁-C₄)-alkoxy-(C₁-C₄)-alkyl, (C₁-C₄)-haloalkoxy-(C₁-C₄)-alkyl, (C₂-C₄)-alkenyl, (C₂-C₄)-alkenyloxy-(C₁-C₄)-alkyl, (C₂-C₄)-haloalkenyloxy-(C₁-C₄)-alkyl, (C₂-C₄)-haloalkenyl, (C₂-C₄)-cyanoalkenyl, (C₂-C₄)-alkynyl, (C₂-C₄)-haloalkynyl, (C₃-C₆)-cycloalkyl, (C₃-C₈)-cycloalkyl-(C₃-C₈)-cycloalkyl, (C₁-C₄)-alkyl-(C₃-C₆)-cycloalkyl, halo-(C₃-C₆)-cycloalkyl, (C₁-C₄)-alkylthio-(C₁-C₄)-alkyl, (C₁-C₄)-alkylsulfinyl-(C₁-C₄)-alkyl or (C₁-C₄)-alkylsulfonyl-(C₁-C₄)-alkyl,
and
n represents 0, 1 or 2.

4. Compound of formula (Ia), (Ib), (Id), (Ie), (Ii) bis (Ik), (In), (Iq) and/or (Ir) according to claim 3, 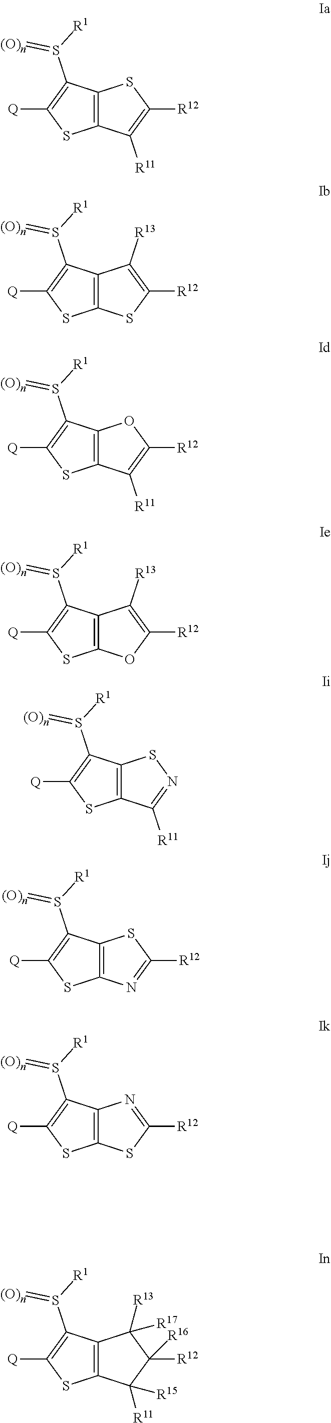 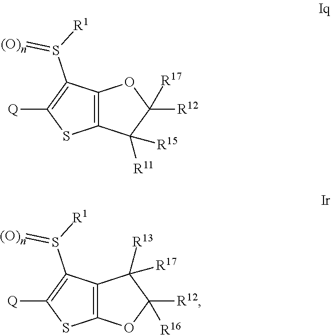
where
R¹ represents (C₁-C₄)-alkyl, (C₃-C₆)-cycloalkyl or (C₁-C₄)-haloalkyl,
R¹³, R¹⁵, R¹⁶, R¹⁷ independently of one another represent hydrogen, halogen, (C₁-C₄)-haloalkyl or (C₁-C₄)-alkyl,
R¹¹, R¹² independently of one another represent hydrogen, cyano, halogen, nitro, hydroxy, (C₃-C₆)-cycloalkyl, (C₁-C₄)-alkyl-(C₃-C₆)-cycloalkyl, halo-(C₃-C₆)-cycloalkyl, (C₁-C₄)-alkyl, (C₁-C₄)-haloalkyl, (C₁-C₄)-alkoxy-(C₁-C₄)-alkyl, (C₁-C₄)-alkoxy, (C₁-C₄)-haloalkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, aminosulfonyl, (C₁-C₄)-alkylaminosulfonyl, di-(C₁-C₄)-alkylaminosulfonyl, aminocarbonyl, (C₁-C₄)-alkylaminocarbonyl, di-(C₁-C₄)-alkylaminocarbonyl, (C₃-C₆)-cycloalkylaminocarbonyl, (C₁-C₄)-alkylcarbonylamino (—NHCO—(C₁-C₄)-alkyl), (C₃-C₆)-cycloalkylcarbonylamino, (C₁-C₄)-alkylcarbonyl-(C₁-C₄)-alkylamino (—N—(C₁-C₄)-alkyl-CO—(C₁-C₆)-alkyl), ((C₁-C₄)-alkoxycarbonyl)amino (—NHCOO—(C₁-C₄)-alkyl), (N—(C₁-C₄)-alkylcarbamoyl)amino (—NHCONH—(C₁-C₄)-alkyl), (N,N-di-(C₁-C₄)-alkylcarbamoyl)amino (—NHCON—(C₁-C₄)-dialkyl), (N—(C₁-C₄)-alkylcarbamoyl)-(C₁-C₄)-alkylamino (—N—(C₁-C₄)-alkyl-CONH—(C₁-C₄)-alkyl), carbamoylamino (—NHCONH₂), (carbamoyl)-N—(C₁-C₄)-alkylamino (—N(C₁-C₄)-alkyl-CONH₂)
or represents phenyl, pyrazole or triazole, each of which is optionally mono- or disubstituted by identical or different substituents, possible substituents being in each case: cyano, halogen, (C₃-C₆)-cycloalkyl, (C₁-C₄)-alkyl-(C₃-C₆)-cycloalkyl, halo-(C₃-C₆)-cycloalkyl, (C₁-C₄)-alkyl, (C₁-C₄)-haloalkyl, (C₁-C₄)-alkoxy, (C₁-C₄)-alkylthio, (C₁-C₄)-haloalkylthio, (C₁-C₄)-alkylsulfinyl, (C₁-C₄)-haloalkylsulfinyl, (C₁-C₄)-alkylsulfonyl, (C₁-C₄)-haloalkylsulfonyl, (C₁-C₄)-alkylsulfonyloxy, aminocarbonyl, (C₁-C₄)-alkylaminocarbonyl, di-(C₁-C₄)-alkylaminocarbonyl, (C₁-C₄)-alkylcarbonylamino,
where at most or two of the radicals R¹¹, R¹², R¹³, R¹⁵, R¹⁶, R¹⁷ represent a substituent other than hydrogen,
and, in the case that neither R¹¹ nor R¹⁵, neither R¹² nor R¹⁶ or neither R¹³ nor R¹⁷ represents hydrogen, R¹⁴, R¹⁵, R¹⁶ and R¹⁷ each independently of one another only represent halogen, (C₁-C₄)-haloalkyl or (C₁-C₄)-alkyl,
or, in the case of formulae of the structures (In), (Iq) and (Ir), R¹¹ and R¹⁵ or R¹³ and R¹⁷ additionally in each case together represent oxygen (═O),
Q represents a heteroaromatic 9-membered fused bicyclic ring system from the group consisting of Q1, Q2, Q3, Q5, Q6, Q12, Q10, Q15, Q20 and Q21 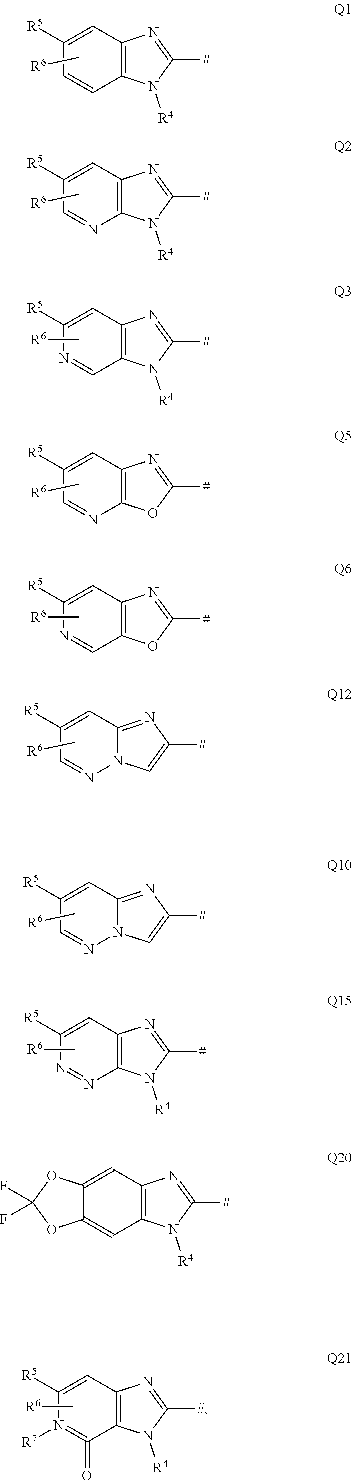
where
R⁴ represents (C₁-C₄)-alkyl,
R⁵ represents halogen, (C₁-C₄)-haloalkyl, (C₁-C₄)-haloalkoxy, (C₁-C₄)-haloalkylthio, (C₁-C₄)-haloalkylsulfonyl, (C₁-C₄)-haloalkylsulfinyl or represents optionally monocyano-substituted (C₃-C₆)-cycloalkyl,
R⁶ represents hydrogen,
R⁷ represents hydrogen, (C₁-C₄)-alkyl, (C₁-C₄)-haloalkyl, (C₁-C₄)-cyanoalkyl, (C₁-C₄)-alkoxy-(C₁-C₄)-alkyl, (C₁-C₄)-haloalkoxy-(C₁-C₄)-alkyl, (C₂-C₄)-alkenyl, (C₂-C₄)-haloalkenyl, (C₂-C₄)-cyanoalkenyl, (C₂-C₄)-alkynyl, (C₂-C₄)-haloalkynyl, (C₃-C₆)-cycloalkyl, (C₃-C₆)-cycloalkyl-(C₃-C₆)-cycloalkyl, (C₁-C₄)-alkyl-(C₃-C₆)-cycloalkyl, halo-(C₃-C₆)-cycloalkyl, (C₁-C₄)-alkylthio-(C₁-C₄)-alkyl, (C₁-C₄)-alkylsulfinyl-(C₁-C₄)-alkyl or (C₁-C₄)-alkylsulfonyl-(C₁-C₄)-alkyl,
and
n represents 0, 1 or 2.

5. Compound of formulae (Ia), (Id), (Ij) and/or (In) according to claim 3, 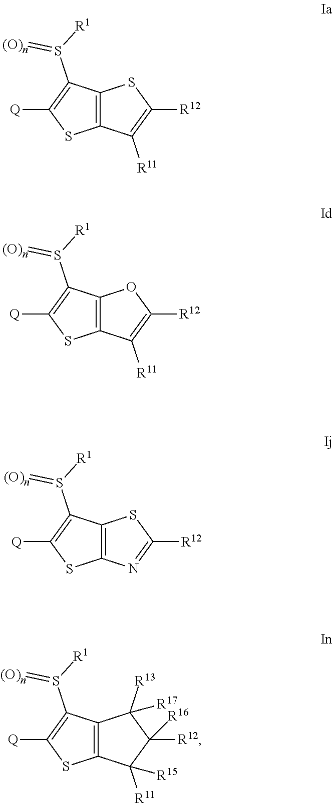
where
R¹ represents (C₁-C₄)-alkyl,
R¹³ represents hydrogen or halogen,
R¹⁵, R¹⁶, R¹⁷ represent hydrogen,
R¹¹, R¹² independently of one another represent hydrogen, hydroxy, cyano, halogen, (C₁-C₄)-alkyl, (C₃-C₆)-cycloalkyl, (C₁-C₄)-haloalkyl, (C₁-C₄)-haloalkoxy, aminocarbonyl, (C₁-C₄)-alkylaminocarbonyl, di-(C₁-C₄)-alkylaminocarbonyl, (C₃-C₆)-cycloalkylaminocarbonyl, (C₁-C₄)-alkylcarbonylamino (—NHCO—(C₁-C₄)-alkyl), (C₃-C₆)-cycloalkylcarbonylamino, (N—(C₁-C₄)-alkylcarbamoyl)amino (—NHCONH—(C₁-C₄)-alkyl), phenyl,
or represents pyrazole or triazole, each of which is optionally monosubstituted by (C₁-C₄)-alkyl, halogen or (C₁-C₄)-haloalkyl,
or, in the case of formulae of the structure (In), R¹¹ and R¹⁵ or R¹³ and R¹⁷ additionally in each case together represent oxygen (═O),
Q represents a heteroaromatic 9-membered fused bicyclic ring system from the group consisting of Q2, Q3, Q15 and Q21, 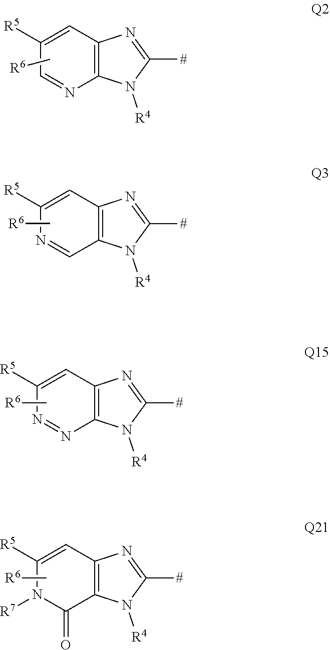
R⁴ represents (C₁-C₄)-alkyl,
R⁵ represents (C₁-C₄)-haloalkyl, (C₁-C₄)-haloalkylthio, (C₁-C₄)-haloalkylsulfonyl, (C₁-C₄)-haloalkylsulfinyl or monocyano-substituted cyclopropyl,
R⁶ represents hydrogen and
R⁷ represents (C₁-C₄)-alkyl, cyclopropyl, methoxymethyl or methoxyethyl and
n represents 0 or 2.

6. Compound of formula (Ia), (Id), (Ij) and/or (In) according to claim 3, 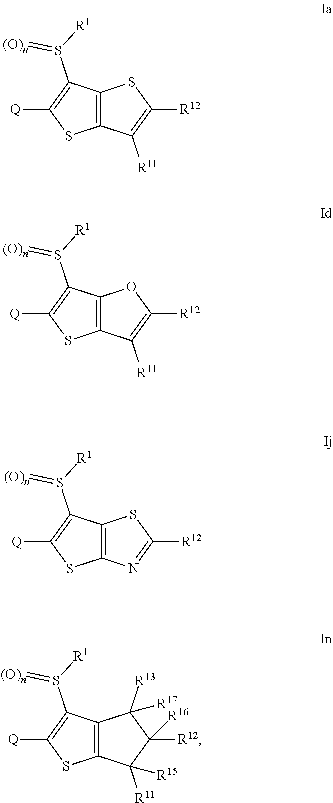
where
R¹ represents ethyl,
R¹³ represents hydrogen or fluorine,
R¹⁵, R¹⁶, R¹⁷ represent hydrogen,
R¹¹ represents hydrogen, hydroxy, cyano, cyclopropyl, trifluoromethyl, bromine, iodine, fluorine, aminocarbonyl, methylaminocarbonyl, methylcarbonylamino, cyclopropylcarbonylamino, (N-methylcarbamoyl)amino (—NHCONHMe), phenyl, N-methylpyrazole, trifluoromethylimidazole or chloropyrazole,
R¹² represents hydrogen, cyclopropyl, methyl, trifluoromethyl or chlorine,
or, in the case of formulae of the structure (In), R¹¹ and R¹⁵ or R¹³ and R¹⁷ additionally in each case together represent oxygen (═O),
Q represents a heteroaromatic 9-membered fused bicyclic ring system from the group consisting of Q2, Q3, Q15 and Q21, 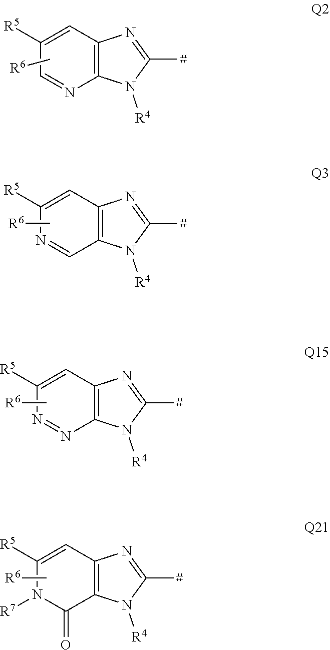
R⁴ represents methyl,
R⁵ represents trifluoromethyl, trifluoromethylsulfonyl, trifluoromethylthio, trifluoromethylsulfinyl or pentafluoroethyl,
R⁶ represents hydrogen,
R⁷ represents methyl
and
n represents 2.

7. At least one Compound of (Ia), (Ib), (Id), (Ie), (Ig) to (Ik) and (Im) to (It) according to claim 3, wherein for a compound of the formula I(a), I(b), I(d), I(e), I(g), I(h), I(j) or I(k), at least one of the radicals R¹¹ or R¹² represents a radical different from hydrogen.

8. Agrochemical formulation comprising at least one compound of formula (I) according to claim 1 and one or more extenders and/or surfactants.

9. Agrochemical formulation according to claim 8, additionally comprising a further agrochemical active ingredient.

10. A Method of controlling one or more animal pests, comprising allowing at least one compound of the formula (Ia), (Ib), (Id), (Ie), (Ig) to (Ik) and (Im) to (It) according to claim 3 and/or an agrochemical formulation thereof to act on the animal pests and/or a habitat thereof.

11. A product comprising a compound of formula (I) according to claim 1 or an agrochemical formulation thereof for controlling one or more animal pests.
